(19) United States
(12) Patent Application Publication
(10) Pub. No.: US 2024/0391979 A1
(43) Pub. Date: Nov. 28, 2024

(54) HEPATITIS B VIRUS ANTIVIRUS (HBV-ANTIVIRUS) COMPOSITIONS AND METHODS OF USE

(71) Applicant: Achelois BioPharma, Inc., Redwood City, CA (US)

(72) Inventors: Michael CHEN, Palo Alto, CA (US); Chang-Zheng CHEN, Palo Alto, CA (US)

(21) Appl. No.: 18/690,913

(22) PCT Filed: Sep. 12, 2022
(86) PCT No.: PCT/US2022/043207

§ 371 Date: Mar. 11, 2024

Related U.S. Application Data
(60) Provisional application No. 63/243,668, filed on Sep. 13, 2021.

(51) Int. Cl. C07K 16/08; A61K 39/00; A61P 31/20; C07K 14/005; C07K 14/715

(52) U.S. Cl. CPC C07K 16/082; A61P 31/20; C07K 14/005; C07K 14/7151; A61K 39/00; A61K 2039/505; C07K 2317/31; C07K 2317/76; C07K 2319/03; C12N 2760/20222

(57) ABSTRACT

Disclosed herein, in some embodiments, is a multivalent HBV-Antivirus comprising a first fusion protein that comprises a transmembrane polypeptide and an antibody that binds to a surface protein of a hepatitis B virus (HBV) wherein the first fusion protein is expressed on a surface of the antivirus. Provided herein are antibody-based antiviruses for HBV comprising a fusion protein that comprises a transmembrane polypeptide and an antibody which binds to a surface protein of a hepatitis B virus. Further provided herein are antibody-based antiviruses for HBV and methods for treating hepatitis B viral infections.

### CROSS-REFERENCE

This application claims the benefit of U.S. Provisional Application No. 63/243,668 filed Sep. 13, 2021, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Disclosed herein, in some embodiments, is a multivalent HBV-Antivirus comprising a first fusion protein that comprises a transmembrane polypeptide and an antibody that binds to a surface protein of a hepatitis B virus (HBV) wherein the first fusion protein is expressed on a surface of the antivirus. In some embodiments, the antivirus neutralizes the HBV when the first fusion protein is bound to a surface protein of the HBV. In some embodiments, the antivirus further comprises a second fusion protein that is expressed on the surface of the antivirus. In some embodiments, the second fusion protein comprises a transmembrane polypeptide and an immune modulating polypeptide. In some embodiments, the immune modulating polypeptide comprises an NK cell activating ligand. In some embodiments, the NK cell activating ligand comprises m157, NKG2D ligand, CD70, or CD40. In some embodiments, the immune modulating polypeptide comprises an immune checkpoint molecule. In some embodiments, the immune checkpoint molecule comprises CTLA4, PD1, OX40, or CD28. In some embodiments, the immune modulating polypeptide comprises an inflammatory cytokine. In some embodiments, the inflammatory cytokine comprises IL-12, IL-18, TNF-α, or TNF-β. In some embodiments, the immune modulating polypeptide comprises a proliferation cytokine. In some embodiments, the proliferation cytokine comprises IL-7, IL-15, or IL-21. In some embodiments, the second fusion protein comprises a transmembrane polypeptide and an antibody that binds to a surface protein of HBV and the antibody has less than 100% sequence identity to the antibody of the first fusion protein that binds to the surface protein of HBV. In some embodiments, the antibody of the first fusion protein binds to Large Hepatitis B surface Antigen (L-HBsAg), Medium Hepatitis B surface Antigen (M-HBsAg), or Small Hepatitis B surface Antigen (S-HBsAg). In some embodiments, the antibody of the first fusion protein binds to L-HBsAg. In some embodiments, the antibody of the first fusion protein binds to M-HBsAg. In some embodiments, the antibody of the first fusion protein binds to S-HBsAg. In some embodiments, the antibody of the second fusion protein binds to Large Hepatitis B surface Antigen (L-HBsAg), Medium Hepatitis B surface Antigen (M-HBsAg), or Small Hepatitis B surface Antigen (S-HBsAg). In some embodiments, the antibody of the second fusion protein binds to L-HBsAg. In some embodiments, the antibody of the second fusion protein binds to M-HBsAg. In some embodiments, the antibody of the second fusion protein binds to S-HBsAg. In some embodiments, the antibody of the first fusion protein comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody of the first fusion protein comprises a neutralizing antibody. In some embodiments, the antibody of the first fusion protein comprises a multi-specific antibody. In some embodiments, the multi-specific antibody comprises a bispecific antibody. In some embodiments, the bispecific antibody binds to two different epitopes of S-HBsAg. In some embodiments, the bispecific antibody binds to S-HBsAg and the Pre-S1 domain of L-HBsAg. In some embodiments, the bispecific antibody binds to S-HBsAg and the Pre-S2 domain of M-HBsAg. In some embodiments, the multi-specific antibody comprises a trispecific antibody. In some embodiments, the trispecific antibody binds to HBsAg, Pre-S1 domain of L-HBsAg, and Pre-S2 domain of M-HBsAg. In some embodiments, the antibody of the first fusion protein comprises an amino acid sequence from at least one complementarity determining region of 2H5, ADRI-2F3, H004, H009, H007, H015, H019, or H020. In some embodiments, the antibody of the second fusion protein comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody of the second fusion protein comprises a neutralizing antibody. In some embodiments, the antibody of the second fusion protein comprises an amino acid sequence from at least one complementarity determining region of 2H5, ADRI-2F3, H004, H009, H00, H015, H019, or H020. In some embodiments, the antibody of the second fusion protein comprises a multi-specific antibody. In some embodiments, the multi-specific antibody comprises a bispecific antibody. In some embodiments, the bi-specific antibody binds to two different epitopes of S-HBsAg. In some embodiments, the bispecific antibody binds to S-HBsAg and Pre-S1 domain of L-HBsAg. In some embodiments, the bispecific antibody binds to S-HBsAg and Pre-S2 domain of M-HBsAg. In some embodiments, the multi-specific antibody comprises a trispecific antibody. In some embodiments, the trispecific antibody binds to S-HBsAg, Pre-S1 domain of L-HBsAg, and Pre-S2 domain of M-HBsAg. In some embodiments, the antibody of the first fusion protein comprises a multi-specific antibody that binds to S-HBsAg and Pre-S1 domain of L and the antibody of the second fusion protein comprises a multi-specific antibody that binds to HBsAg and Pre-S2 domain of M. In some embodiments, the antibody of the first fusion protein comprises a multi-specific antibody that binds to S-HBsAg and Pre-S2 domain of M-HBsAg and the antibody of the second fusion protein comprises a multi-specific antibody that binds to S-HBsAg and Pre-S1 domain of L-HBsAg. In some embodiments, the antibody of the first fusion protein comprises a multi-specific antibody that binds to S-HBsAg, Pre-S1 domain of L-HBsAg, and Pre-S2 domain of M-HBsAg and the antibody of the second fusion protein comprises a multi-specific antibody that binds to S-HBsAg and Pre-S2 domain of M-HBsAg. In some embodiments, the antibody of the first fusion protein comprises a multi-specific antibody that binds to S-HBsAg, Pre-S1 domain of L-HBsAg, and Pre-S2 domain of M-HBsAg and the antibody of the second fusion protein comprises a multi-specific antibody that binds to S-HBsAg and Pre-S1 domain of L-HBsAg.

In some embodiments, the first fusion protein is monomeric. In some embodiments, the first fusion protein comprises an oligomerization domain. In some embodiments, the second fusion protein is monomeric. In some embodiments, the second fusion protein comprises an oligomerization domain. In some embodiments, the oligomerization domain comprises a dimerization domain. In some embodiments, the dimerization domain comprises a leucine zipper dimerization domain. In some embodiments, the oligomerization domain comprises a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain comprises a tetramerization domain. In some embodiments, the tetramerization domain comprises an influenza neuraminidase stem domain. In some embodiments, the oligomerization domain comprises a dimerization domain. In some embodiments, the dimerization domain comprises a leucine zipper dimerization domain. In some embodiments, the oligomerization domain comprises a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain comprises a tetramerization domain. In some embodiments, the tetramerization domain comprises an influenza neuraminidase stem domain. In some embodiments, the oligomerization domain comprises an amino acid sequence that has at least 90% sequence identity to an amino acid sequence according to any one of SEQ ID NOs: 17-30. In some embodiments, the oligomerization domain comprises an amino acid sequence that has at least 90% sequence identity to an amino acid sequence according to any one of SEQ ID NOs: 17-30. In some embodiments, the first fusion protein comprises a signal peptide. In some embodiments, domains of the first fusion protein are arranged from the N-terminus to the C-terminus in the following orders: (a) signal peptide, antibody, oligomerization domain, and transmembrane polypeptide; (b) signal peptide, antibody, transmembrane polypeptide, and oligomerization domain; or (c) signal peptide, oligomerization domain, antibody, and transmembrane polypeptide. In some embodiments, the first fusion protein further comprises a cytosolic domain. In some embodiments, domains of the first fusion protein are arranged from the N-terminus to the C-terminus in the following orders: (a) signal peptide, antibody, oligomerization domain, transmembrane polypeptide, and cytosolic domain; (b) signal peptide, antibody, transmembrane polypeptide, oligomerization domain, and cytosolic domain; or (c) signal peptide, oligomerization domain, antibody, transmembrane polypeptide, and cytosolic domain. In some embodiments, the second fusion protein comprises a signal peptide. In some embodiments, domains of the second fusion protein are arranged from the N-terminus to the C-terminus in the following orders: (a) signal peptide, antibody, oligomerization domain, and transmembrane polypeptide; (b) signal peptide, antibody, transmembrane polypeptide, and oligomerization domain; or (c) signal peptide, oligomerization domain, antibody, and transmembrane polypeptide. In some embodiments, the second fusion protein further comprises a cytosolic domain. In some embodiments, domains of the second fusion protein are arranged from the N-terminus to the C-terminus in the following orders: (a) signal peptide, antibody, oligomerization domain, transmembrane polypeptide, and cytosolic domain; (b) signal peptide, antibody, transmembrane polypeptide, oligomerization domain, and cytosolic domain; or (c) signal peptide, oligomerization domain, antibody, transmembrane polypeptide, and cytosolic domain.

In some embodiments, the transmembrane polypeptide anchors the first fusion protein to a lipid bilayer of the antivirus. In some embodiments, the transmembrane polypeptide anchors the second fusion protein to a lipid bilayer of the antivirus. In some embodiments, the transmembrane polypeptide of the first fusion protein comprises the transmembrane domain of a Vesicular Stomatitis virus glycoprotein (VSV-G). In some embodiments, the transmembrane polypeptide of the first fusion protein comprises the cytosolic tail of VSV-G. In some embodiments, the transmembrane polypeptide of the first fusion protein comprises the transmembrane domain of influenza Neuraminidase (NA). In some embodiments, the transmembrane polypeptide of the first fusion protein comprises the transmembrane domain of influenza Hemagglutinin (HA). In some embodiments, the transmembrane polypeptide of the first fusion protein comprises the transmembrane domain of Dengue E Protein. In some embodiments, the transmembrane polypeptide of the first fusion protein comprises the transmembrane domain of HIV surface glycoprotein GP120 or GP41. In some embodiments, the transmembrane polypeptide of the first fusion protein comprises the transmembrane domain of measles virus surface glycoprotein hemagglutinin (H) protein. In some embodiments, the transmembrane polypeptide of the second fusion protein comprises the transmembrane domain of a Vesicular Stomatitis virus glycoprotein (VSV-G). In some embodiments, the transmembrane polypeptide of the second fusion protein comprises the cytosolic tail of VSV-G. In some embodiments, the transmembrane polypeptide of the second fusion protein comprises the transmembrane domain of influenza Neuraminidase (NA). In some embodiments, the transmembrane polypeptide of the second fusion protein comprises the transmembrane domain of influenza Hemagglutinin (HA). In some embodiments, the transmembrane polypeptide of the second fusion protein comprises the transmembrane domain of Dengue E Protein. In some embodiments, the transmembrane polypeptide of the second fusion protein comprises the transmembrane domain of HIV surface glycoprotein GP120 or GP41. In some embodiments, the transmembrane polypeptide of the second fusion protein comprises the transmembrane domain of measles virus surface glycoprotein hemagglutinin (H) protein. In some embodiments, the transmembrane polypeptide of the first fusion protein comprises an amino acid sequence of at least about 90% sequence identity to any one of SEQ ID NO: 31-39. In some embodiments, the transmembrane polypeptide of the second fusion protein comprises an amino acid sequence of at least about 90% sequence identity to any one of SEQ ID NO: 31-39.

In some embodiments, the antivirus is not a lentiviral particle. In some embodiments, the antivirus does not comprise viral genetic material. In some embodiments, the antivirus comprises a lipid bilayer. In some embodiments, the antivirus comprises an enveloped particle. In some embodiments, the antivirus comprises a virus. In some embodiments, the antivirus comprises a replication incompetent virus. In some embodiments, the antivirus comprises a replication competent virus. In some embodiments, the antivirus comprises a viral-like particle. In some embodiments, the antivirus comprises an extracellular vesicle. In some embodiments, the extracellular vesicle comprises an ectosome. In some embodiments, the extracellular vesicle comprises an exosome. In some embodiments, the first fusion protein is expressed at a valency of about 10 copies on the surface of the antivirus. In some embodiments, the first fusion protein is expressed at a valency of about 10 to 15 copies on the surface of the antivirus. In some embodiments, the first fusion protein is expressed at a valency of at least about 25 copies on the surface of the antivirus. In some embodiments, the first fusion protein is expressed at a valency of at least about 50 copies on the surface of the antivirus. In some embodiments, the first fusion protein is expressed at a valency of at least about 100 copies on the surface of the antivirus. In some embodiments, the first fusion protein is expressed at a valency of at least about 200 copies on the surface of the antivirus. In some embodiments, the first fusion protein is expressed at a valency of at least about 400 copies on the surface of the antivirus. In some embodiments, the first fusion protein is expressed at a valency of at least about 600 copies on the surface of the antivirus. In some embodiments, the first fusion protein is expressed at a valency of at least about 1000 copies on a surface of the antivirus. In some embodiments, the first fusion protein is expressed at a valency of at least about 2000 copies on the surface of the antivirus. In some embodiments, the second fusion protein is expressed at a valency of about 10 copies on the surface of the antivirus. In some embodiments, the second fusion protein is expressed at a valency of about 10 to 15 copies on the surface of the antivirus. In some embodiments, the second fusion protein is expressed at a valency of at least about 25 copies on the surface of the antivirus. In some embodiments, the second fusion protein is expressed at a valency of at least about 50 copies on the surface of the antivirus. In some embodiments, the second fusion protein is expressed at a valency of at least about 100 copies on the surface of the antivirus. In some embodiments, the second fusion protein is expressed at a valency of at least about 200 copies on the surface of the antivirus. In some embodiments, the second fusion protein is expressed at a valency of at least about 400 copies on the surface of the antivirus. In some embodiments, the second fusion protein is expressed at a valency of at least about 600 copies on the surface of the antivirus. In some embodiments, the second fusion protein is expressed at a valency of at least about 1000 copies on the surface of the antivirus. In some embodiments, the second fusion protein is expressed at a valency of at least about 2000 copies on the surface of the antivirus. In some embodiments, the antivirus comprises a fluorophore expressed on the surface of the antivirus. In some embodiments, the fluorophore is conjugated to a membrane-intercalating polypeptide. In some embodiments, the antivirus neutralizes a hepatitis delta virus (HDV) when the first fusion protein is bound to an HBV surface protein located on the surface of HDV.

In some embodiments, the antibody of the first fusion protein comprises: (a) a heavy chain variable region (VH) comprising an amino acid sequence having at least 90% sequence identity to an amino acid sequence of any one of SEQ ID Nos: 1, 3, 5, 7, 9, 11, 13, and 15; and (b) a light chain variable region (VL) comprising an amino acid sequence having at least 90% sequence identity to an amino acid sequence of any one of SEQ ID Nos: 2, 4, 6, 8, 10, 12, 14, and 16. In some embodiments, (a) the antibody of the first fusion protein comprises an amino acid sequence from at least one complementarity determining region of 2H5, ADRI-2F3, H004, H009, H007, H015, H019, or H020; and (b) the transmembrane polypeptide of the first fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, (a) the antibody of the first fusion protein comprises: (i) a heavy chain variable region (VH) comprising an amino acid sequence having at least 90% sequence identity to an amino acid sequence of any one of SEQ ID Nos: 1, 3, 5, 7, 9, 11, 13, and 15; and (ii) a light chain variable region (VL) comprising an amino acid sequence having at least 90% sequence identity to an amino acid sequence of any one of SEQ ID Nos: 2, 4, 6, 8, 10, 12, 14, and 16; and (b) the transmembrane polypeptide of the first fusion protein comprises an amino acid sequence having at least 90% sequence identity to an amino acid sequence of any one of SEQ ID Nos: 31-39. In some embodiments, (a) the antibody of the first fusion protein comprises an amino acid sequence from at least one complementarity determining region of 2H5, ADRI-2F3, H004, H009, H007, H015, H019, or H020; (b) the transmembrane polypeptide of the first fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and (c) the oligomerization domain of the first fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the antibody of the first fusion protein comprises: (i) a heavy chain variable region (VH) comprising an amino acid sequence having at least 90% sequence identity to an amino acid sequence of any one of SEQ ID Nos: 1, 3, 5, 7, 9, 11, 13, and 15; and (ii) a light chain variable region (VL) comprising an amino acid sequence having at least 90% sequence identity to an amino acid sequence of any one of SEQ ID Nos: 2, 4, 6, 8, 10, 12, 14, and 16; (b) the transmembrane polypeptide of the first fusion protein comprises an amino acid sequence having at least 90% sequence identity to an amino acid sequence of any one of SEQ ID Nos: 31-39; and (c) the oligomerization domain of the first fusion protein comprises an amino acid sequence having at least 90% sequence identity to an amino acid sequence of any one of SEQ ID Nos: 17-30. In some embodiments, (a) the immune modulating polypeptide comprises an NK cell activating ligand, an immune checkpoint molecule, an inflammatory cytokine, or a proliferation cytokine; and (b) the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21. In some embodiments, the immune modulating polypeptide comprises TNF-α. In some embodiments, (a) the second fusion protein comprises an immune modulating polypeptide comprising an NK cell activating ligand, an immune checkpoint molecule, an inflammatory cytokine, or a proliferation cytokine; (b) the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and (c) the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21. In some embodiments, the immune modulating polypeptide comprises TNF-α. In some embodiments, (a) the immune modulating polypeptide comprises an NK cell activating ligand, an immune checkpoint molecule, an inflammatory cytokine, or a proliferation cytokine; and (b) the transmembrane polypeptide of the second fusion protein comprises an amino acid sequence having at least 90% sequence identity to an amino acid sequence of any one of SEQ ID Nos: 31-39. In some embodiments, the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21. In some embodiments, the immune modulating polypeptide comprises TNF-α. In some embodiments, (a) the immune modulating polypeptide comprises an NK cell activating ligand, an immune checkpoint molecule, an inflammatory cytokine, or a proliferation cytokine; (b) the transmembrane polypeptide of the second fusion protein comprises an amino acid sequence having at least 90% sequence identity to an amino acid sequence of any one of SEQ ID Nos: 31-39; and (c) the oligomerization domain of the second fusion protein comprises an amino acid sequence having at least 90% sequence identity to an amino acid sequence of any one of SEQ ID Nos: 17-30. In some embodiments, the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21. In some embodiments, the immune modulating polypeptide comprises TNF-α. In some embodiments, the antibody of the first fusion protein comprises a CDR-H1 according to SEQ ID NO: 53, a CDR-H2 according to SEQ ID NO: 54, a CDR-H3 according to SEQ ID NO: 55, a CDR-L1 according to SEQ ID NO: 77, a CDR-L2 according to SEQ ID NO: 78, and a CDR-L3 according to SEQ ID NO: 79. In some embodiments, the antibody of the first fusion protein comprises a CDR-H1 according to SEQ ID NO: 56, a CDR-H2 according to SEQ ID NO: 57, a CDR-H3 according to SEQ ID NO: 58, a CDR-L1 according to SEQ ID NO: 80, a CDR-L2 according to SEQ ID NO: 81, and a CDR-L3 according to SEQ ID NO: 82. In some embodiments, the antibody of the first fusion protein comprises a CDR-H1 according to SEQ ID NO: 59, a CDR-H2 according to SEQ ID NO: 60, a CDR-H3 according to SEQ ID NO: 61, a CDR-L1 according to SEQ ID NO: 83, a CDR-L2 according to SEQ ID NO: 84, and a CDR-L3 according to SEQ ID NO: 85. In some embodiments, the antibody of the first fusion protein comprises a CDR-H1 according to SEQ ID NO: 62, a CDR-H2 according to SEQ ID NO: 63, a CDR-H3 according to SEQ ID NO: 64, a CDR-L1 according to SEQ ID NO: 86, a CDR-L2 according to SEQ ID NO: 87, and a CDR-L3 according to SEQ ID NO: 88. In some embodiments, the antibody of the first fusion protein comprises a CDR-H1 according to SEQ ID NO: 65, a CDR-H2 according to SEQ ID NO: 66, a CDR-H3 according to SEQ ID NO: 67, a CDR-L1 according to SEQ ID NO: 89, a CDR-L2 according to SEQ ID NO: 90, and a CDR-L3 according to SEQ ID NO: 91. In some embodiments, the antibody of the first fusion protein comprises a CDR-H1 according to SEQ ID NO: 68, a CDR-H2 according to SEQ ID NO: 69, a CDR-H3 according to SEQ ID NO: 70, a CDR-L1 according to SEQ ID NO: 92, a CDR-L2 according to SEQ ID NO: 93, and a CDR-L3 according to SEQ ID NO: 94. In some embodiments, the antibody of the first fusion protein comprises a CDR-H1 according to SEQ ID NO: 71, a CDR-H2 according to SEQ ID NO: 72, a CDR-H3 according to SEQ ID NO: 73, a CDR-L1 according to SEQ ID NO: 95, a CDR-L2 according to SEQ ID NO: 96, and a CDR-L3 according to SEQ ID NO: 97. In some embodiments, the antibody of the first fusion protein comprises a CDR-H1 according to SEQ ID NO: 74, a CDR-H2 according to SEQ ID NO: 75, a CDR-H3 according to SEQ ID NO: 76, a CDR-L1 according to SEQ ID NO: 98, a CDR-L2 according to SEQ ID NO: 99, and a CDR-L3 according to SEQ ID NO: 100. In some embodiments, the antibody of the second fusion protein comprises a CDR-H1 according to SEQ ID NO: 53, a CDR-H2 according to SEQ ID NO: 54, a CDR-H3 according to SEQ ID NO: 55, a CDR-L1 according to SEQ ID NO: 77, a CDR-L2 according to SEQ ID NO: 78, and a CDR-L3 according to SEQ ID NO: 79. In some embodiments, the antibody of the second fusion protein comprises a CDR-H1 according to SEQ ID NO: 56, a CDR-H2 according to SEQ ID NO: 57, a CDR-H3 according to SEQ ID NO: 58, a CDR-L1 according to SEQ ID NO: 80, a CDR-L2 according to SEQ ID NO: 81, and a CDR-L3 according to SEQ ID NO: 82. In some embodiments, the antibody of the second fusion protein comprises a CDR-H1 according to SEQ ID NO: 59, a CDR-H2 according to SEQ ID NO: 60, a CDR-H3 according to SEQ ID NO: 61, a CDR-L1 according to SEQ ID NO: 83, a CDR-L2 according to SEQ ID NO: 84, and a CDR-L3 according to SEQ ID NO: 85. In some embodiments, the antibody of the second fusion protein comprises a CDR-H1 according to SEQ ID NO: 62, a CDR-H2 according to SEQ ID NO: 63, a CDR-H3 according to SEQ ID NO: 64, a CDR-L1 according to SEQ ID NO: 86, a CDR-L2 according to SEQ ID NO: 87, and a CDR-L3 according to SEQ ID NO: 88. In some embodiments, the antibody of the second fusion protein comprises a CDR-H1 according to SEQ ID NO: 65, a CDR-H2 according to SEQ ID NO: 66, a CDR-H3 according to SEQ ID NO: 67, a CDR-L1 according to SEQ ID NO: 89, a CDR-L2 according to SEQ ID NO: 90, and a CDR-L3 according to SEQ ID NO: 91. In some embodiments, the antibody of the second fusion protein comprises a CDR-H1 according to SEQ ID NO: 68, a CDR-H2 according to SEQ ID NO: 69, a CDR-H3 according to SEQ ID NO: 70, a CDR-L1 according to SEQ ID NO: 92, a CDR-L2 according to SEQ ID NO: 93, and a CDR-L3 according to SEQ ID NO: 94. In some embodiments, the antibody of the second fusion protein comprises a CDR-H1 according to SEQ ID NO: 71, a CDR-H2 according to SEQ ID NO: 72, a CDR-H3 according to SEQ ID NO: 73, a CDR-L1 according to SEQ ID NO: 95, a CDR-L2 according to SEQ ID NO: 96, and a CDR-L3 according to SEQ ID NO: 97. In some embodiments, the antibody of the second fusion protein comprises a CDR-H1 according to SEQ ID NO: 74, a CDR-H2 according to SEQ ID NO: 75, a CDR-H3 according to SEQ ID NO: 76, a CDR-L1 according to SEQ ID NO: 98, a CDR-L2 according to SEQ ID NO: 99, and a CDR-L3 according to SEQ ID NO: 100.

Disclosed herein, in some embodiments, is a method of treating HBV in a subject in need thereof comprising: (a) selecting an antibody that binds to a surface protein of an HBV; and (b) expressing the antibody on a surface of a multivalent particle, wherein the multivalent particle has a binding affinity to the surface protein of the HBV that is higher than the binding affinity of a soluble version of the antibody to the surface protein of the HBV. In some embodiments, the surface protein of the HBV comprises Large Hepatitis B surface Antigen (L-HBsAg), Medium Hepatitis B surface Antigen (M-HBsAg), or Small Hepatitis B surface Antigen (S-HBsAg). In some embodiments, the method comprises administering the multivalent particle to the subject. In some embodiments, the multivalent particle neutralizes the HBV when the multivalent particle binds to the surface protein of the HBV. In some embodiments, the antibody is expressed in a fusion protein on the surface of the multivalent particle, wherein the fusion protein comprises a transmembrane polypeptide. In some embodiments, the transmembrane polypeptide comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, the fusion protein comprises an oligomerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, the antibody comprises an amino acid sequence from at least one complementarity determining region of 2H5, ADRI-2F3, H004, H009, H007, H015, H019, or H020. In some embodiments, the antibody comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the fusion protein is expressed at a valency of about 10 copies on the surface of the multivalent particle. In some embodiments, the multivalent particle comprises a viral-like particle. In some embodiments, the multivalent particle comprises an extracellular vesicle. In some embodiments, the extracellular vesicle comprises an ectosome. In some embodiments, the extracellular vesicle comprises an exosome.

Disclosed herein, in some embodiments, is a method of using a multivalent HBV-Antivirus as a treatment vaccine to induce antiviral immunity against HBV or HDV in a subject infected with HBV or HDV comprising administering to the subject the HBV-Antivirus, wherein the HBV-Antivirus comprises a first fusion protein that comprises a transmembrane polypeptide and an antibody that binds to an HBV surface protein, wherein the first fusion protein is expressed on a surface of the HBV-Antivirus. In some embodiments, the HBV-Antivirus is administered to the subject through inhalation or intranasal delivery to induce protective immunity against future HBV or HDV infections.

Disclosed herein, in some embodiments, is a method of using a multivalent HBV-Antivirus inactivated vaccine to induce protective immunity against HBV or HDV in a subject in need thereof comprising administering to the subject the HBV-Antivirus inactivated vaccine, wherein the HBV-Antivirus inactivated vaccine comprises an HBV and the HBV-Antivirus, wherein the HBV-Antivirus comprises a first fusion protein that comprises a transmembrane polypeptide and an antibody that binds to a HBV surface protein, wherein the first fusion protein is expressed on a surface of the HBV-Antivirus. In some embodiments, the HBV-Antivirus is administered to the subject through intranasal or intramuscular delivery.

Disclosed herein, in some embodiments, is a composition comprising a multivalent HBV-Antivirus wherein the HBV-Antivirus comprises an enveloped particle that displays at least 10 copies of an antibody on a surface of the HBV-Antivirus, wherein the antibody binds to a surface protein of an HBV.

Disclosed herein, in some embodiments, is a composition comprising a multi-specific, multivalent HBV-Antivirus wherein the HBV-Antivirus comprises an enveloped particle that displays at least 10 copies of a multi-specific antibody on a surface of the HBV-Antivirus, wherein the multi-specific antibody binds to one or more surface proteins of an HBV.

Disclosed herein, in some embodiments, is a composition comprising a multi-specific, multivalent HBV-Antivirus wherein the HBV-Antivirus comprises an enveloped particle that displays at least 10 copies of a first antibody on a surface of the HBV-Antivirus and at least 10 copies of a second antibody on the surface of the HBV-Antivirus, wherein the first antibody binds to a surface protein of an HBV and the second antibody binds to a surface protein of the HBV, wherein the second antibody has less than 100% sequence identity to the first antibody.

Disclosed herein, in some embodiments, is a composition comprising a dual-action, immune-modulating HBV-Antivirus wherein the HBV-Antivirus comprises an enveloped particle that displays at least 10 copies of an antibody on a surface of the HBV-Antivirus and at least 10 copies of an immune modulating polypeptide on the surface of the HBV-Antivirus, wherein the antibody binds to a surface protein of an HBV.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a Hepatitis B virus particle and its surface antigens L-HBsAg, M-HBsAg, and S-HBsAg.

FIG. 1B illustrates the display frequency ratio of L-HBsAg, M-HBsAg, and S-HBsAg.

FIG. 1C illustrates the design of mono-specific HBV-Antiviruses displaying antibodies targeting each of the three different HBV surface antigens L-HBsAg, M-HBsAg, and S-HBsAg.

FIG. 2A illustrates the design of a monomeric HBV-Antivirus display vector encoding an antibody fused to the transmembrane and cytoplasmic tail regions of VSV-G protein.

FIG. 2B illustrates the design of a trimeric HBV-Antivirus display vector encoding an antibody fused to the D4 post-fusion trimerization domain and the transmembrane and cytoplasmic tail regions of VSV-G protein.

FIG. 3A illustrates the design and production of a monomeric HBV-Antivirus as VLPs with viral genome.

FIG. 3B illustrates the design and production of a monomeric HBV-Antivirus as VLPs without viral genome.

FIG. 3C illustrates the design and production of a monomeric HBV-Antivirus as ectosomes or exosomes.

FIG. 4A illustrates the design and production of a trimeric HBV-Antivirus as VLPs with viral genome.

FIG. 4B illustrates the design and production of a trimeric HBV-Antivirus as VLPs without viral genome.

FIG. 4C illustrates the design and production of a trimeric HBV-Antivirus as ectosomes or exosomes.

FIG. 5 illustrates the design of a mixed oligomer, multi-specific HBV-antivirus using distinct oligomeric display vectors encoding two different antibodies.

FIG. 6A illustrates the design and production of a mixed monomeric and trimeric, multi-specific HBV-Antivirus as VLPs with viral genome.

FIG. 6B illustrates the design and production of a mixed monomeric and trimeric, multi-specific HBV-Antivirus as VLPs without viral genome.

FIG. 6C illustrates the design and production of a mixed oligomer, multi-specific HBV-Antivirus as ectosomes or exosomes.

FIG. 7A illustrates the dual-action mechanisms of an immune-modulating IM-HBV-Antivirus through viral neutralization and antibody-guided TNF stimulation.

FIG. 7B illustrates configurations of display vectors for a mixed oligomer, immune-modulating IM-HBV-Antivirus co-displaying antibodies and immune modulating peptides.

FIG. 8A illustrates the design and production of a mixed oligomer, immune-modulating IM-HBV-Antivirus as VLPs with viral genome.

FIG. 8B illustrates the design and production of a mixed oligomer, immune-modulating IM-HBV-Antivirus as VLPs without viral genome.

FIG. 8C illustrates the design and production of a mixed oligomer, immune-modulating IM-HBV-Antivirus as exosomes or ectosomes.

FIG. 9 illustrates quantitative western blot analysis of different HBV-Antiviruses.

FIG. 10A illustrates in vitro neutralization of HBV in HepG2 NTCP cells by monomeric αS:H015/VM HBV-Antivirus targeting HBV S antigen.

FIG. 10B illustrates in vitro neutralization of HBV in HepG2 NTCP cells by trimeric αS:H015/D4 HBV-Antivirus targeting HBV S antigen.

FIG. 11A illustrates in vitro neutralization of HBV in HepG2 NTCP cells by monomeric αS:H004/VM HBV-Antivirus targeting HBV S antigen.

FIG. 11B illustrates in vitro neutralization of HBV in HepG2 NTCP cells by trimeric αS:H004/D4 HBV-Antivirus targeting HBV S antigen.

FIG. 12 illustrates in vitro neutralization of HBV in HepG2 NTCP cells by monomeric αL:2H5/VM HBV-Antivirus targeting the Pre-S1 domain of the HBV L antigen.

FIG. 13 illustrates in vitro neutralization of HBV in HepG2 NTCP cells by tandem bi-specific (αS-αL)/D4 HBV-Antivirus targeting the S antigen and the Pre-S1 domain of the L antigen.

FIG. 14A illustrates in vitro neutralization of HBV as a function of extracellular HBV DNA in primary hepatocytes by αS:H004/VM and αS:H004/D4 HBV-Antiviruses.

FIG. 14B illustrates in vitro neutralization of HBV as a function of secreted S antigen in primary hepatocytes by αS:H004/VM and αS:H004/D4 HBV-Antiviruses.

FIG. 15A illustrates in vitro neutralization of HBV as a function of extracellular HBV DNA in primary hepatocytes by tandem bi-specific (αS-αL)/D4 HBV-Antivirus.

FIG. 15B illustrates in vitro neutralization of HBV as a function of secreted S antigen DNA in primary hepatocytes by tandem bi-specific (αS-αL)/D4 HBV-Antivirus.

FIG. 16A illustrates an exemplary design of a trimeric antibody display vector for an HBV-Antivirus wherein the D4 trimerization domain follows the antibody sequence and precedes the transmembrane domain.

FIG. 16B illustrates another exemplary design of a trimeric antibody display vector for an HBV-Antivirus wherein the D4 trimerization domain follows the transmembrane domain and precedes the cytosolic domain.

FIG. 17A illustrates an exemplary design of a trimeric immune modulator display vector for an IM-HBV-Antivirus wherein the D4 trimerization domain follows the immune modulating peptide and precedes the transmembrane domain.

FIG. 17B illustrates another exemplary design of a trimeric immune modulator display vector for an IM-HBV-Antivirus wherein the D4 trimerization domain follows the transmembrane domain and precedes the cytosolic domain.

FIG. 17C illustrates another exemplary design of a trimeric immune modulator display vector for an IM-HBV-Antivirus wherein the D4 trimerization domain follows the signal peptide and precedes the immune modulator.

FIG. 18A illustrates an exemplary design of an oligomeric antibody display vector for an HBV-Antivirus wherein the generic oligomerization domain follows the antibody sequence and precedes the transmembrane domain.

FIG. 18B illustrates another exemplary design of an oligomeric antibody display vector for an HBV-Antivirus wherein the generic oligomerization domain follows the transmembrane domain and precedes the cytosolic domain.

FIG. 19A illustrates an exemplary design of an oligomeric immune modulator display vector for an IM-HBV-Antivirus wherein the generic oligomerization domain follows the immune modulating peptide and precedes the transmembrane domain.

FIG. 19B illustrates another exemplary design of an oligomeric immune modulator display vector for an IM-HBV-Antivirus wherein the generic oligomerization domain follows the transmembrane domain and precedes the cytosolic domain.

FIG. 19C illustrates another exemplary design of an oligomeric immune modulator display vector for an IM-HBV-Antivirus wherein the generic oligomerization domain follows the signal peptide and precedes the immune modulator.

FIG. 20 illustrates a method for determining HBV antigen-specific binding of IM-HBV-Antiviruses through membrane-intercalating fluorescent dyes and FACS analysis.

FIG. 21 illustrates functional analysis of antigen specific immune modulation by IM-HBV-Antiviruses displaying antibodies and TNF.

FIG. 22 illustrates HBV surface antigen specificity combinations of tandem bi-specific HBV-Antiviruses.

FIG. 23 illustrates HBV surface antigen specificity combinations of mixed bi-specific HBV-Antiviruses.

FIG. 24A illustrates in vitro neutralization of Hepatitis D Virus (HDV) in primary hepatocytes by αS:H015/VM and αS:H015/D4 HBV-Antiviruses as measured by intracellular HDV RNA.

FIG. 24B illustrates cytotoxicity analysis of αS:H015/D4 HBV-Antivirus as determined via CCK-8.

FIG. 24C illustrates in vitro neutralization of HDV in primary hepatocytes by αL:2H5/VM HBV-Antivirus as measured by intracellular HDV RNA.

FIG. 24D illustrates cytotoxicity analysis of αL:2H5/VM HBV-Antivirus as determined via CCK-8.

FIG. 24E illustrates in vitro neutralization of HDV in primary hepatocytes by bi-specific HBV-Antivirus (αS-αL)/D4 as measured by intracellular HDV RNA.

FIG. 24F illustrates cytotoxicity analysis of (αS-αL)/D4 HBV-Antivirus as determined via CCK-8.

### DETAILED DESCRIPTION OF THE INVENTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### HBV-Antivirus Compositions

Approximately 2 billion people are currently infected with Hepatitis B virus (HBV), a DNA virus from the Hepadnaviridae family. Out of the 2 billion infected, around 257 million, or 3.5% of the human population, live with chronic HBV (CHB) and nearly 887,000 people die each year from CHB-related liver complications. HBV infection can lead to both acute and chronic hepatitis, and if left unchecked, greatly increases the risk of fatal liver cirrhosis and hepatocellular carcinoma (HCC). Despite many approved therapies for the treatment of chronic hepatitis B, a cure has not been achieved because of the persistence of a stable form of HBV-covalently closed circular DNA within the nucleus of infected hepatocytes. A functional cure for HBV requires both the neutralization of circulating virions and the clearance of latent, infected cells.

Existing therapeutic strategies to treat HBV generally fall into two categories: direct-acting antivirals that inhibit various steps of the HBV lifecycle, and indirect antivirals that engage the host immune response to suppress HBV. Direct-acting antivirals include nucleoside/nucleotide analogs (NAs) that disrupt HBV viral reverse transcriptase activity and HBV DNA replication, neutralizing antibodies that block HBV entry, and HBsAg release inhibitors that prevent HBV budding. Indirect antivirals induces immune-stimulated genes to both directly inhibit HBV DNA replication and mediate clearance of infected hepatocytes harboring latent HBV covalently closed circular DNA (cccDNA). While these therapeutics may effectively reduce viral load in certain CHB patients, several significant shortcomings exist. Continuous treatment with nucleoside/nucleotide analogs is required to maintain viral suppression, and such extended treatment often results in the development of drug-resistant chronic HBV infection. Additionally, current direct-acting and indirect antivirals cannot fully cure patients and often fail to result in suppression of HBV surface antigen (HBsAg) and clearance of cccDNA from infected hepatocytes. Direct-acting and indirect-acting antiviral treatments also often result in off-target patient toxicity. In the case of indirect antivirals, non-specific immune side effects can range from flu-like symptoms to the development and exacerbation of autoimmune conditions.

Neutralizing antibodies, while heavily relied upon as neutralizing therapeutics for other pathogenic viruses, including coronaviruses such as SARS CoV-2, have had limited success in the treatment of HBV. No currently approved neutralizing antibody therapeutics are available. The high frequency of non-infectious, decoy subviral HBV particles (SVPs) displaying HBsAg may compromise the therapeutic efficacy of neutralizing antibodies. Moreover, the multivalent structure of HBV virions may help HBV virions to overcome neutralization by therapeutic antibodies. Notably, infectious HBV virions, called Dane particles, display nearly 160-200 spikes per particle. The distributed, high density multivalence is critical for low-affinity HBV attachment to cellular heparan sulfate proteoglycans (HSPGs) and for high-affinity virion binding to HBV's functional host cell receptor, sodium taurocholate co-transporting peptide (NTCP). Multivalence also compensates for decreases in the binding affinity of HBsAg resulting from mutations, creating an “affinity buffer” that permits HBsAg mutagenesis and evolution. In contrast, HBsAg mutagenesis can easily compromise or disrupt the binding affinity of monovalent or bivalent neutralizing antibodies.

Hepatitis Delta virus (HDV) is a satellite virus that can only replicate in the presence of HBV, as it relies on borrowed HBV surface antigens to infect hepatocytes and utilizes the same surface antigens—S, M and L—expressed by HBV. HDV co-infection with chronic HBV results in severe complications in HBV disease progression, from acute liver failure to rapid liver cirrhosis and elevated hepatocellular carcinoma risk. Hence, there is a need to develop treatments for both HDV and HBV.

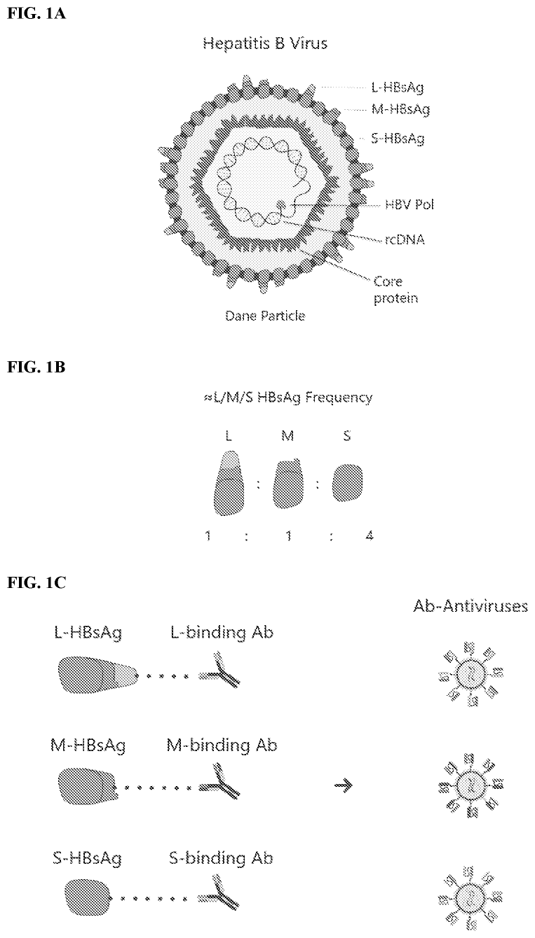

Disclosed herein are compositions and methods of using multivalent particles displaying antibodies specific for HBV surface proteins, dubbed HBV-Antiviruses. FIG. 1A illustrates a Hepatitis B virus particle, which includes Large Hepatitis B surface Antigen (L-HBsAg), Medium Hepatitis B surface Antigen (M-HBsAg), and Small Hepatitis B surface Antigen (S-HBsAg). FIG. 1B illustrates the frequency ratio of L-HBsAg, M-HBsAg, and S-HBsAg at 1:1:4. FIG. 1C illustrates the design of HBV-antiviruses each of which specifically targets L-HBsAg, M-HBsAg, or S-HBsAg. As disclosed herein, are compositions that comprise mRNA that encode the proteins that assemble into HBV-antiviruses.

The HBV-antiviruses of the disclosure can comprise genetically encoded vesicles, including viral-like particles (VLPs), exosomes or ectosomes, displaying many copies of antibodies that bind to HBV surface proteins in varied monomeric and oligomeric forms. HBV-antiviruses are designed to form high affinity, multivalent interactions with both infectious and subviral HBV particles (SVPs) displaying the 3 types of HBsAg (L, M, S). A single HBV-Antivirus can function as a sponge, capturing and mediating neutralization and clearance of multiple infectious HBV particles and SVPs during acute and chronic infection. Moreover, HBV-antiviruses can be genetically programmed to recognize multiple antigens or epitopes by displaying combinations of antibodies. For example, HBV-antiviruses can display a bi-specific tandem-scFv targeting two different HBsAg epitopes. This programmable, multi-specific multivalence makes HBV-antiviruses high avidity and affinity binder and more resistant to HBsAg escape mutations than bivalent neutralizing antibodies, and make them more effective in blocking HBV infection of host hepatocytes. Finally, HBV-antiviruses can be genetically programmed to have both antiviral neutralizing function and immune modulating function by co-displaying antiviral immune-modulating molecules and cytokines, such as TNFs, alongside HBsAg-binding Abs. This allows HBV-antiviruses to serve as multi-purpose antivirals capable of capturing HBV virions, inducing HBsAg-specific immune responses against HBV-infected cells, and modulating damaging inflammatory responses.

Described herein, in some embodiments, is an HBV-Antivirus. In some embodiments, the HBV-Antivirus comprises a multivalent particle displaying at least 10 copies of an antibody that binds to a surface protein of an HBV. Described herein, in some embodiments, is a multi-specific HBV-Antivirus. In some embodiments, the HBV-Antivirus comprises a multivalent particle displaying at least 10 copies of a multi-specific antibody that binds to one or more surface proteins of an HBV. Described herein, in some embodiments, is a multi-specific HBV-Antivirus. In some embodiments, the HBV-Antivirus comprises a multivalent particle displaying at least 10 copies of a first antibody that binds to a surface protein of an HBV. In some embodiments, the HBV-Antivirus comprises at least 10 copies of a second antibody that binds to a surface protein of an HBV. In some embodiments, the second antibody has less than 100% sequence identity to the first antibody. Described herein, in some embodiments, is a dual-action, immune-modulating HBV-Antivirus. In some embodiments, the HBV-Antivirus comprises a multivalent particle displaying at least 10 copies of an antibody that binds to a surface protein of an HBV and at least 10 copies of an immune modulating polypeptide.

Described herein, in some embodiments, are multivalent HBV-antiviruses. In some embodiments, the HBV-antivirus displays multiple copies of antibody that binds to a surface protein of an HBV. In some embodiments, the HBV-antivirus comprises a first fusion protein that comprises a transmembrane polypeptide and an antibody that binds to a surface protein of a hepatitis B virus (HBV). In some embodiments, the fusion protein is expressed on a surface of the HBV-antivirus. In some embodiments, the fusion protein is expressed at a valency of at least about 10 copies on the surface of the HBV-antivirus. Described herein are HBV-antiviruses comprising a first fusion protein that comprises a transmembrane polypeptide and an antibody that binds to a surface protein of HBV wherein the fusion protein is expressed at a valency of at least about 10 copies on a surface of the HBV-antivirus. In some embodiments, the HBV-Antivirus neutralizes the HBV when the first fusion protein is bound to a surface protein of the HBV. In some embodiments, the HBV-Antivirus neutralizes a HDV when the first fusion protein is bound to an HBV surface protein located on a surface of the HDV. In some embodiments, the HBV-Antivirus neutralizes both the HBV and HDV when the first fusion protein is bound to a surface protein of the HBV and an HBV surface protein located on the surface of the HDV. In some embodiments, the HBV-Antivirus comprises a fluorophore expressed on the surface of the antivirus. In some embodiments, the fluorophore is conjugated to a membrane-intercalating polypeptide.

In some embodiments, the HBV-Antivirus further comprises a second fusion protein. In some embodiments, the second fusion protein comprises a transmembrane polypeptide and an immune modulating polypeptide. In some embodiments, the immune modulating polypeptide comprises an NK cell activating ligand. In some embodiments, the NK cell activating ligand comprises m157, NKG2D ligand, CD70, or CD40. In some embodiments, the immune modulating polypeptide comprises an immune checkpoint molecule. In some embodiments, the immune checkpoint molecule comprises CTLA4, PD1, OX40, or CD28. In some embodiments, the immune modulating polypeptide comprises an inflammatory cytokine. In some embodiments, the inflammatory cytokine comprises IL-12, IL-18, TNF-α, or TNF-β. In some embodiments, the immune modulating polypeptide comprises a proliferation cytokine. In some embodiments, the proliferation cytokine comprises IL-7, IL-15, or IL-21.

In some embodiments, the second fusion protein comprises a transmembrane polypeptide and an antibody that binds to a surface protein of HBV and the antibody has an amino acid sequence that has less than 100% sequence identity to the antibody of the first fusion protein that binds to a surface protein of HBV. In some embodiments, the second fusion protein is expressed on a surface of the antivirus. In some embodiments, the second fusion protein is expressed at a valency of at least about 10 copies on the surface of the HBV-antivirus. In some embodiments, the antibody of the first fusion protein and the antibody of the second fusion protein binds to different surface proteins of the HBV. In some embodiments, the antibody of the first fusion protein and the antibody of the second fusion protein binds to the same surface protein of the HBV. In some embodiments, the antibody of the first fusion protein and the antibody of the second fusion protein binds to different epitopes of the same surface protein of the HBV. In some embodiments, the antibody of the first fusion protein and the antibody of the second fusion protein binds to the same epitope of the same surface protein of the HBV.

In some embodiments, the antibody of the first fusion protein binds to Large Hepatitis B surface Antigen (L-HBsAg), Medium Hepatitis B surface Antigen (M-HBsAg), or Small Hepatitis B surface Antigen (S-HBsAg). In some embodiments, the antibody of the first fusion protein binds to L-HBsAg. In some embodiments, the antibody of the first fusion protein binds to M-HBsAg. In some embodiments, the antibody of the first fusion protein binds to S-HBsAg. In some embodiments, the antibody of the second fusion protein binds to Large Hepatitis B surface Antigen (L HBsAg), Medium Hepatitis B surface Antigen (M HBsAg), or Small Hepatitis B surface Antigen (S-HBsAg). In some embodiments, the antibody of the second fusion protein binds to L-HBsAg. In some embodiments, the antibody of the second fusion protein binds to M-HBsAg. In some embodiments, the antibody of the second fusion protein binds to S-HBsAg. In some embodiments, the antibody of the first fusion protein binds to L-HBsAg and the antibody of the second fusion protein binds to L-HBsAg. In some embodiments, the antibody of the first fusion protein binds to M-HBsAg and the antibody of the second fusion protein binds to M-HBsAg. In some embodiments, the antibody of the first fusion protein binds to S-HBsAg and the antibody of the second fusion protein binds to S-HBsAg. In some embodiments, the antibody of the first fusion protein binds to L-HBsAg and the antibody of the second fusion protein binds to M-HBsAg. In some embodiments, the antibody of the first fusion protein binds to M-HBsAg and the antibody of the second fusion protein binds to L-HBsAg. In some embodiments, the antibody of the first fusion protein binds to L-HBsAg and the antibody of the second fusion protein binds to S-HBsAg. In some embodiments, the antibody of the first fusion protein binds to S-HBsAg and the antibody of the second fusion protein binds to L-HBsAg. In some embodiments, the antibody of the first fusion protein binds to M-HBsAg and the antibody of the second fusion protein binds to S-HBsAg. In some embodiments, the antibody of the first fusion protein binds to S-HBsAg and the antibody of the second fusion protein binds to M-HBsAg. In some embodiments, the antibody of the first fusion protein binds to the Pre-S1 domain of L-HBsAg and the antibody of the second fusion protein binds to the Pre-S2 domain of M-HBsAg. In some embodiments, the antibody of the first fusion protein binds to the Pre-S1 domain of L-HBsAg and the antibody of the second fusion protein binds to S-HBsAg. In some embodiments, the antibody of the first fusion protein binds to the Pre-S2 domain of M-HBsAg and the antibody of the second fusion protein binds to S-HBsAg. In some embodiments, the antibody of the first fusion protein binds to the Pre-S2 domain of M-HBsAg and the antibody of the second fusion protein binds to the Pre-S1 domain of L-HBsAg. In some embodiments, the antibody of the first fusion protein binds to S-HBsAg and the antibody of the second fusion protein binds to the Pre-S1 domain of L-HBsAg. In some embodiments, the antibody of the first fusion protein binds to S-HBsAg and the antibody of the second fusion protein binds to the Pre-S2 domain of M-HBsAg.

In some embodiments, the antibody of the first fusion protein comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody of the first fusion protein comprises a scFv. In some embodiments, the antibody of the first fusion protein comprises a Fab fragment. In some embodiments, the antibody of the first fusion protein comprises a neutralizing antibody. In some embodiments, the antibody of the first fusion protein comprises a non-neutralizing antibody. In some embodiments, the antibody of the first fusion protein comprises a multi-specific antibody. In some embodiments, the multi-specific antibody comprises a bispecific antibody. In some embodiments, the bispecific antibody binds to two different epitopes of L-HBsAg. In some embodiments, the bispecific antibody binds to two different epitopes of M-HBsAg. In some embodiments, the bispecific antibody binds to two different epitopes of S-HBsAg. In some embodiments, the bispecific antibody binds to L-HBsAg and M-HBsAg. In some embodiments, the bispecific antibody binds to L-HBsAg and S-HBsAg. In some embodiments, the bispecific antibody binds to M-HBsAg and S-HBsAg. In some embodiments, the bispecific antibody binds to the Pre-S1 domain of L-HBsAg and S-HBsAg. In some embodiments, the bispecific antibody binds to the Pre-S2 domain of M-HBsAg and S-HBsAg. In some embodiments, the bispecific antibody binds to the Pre-S1 domain of L-HBsAg and the Pre-S2 domain of M-HBsAg. In some embodiments, the multi-specific antibody comprises a trispecific antibody. In some embodiments, the trispecific antibody binds to L-HBsAg, M-HBsAg, and S-HBsAg. In some embodiments, the trispecific antibody binds to Pre-S1 domain of L-HBsAg, Pre-S2 domain of M-HBsAg, S-HBsAg. In some embodiments, the antibody of the first fusion protein comprises an amino acid sequence from at least one complementarity determining region of 2H5, ADRI-2F3, H004, H009, H007, H015, H019, or H020. In some embodiments, the antibody of the first fusion protein comprises an amino acid sequence comprising at least two complementarity determining regions of at least two antibodies selected from 2H5, ADRI-2F3, H004, H009, H007, H015, H019, and H020.

In some embodiments, the antibody of the first fusion protein comprises an amino acid sequence from at least one complementarity determining region of 2H5, ADRI-2F3, H004, H009, H007, H015, H019, or H020 and the immune modulating polypeptide of the second fusion protein comprises an NK cell activating ligand, an immune checkpoint molecule, an inflammatory cytokine, or a proliferation cytokine. In some embodiments, the antibody of the first fusion protein comprises an amino acid sequence comprising at least two complementarity determining regions of at least two antibodies selected from 2H5, ADRI-2F3, H004, H009, H007, H015, H019, and H020, and the immune modulating polypeptide of the second fusion protein comprises an NK cell activating ligand, an immune checkpoint molecule, an inflammatory cytokine, or a proliferation cytokine. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-α, or TNF-β. In some embodiments, the proliferation cytokine comprises IL-7, IL-15, or IL-21.

In some embodiments, the antibody of the second fusion protein comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the antibody of the second fusion protein comprises a scFv. In some embodiments, the antibody of the second fusion protein comprises a Fab fragment. In some embodiments, the antibody of the second fusion protein comprises a neutralizing antibody. In some embodiments, the antibody of the second fusion protein comprises a non-neutralizing antibody. In some embodiments, the antibody of the second fusion protein comprises a multi-specific antibody. In some embodiments, the multi-specific antibody comprises a bispecific antibody. In some embodiments, the bispecific antibody binds to two different epitopes of L-HBsAg. In some embodiments, the bispecific antibody binds to two different epitopes of M-HBsAg. In some embodiments, the bispecific antibody binds to two different epitopes of S-HBsAg. In some embodiments, the bispecific antibody binds to L-HBsAg and M-HBsAg. In some embodiments, the bispecific antibody binds to L-HBsAg and S-HBsAg. In some embodiments, the bispecific antibody binds to M-HBsAg and S-HBsAg. In some embodiments, the bispecific antibody binds to S-HBsAg and the Pre-S1 domain of L-HBsAg. In some embodiments, the bispecific antibody binds to S-HBsAg and the Pre-S2 domain of M-HBsAg. In some embodiments, the bispecific antibody binds to the Pre-S1 domain of L-HBsAg and the Pre-S2 domain of M-HBsAg. In some embodiments, the multi-specific antibody comprises a trispecific antibody. In some embodiments, the trispecific antibody binds to L-HBsAg, M-HBsAg, and S-HBsAg. In some embodiments, the trispecific antibody binds to Pre-S1 domain of L-HBsAg, Pre-S2 domain of M-HBsAg, S-HBsAg. In some embodiments, the antibody of the second fusion protein comprises an amino acid sequence from at least one complementarity determining region of 2H5, ADRI-2F3, H004, H009, H007, H015, H019, or H020. In some embodiments, the antibody of the second fusion protein comprises an amino acid sequence comprising at least two complementarity determining regions of at least two antibodies selected from 2H5, ADRI-2F3, H004, H009, H007, H015, H019, and H020.

In some embodiments, the antibody of the first fusion protein comprises a multi-specific antibody that binds to S-HBsAg and Pre-S1 domain of L-HBsAg and the antibody of the second fusion protein comprises a multi-specific antibody that binds to S-HBsAg and Pre-S2 domain of M-HBsAg. In some embodiments, the antibody of the first fusion protein comprises a multi-specific antibody that binds to S-HBsAg and Pre-S1 domain of L-HBsAg and the antibody of the second fusion protein comprises a multi-specific antibody that binds to Pre-S1 domain of L-HBsAg and Pre-S2 domain of M-HBsAg. In some embodiments, the antibody of the first fusion protein comprises a multi-specific antibody that binds to S-HBsAg and Pre-S2 domain of M-HBsAg and the antibody of the second fusion protein comprises a multi-specific antibody that binds to S-HBsAg and Pre-S1 domain of L-HBsAg. In some embodiments, the antibody of the first fusion protein comprises a multi-specific antibody that binds to S-HBsAg and Pre-S2 domain of M-HBsAg and the antibody of the second fusion protein comprises a multi-specific antibody that binds to Pre-S1 domain of L-HBsAg and Pre-S2 domain of M-HBsAg. In some embodiments, the antibody of the first fusion protein comprises a multi-specific antibody that binds to Pre-S1 domain of L-HBsAg and Pre-S2 domain of M-HBsAg and the antibody of the second fusion protein comprises a multi-specific antibody that binds to S-HBsAg and Pre-S1 domain of L-HBsAg. In some embodiments, the antibody of the first fusion protein comprises a multi-specific antibody that binds to Pre-S1 domain of L-HBsAg and Pre-S2 domain of M-HBsAg and the antibody of the second fusion protein comprises a multi-specific antibody that binds to S-HBsAg and Pre-S2 domain of M-HBsAg. In some embodiments, the antibody of the first fusion protein comprises a multi-specific antibody that binds to S-HBsAg and Pre-S2 domain of M-HBsAg, and the antibody of the second fusion protein comprises a multi-specific antibody that binds to S-HBsAg, Pre-S2 domain of M-HBsAg and Pre-S1 domain of L-HBsAg. In some embodiments, the antibody of the first fusion protein comprises a multi-specific antibody that binds to S-HBsAg and Pre-S1 domain of L-HBsAg, and the antibody of the second fusion protein comprises a multi-specific antibody that binds to S-HBsAg, Pre-S2 domain of M-HBsAg and Pre-S1 domain of L-HBsAg. In some embodiments, the antibody of the first fusion protein comprises a multi-specific antibody that binds to the Pre-S1 domain of L-HBsAg and Pre-S2 domain of M-HBsAg, and the antibody of the second fusion protein comprises a multi-specific antibody that binds to S-HBsAg, Pre-S2 domain of M-HBsAg and Pre-S1 domain of L-HBsAg. In some embodiments, the antibody of the first fusion protein comprises a multi-specific antibody that binds to S-HBsAg, Pre-S2 domain of M-HBsAg and Pre-S1 domain of L-HBsAg, and the antibody of the second fusion protein comprises a multi-specific antibody that binds to S-HBsAg and Pre-S2 domain of M-HBsAg. In some embodiments, the antibody of the first fusion protein comprises a multi-specific antibody that binds to S-HBsAg, Pre-S2 domain of M-HBsAg and Pre-S1 domain of L-HBsAg, and the antibody of the second fusion protein comprises a multi-specific antibody that binds to S-HBsAg and Pre-S1 domain of L-HBsAg. In some embodiments, the antibody of the first fusion protein comprises a multi-specific antibody that binds to S-HBsAg, Pre-S2 domain of M-HBsAg and Pre-S1 domain of L-HBsAg, and the antibody of the second fusion protein comprises a multi-specific antibody that binds to Pre-S1 domain of L-HBsAg and Pre-S2 domain of M-HBsAg.

TABLE 1A
Exemplary Antibody Sequences

| | | SEQ | VH | SEQ | VL |
|---|---|---|---|---|---|
| | HBsAg | ID | Amino Acid | ID | Amino Acid |
| Name | Specificity | NO | Sequence | NO | Sequence |
| 2H5 | L PreS1 | 1 | SQVQLQQSGPGLVKPSQTLS | 2 | QSVLTQPPSASGTPGQRVT |
| | | | LTCGISGDSVSSKSAAWNWI | | ISCSGSSSNIGSYYVYWYQ |
| | | | RQSPSRGLEWLGRTYYRSKW | | QFPGTAPKLLIYGNNQRPS |
| | | | HNDYAVSVKSRITINPDTSK | | GVPDRFSGSKSGTSASLAI |
| | | | NQFSLQLNSVTPEDTAVYYC | | TGLQAEDEADYYCQSYDSS |
| | | | ARGQMGALDVWGQGTTVTVS | | LSGVIFGGGTKLTVL |
| | | | S | | |
| ADRI- | S | 3 | EVQVLESGGGLVQPGGSLRL | 4 | SYVLTQPPSVSVAPGQTAR |
| 2F3 | | | SCAASGFRFSSYAMSWVRQA | | MTCGGNNIGSESVHWFQQK |
| | | | PGKGLEWVSGISGTGENTYY | | PGQAPVLVVYDDSDRPSGI |
| | | | ADSVKGRFTISRDNSKNTLY | | PERFSGSNSGNTATLTISR |
| | | | VQMNSLRAEDTAVYYCAKDA | | VEAGDEADYYCQVWDSSSD |
| | | | ILGSGHPWYFHVWGRGTLVT | | HAVFGGGTQLTVL |
| | | | VSS | | |
| H004 | S | 5 | EVQLVESGGGLVQPGGSLRL | 6 | SYVLTQPPSVSVAPGQTAR |
| | | | SCAASGFTFSSYAMSWVRQA | | ITCGGNNIGSKSVHWYQQK |
| | | | PGKGLEWVSAISGSGGSTYY | | PGQAPVLVVYDDSDRPSGI |
| | | | ADSVKGRFTISRDNSKNTLY | | PERFSGSNSGNTATLTISR |
| | | | LQMNSLRAEDTAVYYCAKDG | | VEAGDEADYYCQVWDSNHD |
| | | | YFGSGSLYGIDVWGQGTTVT | | HPGVVFGGGTKLTVL |
| | | | VSS | | |
| H009 | S | 7 | QVQLVESGGGVVQPGRSLRL | 8 | SYVLTQPPSVSVAPGQTAR |
| | | | SCAASGFTFSSYGMHWVRQA | | ITCGGNNIGSKSVHWYQQK |
| | | | PGKGLEWVAVISYDGSNKYY | | PGQAPVLVVYDDSDRPSGI |
| | | | ADSVKGRFTISRDNSKNTLY | | PERFSGSNSGNTATLTISR |
| | | | LQMNSLRAEDTAVYYCAKTD | | VEAGDEADYYCQVWDGTRD |
| | | | IKWGATNYGMDVWGQGTTVT | | HLVVFGGGTKLTVL |
| | | | VSS | | |
| H007 | S | 9 | QVQLVESGGGVVQPGRSLRL | 10 | EIVLTQSPGTLSLSPGERA |
| | | | SCAASGFTFSSYGMHWVRQA | | TLSCRASQSVSSSYLAWYQ |
| | | | PGKGLEWVAVISYDGSNKYY | | QKPGQAPRLLIYGASSRAT |
| | | | ADSVKGRFTISRDNSKNTLY | | GIPDRFSGSGSGTDFTLTI |
| | | | LQMNSLRAEDTAVYYCAKEI | | SRLEPEDFAVYYCQQYSSS |
| | | | GGFDFRSGSQRSYYYYGVDV | | PPGYTFGQGTKLEIK |
| | | | GWQGTTVTVSS | | |
| H015 | S | 11 | QVQLVESGGGVAQPGRSLRL | 12 | DIQVTQSPSSLSAFVGDRV |
| | | | SCAASGFSFSRHGMHWVRQA | | TITCRASQGLTSFINWYQQ |
| | | | PGKGLEWVAGIWFDGTNDYY | | KPGRAPKLLISSASSLQRG |
| | | | TDSVKGRFTISRDNSRSTLY | | VPSRFTASGSGTHFTLTIS |
| | | | LDINSLRAEDTAVYYCARED | | SLQPEDFATYYCQQSYGTP |
| | | | PHLLIATLDLWGLGTLVTVS | | ALAFGGGTKVDIK |
| | | | S | | |
| H019 | S | 13 | QVQLQESGPGLVKPSQTLSL | 14 | DIQMTQSPSSLSASVGDRV |
| | | | TCTVSGGSISSGDYYWSWIR | | TITCRASQSISSYLNWYQQ |
| | | | QPPGKGLEWIGYIYYSGSTY | | KPGKAPKLLIYAASSLQSG |
| | | | YNPSLKSRVTISVDTSKNQF | | VPSRFSGSGSGTDFTLTIS |
| | | | SLKLSSVTAADTAVYYCAIY | | SLQPEDFATYYCQQSYTIS |
| | | | MDEAWAFEIWGQGTMVTVSS | | LFTFGQGTKLEIK |
| H020 | S | 15 | QVQLQESGPGLVKPSETLSL | 16 | DIQMTQSPSSVSASVGDRV |
| | | | TCTVSGGSISSYYWSWIRQP | | TITCRASQGISSWLAWYQQ |
| | | | PGKGLEWIGYIYYSGSTNYN | | KPGKAPKLLIYAASSLQSG |
| | | | PSLKSRVTISVDTSKNQFSL | | VPSRFSGSGSGTDFTLTIS |
| | | | KLSSVTAADTAVYYCARHLY | | SLQPEDFATYYCQQANSFP |
| | | | RYGYRNYFDYWGQGTLVTVS | | LTFGGGTKVEIK |
| | | | S | | |

TABLE 1B
Exemplary Complementarity Determining
Region (CDR*) Sequences
Heavy Chain Complementarity Determining
Region (CDR) Sequences

| | CDR-H1 | SEQ | CDR-H2 | SEQ | CDR-H3 | SEQ |
|---|---|---|---|---|---|---|
| Anti- | Amino Acid | ID | Amino Acid | ID | Amino Acid | ID |
| body | Sequence | NO | Sequence | NO | Sequence | NO |
| 2H5 | SKSAAWN | 53 | RTYYRSKWHN | 54 | GQMGALDV | 55 |
| | | | DYAVSVKS | | | |
| ADRI- | SYAMS | 56 | GISGTGENTY | 57 | DAILGSGHPW | 58 |
| 2F3 | | | YADSVKG | | YFHV | |
| H004 | SYAMS | 59 | AISGSGGSTY | 60 | DGYFGSGSLY | 61 |
| | | | YADSVKG | | GIDV | |
| H009 | SYGMH | 62 | VISYDGSNKY | 63 | TDIKWGATNY | 64 |
| | | | YADSVKG | | GMDV | |
| H007 | SYGMH | 65 | VISYDGSNKY | 66 | EIGGFDFRSG | 67 |
| | | | YADSVKG | | SQRSYYYYGV | |
| | | | | | DV | |
| H015 | RHGMH | 68 | GIWFDGTNDY | 69 | EDPHLLIATL | 70 |
| | | | YTDSVKG | | DL | |
| H019 | SGDYYWS | 71 | YIYYSGSTYY | 72 | YMDEAWAFEI | 73 |
| | | | NPSLKS | | | |
| H020 | SYYWS | 74 | YIYYSGSTNY | 75 | HLYRYGYRNY | 76 |
| | | | NPSLKS | | FDY | |

Light Chain Complementarity Determining
Region (CDR) Sequences

| | CDR-L1 | SEQ | CDR-L2 | SEQ | CDR-L3 | SEQ |
|---|---|---|---|---|---|---|
| Anti- | Amino Acid | ID | Amino Acid | ID | Amino Acid | ID |
| body | Sequence | NO | Sequence | NO | Sequence | NO |
| 2H5 | SGSSSNIG | 77 | GNNQRPS | 78 | QSYDSSLSGV | 79 |
| | SYYVY | | | | I | |
| ADRI- | GGNNIGSE | 80 | DDSDRPS | 81 | QVWDSSSDHA | 82 |
| 2F3 | SVH | | | | V | |
| H004 | GGNNIGSK | 83 | DDSDRPS | 84 | QVWDSNHDHP | 85 |
| | SVH | | | | GVV | |
| H009 | GGNNIGSK | 86 | DDSDRPS | 87 | QVWDGTRDHL | 88 |
| | SVH | | | | VV | |
| H007 | RASQSVSS | 89 | GASSRAT | 90 | QQYSSSPPGY | 91 |
| | SYLA | | | | T | |
| H015 | RASQGLTS | 92 | SASSLQR | 93 | QQSYGTPALA | 94 |
| | FIN | | | | | |
| H019 | RASQSISS | 95 | AASSLQS | 96 | QQSYTISLFT | 97 |
| | YLN | | | | | |
| H020 | RASQGISS | 98 | AASSLQS | 99 | QQANSFPLT | 100 |
| | WLA | | | | | |

*The CDR sequences listed in Table 1B are defined according to the Kabat numbering scheme.

In some embodiments, the antibody of the first fusion protein comprises an amino acid sequence disclosed in Table 1A, or an amino acid sequence that is substantially identical to an amino acid sequence in Table 1A (e.g. 75%, 80%, 85%, 90%, 95%, 96%, 97%, 98%, 99% sequence identity). In some embodiments, the antibody of the second fusion protein comprises an amino acid sequence disclosed in Table 1A, or an amino acid sequence that is substantially identical to an amino acid sequence in Table 1A (e.g. 75%, 80%, 85%, 90%, 95%, 96%, 97%, 98%, 99% sequence identity).

In some embodiments, the antibody of the first fusion protein comprises a heavy chain variable domain (VH). In some embodiments, the antibody of the second fusion protein comprises a heavy chain variable domain (VH). In some embodiments, the antibody of the first fusion protein comprises a light domain variable domain (VL). In some embodiments, the antibody of the second fusion protein comprises a light domain variable domain (VL). In some embodiments, the antibody of the first fusion protein comprises a complementarity determining region (CDR) of any of the sequences in Table 1A and Table 1B. In some embodiments, the antibody of the second fusion protein comprises a complementarity determining region (CDR) of any of the sequences in Table 1A and Table 1B. In some embodiments, a CDR sequence (CDR-H1, CDR-H2, CDR-H3 and CDR-L1, CDR-L2, CDR-L3) of the first fusion protein are determined according to any of the definitions described above. In some embodiments, a CDR sequence (CDR-H1, CDR-H2, CDR-H3 and CDR-L1, CDR-L2, CDR-L3) of the second fusion protein are determined according to any of the definitions described above. In some embodiments, the immune modulating polypeptide of the second fusion protein comprises an NK cell activating ligand, an immune checkpoint molecule, an inflammatory cytokine, or a proliferation cytokine. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-α, or TNF-β. In some embodiments, the proliferation cytokine comprises IL-7, IL-15, or IL-21.

In some embodiments, the antibody of the first fusion protein comprises a heavy chain complementarity determining region 1 (CDR-H1) according to any one of the SEQ ID NOs: 53, 56, 59, 62, 65, 68, 71, and 74. In some embodiments, the antibody of the first fusion protein comprises a heavy chain complementarity determining region 2 (CDR-H2) according to any one of the SEQ ID NOs: 54, 57, 60, 63, 66, 69, 72, and 75. In some embodiments, the antibody of the first fusion protein comprises a heavy chain complementarity determining region 3 (CDR-H3) according to any one of the SEQ ID NOs: 55, 58, 61, 64, 67, 70, 73, and 76. In some embodiments, the antibody of the first fusion protein comprises a light chain complementarity determining region 1 (CDR-L1) according to any one of the SEQ ID NOs: 77, 80, 83, 86, 89, 92, 95, and 98. In some embodiments, the antibody of the first fusion protein comprises a light chain complementarity determining region 2 (CDR-L2) according to any one of the SEQ ID NOs: 78, 81, 84, 87, 90, 93, 96, and 99. In some embodiments, the antibody of the first fusion protein comprises a light chain complementarity determining region 3 (CDR-L3) according to any one of the SEQ ID NOs: 79, 82, 85, 88, 91, 94, 97, and 100. In some embodiments, the antibody of the first fusion protein comprises a CDR-H1 according to SEQ ID NO: 53, a CDR-H2 according to SEQ ID NO: 54, and a CD-H3 according to SEQ ID NO: 55. In some embodiments, the antibody of the first fusion protein comprises a CDR-L1 according to SEQ ID NO: 77, a CDR-L2 according to SEQ ID NO: 78, and a CDR-L3 according to SEQ ID NO: 79. In some embodiments, the antibody of the first fusion protein comprises a CDR-H1 according to SEQ ID NO: 56, a CDR-H2 according to SEQ ID NO: 57, and a CD-H3 according to SEQ ID NO: 58. In some embodiments, the antibody of the first fusion protein comprises a CDR-L1 according to SEQ ID NO: 80, a CDR-L2 according to SEQ ID NO: 81, and a CDR-L3 according to SEQ ID NO: 82. In some embodiments, the antibody of the first fusion protein comprises a CDR-H1 according to SEQ ID NO: 59, a CDR-H2 according to SEQ ID NO: 60, and a CD-H3 according to SEQ ID NO: 61. In some embodiments, the antibody of the first fusion protein comprises a CDR-L1 according to SEQ ID NO: 83, a CDR-L2 according to SEQ ID NO: 84, and a CDR-L3 according to SEQ ID NO: 85. In some embodiments, the antibody of the first fusion protein comprises a CDR-H1 according to SEQ ID NO: 62, a CDR-H2 according to SEQ ID NO: 63, and a CD-H3 according to SEQ ID NO: 64. In some embodiments, the antibody of the first fusion protein comprises a CDR-L1 according to SEQ ID NO: 86, a CDR-L2 according to SEQ ID NO: 87, and a CDR-L3 according to SEQ ID NO: 88. In some embodiments, the antibody of the first fusion protein comprises a CDR-H1 according to SEQ ID NO: 65, a CDR-H2 according to SEQ ID NO: 66, and a CD-H3 according to SEQ ID NO: 67. In some embodiments, the antibody of the first fusion protein comprises a CDR-L1 according to SEQ ID NO: 89, a CDR-L2 according to SEQ ID NO: 90, and a CDR-L3 according to SEQ ID NO: 91. In some embodiments, the antibody of the first fusion protein comprises a CDR-H1 according to SEQ ID NO: 68, a CDR-H2 according to SEQ ID NO: 69, and a CD-H3 according to SEQ ID NO: 70. In some embodiments, the antibody of the first fusion protein comprises a CDR-L1 according to SEQ ID NO: 92, a CDR-L2 according to SEQ ID NO: 93, and a CDR-L3 according to SEQ ID NO: 94. In some embodiments, the antibody of the first fusion protein comprises a CDR-H1 according to SEQ ID NO: 71, a CDR-H2 according to SEQ ID NO: 72, and a CD-H3 according to SEQ ID NO: 73. In some embodiments, the antibody of the first fusion protein comprises a CDR-L1 according to SEQ ID NO: 95, a CDR-L2 according to SEQ ID NO: 96, and a CDR-L3 according to SEQ ID NO: 97. In some embodiments, the antibody of the first fusion protein comprises a CDR-H1 according to SEQ ID NO: 74, a CDR-H2 according to SEQ ID NO: 75, and a CD-H3 according to SEQ ID NO: 76. In some embodiments, the antibody of the first fusion protein comprises a CDR-L1 according to SEQ ID NO: 98, a CDR-L2 according to SEQ ID NO: 99, and a CDR-L3 according to SEQ ID NO: 100.

In some embodiments, the antibody of the first fusion protein comprises a CDR-H1 according to SEQ ID NO: 53, a CDR-H2 according to SEQ ID NO: 54, a CD-H3 according to SEQ ID NO: 55, a CDR-L1 according to SEQ ID NO: 77, a CDR-L2 according to SEQ ID NO: 78, and a CDR-L3 according to SEQ ID NO: 79. In some embodiments, the antibody of the first fusion protein comprises a CDR-H1 according to SEQ ID NO: 56, a CDR-H2 according to SEQ ID NO: 57, a CD-H3 according to SEQ ID NO: 58, a CDR-L1 according to SEQ ID NO: 80, a CDR-L2 according to SEQ ID NO: 81, and a CDR-L3 according to SEQ ID NO: 82. In some embodiments, the antibody of the first fusion protein comprises a CDR-H1 according to SEQ ID NO: 59, a CDR-H2 according to SEQ ID NO: 60, a CD-H3 according to SEQ ID NO: 61, a CDR-L1 according to SEQ ID NO: 83, a CDR-L2 according to SEQ ID NO: 84, and a CDR-L3 according to SEQ ID NO: 85. In some embodiments, the antibody of the first fusion protein comprises a CDR-H1 according to SEQ ID NO: 62, a CDR-H2 according to SEQ ID NO: 63, a CD-H3 according to SEQ ID NO: 64, a CDR-L1 according to SEQ ID NO: 86, a CDR-L2 according to SEQ ID NO: 87, and a CDR-L3 according to SEQ ID NO: 88. In some embodiments, the antibody of the first fusion protein comprises a CDR-H1 according to SEQ ID NO: 65, a CDR-H2 according to SEQ ID NO: 66, a CD-H3 according to SEQ ID NO: 67, a CDR-L1 according to SEQ ID NO: 89, a CDR-L2 according to SEQ ID NO: 90, and a CDR-L3 according to SEQ ID NO: 91. In some embodiments, the antibody of the first fusion protein comprises a CDR-H1 according to SEQ ID NO: 68, a CDR-H2 according to SEQ ID NO: 69, a CD-H3 according to SEQ ID NO: 70, a CDR-L1 according to SEQ ID NO: 92, a CDR-L2 according to SEQ ID NO: 93, and a CDR-L3 according to SEQ ID NO: 94. In some embodiments, the antibody of the first fusion protein comprises a CDR-H1 according to SEQ ID NO: 71, a CDR-H2 according to SEQ ID NO: 72, a CD-H3 according to SEQ ID NO: 73, a CDR-L1 according to SEQ ID NO: 95, a CDR-L2 according to SEQ ID NO: 96, and a CDR-L3 according to SEQ ID NO: 97. In some embodiments, the antibody of the first fusion protein comprises a CDR-H1 according to SEQ ID NO: 74, a CDR-H2 according to SEQ ID NO: 75, a CD-H3 according to SEQ ID NO: 76, a CDR-L1 according to SEQ ID NO: 98, a CDR-L2 according to SEQ ID NO: 99, and a CDR-L3 according to SEQ ID NO: 100.

In some embodiments, the antibody of the second fusion protein comprises a heavy chain complementarity determining region 1 (CDR-H1) according to any one of the SEQ ID NOs: 53, 56, 59, 62, 65, 68, 71, and 74. In some embodiments, the antibody of the second fusion protein comprises a heavy chain complementarity determining region 2 (CDR-H2) according to any one of the SEQ ID NOs: 54, 57, 60, 63, 66, 69, 72, and 75. In some embodiments, the antibody of the second fusion protein comprises a heavy chain complementarity determining region 3 (CDR-H3) according to any one of the SEQ ID NOs: 55, 58, 61, 64, 67, 70, 73, and 76. In some embodiments, the antibody of the second fusion protein comprises a light chain complementarity determining region 1 (CDR-L1) according to any one of the SEQ ID NOs: 77, 80, 83, 86, 89, 92, 95, and 98. In some embodiments, the antibody of the second fusion protein comprises a light chain complementarity determining region 2 (CDR-L2) according to any one of the SEQ ID NOs: 78, 81, 84, 87, 90, 93, 96, and 99. In some embodiments, the antibody of the second fusion protein comprises a light chain complementarity determining region 3 (CDR-L3) according to any one of the SEQ ID NOs: 79, 82, 85, 88, 91, 94, 97, and 100. In some embodiments, the antibody of the second fusion protein comprises a CDR-H1 according to SEQ ID NO: 53, a CDR-H2 according to SEQ ID NO: 54, and a CD-H3 according to SEQ ID NO: 55. In some embodiments, the antibody of the second fusion protein comprises a CDR-L1 according to SEQ ID NO: 77, a CDR-L2 according to SEQ ID NO: 78, and a CDR-L3 according to SEQ ID NO: 79. In some embodiments, the antibody of the second fusion protein comprises a CDR-H1 according to SEQ ID NO: 56, a CDR-H2 according to SEQ ID NO: 57, and a CD-H3 according to SEQ ID NO: 58. In some embodiments, the antibody of the second fusion protein comprises a CDR-L1 according to SEQ ID NO: 80, a CDR-L2 according to SEQ ID NO: 81, and a CDR-L3 according to SEQ ID NO: 82. In some embodiments, the antibody of the second fusion protein comprises a CDR-H1 according to SEQ ID NO: 59, a CDR-H2 according to SEQ ID NO: 60, and a CD-H3 according to SEQ ID NO: 61. In some embodiments, the antibody of the second fusion protein comprises a CDR-L1 according to SEQ ID NO: 83, a CDR-L2 according to SEQ ID NO: 84, and a CDR-L3 according to SEQ ID NO: 85. In some embodiments, the antibody of the second fusion protein comprises a CDR-H1 according to SEQ ID NO: 62, a CDR-H2 according to SEQ ID NO: 63, and a CD-H3 according to SEQ ID NO: 64. In some embodiments, the antibody of the second fusion protein comprises a CDR-L1 according to SEQ ID NO: 86, a CDR-L2 according to SEQ ID NO: 87, and a CDR-L3 according to SEQ ID NO: 88. In some embodiments, the antibody of the second fusion protein comprises a CDR-H1 according to SEQ ID NO: 65, a CDR-H2 according to SEQ ID NO: 66, and a CD-H3 according to SEQ ID NO: 67. In some embodiments, the antibody of the second fusion protein comprises a CDR-L1 according to SEQ ID NO: 89, a CDR-L2 according to SEQ ID NO: 90, and a CDR-L3 according to SEQ ID NO: 91. In some embodiments, the antibody of the second fusion protein comprises a CDR-H1 according to SEQ ID NO: 68, a CDR-H2 according to SEQ ID NO: 69, and a CD-H3 according to SEQ ID NO: 70. In some embodiments, the antibody of the second fusion protein comprises a CDR-L1 according to SEQ ID NO: 92, a CDR-L2 according to SEQ ID NO: 93, and a CDR-L3 according to SEQ ID NO: 94. In some embodiments, the antibody of the second fusion protein comprises a CDR-H1 according to SEQ ID NO: 71, a CDR-H2 according to SEQ ID NO: 72, and a CD-H3 according to SEQ ID NO: 73. In some embodiments, the antibody of the second fusion protein comprises a CDR-L1 according to SEQ ID NO: 95, a CDR-L2 according to SEQ ID NO: 96, and a CDR-L3 according to SEQ ID NO: 97. In some embodiments, the antibody of the second fusion protein comprises a CDR-H1 according to SEQ ID NO: 74, a CDR-H2 according to SEQ ID NO: 75, and a CD-H3 according to SEQ ID NO: 76. In some embodiments, the antibody of the second fusion protein comprises a CDR-L1 according to SEQ ID NO: 98, a CDR-L2 according to SEQ ID NO: 99, and a CDR-L3 according to SEQ ID NO: 100.

In some embodiments, the antibody of the second fusion protein comprises a CDR-H1 according to SEQ ID NO: 53, a CDR-H2 according to SEQ ID NO: 54, a CD-H3 according to SEQ ID NO: 55, a CDR-L1 according to SEQ ID NO: 77, a CDR-L2 according to SEQ ID NO: 78, and a CDR-L3 according to SEQ ID NO: 79. In some embodiments, the antibody of the second fusion protein comprises a CDR-H1 according to SEQ ID NO: 56, a CDR-H2 according to SEQ ID NO: 57, a CD-H3 according to SEQ ID NO: 58, a CDR-L1 according to SEQ ID NO: 80, a CDR-L2 according to SEQ ID NO: 81, and a CDR-L3 according to SEQ ID NO: 82. In some embodiments, the antibody of the second fusion protein comprises a CDR-H1 according to SEQ ID NO: 59, a CDR-H2 according to SEQ ID NO: 60, a CD-H3 according to SEQ ID NO: 61, a CDR-L1 according to SEQ ID NO: 83, a CDR-L2 according to SEQ ID NO: 84, and a CDR-L3 according to SEQ ID NO: 85. In some embodiments, the antibody of the second fusion protein comprises a CDR-H1 according to SEQ ID NO: 62, a CDR-H2 according to SEQ ID NO: 63, a CD-H3 according to SEQ ID NO: 64, a CDR-L1 according to SEQ ID NO: 86, a CDR-L2 according to SEQ ID NO: 87, and a CDR-L3 according to SEQ ID NO: 88. In some embodiments, the antibody of the second fusion protein comprises a CDR-H1 according to SEQ ID NO: 65, a CDR-H2 according to SEQ ID NO: 66, a CD-H3 according to SEQ ID NO: 67, a CDR-L1 according to SEQ ID NO: 89, a CDR-L2 according to SEQ ID NO: 90, and a CDR-L3 according to SEQ ID NO: 91. In some embodiments, the antibody of the second fusion protein comprises a CDR-H1 according to SEQ ID NO: 68, a CDR-H2 according to SEQ ID NO: 69, a CD-H3 according to SEQ ID NO: 70, a CDR-L1 according to SEQ ID NO: 92, a CDR-L2 according to SEQ ID NO: 93, and a CDR-L3 according to SEQ ID NO: 94. In some embodiments, the antibody of the second fusion protein comprises a CDR-H1 according to SEQ ID NO: 71, a CDR-H2 according to SEQ ID NO: 72, a CD-H3 according to SEQ ID NO: 73, a CDR-L1 according to SEQ ID NO: 95, a CDR-L2 according to SEQ ID NO: 96, and a CDR-L3 according to SEQ ID NO: 97. In some embodiments, the antibody of the second fusion protein comprises a CDR-H1 according to SEQ ID NO: 74, a CDR-H2 according to SEQ ID NO: 75, a CD-H3 according to SEQ ID NO: 76, a CDR-L1 according to SEQ ID NO: 98, a CDR-L2 according to SEQ ID NO: 99, and a CDR-L3 according to SEQ ID NO: 100.

In some embodiments, the antibody of the first fusion protein comprises a VH domain comprising an amino acid sequence according to SEQ ID NO: 1 and a VL domain comprising an amino acid sequence according to SEQ ID NO: 2. In some embodiments, the antibody of the first fusion protein comprises a VH domain comprising an amino acid sequence according to SEQ ID NO: 3 and a VL domain comprising an amino acid sequence according to SEQ ID NO: 4. In some embodiments, the antibody of the first fusion protein comprises a VH domain comprising an amino acid sequence according to SEQ ID NO: 5 and a VL domain comprising an amino acid sequence according to SEQ ID NO: 6. In some embodiments, the antibody of the first fusion protein comprises a VH domain comprising an amino acid sequence according to SEQ ID NO: 7 and a VL domain comprising an amino acid sequence according to SEQ ID NO: 8. In some embodiments, the antibody of the first fusion protein comprises a VH domain comprising an amino acid sequence according to SEQ ID NO: 9 and a VL domain comprising an amino acid sequence according to SEQ ID NO: 10. In some embodiments, the antibody of the first fusion protein comprises a VH domain comprising an amino acid sequence according to SEQ ID NO: 11 and a VL domain comprising an amino acid sequence according to SEQ ID NO: 12. In some embodiments, the antibody of the first fusion protein comprises a VH domain comprising an amino acid sequence according to SEQ ID NO: 13 and a VL domain comprising an amino acid sequence according to SEQ ID NO: 14. In some embodiments, the antibody of the first fusion protein comprises a VH domain comprising an amino acid sequence according to SEQ ID NO: 15 and a VL domain comprising an amino acid sequence according to SEQ ID NO: 16.

In some embodiments, the antibody of the second fusion protein comprises a VH domain comprising an amino acid sequence according to SEQ ID NO: 1 and a VL domain comprising an amino acid sequence according to SEQ ID NO: 2. In some embodiments, the antibody of the second fusion protein comprises a VH domain comprising an amino acid sequence according to SEQ ID NO: 3 and a VL domain comprising an amino acid sequence according to SEQ ID NO: 4. In some embodiments, the antibody of the second fusion protein comprises a VH domain comprising an amino acid sequence according to SEQ ID NO: 5 and a VL domain comprising an amino acid sequence according to SEQ ID NO: 6. In some embodiments, the antibody of the second fusion protein comprises a VH domain comprising an amino acid sequence according to SEQ ID NO: 7 and a VL domain comprising an amino acid sequence according to SEQ ID NO: 8. In some embodiments, the antibody of the second fusion protein comprises a VH domain comprising an amino acid sequence according to SEQ ID NO: 9 and a VL domain comprising an amino acid sequence according to SEQ ID NO: 10. In some embodiments, the antibody of the second fusion protein comprises a VH domain comprising an amino acid sequence according to SEQ ID NO: 11 and a VL domain comprising an amino acid sequence according to SEQ ID NO: 12. In some embodiments, the antibody of the second fusion protein comprises a VH domain comprising an amino acid sequence according to SEQ ID NO: 13 and a VL domain comprising an amino acid sequence according to SEQ ID NO: 14. In some embodiments, the antibody of the second fusion protein comprises a VH domain comprising an amino acid sequence according to SEQ ID NO: 15 and a VL domain comprising an amino acid sequence according to SEQ ID NO: 16.

In some embodiments, the antibody of the first fusion protein comprises a VH comprising an amino acid sequence having at least 75% sequence identity to an amino acid sequence according to any one of SEQ ID Nos: 1, 3, 5, 7, 9, 11, 13, and 15, and a VL comprising an amino acid sequence having at least 75% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 76% sequence identity to an amino acid sequence according to any one of SEQ ID Nos: 1, 3, 5, 7, 9, 11, 13, and 15, and a VL comprising an amino acid sequence having at least 76% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 77% sequence identity to an amino acid sequence according to any one of SEQ ID Nos: 1, 3, 5, 7, 9, 11, 13, and 15, and a VL comprising an amino acid sequence having at least 77% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 78% sequence identity to an amino acid sequence according to any one of SEQ ID Nos: 1, 3, 5, 7, 9, 11, 13, and 15, and a VL comprising an amino acid sequence having at least 78% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 79% sequence identity to an amino acid sequence according to any one of SEQ ID Nos: 1, 3, 5, 7, 9, 11, 13, and 15, and a VL comprising an amino acid sequence having at least 79% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 80% sequence identity to an amino acid sequence according to any one of SEQ ID Nos: 1, 3, 5, 7, 9, 11, 13, and 15, and a VL comprising an amino acid sequence having at least 80% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 81% sequence identity to an amino acid sequence according to any one of SEQ ID Nos: 1, 3, 5, 7, 9, 11, 13, and 15, and a VL comprising an amino acid sequence having at least 81% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 82% sequence identity to an amino acid sequence according to any one of SEQ ID Nos: 1, 3, 5, 7, 9, 11, 13, and 15, and a VL comprising an amino acid sequence having at least 82% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 83% sequence identity to an amino acid sequence according to any one of SEQ ID Nos: 1, 3, 5, 7, 9, 11, 13, and 15, and a VL comprising an amino acid sequence having at least 83% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 84% sequence identity to an amino acid sequence according to any one of SEQ ID Nos: 1, 3, 5, 7, 9, 11, 13, and 15, and a VL comprising an amino acid sequence having at least 84% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 85% sequence identity to an amino acid sequence according to any one of SEQ ID Nos: 1, 3, 5, 7, 9, 11, 13, and 15, and a VL comprising an amino acid sequence having at least 85% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 86% sequence identity to an amino acid sequence according to any one of SEQ ID Nos: 1, 3, 5, 7, 9, 11, 13, and 15, and a VL comprising an amino acid sequence having at least 86% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 87% sequence identity to an amino acid sequence according to any one of SEQ ID Nos: 1, 3, 5, 7, 9, 11, 13, and 15, and a VL comprising an amino acid sequence having at least 87% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 88% sequence identity to an amino acid sequence according to any one of SEQ ID Nos: 1, 3, 5, 7, 9, 11, 13, and 15, and a VL comprising an amino acid sequence having at least 88% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 89% sequence identity to an amino acid sequence according to any one of SEQ ID Nos: 1, 3, 5, 7, 9, 11, 13, and 15, and a VL comprising an amino acid sequence having at least 89% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 90% sequence identity to an amino acid sequence according to any one of SEQ ID Nos: 1, 3, 5, 7, 9, 11, 13, and 15, and a VL comprising an amino acid sequence having at least 90% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 91% sequence identity to an amino acid sequence according to any one of SEQ ID Nos: 1, 3, 5, 7, 9, 11, 13, and 15, and a VL comprising an amino acid sequence having at least 91% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 92% sequence identity to an amino acid sequence according to any one of SEQ ID Nos: 1, 3, 5, 7, 9, 11, 13, and 15, and a VL comprising an amino acid sequence having at least 92% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 93% sequence identity to an amino acid sequence according to any one of SEQ ID Nos: 1, 3, 5, 7, 9, 11, 13, and 15, and a VL comprising an amino acid sequence having at least 93% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 94% sequence identity to an amino acid sequence according to any one of SEQ ID Nos: 1, 3, 5, 7, 9, 11, 13, and 15, and a VL comprising an amino acid sequence having at least 94% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 95% sequence identity to an amino acid sequence according to any one of SEQ ID Nos: 1, 3, 5, 7, 9, 11, 13, and 15, and a VL comprising an amino acid sequence having at least 95% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 96% sequence identity to an amino acid sequence according to any one of SEQ ID Nos: 1, 3, 5, 7, 9, 11, 13, and 15, and a VL comprising an amino acid sequence having at least 96% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 97% sequence identity to an amino acid sequence according to any one of SEQ ID Nos: 1, 3, 5, 7, 9, 11, 13, and 15, and a VL comprising an amino acid sequence having at least 97% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 98% sequence identity to an amino acid sequence according to any one of SEQ ID Nos: 1, 3, 5, 7, 9, 11, 13, and 15, and a VL comprising an amino acid sequence having at least 98% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence having at least 99% sequence identity to an amino acid sequence according to any one of SEQ ID Nos: 1, 3, 5, 7, 9, 11, 13, and 15, and a VL comprising an amino acid sequence having at least 99% sequence identity to an amino acid sequence according to any one of SEQ ID Nos: 2, 4, 6, 8, 10, 12, 14, and 16. In some embodiments, the antibody of the first fusion protein comprises a VH comprising an amino acid sequence according to any one of SEQ ID Nos: 1, 3, 5, 7, 9, 11, 13, and 15, and a VL comprising an amino acid sequence according to any one of SEQ ID Nos: 2, 4, 6, 8, 10, 12, 14, and 16.

In some embodiments, the antibody of the second fusion protein comprises a VH comprising an amino acid sequence having at least 75% sequence identity to an amino acid sequence according to any one of SEQ ID Nos: 1, 3, 5, 7, 9, 11, 13, and 15, and a VL comprising an amino acid sequence having at least 75% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 76% sequence identity to an amino acid sequence according to any one of SEQ ID Nos: 1, 3, 5, 7, 9, 11, 13, and 15, and a VL comprising an amino acid sequence having at least 76% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 77% sequence identity to an amino acid sequence according to any one of SEQ ID Nos: 1, 3, 5, 7, 9, 11, 13, and 15, and a VL comprising an amino acid sequence having at least 77% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 78% sequence identity to an amino acid sequence according to any one of SEQ ID Nos: 1, 3, 5, 7, 9, 11, 13, and 15, and a VL comprising an amino acid sequence having at least 78% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 79% sequence identity to an amino acid sequence according to any one of SEQ ID Nos: 1, 3, 5, 7, 9, 11, 13, and 15, and a VL comprising an amino acid sequence having at least 79% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 80% sequence identity to an amino acid sequence according to any one of SEQ ID Nos: 1, 3, 5, 7, 9, 11, 13, and 15, and a VL comprising an amino acid sequence having at least 80% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 81% sequence identity to an amino acid sequence according to any one of SEQ ID Nos: 1, 3, 5, 7, 9, 11, 13, and 15, and a VL comprising an amino acid sequence having at least 81% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 82% sequence identity to an amino acid sequence according to any one of SEQ ID Nos: 1, 3, 5, 7, 9, 11, 13, and 15, and a VL comprising an amino acid sequence having at least 82% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 83% sequence identity to an amino acid sequence according to any one of SEQ ID Nos: 1, 3, 5, 7, 9, 11, 13, and 15, and a VL comprising an amino acid sequence having at least 83% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 84% sequence identity to an amino acid sequence according to any one of SEQ ID Nos: 1, 3, 5, 7, 9, 11, 13, and 15, and a VL comprising an amino acid sequence having at least 84% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 85% sequence identity to an amino acid sequence according to any one of SEQ ID Nos: 1, 3, 5, 7, 9, 11, 13, and 15, and a VL comprising an amino acid sequence having at least 85% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 86% sequence identity to an amino acid sequence according to any one of SEQ ID Nos: 1, 3, 5, 7, 9, 11, 13, and 15, and a VL comprising an amino acid sequence having at least 86% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 87% sequence identity to an amino acid sequence according to any one of SEQ ID Nos: 1, 3, 5, 7, 9, 11, 13, and 15, and a VL comprising an amino acid sequence having at least 87% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 88% sequence identity to an amino acid sequence according to any one of SEQ ID Nos: 1, 3, 5, 7, 9, 11, 13, and 15, and a VL comprising an amino acid sequence having at least 88% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 89% sequence identity to an amino acid sequence according to any one of SEQ ID Nos: 1, 3, 5, 7, 9, 11, 13, and 15, and a VL comprising an amino acid sequence having at least 89% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 90% sequence identity to an amino acid sequence according to any one of SEQ ID Nos: 1, 3, 5, 7, 9, 11, 13, and 15, and a VL comprising an amino acid sequence having at least 90% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 91% sequence identity to an amino acid sequence according to any one of SEQ ID Nos: 1, 3, 5, 7, 9, 11, 13, and 15, and a VL comprising an amino acid sequence having at least 91% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 92% sequence identity to an amino acid sequence according to any one of SEQ ID Nos: 1, 3, 5, 7, 9, 11, 13, and 15, and a VL comprising an amino acid sequence having at least 92% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 93% sequence identity to an amino acid sequence according to any one of SEQ ID Nos: 1, 3, 5, 7, 9, 11, 13, and 15, and a VL comprising an amino acid sequence having at least 93% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 94% sequence identity to an amino acid sequence according to any one of SEQ ID Nos: 1, 3, 5, 7, 9, 11, 13, and 15, and a VL comprising an amino acid sequence having at least 94% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 95% sequence identity to an amino acid sequence according to any one of SEQ ID Nos: 1, 3, 5, 7, 9, 11, 13, and 15, and a VL comprising an amino acid sequence having at least 95% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 96% sequence identity to an amino acid sequence according to any one of SEQ ID Nos: 1, 3, 5, 7, 9, 11, 13, and 15, and a VL comprising an amino acid sequence having at least 96% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 97% sequence identity to an amino acid sequence according to any one of SEQ ID Nos: 1, 3, 5, 7, 9, 11, 13, and 15, and a VL comprising an amino acid sequence having at least 97% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 98% sequence identity to an amino acid sequence according to any one of SEQ ID Nos: 1, 3, 5, 7, 9, 11, 13, and 15, and a VL comprising an amino acid sequence having at least 98% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence having at least 99% sequence identity to an amino acid sequence according to any one of SEQ ID Nos: 1, 3, 5, 7, 9, 11, 13, and 15, and a VL comprising an amino acid sequence having at least 99% sequence identity to an amino acid sequence according to any one of SEQ ID Nos: 2, 4, 6, 8, 10, 12, 14, and 16. In some embodiments, the antibody of the second fusion protein comprises a VH comprising an amino acid sequence according to any one of SEQ ID Nos: 1, 3, 5, 7, 9, 11, 13, and 15, and a VL comprising an amino acid sequence according to any one of SEQ ID Nos: 2, 4, 6, 8, 10, 12, 14, and 16.

In some embodiments, (a) the antibody of the first fusion protein comprises a VH comprising an amino acid sequence having at least 75% sequence identity to an amino acid sequence according to any one of SEQ ID Nos: 1, 3, 5, 7, 9, 11, 13, and 15, and a VL comprising an amino acid sequence having at least 75% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 76% sequence identity to an amino acid sequence according to any one of SEQ ID Nos: 1, 3, 5, 7, 9, 11, 13, and 15, and a VL comprising an amino acid sequence having at least 76% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 77% sequence identity to an amino acid sequence according to any one of SEQ ID Nos: 1, 3, 5, 7, 9, 11, 13, and 15, and a VL comprising an amino acid sequence having at least 77% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 78% sequence identity to an amino acid sequence according to any one of SEQ ID Nos: 1, 3, 5, 7, 9, 11, 13, and 15, and a VL comprising an amino acid sequence having at least 78% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 79% sequence identity to an amino acid sequence according to any one of SEQ ID Nos: 1, 3, 5, 7, 9, 11, 13, and 15, and a VL comprising an amino acid sequence having at least 79% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 80% sequence identity to an amino acid sequence according to any one of SEQ ID Nos: 1, 3, 5, 7, 9, 11, 13, and 15, and a VL comprising an amino acid sequence having at least 80% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 81% sequence identity to an amino acid sequence according to any one of SEQ ID Nos: 1, 3, 5, 7, 9, 11, 13, and 15, and a VL comprising an amino acid sequence having at least 81% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 82% sequence identity to an amino acid sequence according to any one of SEQ ID Nos: 1, 3, 5, 7, 9, 11, 13, and 15, and a VL comprising an amino acid sequence having at least 82% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 83% sequence identity to an amino acid sequence according to any one of SEQ ID Nos: 1, 3, 5, 7, 9, 11, 13, and 15, and a VL comprising an amino acid sequence having at least 83% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 84% sequence identity to an amino acid sequence according to any one of SEQ ID Nos: 1, 3, 5, 7, 9, 11, 13, and 15, and a VL comprising an amino acid sequence having at least 84% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 85% sequence identity to an amino acid sequence according to any one of SEQ ID Nos: 1, 3, 5, 7, 9, 11, 13, and 15, and a VL comprising an amino acid sequence having at least 85% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 86% sequence identity to an amino acid sequence according to any one of SEQ ID Nos: 1, 3, 5, 7, 9, 11, 13, and 15, and a VL comprising an amino acid sequence having at least 86% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 87% sequence identity to an amino acid sequence according to any one of SEQ ID Nos: 1, 3, 5, 7, 9, 11, 13, and 15, and a VL comprising an amino acid sequence having at least 87% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 88% sequence identity to an amino acid sequence according to any one of SEQ ID Nos: 1, 3, 5, 7, 9, 11, 13, and 15, and a VL comprising an amino acid sequence having at least 88% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 89% sequence identity to an amino acid sequence according to any one of SEQ ID Nos: 1, 3, 5, 7, 9, 11, 13, and 15, and a VL comprising an amino acid sequence having at least 89% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 90% sequence identity to an amino acid sequence according to any one of SEQ ID Nos: 1, 3, 5, 7, 9, 11, 13, and 15, and a VL comprising an amino acid sequence having at least 90% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 91% sequence identity to an amino acid sequence according to any one of SEQ ID Nos: 1, 3, 5, 7, 9, 11, 13, and 15, and a VL comprising an amino acid sequence having at least 91% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 92% sequence identity to an amino acid sequence according to any one of SEQ ID Nos: 1, 3, 5, 7, 9, 11, 13, and 15, and a VL comprising an amino acid sequence having at least 92% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 93% sequence identity to an amino acid sequence according to any one of SEQ ID Nos: 1, 3, 5, 7, 9, 11, 13, and 15, and a VL comprising an amino acid sequence having at least 93% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 94% sequence identity to an amino acid sequence according to any one of SEQ ID Nos: 1, 3, 5, 7, 9, 11, 13, and 15, and a VL comprising an amino acid sequence having at least 94% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 95% sequence identity to an amino acid sequence according to any one of SEQ ID Nos: 1, 3, 5, 7, 9, 11, 13, and 15, and a VL comprising an amino acid sequence having at least 95% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 96% sequence identity to an amino acid sequence according to any one of SEQ ID Nos: 1, 3, 5, 7, 9, 11, 13, and 15, and a VL comprising an amino acid sequence having at least 96% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 97% sequence identity to an amino acid sequence according to any one of SEQ ID Nos: 1, 3, 5, 7, 9, 11, 13, and 15, and a VL comprising an amino acid sequence having at least 97% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 98% sequence identity to an amino acid sequence according to any one of SEQ ID Nos: 1, 3, 5, 7, 9, 11, 13, and 15, and a VL comprising an amino acid sequence having at least 98% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence having at least 99% sequence identity to an amino acid sequence according to any one of SEQ ID Nos: 1, 3, 5, 7, 9, 11, 13, and 15, and a VL comprising an amino acid sequence having at least 99% sequence identity to an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, (a) the antibody of the first fusion protein comprises a VH comprising an amino acid sequence according to any one of SEQ ID Nos: 1, 3, 5, 7, 9, 11, 13, and 15, and a VL comprising an amino acid sequence according to any one of SEQ ID Nos: 2, 4, 6, 8, 10, 12, 14, and 16; and (b) the immune modulating polypeptide of the second fusion protein comprises an NK cell activating ligand, an immune checkpoint molecule, an inflammatory cytokine, or a proliferation cytokine. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-α, or TNF-β. In some embodiments, the proliferation cytokine comprises IL-7, IL-15, or IL-21.

### Oligomerization Domain

In some embodiments, the first fusion protein is monomeric. In some embodiments, the first fusion protein comprises an oligomerization domain. In some embodiments, the second fusion protein is monomeric. In some embodiments, the second fusion protein comprises an oligomerization domain.

In some embodiments, the oligomerization domain of the first or second fusion protein comprises a dimerization domain. In some embodiments, the dimerization domain comprises a leucine zipper dimerization domain. In some embodiments, the oligomerization domain of the first or second fusion protein comprises a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain of the first or second fusion protein comprises a tetramerization domain. In some embodiments, the tetramerization domain comprises an influenza neuraminidase stem domain.

TABLE 2
Exemplary Oligomerization Domain Sequences

| Oligomerization | | | SEQ ID |
|---|---|---|---|
| Domain | Oligomer | Amino Acid Sequence | NO. |
| D4 Variation 1 | Trimer | IGTALQVKMPKSHKAIQADGWMCHASKW | 17 |
| | | VTTCDFRWYGPKYITHSIRSFTPSVEQC | |
| | | KESIEQTKQGTWLNPGFPPQSCGYATVT | |
| | | D7AEAVIVQVTPHHVLVDEYTGEWVDSQ | |
| | | FIN8GKCSNYICPTVHNSTTWHSDYKVK | |
| | | GLCDSNLISMDI | |
| D4 Variation 2 | Trimer | IQADGWMCHASKWVTTCDFRWYGPKYIT | 18 |
| | | HSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STTWHSDYKVKGLCDSNL | |
| D4 Variation 3 | Trimer | IQADGWMCHASKWVTTCDFRWYGPKYIT | 19 |
| | | HSIRSFTPSVEQCKESIEQTKQGTWLNP | |
| | | GFPPQSCGYATVIDAEAVIVQVTPHHVL | |
| | | VDEYTGEWVDSQFINGKCSNYICPTVHN | |
| | | STT | |
| D4 Variation 4 | Trimer | IQADGWMCHASKWVTTCDFRWYGPKYIT | 20 |
| | | HSIRSFTPSVEQCKESIEQTKQGTWLNP | |
| | | GFPPQSCGYATVIDAEAVIVQVTPHHVL | |
| | | VDEYTGEWVDSQFING | |
| D4 Variation 5 | Trimer | IQADGWMCHASKWVTTCDFRWYGPKYIT | 21 |
| | | HSIRSFTPSVEQCKESIEQTKQGTWLNP | |
| | | GFPPQSCGYATVTDAEAVIVQVTPHHVL | |
| Foldon | Trimer | GYIPEAPRDGQAYVRKDGEWVLLSTFL | 22 |
| Leucine Zipper | Dimer | RMKQLEDKVEELLSKQYHLENEVARLKK | 23 |
| V1 | | LVGER | |
| Leucine Zipper | Dimer | RMKQLEDKVEELLSKNYHLENEVARLKK | 24 |
| V2 | | LVGER | |
| Neuraminidase | Tetramer | MNPNQKIITIGSICLVVGLISLILQIGN | 25 |
| Stem V1 | | IISIWISHSIQT | |
| Neuraminidase | Tetramer | MNPNQKIITIGSICMVTGIVSLMLQIGN | 26 |
| Stem V2 | | MISIWVSHSIHTGNQHQSEPISNTNFLT | |
| | | EKAVASVKLAGNSSLCPIN | |
| Dengue E Fusion | Trimer | KLCIEAKISNTTTDSRCPTQGEATLVEE | 27 |
| V1 | | QDTNFVCRRTFVDRGHGNGCGLFGKGSL | |
| | | ITCAKFKCVTKL | |
| Dengue E Fusion | Trimer | IELLKTEVTNPAVLRKLCIEAKISNTTT | 28 |
| V2 | | DSRCPTQGEATLVEEQDTNFVCRRTFVD | |
| | | RGHGNGCGLFGKGSLITCAKFKCVTKL | |
| Dengue E Fusion | Trimer | KLCIEAKISNTTTDSRCPTQGEATLVEE | 29 |
| V3 | | QDTNFVCRRTFVDRGHGNGCGLFGKGSL | |
| | | ITCAKFKCVTKLEGKIVQYENLKYSVI | |
| Dengue E Fusion | Trimer | EAKISNTTTDSRCPTQGEATLVEEQDTN | 30 |
| V4 | | FVCRRTFVDRGHGNGCGLFGKGSLITCA | |
| | | KFK | |

In some embodiments, the oligomerization domain of the first or second fusion protein comprises an amino acid sequence disclosed in Table 2, or an amino acid sequence that is substantially identical to an amino acid sequence in Table 2 (e.g. 7500, 8000, 8500, 90%, 95%, 96%, 97%, 98%, 99% sequence identity). In some instances, the oligomerization domain of the first or second fusion protein comprises an amino acid sequence comprising at least a portion having at least or about 10, 20, 30, 40, 50, 60, 70, 80, 90, 100, 110, 120, 130 amino acid sequences of any sequence according to Table 2.

In some embodiments, the oligomerization domain of the first or second fusion protein comprises an amino acid sequence that has at least 75% sequence identity to an amino acid sequence according to any one of SEQ ID NOs: 17-30. In some embodiments, the oligomerization domain of the first or second fusion protein comprises an amino acid sequence that has at least 76% sequence identity to an amino acid sequence according to any one of SEQ ID NOs: 17-30. In some embodiments, the oligomerization domain of the first or second fusion protein comprises an amino acid sequence that has at least 77% sequence identity to an amino acid sequence according to any one of SEQ ID NOs: 17-30. In some embodiments, the oligomerization domain of the first or second fusion protein comprises an amino acid sequence that has at least 78% sequence identity to an amino acid sequence according to any one of SEQ ID NOs: 17-30. In some embodiments, the oligomerization domain of the first or second fusion protein comprises an amino acid sequence that has at least 79% sequence identity to an amino acid sequence according to any one of SEQ ID NOs: 17-30. In some embodiments, the oligomerization domain of the first or second fusion protein comprises an amino acid sequence that has at least 80% sequence identity to an amino acid sequence according to any one of SEQ ID NOs: 17-30. In some embodiments, the oligomerization domain of the first or second fusion protein comprises an amino acid sequence that has at least 81% sequence identity to an amino acid sequence according to any one of SEQ ID NOs: 17-30. In some embodiments, the oligomerization domain of the first or second fusion protein comprises an amino acid sequence that has at least 82% sequence identity to an amino acid sequence according to any one of SEQ ID NOs: 17-30. In some embodiments, the oligomerization domain of the first or second fusion protein comprises an amino acid sequence that has at least 83% sequence identity to an amino acid sequence according to any one of SEQ ID NOs: 17-30. In some embodiments, the oligomerization domain of the first or second fusion protein comprises an amino acid sequence that has at least 84% sequence identity to an amino acid sequence according to any one of SEQ ID NOs: 17-30. In some embodiments, the oligomerization domain of the first or second fusion protein comprises an amino acid sequence that has at least 85% sequence identity to an amino acid sequence according to any one of SEQ ID NOs: 17-30. In some embodiments, the oligomerization domain of the first or second fusion protein comprises an amino acid sequence that has at least 86% sequence identity to an amino acid sequence according to any one of SEQ ID NOs: 17-30. In some embodiments, the oligomerization domain of the first or second fusion protein comprises an amino acid sequence that has at least 87% sequence identity to an amino acid sequence according to any one of SEQ ID NOs: 17-30. In some embodiments, the oligomerization domain of the first or second fusion protein comprises an amino acid sequence that has at least 88% sequence identity to an amino acid sequence according to any one of SEQ ID NOs: 17-30. In some embodiments, the oligomerization domain of the first or second fusion protein comprises an amino acid sequence that has at least 89% sequence identity to an amino acid sequence according to any one of SEQ ID NOs: 17-30. In some embodiments, the oligomerization domain of the first or second fusion protein comprises an amino acid sequence that has at least 90% sequence identity to an amino acid sequence according to any one of SEQ ID NOs: 17-30. In some embodiments, the oligomerization domain of the first or second fusion protein comprises an amino acid sequence that has at least 91% sequence identity to an amino acid sequence according to any one of SEQ ID NOs: 17-30. In some embodiments, the oligomerization domain of the first or second fusion protein comprises an amino acid sequence that has at least 92% sequence identity to an amino acid sequence according to any one of SEQ ID NOs: 17-30. In some embodiments, the oligomerization domain of the first or second fusion protein comprises an amino acid sequence that has at least 93% sequence identity to an amino acid sequence according to any one of SEQ ID NOs: 17-30. In some embodiments, the oligomerization domain of the first or second fusion protein comprises an amino acid sequence that has at least 94% sequence identity to an amino acid sequence according to any one of SEQ ID NOs: 17-30. In some embodiments, the oligomerization domain of the first or second fusion protein comprises an amino acid sequence that has at least 95% sequence identity to an amino acid sequence according to any one of SEQ ID NOs: 17-30. In some embodiments, the oligomerization domain of the first or second fusion protein comprises an amino acid sequence that has at least 96% sequence identity to an amino acid sequence according to any one of SEQ ID NOs: 17-30. In some embodiments, the oligomerization domain of the first or second fusion protein comprises an amino acid sequence that has at least 97% sequence identity to an amino acid sequence according to any one of SEQ ID NOs: 17-30. In some embodiments, the oligomerization domain of the first or second fusion protein comprises an amino acid sequence that has at least 98% sequence identity to an amino acid sequence according to any one of SEQ ID NOs: 17-30. In some embodiments, the oligomerization domain of the first or second fusion protein comprises an amino acid sequence that has at least 99% sequence identity to an amino acid sequence according to any one of SEQ ID NOs: 17-30. In some embodiments, the oligomerization domain of the first or second fusion protein comprises an amino acid sequence according to any one of SEQ ID NOs: 17-30.

In some embodiments, the first fusion protein comprises a signal peptide. In some embodiments, domains of the first fusion protein are arranged from the N-terminus to the C-terminus in the following orders: (a) signal peptide, antibody, oligomerization domain, and transmembrane polypeptide; (b) signal peptide, antibody, transmembrane polypeptide, and oligomerization domain; or (c) signal peptide, oligomerization domain, antibody, and transmembrane polypeptide.

In some embodiments, the first fusion protein further comprises a cytosolic domain. In some embodiments, the domains of the first fusion protein are arranged from the N-terminus to the C-terminus in the following orders: (a) signal peptide, antibody, oligomerization domain, transmembrane polypeptide, and cytosolic domain; (b) signal peptide, antibody, transmembrane polypeptide, oligomerization domain, and cytosolic domain; or (c) signal peptide, oligomerization domain, antibody, transmembrane polypeptide, and cytosolic domain.

In some embodiments, the second fusion protein comprises a signal peptide. In some embodiments, the domains of the second fusion protein are arranged from the N-terminus to the C-terminus in the following orders: (a) signal peptide, antibody, oligomerization domain, and transmembrane polypeptide; (b) signal peptide, antibody, transmembrane polypeptide, and oligomerization domain; or (c) signal peptide, oligomerization domain, antibody, and transmembrane polypeptide.

In some embodiments, the second fusion protein further comprises a cytosolic domain. In some embodiments, domains of the second fusion protein are arranged from the N-terminus to the C-terminus in the following orders: (a) signal peptide, antibody, oligomerization domain, transmembrane polypeptide, and cytosolic domain; (b) signal peptide, antibody, transmembrane polypeptide, oligomerization domain, and cytosolic domain; or (c) signal peptide, oligomerization domain, antibody, transmembrane polypeptide, and cytosolic domain.

### Transmembrane Domain

In some embodiments, the transmembrane polypeptide anchors the first fusion protein to a bilayer of the HBV-antivirus. In some embodiments, the transmembrane polypeptide anchors the second fusion protein to a bilayer of the HBV-antivirus. In some embodiments, the transmembrane polypeptide of the first fusion protein or the second fusion protein comprises the transmembrane domain of a Vesicular Stomatitis virus glycoprotein (VSV-G). In some embodiments, the transmembrane polypeptide of the first fusion protein or the second fusion protein comprises the cytosolic tail of VSV-G. In some embodiments, the transmembrane polypeptide of the first fusion protein or the second fusion protein comprises the transmembrane domain of influenza Neuraminidase (NA). In some embodiments, the transmembrane polypeptide of the first fusion protein or the second fusion protein comprises the transmembrane domain of Dengue E Protein. In some embodiments, the transmembrane polypeptide of the first fusion protein or the second fusion protein comprises the transmembrane domain of influenza Hemagglutinin (HA). In some embodiments, the transmembrane polypeptide comprises the transmembrane domain of HIV surface glycoprotein GP120 or GP41. In some embodiments, the transmembrane polypeptide comprises the transmembrane domain of measles virus surface glycoprotein hemagglutinin (H) protein.

In some embodiments, the transmembrane polypeptide of the first fusion protein or the second fusion protein comprises the transmembrane domain of a Vesicular Stomatitis virus glycoprotein (VSV-G). In some embodiments, the transmembrane polypeptide of the first fusion protein or the second fusion protein comprises the transmembrane domain and cytosolic domain of a Vesicular Stomatitis virus glycoprotein (VSV-G). In some embodiments, the transmembrane polypeptide of the first fusion protein or the second fusion protein comprises the transmembrane domain of a Dengue E protein. In some embodiments, the transmembrane polypeptide of the first fusion protein or the second fusion protein comprises the transmembrane domain and cytosolic domain of a Dengue E protein. In some embodiments, the transmembrane polypeptide of the first fusion protein or the second fusion protein comprises the transmembrane domain of influenza Hemagglutinin (HA). In some embodiments, the transmembrane polypeptide of the first fusion protein or the second fusion protein comprises the transmembrane domain and cytosolic domain of influenza Hemagglutinin (HA). In some embodiments, the transmembrane polypeptide of the first fusion protein or the second fusion protein comprises the transmembrane domain of HIV surface glycoprotein GP120 or GP41. In some embodiments, the transmembrane polypeptide of the first fusion protein or the second fusion protein comprises the transmembrane domain and cytosolic domain of HIV surface glycoprotein GP120 or GP41. In some embodiments, the transmembrane polypeptide of the first fusion protein or the second fusion protein comprises the transmembrane domain of measles virus surface glycoprotein hemagglutinin (H) protein. In some embodiments, the transmembrane polypeptide of the first fusion protein or the second fusion protein comprises the transmembrane domain and cytosolic domain of measles virus surface glycoprotein hemagglutinin (H) protein. In some embodiments, the transmembrane polypeptide of the first fusion protein or the second fusion protein comprises the transmembrane domain of influenza Neuraminidase (NA). In some embodiments, the transmembrane polypeptide of the first fusion protein or the second fusion protein comprises the transmembrane domain and cytosolic domain of influenza Neuraminidase (NA).

TABLE 3
Exemplary Transmembrane Domain Sequences

| | | SEQ ID |
|---|---|---|
| Domain | Amino Acid Sequence | NO: |
| VSV-G | IASFFFIIGLIIGLFLVLRVGI | 31 |
| Transmembrane | | |
| (TM) V1 | | |
| VSV-G | PIELVEGWFSSWKSSIASFFFI | 32 |
| Transmembrane | IGLIIGLFLVLRVGI | |
| (TM) V2 | | |
| VSV-G | DDESLFFGDTGLSKNPIELVEG | 33 |
| Transmembrane | WFSSWKSSIASFFFIIGLIIGL | |
| (TM) V3 | FLVLRVGIH | |
| VSV-G | GMLDSDLHLSSKAQVFEHPHIQ | 34 |
| Transmembrane | DAASQLPDDESLFFGDTGLSKN | |
| (TM) V4 | PIELVEGWFSSWKSSIASFFFI | |
| | IGLIIGLFLVLRVGI | |
| VSV-G | HLCIKLKHTKKRQIYTDIEMNR | 35 |
| Cytosolic Tail | LGK | |
| (CT) | | |
| Influenza | IITIGSVCMTIGMANLILQIGN | 36 |
| Neuraminidase | I | |
| TM (NA) | | |
| Influenza | LAIYSTVASSLVLVVSLGAISF | 37 |
| Hemagglutinin | W | |
| TM (HA) | | |
| Dengue E | AYGVLFSGVSWTMKIGIGILLT | 38 |
| Protein TM | WLGLNSRSTSLSMTCIAVGMVT | |
| | LYLGVMVQ | |
| HIV gp TM | FIMIVGGLVGLRIVFAVLSIV | 39 |

In some embodiments, the transmembrane domain of the first fusion protein or the second fusion protein comprises an amino acid sequence disclosed in Table 3, or an amino acid sequence that is substantially identical to an amino acid sequence in Table 3 (e.g., 7500, 8000, 8500, 9000, 9500, 9600, 9700, 9800, 9900 sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 3.

In some embodiments, the transmembrane domain of the first fusion protein or the second fusion protein comprises an amino acid sequence that has at least 75% sequence identity to an amino acid sequence according to any one of SEQ ID Nos: 31-39. In some embodiments, the transmembrane domain of the first fusion protein or the second fusion protein comprises an amino acid sequence that has at least 76% sequence identity to an amino acid sequence according to any one of SEQ ID Nos: 31-39. In some embodiments, the transmembrane domain of the first fusion protein or the second fusion protein comprises an amino acid sequence that has at least 77% sequence identity to an amino acid sequence according to any one of SEQ ID Nos: 31-39. In some embodiments, the transmembrane domain of the first fusion protein or the second fusion protein comprises an amino acid sequence that has at least 78% sequence identity to an amino acid sequence according to any one of SEQ ID Nos: 31-39. In some embodiments, the transmembrane domain of the first fusion protein or the second fusion protein comprises an amino acid sequence that has at least 79% sequence identity to an amino acid sequence according to any one of SEQ ID Nos: 31-39. In some embodiments, the transmembrane domain of the first fusion protein or the second fusion protein comprises an amino acid sequence that has at least 80% sequence identity to an amino acid sequence according to any one of SEQ ID Nos: 31-39. In some embodiments, the transmembrane domain of the first fusion protein or the second fusion protein comprises an amino acid sequence that has at least 81% sequence identity to an amino acid sequence according to any one of SEQ ID Nos: 31-39. In some embodiments, the transmembrane domain of the first fusion protein or the second fusion protein comprises an amino acid sequence that has at least 82% sequence identity to an amino acid sequence according to any one of SEQ ID Nos: 31-39. In some embodiments, the transmembrane domain of the first fusion protein or the second fusion protein comprises an amino acid sequence that has at least 83% sequence identity to an amino acid sequence according to any one of SEQ ID Nos: 31-39. In some embodiments, the transmembrane domain of the first fusion protein or the second fusion protein comprises an amino acid sequence that has at least 84% sequence identity to an amino acid sequence according to any one of SEQ ID Nos: 31-39. In some embodiments, the transmembrane domain of the first fusion protein or the second fusion protein comprises an amino acid sequence that has at least 85% sequence identity to an amino acid sequence according to any one of SEQ ID Nos: 31-39. In some embodiments, the transmembrane domain of the first fusion protein or the second fusion protein comprises an amino acid sequence that has at least 86% sequence identity to an amino acid sequence according to any one of SEQ ID Nos: 31-39. In some embodiments, the transmembrane domain of the first fusion protein or the second fusion protein comprises an amino acid sequence that has at least 87% sequence identity to an amino acid sequence according to any one of SEQ ID Nos: 31-39. In some embodiments, the transmembrane domain of the first fusion protein or the second fusion protein comprises an amino acid sequence that has at least 88% sequence identity to an amino acid sequence according to any one of SEQ ID Nos: 31-39. In some embodiments, the transmembrane domain of the first fusion protein or the second fusion protein comprises an amino acid sequence that has at least 89% sequence identity to an amino acid sequence according to any one of SEQ ID Nos: 31-39. In some embodiments, the transmembrane domain of the first fusion protein or the second fusion protein comprises an amino acid sequence that has at least 90% sequence identity to an amino acid sequence according to any one of SEQ ID Nos: 31-39. In some embodiments, the transmembrane domain of the first fusion protein or the second fusion protein comprises an amino acid sequence that has at least 91% sequence identity to an amino acid sequence according to any one of SEQ ID Nos: 31-39. In some embodiments, the transmembrane domain of the first fusion protein or the second fusion protein comprises an amino acid sequence that has at least 92% sequence identity to an amino acid sequence according to any one of SEQ ID Nos: 31-39. In some embodiments, the transmembrane domain of the first fusion protein or the second fusion protein comprises an amino acid sequence that has at least 93% sequence identity to an amino acid sequence according to any one of SEQ ID Nos: 31-39. In some embodiments, the transmembrane domain of the first fusion protein or the second fusion protein comprises an amino acid sequence that has at least 94% sequence identity to an amino acid sequence according to any one of SEQ ID Nos: 31-39. In some embodiments, the transmembrane domain of the first fusion protein or the second fusion protein comprises an amino acid sequence that has at least 95% sequence identity to an amino acid sequence according to any one of SEQ ID Nos: 31-39. In some embodiments, the transmembrane domain of the first fusion protein or the second fusion protein comprises an amino acid sequence that has at least 96% sequence identity to an amino acid sequence according to any one of SEQ ID Nos: 31-39. In some embodiments, the transmembrane domain of the first fusion protein or the second fusion protein comprises an amino acid sequence that has at least 97% sequence identity to an amino acid sequence according to any one of SEQ ID Nos: 31-39. In some embodiments, the transmembrane domain of the first fusion protein or the second fusion protein comprises an amino acid sequence that has at least 98% sequence identity to an amino acid sequence according to any one of SEQ ID Nos: 31-39. In some embodiments, the transmembrane domain of the first fusion protein or the second fusion protein comprises an amino acid sequence that has at least 99% sequence identity to an amino acid sequence according to any one of SEQ ID Nos: 31-39. In some embodiments, the transmembrane domain of the first fusion protein or the second fusion protein comprises an amino acid sequence according to any one of SEQ ID Nos: 31-39.

In some embodiments, the antibody of the first fusion protein comprises an amino acid sequence from at least one complementarity determining region of 2H5, ADRI-2F3, H004, H009, H007, H015, H019, or H020, and the transmembrane polypeptide of the first fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, the antibody of the second fusion protein comprises an amino acid sequence from at least one complementarity determining region of 2H5, ADRI-2F3, H004, H009, H007, H015, H019, or H020, and the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, the antibody of the first fusion protein comprises an amino acid sequence from at least one complementarity determining region of 2H5, ADRI-2F3, H004, H009, H007, H015, H019, or H020, and the transmembrane polypeptide of the first fusion protein comprises the transmembrane domain and cytosolic domain of VSV-G, the transmembrane domain and cytosolic domain of influenza Neuraminidase (NA), the transmembrane domain and cytosolic domain of influenza Hemagglutinin (HA), the transmembrane domain and cytosolic domain of Dengue E Protein, the transmembrane domain and cytosolic domain of GP120 or GP41, or the transmembrane domain and cytosolic domain of measles virus surface glycoprotein hemagglutinin (H) protein. In some embodiments, the antibody of the second fusion protein comprises an amino acid sequence from at least one complementarity determining region of 2H5, ADRI-2F3, H004, H009, H007, H015, H019, or H020, and the transmembrane polypeptide of the second fusion protein comprises the transmembrane domain and cytosolic domain of VSV-G, the transmembrane domain and cytosolic domain of influenza Neuraminidase (NA), the transmembrane domain and cytosolic domain of influenza Hemagglutinin (HA), the transmembrane domain and cytosolic domain of Dengue E Protein, the transmembrane domain and cytosolic domain of GP120 or GP41, or the transmembrane domain and cytosolic domain of measles virus surface glycoprotein hemagglutinin (H) protein. In some embodiments, the antibody of the first fusion protein comprises an amino acid sequence from at least one complementarity determining region of 2H5, ADRI-2F3, H004, H009, H007, H015, H019, or H020, and the transmembrane polypeptide of the first fusion protein comprises the transmembrane domain and cytosolic domain of VSV-G. In some embodiments, the antibody of the second fusion protein comprises an amino acid sequence from at least one complementarity determining region of 2H5, ADRI-2F3, H004, H009, H007, H015, H019, or H020, and the transmembrane polypeptide of the second fusion protein comprises the transmembrane domain and cytosolic domain of VSV-G. In some embodiments, the antibody of the first fusion protein comprises an amino acid sequence comprising at least two complementarity determining regions from at least two antibodies selected from the group comprising 2H5, ADRI-2F3, H004, H009, H007, H015, H019, or H020, and the transmembrane polypeptide of the first fusion protein comprises the transmembrane domain and cytosolic domain of VSV-G. In some embodiments, the antibody of the second fusion protein comprises an amino acid sequence comprising at least two complementarity determining regions from at least two antibodies selected from the group comprising 2H5, ADRI-2F3, H004, H009, H007, H015, H019, or H020, and the transmembrane polypeptide of the second fusion protein comprises the transmembrane domain and cytosolic domain of VSV-G.

In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises the transmembrane domain and cytosolic domain of VSV-G, the transmembrane domain and cytosolic domain of influenza Neuraminidase (NA), the transmembrane domain and cytosolic domain of influenza Hemagglutinin (HA), the transmembrane domain and cytosolic domain of Dengue E Protein, the transmembrane domain and cytosolic domain of GP120 or GP41, or the transmembrane domain and cytosolic domain of measles virus surface glycoprotein hemagglutinin (H) protein. In some embodiments, the immune modulating polypeptide of the second fusion protein comprises an NK cell activating ligand, an immune checkpoint molecule, an inflammatory cytokine, or a proliferation cytokine, and the transmembrane polypeptide of the first fusion protein comprises the transmembrane domain and cytosolic domain of VSV-G. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises the transmembrane domain and cytosolic domain of VSV-G. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-α, or TNF-β. In some embodiments, the proliferation cytokine comprises IL-7, IL-15, or IL-21.

In some embodiments, the antibody of the first fusion protein or the second fusion protein comprises a VH comprising an amino acid sequence having at least 75% sequence identity to an amino acid sequence of any one of SEQ ID Nos: 1, 3, 5, 7, 9, 11, 13, and 15 and a VL comprising an amino acid sequence having at least 75% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 75% sequence identity to an amino acid sequence of any one of SEQ ID Nos: 31-39. In some embodiments, the antibody of the first fusion protein or the second fusion protein comprises a VH comprising an amino acid sequence having at least 76% sequence identity to an amino acid sequence of any one of SEQ ID Nos: 1, 3, 5, 7, 9, 11, 13, and 15 and a VL comprising an amino acid sequence having at least 76% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 76% sequence identity to an amino acid sequence of any one of SEQ ID Nos: 31-39. In some embodiments, the antibody of the first fusion protein or the second fusion protein comprises a VH comprising an amino acid sequence having at least 77% sequence identity to an amino acid sequence of any one of SEQ ID Nos: 1, 3, 5, 7, 9, 11, 13, and 15 and a VL comprising an amino acid sequence having at least 77% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 77% sequence identity to an amino acid sequence of any one of SEQ ID Nos: 31-39. In some embodiments, the antibody of the first fusion protein or the second fusion protein comprises a VH comprising an amino acid sequence having at least 78% sequence identity to an amino acid sequence of any one of SEQ ID Nos: 1, 3, 5, 7, 9, 11, 13, and 15 and a VL comprising an amino acid sequence having at least 78% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 78% sequence identity to an amino acid sequence of any one of SEQ ID Nos: 31-39. In some embodiments, the antibody of the first fusion protein or the second fusion protein comprises a VH comprising an amino acid sequence having at least 79% sequence identity to an amino acid sequence of any one of SEQ ID Nos: 1, 3, 5, 7, 9, 11, 13, and 15 and a VL comprising an amino acid sequence having at least 79% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 79% sequence identity to an amino acid sequence of any one of SEQ ID Nos: 31-39. In some embodiments, the antibody of the first fusion protein or the second fusion protein comprises a VH comprising an amino acid sequence having at least 80% sequence identity to an amino acid sequence of any one of SEQ ID Nos: 1, 3, 5, 7, 9, 11, 13, and 15 and a VL comprising an amino acid sequence having at least 80% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 80% sequence identity to an amino acid sequence of any one of SEQ ID Nos: 31-39. In some embodiments, the antibody of the first fusion protein or the second fusion protein comprises a VH comprising an amino acid sequence having at least 81% sequence identity to an amino acid sequence of any one of SEQ ID Nos: 1, 3, 5, 7, 9, 11, 13, and 15 and a VL comprising an amino acid sequence having at least 81% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 81% sequence identity to an amino acid sequence of any one of SEQ ID Nos: 31-39. In some embodiments, the antibody of the first fusion protein or the second fusion protein comprises a VH comprising an amino acid sequence having at least 82% sequence identity to an amino acid sequence of any one of SEQ ID Nos: 1, 3, 5, 7, 9, 11, 13, and 15 and a VL comprising an amino acid sequence having at least 82% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 82% sequence identity to an amino acid sequence of any one of SEQ ID Nos: 31-39. In some embodiments, the antibody of the first fusion protein or the second fusion protein comprises a VH comprising an amino acid sequence having at least 83% sequence identity to an amino acid sequence of any one of SEQ ID Nos: 1, 3, 5, 7, 9, 11, 13, and 15 and a VL comprising an amino acid sequence having at least 83% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 83% sequence identity to an amino acid sequence of any one of SEQ ID Nos: 31-39. In some embodiments, the antibody of the first fusion protein or the second fusion protein comprises a VH comprising an amino acid sequence having at least 84% sequence identity to an amino acid sequence of any one of SEQ ID Nos: 1, 3, 5, 7, 9, 11, 13, and 15 and a VL comprising an amino acid sequence having at least 84% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 84% sequence identity to an amino acid sequence of any one of SEQ ID Nos: 31-39. In some embodiments, the antibody of the first fusion protein or the second fusion protein comprises a VH comprising an amino acid sequence having at least 85% sequence identity to an amino acid sequence of any one of SEQ ID Nos: 1, 3, 5, 7, 9, 11, 13, and 15 and a VL comprising an amino acid sequence having at least 85% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 85% sequence identity to an amino acid sequence of any one of SEQ ID Nos: 31-39. In some embodiments, the antibody of the first fusion protein or the second fusion protein comprises a VH comprising an amino acid sequence having at least 86% sequence identity to an amino acid sequence of any one of SEQ ID Nos: 1, 3, 5, 7, 9, 11, 13, and 15 and a VL comprising an amino acid sequence having at least 86% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 86% sequence identity to an amino acid sequence of any one of SEQ ID Nos: 31-39. In some embodiments, the antibody of the first fusion protein or the second fusion protein comprises a VH comprising an amino acid sequence having at least 87% sequence identity to an amino acid sequence of any one of SEQ ID Nos: 1, 3, 5, 7, 9, 11, 13, and 15 and a VL comprising an amino acid sequence having at least 87% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 87% sequence identity to an amino acid sequence of any one of SEQ ID Nos: 31-39. In some embodiments, the antibody of the first fusion protein or the second fusion protein comprises a VH comprising an amino acid sequence having at least 88% sequence identity to an amino acid sequence of any one of SEQ ID Nos: 1, 3, 5, 7, 9, 11, 13, and 15 and a VL comprising an amino acid sequence having at least 88% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 88% sequence identity to an amino acid sequence of any one of SEQ ID Nos: 31-39. In some embodiments, the antibody of the first fusion protein or the second fusion protein comprises a VH comprising an amino acid sequence having at least 88% sequence identity to an amino acid sequence of any one of SEQ ID Nos: 1, 3, 5, 7, 9, 11, 13, and 15 and a VL comprising an amino acid sequence having at least 88% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 88% sequence identity to an amino acid sequence of any one of SEQ ID Nos: 31-39. In some embodiments, the antibody of the first fusion protein or the second fusion protein comprises a VH comprising an amino acid sequence having at least 90% sequence identity to an amino acid sequence of any one of SEQ ID Nos: 1, 3, 5, 7, 9, 11, 13, and 15 and a VL comprising an amino acid sequence having at least 90% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 90% sequence identity to an amino acid sequence of any one of SEQ ID Nos: 31-39. In some embodiments, the antibody of the first fusion protein or the second fusion protein comprises a VH comprising an amino acid sequence having at least 91% sequence identity to an amino acid sequence of any one of SEQ ID Nos: 1, 3, 5, 7, 9, 11, 13, and 15 and a VL comprising an amino acid sequence having at least 91% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 91% sequence identity to an amino acid sequence of any one of SEQ ID Nos: 31-39. In some embodiments, the antibody of the first fusion protein or the second fusion protein comprises a VH comprising an amino acid sequence having at least 92% sequence identity to an amino acid sequence of any one of SEQ ID Nos: 1, 3, 5, 7, 9, 11, 13, and 15 and a VL comprising an amino acid sequence having at least 92% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 92% sequence identity to an amino acid sequence of any one of SEQ ID Nos: 31-39. In some embodiments, the antibody of the first fusion protein or the second fusion protein comprises a VH comprising an amino acid sequence having at least 93% sequence identity to an amino acid sequence of any one of SEQ ID Nos: 1, 3, 5, 7, 9, 11, 13, and 15 and a VL comprising an amino acid sequence having at least 93% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 93% sequence identity to an amino acid sequence of any one of SEQ ID Nos: 31-39. In some embodiments, the antibody of the first fusion protein or the second fusion protein comprises a VH comprising an amino acid sequence having at least 94% sequence identity to an amino acid sequence of any one of SEQ ID Nos: 1, 3, 5, 7, 9, 11, 13, and 15 and a VL comprising an amino acid sequence having at least 94% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 94% sequence identity to an amino acid sequence of any one of SEQ ID Nos: 31-39. In some embodiments, the antibody of the first fusion protein or the second fusion protein comprises a VH comprising an amino acid sequence having at least 95% sequence identity to an amino acid sequence of any one of SEQ ID Nos: 1, 3, 5, 7, 9, 11, 13, and 15 and a VL comprising an amino acid sequence having at least 95% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 95% sequence identity to an amino acid sequence of any one of SEQ ID Nos: 31-39. In some embodiments, the antibody of the first fusion protein or the second fusion protein comprises a VH comprising an amino acid sequence having at least 96% sequence identity to an amino acid sequence of any one of SEQ ID Nos: 1, 3, 5, 7, 9, 11, 13, and 15 and a VL comprising an amino acid sequence having at least 96% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 96% sequence identity to an amino acid sequence of any one of SEQ ID Nos: 31-39. In some embodiments, the antibody of the first fusion protein or the second fusion protein comprises a VH comprising an amino acid sequence having at least 97% sequence identity to an amino acid sequence of any one of SEQ ID Nos: 1, 3, 5, 7, 9, 11, 13, and 15 and a VL comprising an amino acid sequence having at least 97% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 97% sequence identity to an amino acid sequence of any one of SEQ ID Nos: 31-39. In some embodiments, the antibody of the first fusion protein or the second fusion protein comprises a VH comprising an amino acid sequence having at least 98% sequence identity to an amino acid sequence of any one of SEQ ID Nos: 1, 3, 5, 7, 9, 11, 13, and 15 and a VL comprising an amino acid sequence having at least 98% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 98% sequence identity to an amino acid sequence of any one of SEQ ID Nos: 31-39. In some embodiments, the antibody of the first fusion protein or the second fusion protein comprises a VH comprising an amino acid sequence having at least 99% sequence identity to an amino acid sequence of any one of SEQ ID Nos: 1, 3, 5, 7, 9, 11, 13, and 15 and a VL comprising an amino acid sequence having at least 99% sequence identity to an amino acid sequence of any one of SEQ ID Nos: 2, 4, 6, 8, 10, 12, 14, and 16, and the transmembrane polypeptide of the first fusion protein or the second fusion protein comprises an amino acid sequence having at least 99% sequence identity to an amino acid sequence of any one of SEQ ID Nos: 31-39. In some embodiments, the antibody of the first fusion protein or the second fusion protein comprises a VH comprising an amino acid sequence of any one of SEQ ID Nos: 1, 3, 5, 7, 9, 11, 13, and 15 and a VL comprising an amino acid sequence of any one of SEQ ID Nos: 2, 4, 6, 8, 10, 12, 14, and 16, and the transmembrane polypeptide of the first fusion protein or the second fusion protein comprises an amino acid sequence of any one of SEQ ID Nos: 31-39.

In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 75% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 76% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 77% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 78% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 79% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 80% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 81% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 82% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 83% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 84% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 85% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 86% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 87% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 88% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 89% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 90% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 91% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 92% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 93% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 94% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 95% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 96% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 97% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 98% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence having at least 99% sequence identity to an amino acid sequence of any one of SEQ ID Nos: 31-39. In some embodiments, the immune modulating polypeptide of the second fusion protein comprises an NK cell activating ligand, an immune checkpoint molecule, an inflammatory cytokine, or a proliferation cytokine, and the transmembrane polypeptide of the second fusion protein comprises an amino acid sequence of any one of SEQ ID Nos: 31-39. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-α, or TNF-β. In some embodiments, the proliferation cytokine comprises TL-7, IL-15, or IL-21.

In some embodiments, the antibody of the first fusion protein or the second fusion protein comprises a VH comprising an amino acid sequence according to SEQ ID No: 1 and a VL comprising an amino acid sequence according to SEQ ID No: 2, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 3 and a VL comprising an amino acid sequence according to SEQ ID No: 4, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 5 and a VL comprising an amino acid sequence according to SEQ ID No: 6, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 7 and a VL comprising an amino acid sequence according to SEQ ID No: 8, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 9 and a VL comprising an amino acid sequence according to SEQ ID No: 10, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 11 and a VL comprising an amino acid sequence according to SEQ ID No: 12, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 13 and a VL comprising an amino acid sequence according to SEQ ID No: 14, and the transmembrane polypeptide of the first fusion protein or the second fusion protein comprises an amino acid sequence of any one of SEQ ID Nos: 31-39. In some embodiments, the antibody of the first fusion protein or the second fusion protein comprises a VH comprising an amino acid sequence according to SEQ ID No: 15 and a VL comprising an amino acid sequence according to SEQ ID No: 16, and the transmembrane polypeptide of the first fusion protein or the second fusion protein comprises an amino acid sequence of any one of SEQ ID Nos: 31-39.

In some embodiments, (a) the antibody of the first fusion protein comprises an amino acid sequence from at least one complementarity determining region of 2H5, ADRI-2F3, H004, H009, H007, H015, H019, or H020; (b) the transmembrane polypeptide of the first fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and (c) the oligomerization domain of the first fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the antibody of the first fusion protein comprises an amino acid sequence from at least one complementarity determining region of 2H5, ADRI-2F3, H004, H009, H007, H015, H019, or H020; (b) the transmembrane polypeptide of the first fusion protein comprises the transmembrane domain and cytosolic domain of VSV-G, the transmembrane domain and cytosolic domain of influenza Neuraminidase (NA), the transmembrane domain and cytosolic domain of influenza Hemagglutinin (HA), the transmembrane domain and cytosolic domain of Dengue E Protein, the transmembrane domain and cytosolic domain of GP120 or GP41, or the transmembrane domain and cytosolic domain of measles virus surface glycoprotein hemagglutinin (H) protein; and (c) the oligomerization domain of the first fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the antibody of the first fusion protein comprises an amino acid sequence from at least one complementarity determining region of 2H5, ADRI-2F3, H004, H009, H007, H015, H019, or H020; (b) the transmembrane polypeptide of the first fusion protein comprises the transmembrane domain of VSV-G; and (c) the oligomerization domain of the first fusion protein comprises a D4 post-fusion trimerization domain of VSV-G protein. In some embodiments, (a) the antibody of the first fusion protein comprises an amino acid sequence from at least one complementarity determining region of 2H5, ADRI-2F3, H004, H009, H007, H015, H019, or H020; (b) the transmembrane polypeptide of the first fusion protein comprises the transmembrane domain and cytosolic domain of VSV-G; and (c) the oligomerization domain of the first fusion protein comprises a D4 post-fusion trimerization domain of VSV-G protein. In some embodiments, (a) the antibody of the second fusion protein comprises an amino acid sequence from at least one complementarity determining region of 2H5, ADRI-2F3, H004, H009, H007, H015, H019, or H020; (b) the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and (c) the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the antibody of the second fusion protein comprises an amino acid sequence from at least one complementarity determining region of 2H5, ADRI-2F3, H004, H009, H007, H015, H019, or H020; (b) the transmembrane polypeptide of the second fusion protein comprises the transmembrane domain and cytosolic domain of VSV-G, the transmembrane domain and cytosolic domain of influenza Neuraminidase (NA), the transmembrane domain and cytosolic domain of influenza Hemagglutinin (HA), the transmembrane domain and cytosolic domain of Dengue E Protein, the transmembrane domain and cytosolic domain of GP120 or GP41, or the transmembrane domain and cytosolic domain of measles virus surface glycoprotein hemagglutinin (H) protein; and (c) the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the antibody of the second fusion protein comprises an amino acid sequence from at least one complementarity determining region of 2H5, ADRI-2F3, H004, H009, H007, H015, H019, or H020; (b) the transmembrane polypeptide of the second fusion protein comprises the transmembrane domain of VSV-G; and (c) the oligomerization domain of the second fusion protein comprises a D4 post-fusion trimerization domain of VSV-G protein. In some embodiments, (a) the antibody of the second fusion protein comprises an amino acid sequence from at least one complementarity determining region of 2H5, ADRI-2F3, H004, H009, H007, H015, H019, or H020; (b) the transmembrane polypeptide of the second fusion protein comprises the transmembrane domain and cytosolic domain of VSV-G; and (c) the oligomerization domain of the second fusion protein comprises a D4 post-fusion trimerization domain of VSV-G protein.

In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and (c) the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises the transmembrane domain and cytosolic domain of VSV-G, the transmembrane domain and cytosolic domain of influenza Neuraminidase (NA), the transmembrane domain and cytosolic domain of influenza Hemagglutinin (HA), the transmembrane domain and cytosolic domain of Dengue E Protein, the transmembrane domain and cytosolic domain of GP120 or GP41, or the transmembrane domain and cytosolic domain of measles virus surface glycoprotein hemagglutinin (H) protein; and (c) the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises the transmembrane domain of VSV-G; and (c) the oligomerization domain of the second fusion protein comprises a D4 post-fusion trimerization domain of VSV-G protein. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises the transmembrane domain and cytosolic domain of VSV-G; and (c) the oligomerization domain of the second fusion protein comprises a D4 post-fusion trimerization domain of VSV-G protein. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-α, or TNF-β. In some embodiments, the proliferation cytokine comprises IL-7, IL-15, or IL-21. In some embodiments, (a) the immune modulating polypeptide of the second fusion protein comprises TNF-α; (b) the transmembrane polypeptide of the second fusion protein comprises the transmembrane domain and cytosolic domain of VSV-G; and (c) the oligomerization domain of the second fusion protein comprises a D4 post-fusion trimerization domain of VSV-G protein. In some embodiments, (a) the immune modulating polypeptide of the second fusion protein comprises TNF-α; (b) the transmembrane polypeptide of the second fusion protein comprises the transmembrane domain and cytosolic domain of VSV-G; and (c) the oligomerization domain of the second fusion protein comprises a D4 post-fusion trimerization domain of VSV-G protein.

In some embodiments, (a) the antibody of the first fusion protein or the second fusion protein comprises a VH comprising an amino acid sequence having at least 75% sequence identity to an amino acid sequence of any one of SEQ ID Nos: 1, 3, 5, 7, 9, 11, 13, and 15 and a VL comprising an amino acid sequence having at least 75% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 75% sequence identity to an amino acid sequence of any one of SEQ ID Nos: 31-39; and (c) the oligomerization domain of the first fusion protein or the second fusion protein comprises an amino acid sequence having at least 75% sequence identity to an amino acid sequence of any one of SEQ ID Nos: 17-30. In some embodiments, (a) the antibody of the first fusion protein or the second fusion protein comprises a VH comprising an amino acid sequence having at least 76% sequence identity to an amino acid sequence of any one of SEQ ID Nos: 1, 3, 5, 7, 9, 11, 13, and 15 and a VL comprising an amino acid sequence having at least 76% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 76% sequence identity to an amino acid sequence of any one of SEQ ID Nos: 31-39; and (c) the oligomerization domain of the first fusion protein or the second fusion protein comprises an amino acid sequence having at least 76% sequence identity to an amino acid sequence of any one of SEQ ID Nos: 17-30. In some embodiments, (a) the antibody of the first fusion protein or the second fusion protein comprises a VH comprising an amino acid sequence having at least 77% sequence identity to an amino acid sequence of any one of SEQ ID Nos: 1, 3, 5, 7, 9, 11, 13, and 15 and a VL comprising an amino acid sequence having at least 77% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 77% sequence identity to an amino acid sequence of any one of SEQ ID Nos: 31-39; and (c) the oligomerization domain of the first fusion protein or the second fusion protein comprises an amino acid sequence having at least 77% sequence identity to an amino acid sequence of any one of SEQ ID Nos: 17-30. In some embodiments, (a) the antibody of the first fusion protein or the second fusion protein comprises a VH comprising an amino acid sequence having at least 78% sequence identity to an amino acid sequence of any one of SEQ ID Nos: 1, 3, 5, 7, 9, 11, 13, and 15 and a VL comprising an amino acid sequence having at least 78% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 78% sequence identity to an amino acid sequence of any one of SEQ ID Nos: 31-39; and (c) the oligomerization domain of the first fusion protein or the second fusion protein comprises an amino acid sequence having at least 78% sequence identity to an amino acid sequence of any one of SEQ ID Nos: 17-30. In some embodiments, (a) the antibody of the first fusion protein or the second fusion protein comprises a VH comprising an amino acid sequence having at least 79% sequence identity to an amino acid sequence of any one of SEQ ID Nos: 1, 3, 5, 7, 9, 11, 13, and 15 and a VL comprising an amino acid sequence having at least 79% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 79% sequence identity to an amino acid sequence of any one of SEQ ID Nos: 31-39; and (c) the oligomerization domain of the first fusion protein or the second fusion protein comprises an amino acid sequence having at least 79% sequence identity to an amino acid sequence of any one of SEQ ID Nos: 17-30. In some embodiments, (a) the antibody of the first fusion protein or the second fusion protein comprises a VH comprising an amino acid sequence having at least 80% sequence identity to an amino acid sequence of any one of SEQ ID Nos: 1, 3, 5, 7, 9, 11, 13, and 15 and a VL comprising an amino acid sequence having at least 80% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 80% sequence identity to an amino acid sequence of any one of SEQ ID Nos: 31-39; and (c) the oligomerization domain of the first fusion protein or the second fusion protein comprises an amino acid sequence having at least 80% sequence identity to an amino acid sequence of any one of SEQ ID Nos: 17-30. In some embodiments, (a) the antibody of the first fusion protein or the second fusion protein comprises a VH comprising an amino acid sequence having at least 81% sequence identity to an amino acid sequence of any one of SEQ ID Nos: 1, 3, 5, 7, 9, 11, 13, and 15 and a VL comprising an amino acid sequence having at least 81% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 81% sequence identity to an amino acid sequence of any one of SEQ ID Nos: 31-39; and (c) the oligomerization domain of the first fusion protein or the second fusion protein comprises an amino acid sequence having at least 81% sequence identity to an amino acid sequence of any one of SEQ ID Nos: 17-30. In some embodiments, (a) the antibody of the first fusion protein or the second fusion protein comprises a VH comprising an amino acid sequence having at least 82% sequence identity to an amino acid sequence of any one of SEQ ID Nos: 1, 3, 5, 7, 9, 11, 13, and 15 and a VL comprising an amino acid sequence having at least 82% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 82% sequence identity to an amino acid sequence of any one of SEQ ID Nos: 31-39; and (c) the oligomerization domain of the first fusion protein or the second fusion protein comprises an amino acid sequence having at least 82% sequence identity to an amino acid sequence of any one of SEQ ID Nos: 17-30. In some embodiments, (a) the antibody of the first fusion protein or the second fusion protein comprises a VH comprising an amino acid sequence having at least 83% sequence identity to an amino acid sequence of any one of SEQ ID Nos: 1, 3, 5, 7, 9, 11, 13, and 15 and a VL comprising an amino acid sequence having at least 83% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 83% sequence identity to an amino acid sequence of any one of SEQ ID Nos: 31-39; and (c) the oligomerization domain of the first fusion protein or the second fusion protein comprises an amino acid sequence having at least 83% sequence identity to an amino acid sequence of any one of SEQ ID Nos: 17-30. In some embodiments, (a) the antibody of the first fusion protein or the second fusion protein comprises a VH comprising an amino acid sequence having at least 84% sequence identity to an amino acid sequence of any one of SEQ ID Nos: 1, 3, 5, 7, 9, 11, 13, and 15 and a VL comprising an amino acid sequence having at least 84% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 84% sequence identity to an amino acid sequence of any one of SEQ ID Nos: 31-39; and (c) the oligomerization domain of the first fusion protein or the second fusion protein comprises an amino acid sequence having at least 84% sequence identity to an amino acid sequence of any one of SEQ ID Nos: 17-30. In some embodiments, (a) the antibody of the first fusion protein or the second fusion protein comprises a VH comprising an amino acid sequence having at least 85% sequence identity to an amino acid sequence of any one of SEQ ID Nos: 1, 3, 5, 7, 9, 11, 13, and 15 and a VL comprising an amino acid sequence having at least 85% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 85% sequence identity to an amino acid sequence of any one of SEQ ID Nos: 31-39; and (c) the oligomerization domain of the first fusion protein or the second fusion protein comprises an amino acid sequence having at least 85% sequence identity to an amino acid sequence of any one of SEQ ID Nos: 17-30. In some embodiments, (a) the antibody of the first fusion protein or the second fusion protein comprises a VH comprising an amino acid sequence having at least 86% sequence identity to an amino acid sequence of any one of SEQ ID Nos: 1, 3, 5, 7, 9, 11, 13, and 15 and a VL comprising an amino acid sequence having at least 86% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 86% sequence identity to an amino acid sequence of any one of SEQ ID Nos: 31-39; and (c) the oligomerization domain of the first fusion protein or the second fusion protein comprises an amino acid sequence having at least 86% sequence identity to an amino acid sequence of any one of SEQ ID Nos: 17-30. In some embodiments, (a) the antibody of the first fusion protein or the second fusion protein comprises a VH comprising an amino acid sequence having at least 87% sequence identity to an amino acid sequence of any one of SEQ ID Nos: 1, 3, 5, 7, 9, 11, 13, and 15 and a VL comprising an amino acid sequence having at least 87% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 87% sequence identity to an amino acid sequence of any one of SEQ ID Nos: 31-39; and (c) the oligomerization domain of the first fusion protein or the second fusion protein comprises an amino acid sequence having at least 87% sequence identity to an amino acid sequence of any one of SEQ ID Nos: 17-30. In some embodiments, (a) the antibody of the first fusion protein or the second fusion protein comprises a VH comprising an amino acid sequence having at least 88% sequence identity to an amino acid sequence of any one of SEQ ID Nos: 1, 3, 5, 7, 9, 11, 13, and 15 and a VL comprising an amino acid sequence having at least 88% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 88% sequence identity to an amino acid sequence of any one of SEQ ID Nos: 31-39; and (c) the oligomerization domain of the first fusion protein or the second fusion protein comprises an amino acid sequence having at least 88% sequence identity to an amino acid sequence of any one of SEQ ID Nos: 17-30. In some embodiments, (a) the antibody of the first fusion protein or the second fusion protein comprises a VH comprising an amino acid sequence having at least 89% sequence identity to an amino acid sequence of any one of SEQ ID Nos: 1, 3, 5, 7, 9, 11, 13, and 15 and a VL comprising an amino acid sequence having at least 89% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 89% sequence identity to an amino acid sequence of any one of SEQ ID Nos: 31-39; and (c) the oligomerization domain of the first fusion protein or the second fusion protein comprises an amino acid sequence having at least 89% sequence identity to an amino acid sequence of any one of SEQ ID Nos: 17-30. In some embodiments, (a) the antibody of the first fusion protein or the second fusion protein comprises a VH comprising an amino acid sequence having at least 90% sequence identity to an amino acid sequence of any one of SEQ ID Nos: 1, 3, 5, 7, 9, 11, 13, and 15 and a VL comprising an amino acid sequence having at least 90% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 90% sequence identity to an amino acid sequence of any one of SEQ ID Nos: 31-39; and (c) the oligomerization domain of the first fusion protein or the second fusion protein comprises an amino acid sequence having at least 90% sequence identity to an amino acid sequence of any one of SEQ ID Nos: 17-30. In some embodiments, (a) the antibody of the first fusion protein or the second fusion protein comprises a VH comprising an amino acid sequence having at least 91% sequence identity to an amino acid sequence of any one of SEQ ID Nos: 1, 3, 5, 7, 9, 11, 13, and 15 and a VL comprising an amino acid sequence having at least 91% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 91% sequence identity to an amino acid sequence of any one of SEQ ID Nos: 31-39; and (c) the oligomerization domain of the first fusion protein or the second fusion protein comprises an amino acid sequence having at least 91% sequence identity to an amino acid sequence of any one of SEQ ID Nos: 17-30. In some embodiments, (a) the antibody of the first fusion protein or the second fusion protein comprises a VH comprising an amino acid sequence having at least 92% sequence identity to an amino acid sequence of any one of SEQ ID Nos: 1, 3, 5, 7, 9, 11, 13, and 15 and a VL comprising an amino acid sequence having at least 92% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 92% sequence identity to an amino acid sequence of any one of SEQ ID Nos: 31-39; and (c) the oligomerization domain of the first fusion protein or the second fusion protein comprises an amino acid sequence having at least 92% sequence identity to an amino acid sequence of any one of SEQ ID Nos: 17-30. In some embodiments, (a) the antibody of the first fusion protein or the second fusion protein comprises a VH comprising an amino acid sequence having at least 93% sequence identity to an amino acid sequence of any one of SEQ ID Nos: 1, 3, 5, 7, 9, 11, 13, and 15 and a VL comprising an amino acid sequence having at least 93% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 93% sequence identity to an amino acid sequence of any one of SEQ ID Nos: 31-39; and (c) the oligomerization domain of the first fusion protein or the second fusion protein comprises an amino acid sequence having at least 93% sequence identity to an amino acid sequence of any one of SEQ ID Nos: 17-30. In some embodiments, (a) the antibody of the first fusion protein or the second fusion protein comprises a VH comprising an amino acid sequence having at least 94% sequence identity to an amino acid sequence of any one of SEQ ID Nos: 1, 3, 5, 7, 9, 11, 13, and 15 and a VL comprising an amino acid sequence having at least 94% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 94% sequence identity to an amino acid sequence of any one of SEQ ID Nos: 31-39; and (c) the oligomerization domain of the first fusion protein or the second fusion protein comprises an amino acid sequence having at least 94% sequence identity to an amino acid sequence of any one of SEQ ID Nos: 17-30. In some embodiments, (a) the antibody of the first fusion protein or the second fusion protein comprises a VH comprising an amino acid sequence having at least 95% sequence identity to an amino acid sequence of any one of SEQ ID Nos: 1, 3, 5, 7, 9, 11, 13, and 15 and a VL comprising an amino acid sequence having at least 95% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 95% sequence identity to an amino acid sequence of any one of SEQ ID Nos: 31-39; and (c) the oligomerization domain of the first fusion protein or the second fusion protein comprises an amino acid sequence having at least 95% sequence identity to an amino acid sequence of any one of SEQ ID Nos: 17-30. In some embodiments, (a) the antibody of the first fusion protein or the second fusion protein comprises a VH comprising an amino acid sequence having at least 96% sequence identity to an amino acid sequence of any one of SEQ ID Nos: 1, 3, 5, 7, 9, 11, 13, and 15 and a VL comprising an amino acid sequence having at least 96% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 96% sequence identity to an amino acid sequence of any one of SEQ ID Nos: 31-39; and (c) the oligomerization domain of the first fusion protein or the second fusion protein comprises an amino acid sequence having at least 96% sequence identity to an amino acid sequence of any one of SEQ ID Nos: 17-30. In some embodiments, (a) the antibody of the first fusion protein or the second fusion protein comprises a VH comprising an amino acid sequence having at least 97% sequence identity to an amino acid sequence of any one of SEQ ID Nos: 1, 3, 5, 7, 9, 11, 13, and 15 and a VL comprising an amino acid sequence having at least 97% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 97% sequence identity to an amino acid sequence of any one of SEQ ID Nos: 31-39; and (c) the oligomerization domain of the first fusion protein or the second fusion protein comprises an amino acid sequence having at least 97% sequence identity to an amino acid sequence of any one of SEQ ID Nos: 17-30. In some embodiments, (a) the antibody of the first fusion protein or the second fusion protein comprises a VH comprising an amino acid sequence having at least 98% sequence identity to an amino acid sequence of any one of SEQ ID Nos: 1, 3, 5, 7, 9, 11, 13, and 15 and a VL comprising an amino acid sequence having at least 98% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 98% sequence identity to an amino acid sequence of any one of SEQ ID Nos: 31-39; and (c) the oligomerization domain of the first fusion protein or the second fusion protein comprises an amino acid sequence having at least 98% sequence identity to an amino acid sequence of any one of SEQ ID Nos: 17-30. In some embodiments, (a) the antibody of the first fusion protein or the second fusion protein comprises a VH comprising an amino acid sequence having at least 99% sequence identity to an amino acid sequence of any one of SEQ ID Nos: 1, 3, 5, 7, 9, 11, 13, and 15 and a VL comprising an amino acid sequence having at least 99% sequence identity to an amino acid sequence of any one of SEQ ID Nos: 2, 4, 6, 8, 10, 12, 14, and 16; (b) the transmembrane polypeptide of the first fusion protein or the second fusion protein comprises an amino acid sequence having at least 99% sequence identity to an amino acid sequence of any one of SEQ ID Nos: 31-39; and (c) the oligomerization domain of the first fusion protein or the second fusion protein comprises an amino acid sequence having at least 99% sequence identity to an amino acid sequence of any one of SEQ ID Nos: 17-30. In some embodiments, (a) the antibody of the first fusion protein or the second fusion protein comprises a VH comprising an amino acid sequence according to any one of SEQ ID Nos: 1, 3, 5, 7, 9, 11, 13, and 15 and a VL comprising an amino acid sequence according to any one of SEQ ID Nos: 2, 4, 6, 8, 10, 12, 14, and 16; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30.

In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 75% sequence identity to an amino acid sequence of any one of SEQ ID Nos: 31-39; and (c) the oligomerization domain of the second fusion protein comprises an amino acid sequence having at least 75% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 76% sequence identity to an amino acid sequence of any one of SEQ ID Nos: 31-39; and (c) the oligomerization domain of the second fusion protein comprises an amino acid sequence having at least 76% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 77% sequence identity to an amino acid sequence of any one of SEQ ID Nos: 31-39; and (c) the oligomerization domain of the second fusion protein comprises an amino acid sequence having at least 77% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 78% sequence identity to an amino acid sequence of any one of SEQ ID Nos: 31-39; and (c) the oligomerization domain of the second fusion protein comprises an amino acid sequence having at least 78% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 79% sequence identity to an amino acid sequence of any one of SEQ ID Nos: 31-39; and (c) the oligomerization domain of the second fusion protein comprises an amino acid sequence having at least 79% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 80% sequence identity to an amino acid sequence of any one of SEQ ID Nos: 31-39; and (c) the oligomerization domain of the second fusion protein comprises an amino acid sequence having at least 80% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 81% sequence identity to an amino acid sequence of any one of SEQ ID Nos: 31-39; and (c) the oligomerization domain of the second fusion protein comprises an amino acid sequence having at least 81% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 82% sequence identity to an amino acid sequence of any one of SEQ ID Nos: 31-39; and (c) the oligomerization domain of the second fusion protein comprises an amino acid sequence having at least 82% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 83% sequence identity to an amino acid sequence of any one of SEQ ID Nos: 31-39; and (c) the oligomerization domain of the second fusion protein comprises an amino acid sequence having at least 83% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 84% sequence identity to an amino acid sequence of any one of SEQ ID Nos: 31-39; and (c) the oligomerization domain of the second fusion protein comprises an amino acid sequence having at least 84% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 85% sequence identity to an amino acid sequence of any one of SEQ ID Nos: 31-39; and (c) the oligomerization domain of the second fusion protein comprises an amino acid sequence having at least 85% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 86% sequence identity to an amino acid sequence of any one of SEQ ID Nos: 31-39; and (c) the oligomerization domain of the second fusion protein comprises an amino acid sequence having at least 86% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 87% sequence identity to an amino acid sequence of any one of SEQ ID Nos: 31-39; and (c) the oligomerization domain of the second fusion protein comprises an amino acid sequence having at least 87% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 88% sequence identity to an amino acid sequence of any one of SEQ ID Nos: 31-39; and (c) the oligomerization domain of the second fusion protein comprises an amino acid sequence having at least 88% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 89% sequence identity to an amino acid sequence of any one of SEQ ID Nos: 31-39; and (c) the oligomerization domain of the second fusion protein comprises an amino acid sequence having at least 89% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 90% sequence identity to an amino acid sequence of any one of SEQ ID Nos: 31-39; and (c) the oligomerization domain of the second fusion protein comprises an amino acid sequence having at least 90% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 91% sequence identity to an amino acid sequence of any one of SEQ ID Nos: 31-39; and (c) the oligomerization domain of the second fusion protein comprises an amino acid sequence having at least 91% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 92% sequence identity to an amino acid sequence of any one of SEQ ID Nos: 31-39; and (c) the oligomerization domain of the second fusion protein comprises an amino acid sequence having at least 92% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 93% sequence identity to an amino acid sequence of any one of SEQ ID Nos: 31-39; and (c) the oligomerization domain of the second fusion protein comprises an amino acid sequence having at least 93% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 94% sequence identity to an amino acid sequence of any one of SEQ ID Nos: 31-39; and (c) the oligomerization domain of the second fusion protein comprises an amino acid sequence having at least 94% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 95% sequence identity to an amino acid sequence of any one of SEQ ID Nos: 31-39; and (c) the oligomerization domain of the second fusion protein comprises an amino acid sequence having at least 95% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 96% sequence identity to an amino acid sequence of any one of SEQ ID Nos: 31-39; and (c) the oligomerization domain of the second fusion protein comprises an amino acid sequence having at least 96% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 97% sequence identity to an amino acid sequence of any one of SEQ ID Nos: 31-39; and (c) the oligomerization domain of the second fusion protein comprises an amino acid sequence having at least 97% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 98% sequence identity to an amino acid sequence of any one of SEQ ID Nos: 31-39; and (c) the oligomerization domain of the second fusion protein comprises an amino acid sequence having at least 98% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence having at least 99% sequence identity to an amino acid sequence of any one of SEQ ID Nos: 31-39; and (c) the oligomerization domain of the second fusion protein comprises an amino acid sequence having at least 99% sequence identity to an amino acid sequence of any one of SEQ ID Nos: 17-30. In some embodiments, (a) the immune modulating polypeptide of the second fusion protein comprises an NK cell activating ligand, an immune checkpoint molecule, an inflammatory cytokine, or a proliferation cytokine; (b) the transmembrane polypeptide of the second fusion protein comprises an amino acid sequence of any one of SEQ ID Nos: 31-39; and (c) the oligomerization domain of the second fusion protein comprises an amino acid sequence of any one of SEQ ID Nos: 17-30. In some embodiments, (a) the antibody of the first fusion protein comprises a VH comprising an amino acid sequence according to any one of SEQ ID Nos: 1, 3, 5, 7, 9, 11, 13, and 15 and a VL comprising an amino acid sequence according to any one of SEQ ID Nos: 2, 4, 6, 8, 10, 12, 14, and 16; (b) the transmembrane polypeptide of the first fusion protein comprises an amino acid sequence according to any one of SEQ ID Nos: 31-39; (c) the oligomerization domain of the first fusion protein comprises an amino acid sequence according to any one of SEQ ID Nos: 17-30; (d) the immune modulating polypeptide of the second fusion protein comprises an NK cell activating ligand, an immune checkpoint molecule, an inflammatory cytokine, or a proliferation cytokine; (e) the transmembrane polypeptide of the second fusion protein comprises an amino acid sequence of any one of SEQ ID Nos: 31-39; and (f) the oligomerization domain of the second fusion protein comprises an amino acid sequence of any one of SEQ ID Nos: 17-30. In some embodiments, the NK cell activating ligand comprises m157, NKG2D ligand, CD70, or CD40. In some embodiments, the immune checkpoint molecule comprises CTLA4, PD1, OX40, or CD28. In some embodiments, the inflammatory cytokine comprises IL-12, IL-18, TNF-alpha, or TNF-β. In some embodiments, the proliferation cytokine comprises IL-7, IL-15, or IL-21.

In some embodiments, (a) the antibody of the first fusion protein or the second fusion protein comprises a VH comprising an amino acid sequence according to SEQ ID No: 1 and a VL comprising an amino acid sequence according to SEQ ID No: 2; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 3 and a VL comprising an amino acid sequence according to SEQ ID No: 4; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 5 and a VL comprising an amino acid sequence according to SEQ ID No: 6; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 7 and a VL comprising an amino acid sequence according to SEQ ID No: 8; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 9 and a VL comprising an amino acid sequence according to SEQ ID No: 10; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 11 and a VL comprising an amino acid sequence according to SEQ ID No: 12; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 13 and a VL comprising an amino acid sequence according to SEQ ID No: 14; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. In some embodiments, (a) the antibody of the first fusion protein or the second fusion protein comprises a VH comprising an amino acid sequence according to SEQ ID No: 15 and a VL comprising an amino acid sequence according to SEQ ID No: 16; (b) the transmembrane polypeptide of the first fusion protein or the second fusion protein comprises an amino acid sequence according to any one of SEQ ID Nos: 31-39; and (c) the oligomerization domain of the first fusion protein or the second fusion protein comprises an amino acid sequence according to any one of SEQ ID Nos: 17-30. Exemplary fusion protein sequences of the antivirus are listed in Table 4.

TABLE 4
Exemplary Fusion Protein Sequences

| HBV- | | | | | SEQ ID |
|---|---|---|---|---|---|
| Antivirus | AB¹ | TM² | OD³ | Amino Acid Sequence | No: |
| αS: | H015 | VSV-G | None | MEFGLSWVFLVALFRGVQSQVQLVESG | 43 |
| H015/VM | | | | GGVAQPGRSLRLSCAASGFSFSRHGMH | |
| | | | | WVRQAPGKGLEWVAGIWFDGTNDYYTD | |
| | | | | SVKGRFTISRDNSRSTLYLDINSLRAE | |
| | | | | DTAVYYCAREDPHLLIATLDLWGLGTL | |
| | | | | VTVSSASTKGPSVFPLAPSSKSTSGGT | |
| | | | | AALGCLVKDYFPEPVTVSWNSGALTSG | |
| | | | | VHTFPAVLQSSGLYSLSSVVTVPSSSL | |
| | | | | GTQTYICNVNHKPSNTKVDKRVEPKSC | |
| | | | | DGGGGSGGGGSGGGGSGGGGSDIQVTQ | |
| | | | | SPSSLSAFVGDRVTITCRASQGLTSFI | |
| | | | | NWYQQKPGRAPKLLISSASSLQRGVPS | |
| | | | | RFTASGSGTHFTLTISSLQPEDFATYY | |
| | | | | CQQSYGTPALAFGGGTKVDIKRTVAAP | |
| | | | | SVFIFPPSDEQLKSGTASVVCLLNNFY | |
| | | | | PREAKVQWKVDNALQSGNSQESVTEQD | |
| | | | | SKDSTYSLSSTLTLSKADYEKHKVYAC | |
| | | | | EVTHQGLSSPVTKSFNRGECRGMLDSD | |
| | | | | LHLSSKAQVFEHPHIQDAASQLPDDES | |
| | | | | LFFGDTGLSKNPIELVEGWFSSWKSSI | |
| | | | | ASFFFIIGLIIGLFLVLRVGIHLCIKL | |
| | | | | KHTKKRQIYPYDVPDYA | |
| αS: | H015 | VSV-G | D4 | MEFGLSWVFLVALFRGVQSQVQLVESG | 44 |
| H015/D4 | | | | GGVAQPGRSLRLSCAASGFSFSRHGMH | |
| | | | | WVRQAPGKGLEWVAGIWFDGTNDYYTD | |
| | | | | SVKGRFTISRDNSRSTLYLDINSLRAE | |
| | | | | DTAVYYCAREDPHLLIATLDLWGLGTL | |
| | | | | VTVSSASTKGPSVFPLAPSSKSTSGGT | |
| | | | | AALGCLVKDYFPEPVTVSWNSGALTSG | |
| | | | | VHTFPAVLQSSGLYSLSSVVTVPSSSL | |
| | | | | GTQTYICNVNHKPSNTKVDKRVEPKSC | |
| | | | | DGGGGSGGGGSGGGGSGGGGSDIQVTQ | |
| | | | | SPSSLSAFVGDRVTITCRASQGLTSFI | |
| | | | | NWYQQKPGRAPKLLISSASSLQRGVPS | |
| | | | | RFTASGSGTHFTLTISSLQPEDFATYY | |
| | | | | CQQSYGTPALAFGGGTKVDIKRTVAAP | |
| | | | | SVFIFPPSDEQLKSGTASVVCLLNNFY | |
| | | | | PREAKVQWKVDNALQSGNSQESVTEQD | |
| | | | | SKDSTYSLSSTLTLSKADYEKHKVYAC | |
| | | | | EVTHQGLSSPVTKSFNRGECVIQADGW | |
| | | | | MCHASKWVTTCDFRWYGPKYITHSIRS | |
| | | | | FTPSVEQCKESIEQTKQGTWLNPGFPP | |
| | | | | QSCGYATVTDAEAVIVQVTPHHVLVDE | |
| | | | | YTGEWVDSQFINGKCSNYICPTVHNST | |
| | | | | TWHSDYKVKGLCDSNLGMLDSDLHLSS | |
| | | | | KAQVFEHPHIQDAASQLPDDESLFFGD | |
| | | | | TGLSKNPIELVEGWFSSWKSSIASFFF | |
| | | | | IIGLIIGLFLVLRVGIHLCIKLKHTKK | |
| | | | | RQIYPYDVPDYA | |
| αL: | 2H5 | VSV-G | None | MEFGLSWVFLVALFRGVQSSQVQLQQS | 45 |
| 2H5/VM | | | | GPGLVKPSQTLSLTCGISGDSVSSKSA | |
| | | | | AWNWIRQSPSRGLEWLGRTYYRSKWHN | |
| | | | | DYAVSVKSRITINPDTSKNQFSLQLNS | |
| | | | | VTPEDTAVYYCARGQMGALDVWGQGTT | |
| | | | | VTVSSGGGGSGGGGSGGGGSQSVLTQP | |
| | | | | PSASGTPGQRVTISCSGSSSNIGSYYV | |
| | | | | YWYQQFPGTAPKLLIYGNNQRPSGVPD | |
| | | | | RFSGSKSGTSASLAITGLQAEDEADYY | |
| | | | | CQSYDSSLSGVIFGGGTKLTVLRGMLD | |
| | | | | SDLHLSSKAQVFEHPHIQDAASQLPDD | |
| | | | | ESLFFGDTGLSKNPIELVEGWFSSWKS | |
| | | | | SIASFFFIIGLIIGLFLVLRVGIHLCI | |
| | | | | KLKHTKKRQIYPYDVPDYA | |
| αS: | ADRI | VSV-G | D4 | MEFGLSWVFLVALFRGVQSEVQVLESG | 46 |
| ADRI/D4 | | | | GGLVQPGGSLRLSCAASGFRFSSYAMS | |
| | | | | WVRQAPGKGLEWVSGISGTGENTYYAD | |
| | | | | SVKGRFTISRDNSKNTLYVQMNSLRAE | |
| | | | | DTAVYYCAKDAILGSGHPWYFHVWGRG | |
| | | | | TLVTVSSGGGGSGGGGSGGGGSGGGGS | |
| | | | | SYVLTQPPSVSVAPGQTARMTCGGNNI | |
| | | | | GSESVHWFQQKPGQAPVLVVYDDSDRP | |
| | | | | SGIPERFSGSNSGNTATLTISRVEAGD | |
| | | | | EADYYCQVWDSSSDHAVFGGGTQLTVL | |
| | | | | VIQADGWMCHASKWVTTCDFRWYGPKY | |
| | | | | ITHSIRSFTPSVEQCKESIEQTKQGTW | |
| | | | | LNPGFPPQSCGYATVTDAEAVIVQVTP | |
| | | | | HHVLVDEYTGEWVDSQFINGKCSNYIC | |
| | | | | PTVHNSTTWHSDYKVKGLCDSNLGMLD | |
| | | | | SDLHLSSKAQVFEHPHIQDAASQLPDD | |
| | | | | ESLFFGDTGLSKNPIELVEGWFSSWKS | |
| | | | | SIASFFFIIGLIIGLFLVLRVGIHLCI | |
| | | | | KLKHTKKRQIYPYDVPDYA | |
| Tandem | H015/ | VSV-G | D4 | MEFGLSWVFLVALFRGVQSSQVQLQQS | 47 |
| (αS-αL)/ | 2H5 | | | GPGLVKPSQTLSLTCGISGDSVSSKSA | |
| D4 | | | | AWNWIRQSPSRGLEWLGRTYYRSKWHN | |
| | | | | DYAVSVKSRITINPDTSKNQFSLQLNS | |
| | | | | VTPEDTAVYYCARGQMGALDVWGQGTT | |
| | | | | VTVSSGGGGSGGGGSGGGGSQSVLTQP | |
| | | | | PSASGTPGQRVTISCSGSSSNIGSYYV | |
| | | | | YWYQQFPGTAPKLLIYGNNQRPSGVPD | |
| | | | | RFSGSKSGTSASLAITGLQAEDEADYY | |
| | | | | CQSYDSSLSGVIFGGGTKLTVLGGGGS | |
| | | | | GGGGSGGGGSGGGGSQVQLVESGGGVA | |
| | | | | QPGRSLRLSCAASGFSFSRHGMHWVRQ | |
| | | | | APGKGLEWVAGIWFDGTNDYYTDSVKG | |
| | | | | RFTISRDNSRSTLYLDINSLRAEDTAV | |
| | | | | YYCAREDPHLLIATLDLWGLGTLVTVS | |
| | | | | SASTKGPSVFPLAPSSKSTSGGTAALG | |
| | | | | CLVKDYFPEPVTVSWNSGALTSGVHTF | |
| | | | | PAVLQSSGLYSLSSVVTVPSSSLGTQT | |
| | | | | YICNVNHKPSNTKVDKRVEPKSCDGGG | |
| | | | | GSGGGGSGGGGSGGGGSDIQVTQSPSS | |
| | | | | LSAFVGDRVTITCRASQGLTSFINWYQ | |
| | | | | QKPGRAPKLLISSASSLQRGVPSRFTA | |
| | | | | SGSGTHFTLTISSLQPEDFATYYCQQS | |
| | | | | YGTPALAFGGGTKVDIKRTVAAPSVFI | |
| | | | | FPPSDEQLKSGTASVVCLLNNFYPREA | |
| | | | | KVQWKVDNALQSGNSQESVTEQDSKDS | |
| | | | | TYSLSSTLTLSKADYEKHKVYACEVTH | |
| | | | | QGLSSPVTKSFNRGECVIQADGWMCHA | |
| | | | | SKWVTTCDFRWYGPKYITHSIRSFTPS | |
| | | | | VEQCKESIEQTKQGTWLNPGFPPQSCG | |
| | | | | YATVIDAEAVIVQVTPHHVLVDEYTGE | |
| | | | | WVDSQFINGKCSNYICPTVHNSTTWHS | |
| | | | | DYKVKGLCDSNLGMLDSDLHLSSKAQV | |
| | | | | FEHPHIQDAASQLPDDESLFFGDTGLS | |
| | | | | KNPIELVEGWFSSWKSSIASFFFIIGL | |
| | | | | IIGLFLVLRVGIHLCIKLKHTKKRQIY | |
| | | | | PYDVPDYA | |
| αS: | H004 | VSV-G | None | MEFGLSWVFLVALFRGVQSEVOLVESG | 48 |
| H004/VM | | | | GGLVQPGGSLRLSCAASGFTFSSYAMS | |
| | | | | WVRQAPGKGLEWVSAISGSGGSTYYAD | |
| | | | | SVKGRFTISRDNSKNTLYLQMNSLRAE | |
| | | | | DTAVYYCAKDGYFGSGSLYGIDVWGQG | |
| | | | | TTVTVSSGGGGSGGGGSGGGGSGGGGS | |
| | | | | SYVLTQPPSVSVAPGQTARITCGGNNI | |
| | | | | GSKSVHWYQQKPGQAPVLVVYDDSDRP | |
| | | | | SGIPERFSGSNSGNTATLTISRVEAGD | |
| | | | | EADYYCQVWDSNHDHPGVVFGGGTKLT | |
| | | | | VLRGMLDSDLHLSSKAQVFEHPHIQDA | |
| | | | | ASQLPDDESLFFGDTGLSKNPIELVEG | |
| | | | | WFSSWKSSIASFFFIIGLIIGLFLVLR | |
| | | | | VGIHLCIKLKHTKKRQIYPYDVPDYA | |
| αS: | H004 | VSV-G | D4 | MEFGLSWVFLVALFRGVQSEVQLVESG | 49 |
| H004/D4 | | | | GGLVQPGGSLRLSCAASGFTFSSYAMS | |
| | | | | WVRQAPGKGLEWVSAISGSGGSTYYAD | |
| | | | | SVKGRFTISRDNSKNTLYLQMNSLRAE | |
| | | | | DTAVYYCAKDGYFGSGSLYGIDVWGQG | |
| | | | | TTVTVSSGGGGSGGGGSGGGGSGGGGS | |
| | | | | SYVLTQPPSVSVAPGQTARITCGGNNI | |
| | | | | GSKSVHWYQQKPGQAPVLVVYDDSDRP | |
| | | | | SGIPERFSGSNSGNTATLTISRVEAGD | |
| | | | | EADYYCQVWDSNHDHPGVVFGGGTKLT | |
| | | | | VLVIQADGWMCHASKWVTTCDFRWYGP | |
| | | | | KYITHSIRSFTPSVEQCKESIEQTKQG | |
| | | | | TWLNPGFPPQSCGYATVTDAEAVIVQV | |
| | | | | TPHHVLVDEYTGEWVDSQFINGKCSNY | |
| | | | | ICPTVHNSTTWHSDYKVKGLCDSNLGM | |
| | | | | LDSDLHLSSKAQVFEHPHIQDAASQLP | |
| | | | | DDESLFFGDTGLSKNPIELVEGWFSSW | |
| | | | | KSSIASFFFIIGLIIGLFLVLRVGIHL | |
| | | | | CIKLKHTKKRQIYPYDVPDYA | |

AB¹: antibody
TM²: transmembrane domain
OD³: oligomerization domain

In some embodiments, the first fusion protein or the second fusion protein comprises an amino acid sequence having at least 75% sequence identity to an amino acid sequence according to any one of SEQ ID Nos. 43-49. In some embodiments, the first fusion protein or the second fusion protein comprises an amino acid sequence having at least 76% sequence identity to an amino acid sequence according to any one of SEQ ID Nos. 43-49. In some embodiments, the first fusion protein or the second fusion protein comprises an amino acid sequence having at least 77% sequence identity to an amino acid sequence according to any one of SEQ ID Nos. 43-49. In some embodiments, the first fusion protein or the second fusion protein comprises an amino acid sequence having at least 78% sequence identity to an amino acid sequence according to any one of SEQ ID Nos. 43-49. In some embodiments, the first fusion protein or the second fusion protein comprises an amino acid sequence having at least 79% sequence identity to an amino acid sequence according to any one of SEQ ID Nos. 43-49. In some embodiments, the first fusion protein or the second fusion protein comprises an amino acid sequence having at least 80% sequence identity to an amino acid sequence according to any one of SEQ ID Nos. 43-49. In some embodiments, the first fusion protein or the second fusion protein comprises an amino acid sequence having at least 81% sequence identity to an amino acid sequence according to any one of SEQ ID Nos. 43-49. In some embodiments, the first fusion protein or the second fusion protein comprises an amino acid sequence having at least 82% sequence identity to an amino acid sequence according to any one of SEQ ID Nos. 43-49. In some embodiments, the first fusion protein or the second fusion protein comprises an amino acid sequence having at least 83% sequence identity to an amino acid sequence according to any one of SEQ ID Nos. 43-49. In some embodiments, the first fusion protein or the second fusion protein comprises an amino acid sequence having at least 84% sequence identity to an amino acid sequence according to any one of SEQ ID Nos. 43-49. In some embodiments, the first fusion protein or the second fusion protein comprises an amino acid sequence having at least 85% sequence identity to an amino acid sequence according to any one of SEQ ID Nos. 43-49. In some embodiments, the first fusion protein or the second fusion protein comprises an amino acid sequence having at least 86% sequence identity to an amino acid sequence according to any one of SEQ ID Nos. 43-49. In some embodiments, the first fusion protein or the second fusion protein comprises an amino acid sequence having at least 87% sequence identity to an amino acid sequence according to any one of SEQ ID Nos. 43-49. In some embodiments, the first fusion protein or the second fusion protein comprises an amino acid sequence having at least 88% sequence identity to an amino acid sequence according to any one of SEQ ID Nos. 43-49. In some embodiments, the first fusion protein or the second fusion protein comprises an amino acid sequence having at least 89% sequence identity to an amino acid sequence according to any one of SEQ ID Nos. 43-49. In some embodiments, the first fusion protein or the second fusion protein comprises an amino acid sequence having at least 90% sequence identity to an amino acid sequence according to any one of SEQ ID Nos. 43-49. In some embodiments, the first fusion protein or the second fusion protein comprises an amino acid sequence having at least 91% sequence identity to an amino acid sequence according to any one of SEQ ID Nos. 43-49. In some embodiments, the first fusion protein or the second fusion protein comprises an amino acid sequence having at least 92% sequence identity to an amino acid sequence according to any one of SEQ ID Nos. 43-49. In some embodiments, the first fusion protein or the second fusion protein comprises an amino acid sequence having at least 93% sequence identity to an amino acid sequence according to any one of SEQ ID Nos. 43-49. In some embodiments, the first fusion protein or the second fusion protein comprises an amino acid sequence having at least 94% sequence identity to an amino acid sequence according to any one of SEQ ID Nos. 43-49. In some embodiments, the first fusion protein or the second fusion protein comprises an amino acid sequence having at least 95% sequence identity to an amino acid sequence according to any one of SEQ ID Nos. 43-49. In some embodiments, the first fusion protein or the second fusion protein comprises an amino acid sequence having at least 96% sequence identity to an amino acid sequence according to any one of SEQ ID Nos. 43-49. In some embodiments, the first fusion protein or the second fusion protein comprises an amino acid sequence having at least 97% sequence identity to an amino acid sequence according to any one of SEQ ID Nos. 43-49. In some embodiments, the first fusion protein or the second fusion protein comprises an amino acid sequence having at least 98% sequence identity to an amino acid sequence according to any one of SEQ ID Nos. 43-49. In some embodiments, the first fusion protein or the second fusion protein comprises an amino acid sequence having at least 99% sequence identity to an amino acid sequence according to any one of SEQ ID Nos. 43-49. In some embodiments, the first fusion protein or the second fusion protein comprises an amino acid sequence according to any one of SEQ ID Nos. 43-49.

Described herein, in some embodiments, are HBV-antiviruses comprising a fusion protein that comprises a transmembrane polypeptide and an antibody. In some embodiments, the transmembrane polypeptide anchors the fusion protein to a bilayer of the HBV-antivirus. In some embodiments, the transmembrane polypeptide comprises the transmembrane domain of a Vesicular Stomatitis virus glycoprotein (VSV-G). In some embodiments, the transmembrane polypeptide comprises the transmembrane domain of influenza Hemagglutinin (HA). In some embodiments, the transmembrane polypeptide comprises the transmembrane domain of HIV surface glycoprotein GP120 or GP41. In some embodiments, the transmembrane polypeptide comprises the transmembrane domain of measles virus surface glycoprotein hemagglutinin (H) protein. In some embodiments, the transmembrane polypeptide comprises the transmembrane domain of influenza Neuraminidase (NA). In some embodiments, the transmembrane polypeptide comprises the transmembrane domain of Dengue E Protein.

In some embodiments, the HBV-Antivirus comprises a reporter, a therapeutic molecule, or combinations thereof. In some embodiments, the reporter protein comprises a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter comprises a fluorescent protein. In some embodiments, the fluorescent protein comprises green fluorescent protein. In some embodiments, the reporter protein emits green fluorescence, yellow fluorescence, or red fluorescence. In some embodiments, the reporter comprises an enzyme. In some embodiments, the enzyme is β-galactosidase, alkaline phosphatase, β-lactamase, or luciferase.

Various HBV-antiviruses are contemplated herein. In some embodiments, the HBV-Antivirus is recombinant and genetically encoded. In some embodiments, the HBV-Antivirus comprises an enveloped particle. In some embodiments, the HBV-Antivirus comprises a lentiviral particle. In some embodiments, the HBV-Antivirus does not comprise a lentiviral particle. In some embodiments, the HBV-Antivirus comprises viral genetic material. In some embodiments, the HBV-Antivirus does not comprise viral genetic material. In some embodiments, the HBV-Antivirus comprises a lipid bilayer. In some embodiments, the HBV-Antivirus comprises a virus. In some embodiments, the HBV-Antivirus comprises a replication incompetent virus. In some embodiments, the HBV-Antivirus comprises a replication competent virus. In some embodiments, the HBV-Antivirus comprises a viral-like particle. In some embodiments, the HBV-Antivirus comprises an extracellular vesicle. In some embodiments, the HBV-Antivirus comprises an exosome. In some embodiments, the HBV-Antivirus comprises an ectosome.

An HBV-antivirus as described herein, in some embodiments, comprises a first fusion protein, wherein the first fusion protein is expressed at multiple copies on a surface of the HBV-antivirus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, 3000, 4000, 5000, or more than 5000 copies on a surface of the HBV-antivirus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the HBV-antivirus. In some embodiments, the first fusion protein is expressed at a valency of at least or about 10 copies on a surface of the HBV-antivirus. In some embodiments, the first fusion protein is expressed at a valency of at least or about 25 copies on a surface of the HBV-antivirus. In some embodiments, the first fusion protein is expressed at a valency of at least or about 50 copies on a surface of the HBV-antivirus. In some embodiments, the first fusion protein is expressed at a valency of at least or about 75 copies on a surface of the HBV-antivirus. In some embodiments, the first fusion protein is expressed at a valency of at least or about 100 copies on a surface of the HBV-antivirus. In some embodiments, the first fusion protein is expressed at a valency of at least or about 125 copies on a surface of the HBV-antivirus. In some embodiments, the first fusion protein is expressed at a valency of at least or about 150 copies on a surface of the HBV-antivirus. In some embodiments, the first fusion protein is expressed at a valency of at least or about 175 copies on a surface of the HBV-antivirus. In some embodiments, the first fusion protein is expressed at a valency of at least or about 200 copies on a surface of the HBV-antivirus. In some embodiments, the first fusion protein is expressed at a valency of at least or about 225 copies on a surface of the HBV-antivirus. In some embodiments, the first fusion protein is expressed at a valency of at least or about 250 copies on a surface of the HBV-antivirus. In some embodiments, the first fusion protein is expressed at a valency of at least or about 275 copies on a surface of the HBV-antivirus. In some embodiments, the first fusion protein is expressed at a valency of at least or about 300 copies on a surface of the HBV-antivirus. In some embodiments, the first fusion protein is expressed at a valency of at least or about 400 copies on a surface of the HBV-antivirus. In some embodiments, the first fusion protein is expressed at a valency of at least or about 500 copies on a surface of the HBV-antivirus. In some embodiments, the first fusion protein is expressed at a valency of at least or about 600 copies on a surface of the HBV-antivirus. In some embodiments, the first fusion protein is expressed at a valency of at least or about 700 copies on a surface of the HBV-antivirus. In some embodiments, the first fusion protein is expressed at a valency of at least or about 800 copies on a surface of the HBV-antivirus. In some embodiments, the first fusion protein is expressed at a valency of at least or about 900 copies on a surface of the HBV-antivirus. In some embodiments, the first fusion protein is expressed at a valency of at least or about 1000 copies on a surface of the HBV-antivirus. In some embodiments, the first fusion protein is expressed at a valency of at least or about 2000 copies on a surface of the HBV-antivirus. In some embodiments, the first fusion protein is expressed at a valency of at least or about 3000 copies on a surface of the HBV-antivirus. In some embodiments, the first fusion protein is expressed at a valency of at least or about 4000 copies on a surface of the HBV-antivirus. In some embodiments, the first fusion protein is expressed at a valency of at least or about 5000 copies on a surface of the HBV-antivirus.

In some embodiments, the HBV-Antivirus comprises an enveloped particle. The enveloped particle as described herein, in some embodiments, comprise a first fusion protein, wherein the first fusion protein is expressed at multiple copies on a surface of the enveloped particle. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the enveloped particle. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the enveloped particle. In some embodiments, the first fusion protein is expressed at a valency of at least or about 10 copies on a surface of the enveloped particle. In some embodiments, the first fusion protein is expressed at a valency of at least or about 25 copies on a surface of the enveloped particle. In some embodiments, the first fusion protein is expressed at a valency of at least or about 50 copies on a surface of the enveloped particle. In some embodiments, the first fusion protein is expressed at a valency of at least or about 75 copies on a surface of the enveloped particle. In some embodiments, the first fusion protein is expressed at a valency of at least or about 100 copies on a surface of the enveloped particle. In some embodiments, the first fusion protein is expressed at a valency of at least or about 125 copies on a surface of the enveloped particle. In some embodiments, the first fusion protein is expressed at a valency of at least or about 150 copies on a surface of the enveloped particle. In some embodiments, the first fusion protein is expressed at a valency of at least or about 175 copies on a surface of the enveloped particle. In some embodiments, the first fusion protein is expressed at a valency of at least or about 200 copies on a surface of the enveloped particle. In some embodiments, the first fusion protein is expressed at a valency of at least or about 225 copies on a surface of the enveloped particle. In some embodiments, the first fusion protein is expressed at a valency of at least or about 250 copies on a surface of the enveloped particle. In some embodiments, the first fusion protein is expressed at a valency of at least or about 275 copies on a surface of the enveloped particle. In some embodiments, the first fusion protein is expressed at a valency of at least or about 300 copies on a surface of the enveloped particle. In some embodiments, the first fusion protein is expressed at a valency of at least or about 400 copies on a surface of the enveloped particle. In some embodiments, the first fusion protein is expressed at a valency of at least or about 500 copies on a surface of the enveloped particle. In some embodiments, the first fusion protein is expressed at a valency of at least or about 600 copies on a surface of the enveloped particle. In some embodiments, the first fusion protein is expressed at a valency of at least or about 700 copies on a surface of the enveloped particle. In some embodiments, the first fusion protein is expressed at a valency of at least or about 800 copies on a surface of the enveloped particle. In some embodiments, the first fusion protein is expressed at a valency of at least or about 900 copies on a surface of the enveloped particle. In some embodiments, the first fusion protein is expressed at a valency of at least or about 1000 copies on a surface of the enveloped particle. In some embodiments, the first fusion protein is expressed at a valency of at least or about 2000 copies on a surface of the enveloped particle. In some embodiments, the first fusion protein is expressed at a valency of at least or about 3000 copies on a surface of the enveloped particle. In some embodiments, the first fusion protein is expressed at a valency of at least or about 4000 copies on a surface of the enveloped particle. In some embodiments, the first fusion protein is expressed at a valency of at least or about 5000 copies on a surface of the enveloped particle.

In some embodiments, the HBV-Antivirus comprises a viral-like particle (VLP). The viral-like particle as described herein, in some embodiments, comprise a first fusion protein, wherein the first fusion protein is expressed at multiple copies on a surface of the viral-like particle. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the viral-like particle. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the first fusion protein is expressed at a valency of at least or about 10 copies on a surface of the viral-like particle. In some embodiments, the first fusion protein is expressed at a valency of at least or about 25 copies on a surface of the viral-like particle. In some embodiments, the first fusion protein is expressed at a valency of at least or about 50 copies on a surface of the viral-like particle. In some embodiments, the first fusion protein is expressed at a valency of at least or about 75 copies on a surface of the viral-like particle. In some embodiments, the first fusion protein is expressed at a valency of at least or about 100 copies on a surface of the viral-like particle. In some embodiments, the first fusion protein is expressed at a valency of at least or about 125 copies on a surface of the viral-like particle. In some embodiments, the first fusion protein is expressed at a valency of at least or about 150 copies on a surface of the viral-like particle. In some embodiments, the first fusion protein is expressed at a valency of at least or about 175 copies on a surface of the viral-like particle. In some embodiments, the first fusion protein is expressed at a valency of at least or about 200 copies on a surface of the viral-like particle. In some embodiments, the first fusion protein is expressed at a valency of at least or about 225 copies on a surface of the viral-like particle. In some embodiments, the first fusion protein is expressed at a valency of at least or about 250 copies on a surface of the viral-like particle. In some embodiments, the first fusion protein is expressed at a valency of at least or about 275 copies on a surface of the viral-like particle. In some embodiments, the first fusion protein is expressed at a valency of at least or about 300 copies on a surface of the viral-like particle. In some embodiments, the first fusion protein is expressed at a valency of at least or about 400 copies on a surface of the viral-like particle. In some embodiments, the first fusion protein is expressed at a valency of at least or about 500 copies on a surface of the viral-like particle. In some embodiments, the first fusion protein is expressed at a valency of at least or about 600 copies on a surface of the viral-like particle. In some embodiments, the first fusion protein is expressed at a valency of at least or about 700 copies on a surface of the viral-like particle. In some embodiments, the first fusion protein is expressed at a valency of at least or about 800 copies on a surface of the viral-like particle. In some embodiments, the first fusion protein is expressed at a valency of at least or about 900 copies on a surface of the viral-like particle. In some embodiments, the first fusion protein is expressed at a valency of at least or about 1000 copies on a surface of the viral-like particle. In some embodiments, the first fusion protein is expressed at a valency of at least or about 2000 copies on a surface of the viral-like particle. In some embodiments, the first fusion protein is expressed at a valency of at least or about 3000 copies on a surface of the viral-like particle. In some embodiments, the first fusion protein is expressed at a valency of at least or about 4000 copies on a surface of the viral-like particle. In some embodiments, the first fusion protein is expressed at a valency of at least or about 5000 copies on a surface of the viral-like particle.

In some embodiments, the HBV-Antivirus comprises an extracellular vesicle. The extracellular vesicle as described herein, in some embodiments, comprise a first fusion protein, wherein the first fusion protein is expressed at multiple copies on a surface of the extracellular vesicle. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the extracellular vesicle. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the first fusion protein is expressed at a valency of at least or about 10 copies on a surface of the extracellular vesicle. In some embodiments, the first fusion protein is expressed at a valency of at least or about 25 copies on a surface of the extracellular vesicle. In some embodiments, the first fusion protein is expressed at a valency of at least or about 50 copies on a surface of the extracellular vesicle. In some embodiments, the first fusion protein is expressed at a valency of at least or about 75 copies on a surface of the extracellular vesicle. In some embodiments, the first fusion protein is expressed at a valency of at least or about 100 copies on a surface of the extracellular vesicle. In some embodiments, the first fusion protein is expressed at a valency of at least or about 125 copies on a surface of the extracellular vesicle. In some embodiments, the first fusion protein is expressed at a valency of at least or about 150 copies on a surface of the extracellular vesicle. In some embodiments, the first fusion protein is expressed at a valency of at least or about 175 copies on a surface of the extracellular vesicle. In some embodiments, the first fusion protein is expressed at a valency of at least or about 200 copies on a surface of the extracellular vesicle. In some embodiments, the first fusion protein is expressed at a valency of at least or about 225 copies on a surface of the extracellular vesicle. In some embodiments, the first fusion protein is expressed at a valency of at least or about 250 copies on a surface of the extracellular vesicle. In some embodiments, the first fusion protein is expressed at a valency of at least or about 275 copies on a surface of the extracellular vesicle. In some embodiments, the first fusion protein is expressed at a valency of at least or about 300 copies on a surface of the extracellular vesicle. In some embodiments, the first fusion protein is expressed at a valency of at least or about 400 copies on a surface of the extracellular vesicle. In some embodiments, the first fusion protein is expressed at a valency of at least or about 500 copies on a surface of the extracellular vesicle. In some embodiments, the first fusion protein is expressed at a valency of at least or about 600 copies on a surface of the extracellular vesicle. In some embodiments, the first fusion protein is expressed at a valency of at least or about 700 copies on a surface of the extracellular vesicle. In some embodiments, the first fusion protein is expressed at a valency of at least or about 800 copies on a surface of the extracellular vesicle. In some embodiments, the first fusion protein is expressed at a valency of at least or about 900 copies on a surface of the extracellular vesicle. In some embodiments, the first fusion protein is expressed at a valency of at least or about 1000 copies on a surface of the extracellular vesicle. In some embodiments, the first fusion protein is expressed at a valency of at least or about 2000 copies on a surface of the extracellular vesicle. In some embodiments, the first fusion protein is expressed at a valency of at least or about 3000 copies on a surface of the extracellular vesicle. In some embodiments, the first fusion protein is expressed at a valency of at least or about 4000 copies on a surface of the extracellular vesicle. In some embodiments, the first fusion protein is expressed at a valency of at least or about 5000 copies on a surface of the extracellular vesicle.

In some embodiments, the HBV-Antivirus comprises an exosome. The exosome as described herein, in some embodiments, comprise a first fusion protein, wherein the first fusion protein is expressed at multiple copies on a surface of the exosome. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the exosome. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the first fusion protein is expressed at a valency of at least or about 10 copies on a surface of the exosome. In some embodiments, the first fusion protein is expressed at a valency of at least or about 25 copies on a surface of the exosome. In some embodiments, the first fusion protein is expressed at a valency of at least or about 50 copies on a surface of the exosome. In some embodiments, the first fusion protein is expressed at a valency of at least or about 75 copies on a surface of the exosome. In some embodiments, the first fusion protein is expressed at a valency of at least or about 100 copies on a surface of the exosome. In some embodiments, the first fusion protein is expressed at a valency of at least or about 125 copies on a surface of the exosome. In some embodiments, the first fusion protein is expressed at a valency of at least or about 150 copies on a surface of the exosome. In some embodiments, the first fusion protein is expressed at a valency of at least or about 175 copies on a surface of the exosome. In some embodiments, the first fusion protein is expressed at a valency of at least or about 200 copies on a surface of the exosome. In some embodiments, the first fusion protein is expressed at a valency of at least or about 225 copies on a surface of the exosome. In some embodiments, the first fusion protein is expressed at a valency of at least or about 250 copies on a surface of the exosome. In some embodiments, the first fusion protein is expressed at a valency of at least or about 275 copies on a surface of the exosome. In some embodiments, the first fusion protein is expressed at a valency of at least or about 300 copies on a surface of the exosome. In some embodiments, the first fusion protein is expressed at a valency of at least or about 400 copies on a surface of the exosome. In some embodiments, the first fusion protein is expressed at a valency of at least or about 500 copies on a surface of the exosome. In some embodiments, the first fusion protein is expressed at a valency of at least or about 600 copies on a surface of the exosome. In some embodiments, the first fusion protein is expressed at a valency of at least or about 700 copies on a surface of the exosome. In some embodiments, the first fusion protein is expressed at a valency of at least or about 800 copies on a surface of the exosome. In some embodiments, the first fusion protein is expressed at a valency of at least or about 900 copies on a surface of the exosome. In some embodiments, the first fusion protein is expressed at a valency of at least or about 1000 copies on a surface of the exosome. In some embodiments, the first fusion protein is expressed at a valency of at least or about 2000 copies on a surface of the exosome. In some embodiments, the first fusion protein is expressed at a valency of at least or about 3000 copies on a surface of the exosome. In some embodiments, the first fusion protein is expressed at a valency of at least or about 4000 copies on a surface of the exosome. In some embodiments, the first fusion protein is expressed at a valency of at least or about 5000 copies on a surface of the exosome.

In some embodiments, the HBV-Antivirus comprises an ectosome. The ectosome as described herein, in some embodiments, comprise a first fusion protein, wherein the first fusion protein is expressed at multiple copies on a surface of the ectosome. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the ectosome. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the first fusion protein is expressed at a valency of at least or about 10 copies on a surface of the ectosome. In some embodiments, the first fusion protein is expressed at a valency of at least or about 25 copies on a surface of the ectosome. In some embodiments, the first fusion protein is expressed at a valency of at least or about 50 copies on a surface of the ectosome. In some embodiments, the first fusion protein is expressed at a valency of at least or about 75 copies on a surface of the ectosome. In some embodiments, the first fusion protein is expressed at a valency of at least or about 100 copies on a surface of the ectosome. In some embodiments, the first fusion protein is expressed at a valency of at least or about 125 copies on a surface of the ectosome. In some embodiments, the first fusion protein is expressed at a valency of at least or about 150 copies on a surface of the ectosome. In some embodiments, the first fusion protein is expressed at a valency of at least or about 175 copies on a surface of the ectosome. In some embodiments, the first fusion protein is expressed at a valency of at least or about 200 copies on a surface of the ectosome. In some embodiments, the first fusion protein is expressed at a valency of at least or about 225 copies on a surface of the ectosome. In some embodiments, the first fusion protein is expressed at a valency of at least or about 250 copies on a surface of the ectosome. In some embodiments, the first fusion protein is expressed at a valency of at least or about 275 copies on a surface of the ectosome. In some embodiments, the first fusion protein is expressed at a valency of at least or about 300 copies on a surface of the ectosome. In some embodiments, the first fusion protein is expressed at a valency of at least or about 400 copies on a surface of the ectosome. In some embodiments, the first fusion protein is expressed at a valency of at least or about 500 copies on a surface of the ectosome. In some embodiments, the first fusion protein is expressed at a valency of at least or about 600 copies on a surface of the ectosome. In some embodiments, the first fusion protein is expressed at a valency of at least or about 700 copies on a surface of the ectosome. In some embodiments, the first fusion protein is expressed at a valency of at least or about 800 copies on a surface of the ectosome. In some embodiments, the first fusion protein is expressed at a valency of at least or about 900 copies on a surface of the ectosome. In some embodiments, the first fusion protein is expressed at a valency of at least or about 1000 copies on a surface of the ectosome. In some embodiments, the first fusion protein is expressed at a valency of at least or about 2000 copies on a surface of the ectosome. In some embodiments, the first fusion protein is expressed at a valency of at least or about 3000 copies on a surface of the ectosome. In some embodiments, the first fusion protein is expressed at a valency of at least or about 4000 copies on a surface of the ectosome. In some embodiments, the first fusion protein is expressed at a valency of at least or about 5000 copies on a surface of the ectosome.

In some embodiments, the HBV-Antivirus comprises a virus. The virus as described herein, in some embodiments, comprise a first fusion protein, wherein the first fusion protein is expressed at multiple copies on a surface of the virus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the virus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the virus. In some embodiments, the first fusion protein is expressed at a valency of at least or about 10 copies on a surface of the virus. In some embodiments, the first fusion protein is expressed at a valency of at least or about 25 copies on a surface of the virus. In some embodiments, the first fusion protein is expressed at a valency of at least or about 50 copies on a surface of the virus. In some embodiments, the first fusion protein is expressed at a valency of at least or about 75 copies on a surface of the virus. In some embodiments, the first fusion protein is expressed at a valency of at least or about 100 copies on a surface of the virus. In some embodiments, the first fusion protein is expressed at a valency of at least or about 125 copies on a surface of the virus. In some embodiments, the first fusion protein is expressed at a valency of at least or about 150 copies on a surface of the virus. In some embodiments, the first fusion protein is expressed at a valency of at least or about 175 copies on a surface of the virus. In some embodiments, the first fusion protein is expressed at a valency of at least or about 200 copies on a surface of the virus. In some embodiments, the first fusion protein is expressed at a valency of at least or about 225 copies on a surface of the virus. In some embodiments, the first fusion protein is expressed at a valency of at least or about 250 copies on a surface of the virus. In some embodiments, the first fusion protein is expressed at a valency of at least or about 275 copies on a surface of the virus. In some embodiments, the first fusion protein is expressed at a valency of at least or about 300 copies on a surface of the virus. In some embodiments, the first fusion protein is expressed at a valency of at least or about 400 copies on a surface of the virus. In some embodiments, the first fusion protein is expressed at a valency of at least or about 500 copies on a surface of the virus. In some embodiments, the first fusion protein is expressed at a valency of at least or about 600 copies on a surface of the virus. In some embodiments, the first fusion protein is expressed at a valency of at least or about 700 copies on a surface of the virus. In some embodiments, the first fusion protein is expressed at a valency of at least or about 800 copies on a surface of the virus. In some embodiments, the first fusion protein is expressed at a valency of at least or about 900 copies on a surface of the virus. In some embodiments, the first fusion protein is expressed at a valency of at least or about 1000 copies on a surface of the virus. In some embodiments, the first fusion protein is expressed at a valency of at least or about 2000 copies on a surface of the virus. In some embodiments, the first fusion protein is expressed at a valency of at least or about 3000 copies on a surface of the virus. In some embodiments, the first fusion protein is expressed at a valency of at least or about 4000 copies on a surface of the virus. In some embodiments, the first fusion protein is expressed at a valency of at least or about 5000 copies on a surface of the virus.

In some embodiments, the HBV-Antivirus comprises a replication incompetent virus. The replication incompetent virus as described herein, in some embodiments, comprise a first fusion protein, wherein the first fusion protein is expressed at multiple copies on a surface of the replication incompetent virus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication incompetent virus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication incompetent virus. In some embodiments, the first fusion protein is expressed at a valency of at least or about 10 copies on a surface of the replication incompetent virus. In some embodiments, the first fusion protein is expressed at a valency of at least or about 25 copies on a surface of the replication incompetent virus. In some embodiments, the first fusion protein is expressed at a valency of at least or about 50 copies on a surface of the replication incompetent virus. In some embodiments, the first fusion protein is expressed at a valency of at least or about 75 copies on a surface of the replication incompetent virus. In some embodiments, the first fusion protein is expressed at a valency of at least or about 100 copies on a surface of the replication incompetent virus. In some embodiments, the first fusion protein is expressed at a valency of at least or about 125 copies on a surface of the replication incompetent virus. In some embodiments, the first fusion protein is expressed at a valency of at least or about 150 copies on a surface of the replication incompetent virus. In some embodiments, the first fusion protein is expressed at a valency of at least or about 175 copies on a surface of the replication incompetent virus. In some embodiments, the first fusion protein is expressed at a valency of at least or about 200 copies on a surface of the replication incompetent virus. In some embodiments, the first fusion protein is expressed at a valency of at least or about 225 copies on a surface of the replication incompetent virus. In some embodiments, the first fusion protein is expressed at a valency of at least or about 250 copies on a surface of the replication incompetent virus. In some embodiments, the first fusion protein is expressed at a valency of at least or about 275 copies on a surface of the replication incompetent virus. In some embodiments, the first fusion protein is expressed at a valency of at least or about 300 copies on a surface of the replication incompetent virus. In some embodiments, the first fusion protein is expressed at a valency of at least or about 400 copies on a surface of the replication incompetent virus. In some embodiments, the first fusion protein is expressed at a valency of at least or about 500 copies on a surface of the replication incompetent virus. In some embodiments, the first fusion protein is expressed at a valency of at least or about 600 copies on a surface of the replication incompetent virus. In some embodiments, the first fusion protein is expressed at a valency of at least or about 700 copies on a surface of the replication incompetent virus. In some embodiments, the first fusion protein is expressed at a valency of at least or about 800 copies on a surface of the replication incompetent virus. In some embodiments, the first fusion protein is expressed at a valency of at least or about 900 copies on a surface of the replication incompetent virus. In some embodiments, the first fusion protein is expressed at a valency of at least or about 1000 copies on a surface of the replication incompetent virus. In some embodiments, the first fusion protein is expressed at a valency of at least or about 2000 copies on a surface of the replication incompetent virus. In some embodiments, the first fusion protein is expressed at a valency of at least or about 3000 copies on a surface of the replication incompetent virus. In some embodiments, the first fusion protein is expressed at a valency of at least or about 4000 copies on a surface of the replication incompetent virus. In some embodiments, the first fusion protein is expressed at a valency of at least or about 5000 copies on a surface of the replication incompetent virus.

In some embodiments, the HBV-Antivirus comprises a replication competent virus. The replication competent virus as described herein, in some embodiments, comprise a first fusion protein, wherein the first fusion protein is expressed at multiple copies on a surface of the replication competent virus. In some embodiments, the first fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication competent virus. In some embodiments, the first fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the first fusion protein is expressed at a valency of at least or about 10 copies on a surface of the replication competent virus. In some embodiments, the first fusion protein is expressed at a valency of at least or about 25 copies on a surface of the replication competent virus. In some embodiments, the first fusion protein is expressed at a valency of at least or about 50 copies on a surface of the replication competent virus. In some embodiments, the first fusion protein is expressed at a valency of at least or about 75 copies on a surface of the replication competent virus. In some embodiments, the first fusion protein is expressed at a valency of at least or about 100 copies on a surface of the replication competent virus. In some embodiments, the first fusion protein is expressed at a valency of at least or about 125 copies on a surface of the replication competent virus. In some embodiments, the first fusion protein is expressed at a valency of at least or about 150 copies on a surface of the replication competent virus. In some embodiments, the first fusion protein is expressed at a valency of at least or about 175 copies on a surface of the replication competent virus. In some embodiments, the first fusion protein is expressed at a valency of at least or about 200 copies on a surface of the replication competent virus. In some embodiments, the first fusion protein is expressed at a valency of at least or about 225 copies on a surface of the replication competent virus. In some embodiments, the first fusion protein is expressed at a valency of at least or about 250 copies on a surface of the replication competent virus. In some embodiments, the first fusion protein is expressed at a valency of at least or about 275 copies on a surface of the replication competent virus. In some embodiments, the first fusion protein is expressed at a valency of at least or about 300 copies on a surface of the replication competent virus. In some embodiments, the first fusion protein is expressed at a valency of at least or about 400 copies on a surface of the replication competent virus. In some embodiments, the first fusion protein is expressed at a valency of at least or about 500 copies on a surface of the replication competent virus. In some embodiments, the first fusion protein is expressed at a valency of at least or about 600 copies on a surface of the replication competent virus. In some embodiments, the first fusion protein is expressed at a valency of at least or about 700 copies on a surface of the replication competent virus. In some embodiments, the first fusion protein is expressed at a valency of at least or about 800 copies on a surface of the replication competent virus. In some embodiments, the first fusion protein is expressed at a valency of at least or about 900 copies on a surface of the replication competent virus. In some embodiments, the first fusion protein is expressed at a valency of at least or about 1000 copies on a surface of the replication competent virus. In some embodiments, the first fusion protein is expressed at a valency of at least or about 2000 copies on a surface of the replication competent virus. In some embodiments, the first fusion protein is expressed at a valency of at least or about 3000 copies on a surface of the replication competent virus. In some embodiments, the first fusion protein is expressed at a valency of at least or about 4000 copies on a surface of the replication competent virus. In some embodiments, the first fusion protein is expressed at a valency of at least or about 5000 copies on a surface of the replication competent virus.

HBV-antiviruses as described herein, in some embodiments, comprise a second fusion protein to further increase the valency of HBV-antivirus, wherein the second fusion protein is expressed at multiple copies on a surface of the HBV-antivirus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, 3000, 4000, 5000, or more than 5000 copies on a surface of the HBV-antivirus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the HBV-antivirus. In some embodiments, the second fusion protein is expressed at a valency of at least or about 10 copies on a surface of the HBV-antivirus. In some embodiments, the second fusion protein is expressed at a valency of at least or about 25 copies on a surface of the HBV-antivirus. In some embodiments, the second fusion protein is expressed at a valency of at least or about 50 copies on a surface of the HBV-antivirus. In some embodiments, the second fusion protein is expressed at a valency of at least or about 75 copies on a surface of the multivalent particle. In some embodiments, the second fusion protein is expressed at a valency of at least or about 100 copies on a surface of the HBV-antivirus. In some embodiments, the second fusion protein is expressed at a valency of at least or about 125 copies on a surface of the HBV-antivirus. In some embodiments, the second fusion protein is expressed at a valency of at least or about 150 copies on a surface of the HBV-antivirus. In some embodiments, the second fusion protein is expressed at a valency of at least or about 175 copies on a surface of the HBV-antivirus. In some embodiments, the second fusion protein is expressed at a valency of at least or about 200 copies on a surface of the HBV-antivirus. In some embodiments, the second fusion protein is expressed at a valency of at least or about 225 copies on a surface of the HBV-antivirus. In some embodiments, the second fusion protein is expressed at a valency of at least or about 250 copies on a surface of the HBV-antivirus. In some embodiments, the second fusion protein is expressed at a valency of at least or about 275 copies on a surface of the HBV-antivirus. In some embodiments, the second fusion protein is expressed at a valency of at least or about 300 copies on a surface of the HBV-antivirus. In some embodiments, the second fusion protein is expressed at a valency of at least or about 400 copies on a surface of the HBV-antivirus. In some embodiments, the second fusion protein is expressed at a valency of at least or about 500 copies on a surface of the HBV-antivirus. In some embodiments, the second fusion protein is expressed at a valency of at least or about 600 copies on a surface of the HBV-antivirus. In some embodiments, the second fusion protein is expressed at a valency of at least or about 700 copies on a surface of the HBV-antivirus. In some embodiments, the second fusion protein is expressed at a valency of at least or about 800 copies on a surface of the HBV-antivirus. In some embodiments, the second fusion protein is expressed at a valency of at least or about 900 copies on a surface of the HBV-antivirus. In some embodiments, the second fusion protein is expressed at a valency of at least or about 1000 copies on a surface of the HBV-antivirus. In some embodiments, the second fusion protein is expressed at a valency of at least or about 2000 copies on a surface of the HBV-antivirus. In some embodiments, the second fusion protein is expressed at a valency of at least or about 3000 copies on a surface of the HBV-antivirus. In some embodiments, the second fusion protein is expressed at a valency of at least or about 4000 copies on a surface of the HBV-antivirus. In some embodiments, the second fusion protein is expressed at a valency of at least or about 5000 copies on a surface of the HBV-antivirus.

The enveloped particle, as described herein, in some embodiments, comprise a second fusion protein, wherein the second fusion protein is expressed at multiple copies on a surface of the enveloped particle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the enveloped particle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the enveloped particle. In some embodiments, the second fusion protein is expressed at a valency of at least or about 10 copies on a surface of the enveloped particle. In some embodiments, the second fusion protein is expressed at a valency of at least or about 25 copies on a surface of the enveloped particle. In some embodiments, the second fusion protein is expressed at a valency of at least or about 50 copies on a surface of the enveloped particle. In some embodiments, the second fusion protein is expressed at a valency of at least or about 75 copies on a surface of the enveloped particle. In some embodiments, the second fusion protein is expressed at a valency of at least or about 100 copies on a surface of the enveloped particle. In some embodiments, the second fusion protein is expressed at a valency of at least or about 125 copies on a surface of the enveloped particle. In some embodiments, the second fusion protein is expressed at a valency of at least or about 150 copies on a surface of the enveloped particle. In some embodiments, the second fusion protein is expressed at a valency of at least or about 175 copies on a surface of the enveloped particle. In some embodiments, the second fusion protein is expressed at a valency of at least or about 200 copies on a surface of the enveloped particle. In some embodiments, the second fusion protein is expressed at a valency of at least or about 225 copies on a surface of the enveloped particle. In some embodiments, the second fusion protein is expressed at a valency of at least or about 250 copies on a surface of the enveloped particle. In some embodiments, the second fusion protein is expressed at a valency of at least or about 275 copies on a surface of the enveloped particle. In some embodiments, the second fusion protein is expressed at a valency of at least or about 300 copies on a surface of the enveloped particle. In some embodiments, the second fusion protein is expressed at a valency of at least or about 400 copies on a surface of the enveloped particle. In some embodiments, the second fusion protein is expressed at a valency of at least or about 500 copies on a surface of the enveloped particle. In some embodiments, the second fusion protein is expressed at a valency of at least or about 600 copies on a surface of the enveloped particle. In some embodiments, the second fusion protein is expressed at a valency of at least or about 700 copies on a surface of the enveloped particle. In some embodiments, the second fusion protein is expressed at a valency of at least or about 800 copies on a surface of the enveloped particle. In some embodiments, the second fusion protein is expressed at a valency of at least or about 900 copies on a surface of the enveloped particle. In some embodiments, the second fusion protein is expressed at a valency of at least or about 1000 copies on a surface of the enveloped particle. In some embodiments, the second fusion protein is expressed at a valency of at least or about 2000 copies on a surface of the enveloped particle. In some embodiments, the second fusion protein is expressed at a valency of at least or about 3000 copies on a surface of the enveloped particle. In some embodiments, the second fusion protein is expressed at a valency of at least or about 4000 copies on a surface of the enveloped particle. In some embodiments, the second fusion protein is expressed at a valency of at least or about 5000 copies on a surface of the enveloped particle.

The viral-like particle as described herein, in some embodiments, comprise a second fusion protein, wherein the second fusion protein is expressed at multiple copies on a surface of the viral-like particle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the viral-like particle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the viral-like particle. In some embodiments, the second fusion protein is expressed at a valency of at least or about 10 copies on a surface of the viral-like particle. In some embodiments, the second fusion protein is expressed at a valency of at least or about 25 copies on a surface of the viral-like particle. In some embodiments, the second fusion protein is expressed at a valency of at least or about 50 copies on a surface of the viral-like particle. In some embodiments, the second fusion protein is expressed at a valency of at least or about 75 copies on a surface of the viral-like particle. In some embodiments, the second fusion protein is expressed at a valency of at least or about 100 copies on a surface of the viral-like particle. In some embodiments, the second fusion protein is expressed at a valency of at least or about 125 copies on a surface of the viral-like particle. In some embodiments, the second fusion protein is expressed at a valency of at least or about 150 copies on a surface of the viral-like particle. In some embodiments, the second fusion protein is expressed at a valency of at least or about 175 copies on a surface of the viral-like particle. In some embodiments, the second fusion protein is expressed at a valency of at least or about 200 copies on a surface of the viral-like particle. In some embodiments, the second fusion protein is expressed at a valency of at least or about 225 copies on a surface of the viral-like particle. In some embodiments, the second fusion protein is expressed at a valency of at least or about 250 copies on a surface of the viral-like particle. In some embodiments, the second fusion protein is expressed at a valency of at least or about 275 copies on a surface of the viral-like particle. In some embodiments, the second fusion protein is expressed at a valency of at least or about 300 copies on a surface of the viral-like particle. In some embodiments, the second fusion protein is expressed at a valency of at least or about 300 copies on a surface of the viral-like particle. In some embodiments, the second fusion protein is expressed at a valency of at least or about 400 copies on a surface of the viral-like particle. In some embodiments, the second fusion protein is expressed at a valency of at least or about 500 copies on a surface of the viral-like particle. In some embodiments, the second fusion protein is expressed at a valency of at least or about 600 copies on a surface of the viral-like particle. In some embodiments, the second fusion protein is expressed at a valency of at least or about 700 copies on a surface of the viral-like particle. In some embodiments, the second fusion protein is expressed at a valency of at least or about 800 copies on a surface of the viral-like particle. In some embodiments, the second fusion protein is expressed at a valency of at least or about 900 copies on a surface of the viral-like particle. In some embodiments, the second fusion protein is expressed at a valency of at least or about 1000 copies on a surface of the viral-like particle. In some embodiments, the second fusion protein is expressed at a valency of at least or about 2000 copies on a surface of the viral-like particle. In some embodiments, the second fusion protein is expressed at a valency of at least or about 3000 copies on a surface of the viral-like particle. In some embodiments, the second fusion protein is expressed at a valency of at least or about 4000 copies on a surface of the viral-like particle. In some embodiments, the second fusion protein is expressed at a valency of at least or about 5000 copies on a surface of the viral-like particle.

The extracellular vesicle, as described herein, in some embodiments, comprise a second fusion protein, wherein the second fusion protein is expressed at multiple copies on a surface of the extracellular vesicle. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the extracellular vesicle. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the extracellular vesicle. In some embodiments, the second fusion protein is expressed at a valency of at least or about 10 copies on a surface of the extracellular vesicle. In some embodiments, the second fusion protein is expressed at a valency of at least or about 25 copies on a surface of the extracellular vesicle. In some embodiments, the second fusion protein is expressed at a valency of at least or about 50 copies on a surface of the extracellular vesicle. In some embodiments, the second fusion protein is expressed at a valency of at least or about 75 copies on a surface of the extracellular vesicle. In some embodiments, the second fusion protein is expressed at a valency of at least or about 100 copies on a surface of the extracellular vesicle. In some embodiments, the second fusion protein is expressed at a valency of at least or about 125 copies on a surface of the extracellular vesicle. In some embodiments, the second fusion protein is expressed at a valency of at least or about 150 copies on a surface of the extracellular vesicle. In some embodiments, the second fusion protein is expressed at a valency of at least or about 175 copies on a surface of the extracellular vesicle. In some embodiments, the second fusion protein is expressed at a valency of at least or about 200 copies on a surface of the extracellular vesicle. In some embodiments, the second fusion protein is expressed at a valency of at least or about 225 copies on a surface of the extracellular vesicle. In some embodiments, the second fusion protein is expressed at a valency of at least or about 250 copies on a surface of the extracellular vesicle. In some embodiments, the second fusion protein is expressed at a valency of at least or about 275 copies on a surface of the extracellular vesicle. In some embodiments, the second fusion protein is expressed at a valency of at least or about 300 copies on a surface of the extracellular vesicle. In some embodiments, the second fusion protein is expressed at a valency of at least or about 400 copies on a surface of the extracellular vesicle. In some embodiments, the second fusion protein is expressed at a valency of at least or about 500 copies on a surface of the extracellular vesicle. In some embodiments, the second fusion protein is expressed at a valency of at least or about 600 copies on a surface of the extracellular vesicle. In some embodiments, the second fusion protein is expressed at a valency of at least or about 700 copies on a surface of the extracellular vesicle. In some embodiments, the second fusion protein is expressed at a valency of at least or about 800 copies on a surface of the extracellular vesicle. In some embodiments, the second fusion protein is expressed at a valency of at least or about 900 copies on a surface of the extracellular vesicle. In some embodiments, the second fusion protein is expressed at a valency of at least or about 1000 copies on a surface of the extracellular vesicle. In some embodiments, the second fusion protein is expressed at a valency of at least or about 2000 copies on a surface of the extracellular vesicle. In some embodiments, the second fusion protein is expressed at a valency of at least or about 3000 copies on a surface of the extracellular vesicle. In some embodiments, the second fusion protein is expressed at a valency of at least or about 4000 copies on a surface of the extracellular vesicle. In some embodiments, the second fusion protein is expressed at a valency of at least or about 5000 copies on a surface of the extracellular vesicle.

The exosome, as described herein, in some embodiments, comprise a second fusion protein, wherein the second fusion protein is expressed at multiple copies on a surface of the exosome. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the exosome. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the exosome. In some embodiments, the second fusion protein is expressed at a valency of at least or about 10 copies on a surface of the exosome. In some embodiments, the second fusion protein is expressed at a valency of at least or about 25 copies on a surface of the exosome. In some embodiments, the second fusion protein is expressed at a valency of at least or about 50 copies on a surface of the exosome. In some embodiments, the second fusion protein is expressed at a valency of at least or about 75 copies on a surface of the exosome. In some embodiments, the second fusion protein is expressed at a valency of at least or about 100 copies on a surface of the exosome. In some embodiments, the second fusion protein is expressed at a valency of at least or about 125 copies on a surface of the exosome. In some embodiments, the second fusion protein is expressed at a valency of at least or about 150 copies on a surface of the exosome. In some embodiments, the second fusion protein is expressed at a valency of at least or about 175 copies on a surface of the exosome. In some embodiments, the second fusion protein is expressed at a valency of at least or about 200 copies on a surface of the exosome. In some embodiments, the second fusion protein is expressed at a valency of at least or about 225 copies on a surface of the exosome. In some embodiments, the second fusion protein is expressed at a valency of at least or about 250 copies on a surface of the exosome. In some embodiments, the second fusion protein is expressed at a valency of at least or about 275 copies on a surface of the exosome. In some embodiments, the second fusion protein is expressed at a valency of at least or about 300 copies on a surface of the exosome. In some embodiments, the second fusion protein is expressed at a valency of at least or about 400 copies on a surface of the exosome. In some embodiments, the second fusion protein is expressed at a valency of at least or about 500 copies on a surface of the exosome. In some embodiments, the second fusion protein is expressed at a valency of at least or about 600 copies on a surface of the exosome. In some embodiments, the second fusion protein is expressed at a valency of at least or about 700 copies on a surface of the exosome. In some embodiments, the second fusion protein is expressed at a valency of at least or about 800 copies on a surface of the exosome. In some embodiments, the second fusion protein is expressed at a valency of at least or about 900 copies on a surface of the exosome. In some embodiments, the second fusion protein is expressed at a valency of at least or about 1000 copies on a surface of the exosome. In some embodiments, the second fusion protein is expressed at a valency of at least or about 2000 copies on a surface of the exosome. In some embodiments, the second fusion protein is expressed at a valency of at least or about 3000 copies on a surface of the exosome. In some embodiments, the second fusion protein is expressed at a valency of at least or about 4000 copies on a surface of the exosome. In some embodiments, the second fusion protein is expressed at a valency of at least or about 5000 copies on a surface of the exosome.

The ectosome, as described herein, in some embodiments, comprises a second fusion protein, wherein the second fusion protein is expressed at multiple copies on a surface of the ectosome. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the ectosome. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the ectosome. In some embodiments, the second fusion protein is expressed at a valency of at least or about 10 copies on a surface of the ectosome. In some embodiments, the second fusion protein is expressed at a valency of at least or about 25 copies on a surface of the ectosome. In some embodiments, the second fusion protein is expressed at a valency of at least or about 50 copies on a surface of the ectosome. In some embodiments, the second fusion protein is expressed at a valency of at least or about 75 copies on a surface of the ectosome. In some embodiments, the second fusion protein is expressed at a valency of at least or about 100 copies on a surface of the ectosome. In some embodiments, the second fusion protein is expressed at a valency of at least or about 125 copies on a surface of the ectosome. In some embodiments, the second fusion protein is expressed at a valency of at least or about 150 copies on a surface of the ectosome. In some embodiments, the second fusion protein is expressed at a valency of at least or about 175 copies on a surface of the ectosome. In some embodiments, the second fusion protein is expressed at a valency of at least or about 200 copies on a surface of the ectosome. In some embodiments, the second fusion protein is expressed at a valency of at least or about 225 copies on a surface of the ectosome. In some embodiments, the second fusion protein is expressed at a valency of at least or about 250 copies on a surface of the ectosome. In some embodiments, the second fusion protein is expressed at a valency of at least or about 275 copies on a surface of the ectosome. In some embodiments, the second fusion protein is expressed at a valency of at least or about 300 copies on a surface of the ectosome. In some embodiments, the second fusion protein is expressed at a valency of at least or about 400 copies on a surface of the ectosome. In some embodiments, the second fusion protein is expressed at a valency of at least or about 500 copies on a surface of the ectosome. In some embodiments, the second fusion protein is expressed at a valency of at least or about 600 copies on a surface of the ectosome. In some embodiments, the second fusion protein is expressed at a valency of at least or about 700 copies on a surface of the ectosome. In some embodiments, the second fusion protein is expressed at a valency of at least or about 800 copies on a surface of the ectosome. In some embodiments, the second fusion protein is expressed at a valency of at least or about 900 copies on a surface of the ectosome. In some embodiments, the second fusion protein is expressed at a valency of at least or about 1000 copies on a surface of the ectosome. In some embodiments, the second fusion protein is expressed at a valency of at least or about 2000 copies on a surface of the ectosome. In some embodiments, the second fusion protein is expressed at a valency of at least or about 3000 copies on a surface of the ectosome. In some embodiments, the second fusion protein is expressed at a valency of at least or about 4000 copies on a surface of the ectosome. In some embodiments, the second fusion protein is expressed at a valency of at least or about 5000 copies on a surface of the ectosome.

The virus as described herein, in some embodiments, comprise a second fusion protein, wherein the second fusion protein is expressed at multiple copies on a surface of the virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the virus. In some embodiments, the second fusion protein is expressed at a valency of at least or about 10 copies on a surface of the virus. In some embodiments, the second fusion protein is expressed at a valency of at least or about 25 copies on a surface of the virus. In some embodiments, the second fusion protein is expressed at a valency of at least or about 50 copies on a surface of the virus. In some embodiments, the second fusion protein is expressed at a valency of at least or about 75 copies on a surface of the virus. In some embodiments, the second fusion protein is expressed at a valency of at least or about 100 copies on a surface of the virus. In some embodiments, the second fusion protein is expressed at a valency of at least or about 125 copies on a surface of the virus. In some embodiments, the second fusion protein is expressed at a valency of at least or about 150 copies on a surface of the virus. In some embodiments, the second fusion protein is expressed at a valency of at least or about 175 copies on a surface of the virus. In some embodiments, the second fusion protein is expressed at a valency of at least or about 200 copies on a surface of the virus. In some embodiments, the second fusion protein is expressed at a valency of at least or about 225 copies on a surface of the virus. In some embodiments, the second fusion protein is expressed at a valency of at least or about 250 copies on a surface of the virus. In some embodiments, the second fusion protein is expressed at a valency of at least or about 275 copies on a surface of the virus. In some embodiments, the second fusion protein is expressed at a valency of at least or about 300 copies on a surface of the virus. In some embodiments, the second fusion protein is expressed at a valency of at least or about 300 copies on a surface of the virus. In some embodiments, the second fusion protein is expressed at a valency of at least or about 400 copies on a surface of the virus. In some embodiments, the second fusion protein is expressed at a valency of at least or about 500 copies on a surface of the virus. In some embodiments, the second fusion protein is expressed at a valency of at least or about 600 copies on a surface of the virus. In some embodiments, the second fusion protein is expressed at a valency of at least or about 700 copies on a surface of the virus. In some embodiments, the second fusion protein is expressed at a valency of at least or about 800 copies on a surface of the virus. In some embodiments, the second fusion protein is expressed at a valency of at least or about 900 copies on a surface of the virus. In some embodiments, the second fusion protein is expressed at a valency of at least or about 1000 copies on a surface of the virus. In some embodiments, the second fusion protein is expressed at a valency of at least or about 2000 copies on a surface of the virus. In some embodiments, the second fusion protein is expressed at a valency of at least or about 3000 copies on a surface of the virus. In some embodiments, the second fusion protein is expressed at a valency of at least or about 4000 copies on a surface of the virus. In some embodiments, the second fusion protein is expressed at a valency of at least or about 5000 copies on a surface of the virus.

The replication incompetent virus as described herein, in some embodiments, comprise a second fusion protein, wherein the second fusion protein is expressed at multiple copies on a surface of the replication incompetent virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication incompetent virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication incompetent virus. In some embodiments, the second fusion protein is expressed at a valency of at least or about 10 copies on a surface of the replication incompetent virus. In some embodiments, the second fusion protein is expressed at a valency of at least or about 25 copies on a surface of the replication incompetent virus. In some embodiments, the second fusion protein is expressed at a valency of at least or about 50 copies on a surface of the replication incompetent virus. In some embodiments, the second fusion protein is expressed at a valency of at least or about 75 copies on a surface of the replication incompetent virus. In some embodiments, the second fusion protein is expressed at a valency of at least or about 100 copies on a surface of the replication incompetent virus. In some embodiments, the second fusion protein is expressed at a valency of at least or about 125 copies on a surface of the replication incompetent virus. In some embodiments, the second fusion protein is expressed at a valency of at least or about 150 copies on a surface of the replication incompetent virus. In some embodiments, the second fusion protein is expressed at a valency of at least or about 175 copies on a surface of the replication incompetent virus. In some embodiments, the second fusion protein is expressed at a valency of at least or about 200 copies on a surface of the replication incompetent virus. In some embodiments, the second fusion protein is expressed at a valency of at least or about 225 copies on a surface of the replication incompetent virus. In some embodiments, the second fusion protein is expressed at a valency of at least or about 250 copies on a surface of the replication incompetent virus. In some embodiments, the second fusion protein is expressed at a valency of at least or about 275 copies on a surface of the replication incompetent virus. In some embodiments, the second fusion protein is expressed at a valency of at least or about 300 copies on a surface of the replication incompetent virus. In some embodiments, the second fusion protein is expressed at a valency of at least or about 300 copies on a surface of the replication incompetent virus. In some embodiments, the second fusion protein is expressed at a valency of at least or about 400 copies on a surface of the replication incompetent virus. In some embodiments, the second fusion protein is expressed at a valency of at least or about 500 copies on a surface of the replication incompetent virus. In some embodiments, the second fusion protein is expressed at a valency of at least or about 600 copies on a surface of the replication incompetent virus. In some embodiments, the second fusion protein is expressed at a valency of at least or about 700 copies on a surface of the replication incompetent virus. In some embodiments, the second fusion protein is expressed at a valency of at least or about 800 copies on a surface of the replication incompetent virus. In some embodiments, the second fusion protein is expressed at a valency of at least or about 900 copies on a surface of the replication incompetent virus. In some embodiments, the second fusion protein is expressed at a valency of at least or about 1000 copies on a surface of the replication incompetent virus. In some embodiments, the second fusion protein is expressed at a valency of at least or about 2000 copies on a surface of the replication incompetent virus. In some embodiments, the second fusion protein is expressed at a valency of at least or about 3000 copies on a surface of the replication incompetent virus. In some embodiments, the second fusion protein is expressed at a valency of at least or about 4000 copies on a surface of the replication incompetent virus. In some embodiments, the second fusion protein is expressed at a valency of at least or about 5000 copies on a surface of the replication incompetent virus.

The replication competent virus as described herein, in some embodiments, comprise a second fusion protein, wherein the second fusion protein is expressed at multiple copies on a surface of the replication competent virus. In some embodiments, the first fusion protein is monomeric. In some embodiments, the first fusion protein is dimeric. In some embodiments, the first fusion protein is trimeric. In some embodiments, the first fusion protein is tetrameric. In some embodiments, the second fusion protein is monomeric. In some embodiments, the second fusion protein is dimeric. In some embodiments, the second fusion protein is trimeric. In some embodiments, the second fusion protein is tetrameric. In some embodiments, the second fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 500, 600, 700, 800, 900, 1000, 1200, 1400, 1600, 1800, 2000, or more than 2000 copies on a surface of the replication competent virus. In some embodiments, the second fusion protein is expressed at a valency of at least or about 5 to about 400, about 20 to about 400, about 10 to about 300, about 20 to about 300, about 20 to about 200, about 50 to about 150, about 20 to about 100, or about 50 to about 100 copies on a surface of the replication competent virus. In some embodiments, the second fusion protein is expressed at a valency of at least or about 10 copies on a surface of the replication competent virus. In some embodiments, the second fusion protein is expressed at a valency of at least or about 25 copies on a surface of the replication competent virus. In some embodiments, the second fusion protein is expressed at a valency of at least or about 50 copies on a surface of the replication competent virus. In some embodiments, the second fusion protein is expressed at a valency of at least or about 75 copies on a surface of the replication competent virus. In some embodiments, the second fusion protein is expressed at a valency of at least or about 100 copies on a surface of the replication competent virus. In some embodiments, the second fusion protein is expressed at a valency of at least or about 125 copies on a surface of the replication competent virus. In some embodiments, the second fusion protein is expressed at a valency of at least or about 150 copies on a surface of the replication competent virus. In some embodiments, the second fusion protein is expressed at a valency of at least or about 175 copies on a surface of the replication competent virus. In some embodiments, the second fusion protein is expressed at a valency of at least or about 200 copies on a surface of the replication competent virus. In some embodiments, the second fusion protein is expressed at a valency of at least or about 225 copies on a surface of the replication competent virus. In some embodiments, the second fusion protein is expressed at a valency of at least or about 250 copies on a surface of the replication competent virus. In some embodiments, the second fusion protein is expressed at a valency of at least or about 275 copies on a surface of the replication competent virus. In some embodiments, the second fusion protein is expressed at a valency of at least or about 300 copies on a surface of the replication competent virus. In some embodiments, the second fusion protein is expressed at a valency of at least or about 300 copies on a surface of the replication competent virus. In some embodiments, the second fusion protein is expressed at a valency of at least or about 400 copies on a surface of the replication competent virus. In some embodiments, the second fusion protein is expressed at a valency of at least or about 500 copies on a surface of the replication competent virus. In some embodiments, the second fusion protein is expressed at a valency of at least or about 600 copies on a surface of the replication competent virus. In some embodiments, the second fusion protein is expressed at a valency of at least or about 700 copies on a surface of the replication competent virus. In some embodiments, the second fusion protein is expressed at a valency of at least or about 800 copies on a surface of the replication competent virus. In some embodiments, the second fusion protein is expressed at a valency of at least or about 900 copies on a surface of the replication competent virus. In some embodiments, the second fusion protein is expressed at a valency of at least or about 1000 copies on a surface of the replication competent virus. In some embodiments, the second fusion protein is expressed at a valency of at least or about 2000 copies on a surface of the replication competent virus. In some embodiments, the second fusion protein is expressed at a valency of at least or about 3000 copies on a surface of the replication competent virus. In some embodiments, the second fusion protein is expressed at a valency of at least or about 4000 copies on a surface of the replication competent virus. In some embodiments, the second fusion protein is expressed at a valency of at least or about 5000 copies on a surface of the replication competent virus.

Described herein, in some embodiments, are HBV-antiviruses comprising improved binding properties. In some embodiments, the HBV-antiviruses comprise a binding affinity (e.g., K_{D}) to the viral protein of less than 100 pM, less than 200 pM, less than 300 pM, less than 400 pM, less than 500 pM, less than 600 pM, less than 700 pM, less than 800 pM, or less than 900 pM In some embodiments, the HBV-Antivirus comprises a K_{D} of less than 1 nM, less than 1.2 nM, less than 2 nM, less than 5 nM, or less than 10 nM. In some instances, the HBV-Antivirus comprises a K_{D} of less than 1 nM. In some instances, the HBV-Antivirus comprises a K_{D} of less than 1.2 nM. In some instances, the HBV-Antivirus comprises a K_{D} of less than 2 nM. In some instances, the HBV-Antivirus comprises a K_{D} of less than 5 nM. In some instances, the HBV-Antivirus comprises a K_{D} of less than 10 nM.

In some embodiments, the HBV-Antivirus comprises an IC₅₀ of less than 20 picomolar (pM) in a neutralization assay. In some embodiments, the HBV-Antivirus comprises an IC₅₀ of less than 15 picomolar (pM) in a neutralization assay. In some embodiments, the HBV-Antivirus comprises an IC₅₀ of less than 10 picomolar (pM) in a neutralization assay. In some embodiments, the HBV-Antivirus comprises an IC₅₀ of less than 5 picomolar (pM) in a neutralization assay. In some embodiments, the HBV-Antivirus comprises an IC₅₀ of less than 2.5 picomolar (pM) in a neutralization assay. In some embodiments, the HBV-Antivirus comprises an IC₅₀ of less than 1 picomolar (pM) in a neutralization assay. In some embodiments, the HBV-Antivirus comprises an IC₅₀ of less than 0.5 picomolar (pM) in a neutralization assay.

### Compositions and Methods for Generation of HBV-Antiviruses

Disclosed herein are compositions comprising a nucleic acid sequence that encodes any of the first fusion proteins disclosed herein. Disclosed herein are compositions comprising a nucleic acid sequence that encodes the first fusion proteins disclosed herein and any of the second fusion proteins disclosed herein. Disclosed herein are compositions comprising a DNA sequence that encodes the first fusion proteins disclosed herein. Disclosed herein are compositions comprising an mRNA sequence that encodes the first fusion proteins disclosed herein. Disclosed herein are compositions comprising a DNA sequence that encodes the first fusion proteins disclosed herein and any of the second fusion proteins disclosed herein. Disclosed herein are compositions comprising an mRNA sequence that encodes the first fusion proteins disclosed herein and any of the second fusion proteins disclosed herein.

In some embodiments, the composition further comprises a second nucleic acid sequence that encodes one or more packaging viral proteins. In some embodiments, the one or more packaging viral proteins comprises a lentiviral protein, a retroviral protein, an adenoviral protein, or combinations thereof. In some embodiments, the one or more packaging viral proteins comprises gag, pol, pre, tat, rev, or combinations thereof. In some embodiments, the composition further comprises a third nucleic acid sequence that encodes a reporter, a therapeutic molecule, or combinations thereof. In some embodiments, the composition further comprises a fourth nucleic acid sequence that encodes the second fusion protein.

In some embodiments, the reporter protein comprises a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter comprises a fluorescent protein. In some embodiments, the fluorescent protein is green fluorescent protein. In some embodiments, the reporter protein emits green fluorescence, yellow fluorescence, or red fluorescence. In some embodiments, the reporter comprises an enzyme. In some embodiments, the enzyme comprises β-galactosidase, alkaline phosphatase, (3-lactamase, or luciferase.

In some embodiments, the therapeutic molecule comprises an immune modulating protein, a cellular signal modulating molecule, a proliferation modulating molecule, or combinations thereof. In some embodiments, the therapeutic molecule comprises an immune checkpoint molecule. Exemplary immune checkpoint molecules include, but are not limited to, CTLA4, PD1, OX40, and CD28. In some embodiments, the therapeutic molecule comprises an inflammatory cytokine. In some embodiments, the inflammatory cytokine comprises IL-12, IL-18, TNF-α, or TNF-β. In some embodiments, the therapeutic molecule comprises a proliferation cytokine. In some embodiments, the proliferation cytokine comprises IL-7, IL-15, or IL-21.

In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors. In some embodiments, the first nucleic acid sequence, the second nucleic acid sequence, the third nucleic acid sequence, and the fourth nucleic acid sequence are within a same vector. In some embodiments, the first nucleic acid sequence, the second nucleic acid sequence, third nucleic acid sequence, and the fourth nucleic acid sequence are within different vectors. In some embodiments, the first nucleic acid sequence, the second nucleic acid sequence, the third nucleic acid sequence, and the fourth nucleic acid sequence are DNA sequences. In some embodiments, the first nucleic acid sequence, the second nucleic acid sequence, the third nucleic acid sequence, and the fourth nucleic acid sequence are mRNA sequences. In some embodiments, the first nucleic acid sequence that encodes the first fusion protein, the second nucleic acid sequence and the third nucleic acid sequence are within a same vector. In some embodiments, the first nucleic acid sequence that encodes the first fusion protein and the second fusion protein, the second nucleic acid sequence and the third nucleic acid sequence are within a same vector. In some embodiments, the first nucleic acid sequence that encodes the first fusion protein, the second nucleic acid sequence and the third nucleic acid sequence are within different vectors. In some embodiments, the first nucleic acid sequence that encodes the first fusion protein and the second fusion protein, the second nucleic acid sequence and the third nucleic acid sequence are within different vectors. In some embodiments, a nucleic acid sequence that encodes the first fusion protein and a nucleic acid sequence that encodes the second fusion protein are within a same vector. In some embodiments, a nucleic acid sequence that encodes the first fusion protein and a nucleic acid sequence that encodes the second fusion protein are within different vectors. In some embodiments, the first nucleic acid sequence that encodes the first fusion protein, the second nucleic acid sequence, the third nucleic acid sequence, and the fourth nucleic acid sequence that encodes the second fusion protein are within a same vector. In some embodiments, the first nucleic acid sequence that encodes the first fusion protein, the second nucleic acid sequence, the third nucleic acid sequence, and the fourth nucleic acid sequence that encodes the second fusion protein are within different vectors.

Various vectors, in some embodiments, are used herein. In some embodiments, the vector comprises a eukaryotic or prokaryotic vector. In some embodiments, the vector comprises a viral vector. In some embodiments, the vector comprises a lentivirus vector, an adenovirus vector, or an adeno-associated virus vector. Exemplary vectors include, without limitation, mammalian expression vectors: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8.

Described herein, in some embodiments, are methods of treating hepatitis B virus in a subject in need thereof comprising administering to the subject an HBV-Antivirus disclosed herein. In some embodiments, the HBV-Antivirus comprises a first fusion protein disclosed herein. In some embodiments, the HBV-Antivirus comprises a second fusion protein disclosed herein. Described herein, in some embodiments, are methods of treating hepatitis B virus in a subject in need thereof comprising administering to the subject a composition comprising an HBV-Antivirus disclosed herein. Such compositions may be prepared by any method known in the art of pharmacy, for example by mixing the active ingredient with the carrier(s) or excipient(s) under sterile conditions.

Described herein, in some embodiments, are methods of treating hepatitis B virus in a subject in need thereof comprising administering to the subject a composition, wherein the composition comprises a nucleic acid sequence that encodes a first fusion protein disclosed herein. Described herein, in some embodiments, are methods of treating hepatitis B virus in a subject in need thereof comprising administering to the subject a composition, wherein the composition comprises a nucleic acid sequence that encodes a first fusion protein disclosed herein and a second fusion protein disclosed herein. In some embodiments, the nucleic acid sequence comprises mRNA. In some embodiments, the nucleic acid sequence comprises DNA.

In some embodiments, the HBV-Antivirus or composition is administered through a parenteral route. In some embodiments, the HBV-Antivirus or composition is administered to the subject intravenously. In some embodiments, the HBV-Antivirus or composition is administered to the subject through inhalation. In some embodiments, the HBV-Antivirus or composition is administered to the subject through intranasal delivery. In some embodiments, the HBV-Antivirus or composition is administered to the subject through intratracheal delivery. In some embodiments, the HBV-Antivirus or composition is administered to the subject by an intraperitoneal injection. In some embodiments, the HBV-Antivirus or composition is administered to the subject by a subcutaneous injection. In some embodiments, the HBV-Antivirus or composition is administered to the subject through intramuscular injection.

In some embodiments, the HBV-antivirus induces T cell mediated cytotoxicity against viral infected cells after being administered. In some embodiments, the HBV-antivirus is taken up by an antigen-presenting cell when the HBV-antivirus is bound to the surface protein of the HBV. In some embodiments, the HBV-antivirus increases adaptive immune responses against HBV. In some embodiments, the administering to the subject of the HBV-antivirus is sufficient to reduce or eliminate the HBV as compared to a baseline measurement of the HBV taken from the subject prior to the administering of the HBV-antivirus or composition. In some embodiments, the administering to the subject of the composition is sufficient to reduce or eliminate a cancer as compared to a baseline measurement of the infection taken from the subject prior to the administering of the composition. In some embodiments, the reduction is at least about 1-fold, 5-fold, 10-fold, 20-fold, 40-fold, 60-fold, 80-fold, or up to about 100-fold.

In some embodiments, the composition is administered with a liposome. In some embodiments, the liposome is a protamine liposome. In some embodiments, the liposome is a cationic polymer liposome. In some embodiments, the composition is administered with a lipid nanoparticle. In some embodiments, the composition is administered with a cationic lipid nanoparticle. In some embodiments, the composition is administered with a cationic lipid, cholesterol nanoparticle. In some embodiments, the composition is administered with a cationic lipid, cholesterol, PEG nanoparticle. In some embodiments, the composition is administered with a dendrimer nanoparticle. In some embodiments, the composition is administered as a naked nucleic acid sequence. In some embodiments, the composition is administered as a naked DNA sequence. In some embodiments, the composition is administered as a naked mRNA sequence.

In some embodiments, the composition is administered with an adeno-associated virus (AAV). In some embodiments, the composition is administered with a polymer. In some embodiments, the composition is administered with protamine. In some embodiments, the composition is administered with a polysaccharide particle. In some embodiments, the composition is administered with a cationic polymer. In some embodiments, the composition is administered with a cationic nano-emulsion. In some embodiments, the composition is administered with a transfection reagent. In some embodiments, the composition is administered with a dendritic cell.

### Pharmaceutical Compositions

Described herein, in some embodiments, are compositions comprising HBV-antiviruses described herein. For administration to a subject, the HBV-antiviruses as disclosed herein, may be provided in a pharmaceutical composition together with one or more pharmaceutically acceptable carriers or excipients. In some embodiments, the HBV-antiviruses as disclosed herein, may be provided in a composition together with one or more carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Described herein, in some embodiments, is a composition comprising a multivalent HBV-Antivirus. In some embodiments, the HBV-Antivirus comprises an enveloped particle that displays at least 10 copies of an antibody on a surface of the HBV-Antivirus. In some embodiments, the antibody binds to a surface protein of an HBV.

Described herein, in some embodiments, is a composition comprising a multi-specific, multivalent HBV-Antivirus. In some embodiments, the HBV-Antivirus comprises an enveloped particle that displays at least 10 copies of a multi-specific antibody on a surface of the HBV-Antivirus. In some embodiments, the multi-specific antibody binds to one or more surface proteins of an HBV.

Described herein, in some embodiments, is a composition comprising a multi-specific, multivalent HBV-Antivirus. In some embodiments, the HBV-Antivirus comprises an enveloped particle that displays at least 10 copies of a first antibody on a surface of the HBV-Antivirus and at least 10 copies of a second antibody on the surface of the HBV-Antivirus. In some embodiments, the first antibody binds to a surface protein of an HBV and the second antibody binds to a surface protein of the HBV. In some embodiments, the second antibody has less than 100% sequence identity to the first antibody.

Described herein, in some embodiments, is a composition comprising a dual-action, immune-modulating HBV-Antivirus. In some embodiments, the HBV-Antivirus comprises an enveloped particle that displays at least 10 copies of an antibody on a surface of the HBV-Antivirus and at least 10 copies of an immune modulating polypeptide on the surface of the HBV-Antivirus. In some embodiments, the antibody binds to a surface protein of an HBV.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms. The pharmaceutical composition may be used in any of the methods disclosed herein, including but not limited to, methods of treating hepatitis B.

The pharmaceutical composition may be adapted for administration by any appropriate route, including a parenteral (e.g., subcutaneous, intramuscular, intravenous, or inhalation) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Treatment

Described herein are methods of treating hepatitis B virus in a subject in need thereof comprising selecting an antibody that binds to a surface protein of an HBV. In some embodiments, the method may comprise expressing the antibody on a surface of a multivalent particle. In some embodiments, the multivalent particle has a binding affinity to the surface protein of the HBV that is higher than the binding affinity of a soluble version of the antibody to the surface protein of the HBV. In some embodiments, the multivalent particle comprises an HBV-Antivirus described herein. In some embodiments, the surface protein of the HBV comprises Large Hepatitis B surface Antigen (L-HBsAg), Medium Hepatitis B surface Antigen (M-HBsAg), or Small Hepatitis B surface Antigen (S-HBsAg). In some embodiments, the method comprises administering the multivalent particle to the subject. In some embodiments, the multivalent particle neutralizes the HBV when the multivalent particle binds to the surface protein of the HBV. In some embodiments, the antibody is expressed in a fusion protein on the surface of the multivalent particle, wherein the fusion protein comprises a transmembrane polypeptide. In some embodiments, the transmembrane polypeptide comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein. In some embodiments, the fusion protein comprises an oligomerization domain. In some embodiments, the oligomerization domain comprises a leucine zipper dimerization domain, a D4 post-fusion trimerization domain of VSV-G protein, a Dengue E protein post-fusion trimerization domain, a foldon trimerization domain, or an influenza neuraminidase stem domain. In some embodiments, the antibody comprises an amino acid sequence from at least one complementarity determining region of 2H5, ADRI-2F3, H004, H009, H007, H015, H019, or H020. In some embodiments, the antibody comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc. In some embodiments, the fusion protein is expressed at a valency of about 10 copies on the surface of the multivalent particle. In some embodiments, the multivalent particle comprises a viral-like particle. In some embodiments, the multivalent particle comprises an extracellular vesicle. In some embodiments, the extracellular vesicle comprises an ectosome. In some embodiments, the extracellular vesicle comprises an exosome.

Described herein is a method of using a multivalent HBV-Antivirus disclosed herein as a treatment vaccine to induce antiviral immunity against a virus in a subject in need thereof. In some embodiments, the virus comprises HBV. In some embodiments, the virus comprises HDV. In some embodiments, the subject is infected with HBV. In some embodiments, the subject is infected with HDV. In some embodiments, the method comprises administering to an infected subject an HBV-Antivirus disclosed herein to induce protective immunity against future infections. In some embodiments, the HBV-Antivirus is administered to the subject by any appropriate route, including a parenteral (e.g., subcutaneous, intramuscular, intravenous, or inhalation) route. In some embodiments, the HBV-Antivirus is administered to the subject intravenously. In some embodiments, the HBV-Antivirus is administered to the subject through inhalation. In some embodiments, the HBV-Antivirus is administered to the subject through intranasal delivery. In some embodiments, the HBV-Antivirus is administered to the subject by an intraperitoneal injection. In some embodiments, the HBV-Antivirus is administered to the subject by a subcutaneous injection. In some embodiments, the HBV-Antivirus induces T cell mediated cytotoxicity against viral infected cells. In some embodiments, the HBV-antivirus-bound viruses are taken up by antigen-presenting cells and further boosting adaptive immune responses against HBV. In some embodiments, the HBV-antivirus-bound viruses are taken up by antigen-presenting cells and further boosting adaptive immune responses against HDV.

Described herein is a method of using a multivalent HBV-Antivirus inactivated vaccine to induce protective immunity against a virus in a subject in need thereof. In some embodiments, the HBV-Antivirus comprises an HBV-Antivirus disclosed herein. In some embodiments, the HBV-Antivirus inactivated vaccine comprises a virus and an HBV-Antivirus disclosed herein. In some embodiments, the virus comprises HBV. In some embodiments, the virus comprises HDV. In some embodiments, the method comprises administering to the subject the HBV-Antivirus inactivated vaccine to induce protective immunity against the virus. In some embodiments, the HBV-Antivirus is administered to the subject by any appropriate route, including a parenteral (e.g., subcutaneous, intramuscular, intravenous, or inhalation) route. In some embodiments, the HBV-Antivirus is administered to the subject intravenously. In some embodiments, the HBV-Antivirus is administered to the subject through inhalation. In some embodiments, the HBV-Antivirus is administered to the subject through intranasal delivery. In some embodiments, the HBV-Antivirus is administered to the subject by an intraperitoneal injection. In some embodiments, the HBV-Antivirus is administered to the subject by a subcutaneous injection. In some embodiments, the HBV-Antivirus induces T cell mediated cytotoxicity against viral infected cells. In some embodiments, the HBV-antivirus-bound viruses are taken up by antigen-presenting cells and further boosting adaptive immune responses against HBV. In some embodiments, the HBV-antivirus-bound viruses are taken up by antigen-presenting cells and further boosting adaptive immune responses against HDV.

Described herein are methods of treating hepatitis B virus in a subject in need thereof comprising administering to the subject an HBV-Antivirus described herein. In some embodiments, the HBV-Antivirus is administered to the subject by any appropriate route, including a parenteral (e.g., subcutaneous, intramuscular, intravenous, or inhalation) route. In some embodiments, the HBV-Antivirus is administered to the subject intravenously. In some embodiments, the HBV-Antivirus is administered to the subject through inhalation. In some embodiments, the HBV-Antivirus is administered to the subject through intranasal delivery. In some embodiments, the HBV-Antivirus is administered to the subject by an intraperitoneal injection. In some embodiments, the HBV-Antivirus is administered to the subject by a subcutaneous injection. In some embodiments, the HBV-Antivirus induces T cell mediated cytotoxicity against viral infected cells. In some embodiments, the HBV-antivirus-bound viruses are taken up by antigen-presenting cells and further boosting adaptive immune responses against HBV. In some embodiments, the administering to the subject of the HBV-Antivirus is sufficient to reduce or eliminate the HBV as compared to a baseline measurement of the HBV taken from the subject prior to the administering of the HBV-antivirus. In some embodiments, the reduction is at least about 1-fold, 5-fold, 10-fold, 20-fold, 40-fold, 60-fold, 80-fold, or up to about 100-fold.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Materials and Methods

Antibody and immune-modulating molecule display constructs: Codon-optimized antibody (Ab) and immune engager (IE) sequences were synthesized (Twist) and cloned into specialized display constructs expressing fusion peptides consisting of the displayed Ab or IE ectodomain fused to a sequence encoding the transmembrane and cytoplasmic tail domains of VSV-G protein. To generate HBV-Antiviruses displaying oligomerized Abs and IEs, the extracellular domains of displayed peptides were fused to a synthetic VSV-G sequence encoding the D4 post-fusion trimerization domain and the transmembrane and cytoplasmic tail domains.

Production of HBV-Antiviruses as VLPs and extracellular vesicles: HBV-Antiviruses based on VLPs and EVs can be produced from transfected HEK 293T cells. To produce lentiviral VLP-based HBV-Antiviruses with viral genomes, HEK 293T cells were co-transfected with Ab and IE display constructs, along with a lentiviral packaging vector (i.e., psPAX2) and a lentiviral genome transfer vector. To produce lentiviral VLP-based HBV-Antiviruses without viral genomes, HEK 293T cells were co-transfected with Ab and IE display constructs, along with a lentiviral packaging vector (i.e., psPAX2). To produce extracellular vesicle-based HBV-Antiviruses, HEK 293T cells were transfected with Ab and IE display constructs alone. Transfections were prepared by culturing 7.5×10⁶ HEK293T cells (ATCC CRL-3216) overnight in 10-cm dishes (Corning) containing DMEM media with glucose, L-glutamine and sodium pyruvate (Corning) supplemented with 10% fetal bovine serum (Sigma) and 1% Penicillin Streptomycin (Life Technologies), referred to as “293T Growth Media.” Cells should reach around 90% confluence after 24 hours. Transfection DNA mixture was prepared with polyethylenimine (PEI) in OPTI-MEM reduced serum medium (Gibco). Transfection mixture was incubated at room temperature for 15 minutes before being added to cells, which were then incubated at 37° C. in 5% CO₂. 6 hours post-transfection, 293T Growth Media was changed to 293T Growth Media supplemented with 0.1% sodium butyrate (referred to as “Transfection Media”) before being returned to incubation. After incubating for 24 hours at 37° C. with 5% CO₂ in Transfection Media, supernatant containing VLPs or EVs was collected, centrifuged at 1680 rpm for 5 minutes to remove cellular debris and mixed with 1× polyethylene glycol 8000 solution (PEG, Hampton Research), before being stored at 4° C. for 24 hours to allow fractionation. Cells were replenished with fresh Transfection Media, and a second particle supernatant collection was performed at 48 hours. Supernatant collections were then pooled, PEG precipitated and purified by size exclusion chromatography using Sephacryl S-300 High Resolution Beads (Sigma Aldrich).

Lentiviral and EV particle quantification by p24 ELISA and Tunable Resistive Pulse Sensing: P24 concentrations in VLP samples of HBV-Antivirus particles were determined using an HIV p24 SimpleStep ELISA kit (Abcam) per the manufacturer's protocol. Concentrations of lentiviral particles were extrapolated from the assumption that each lentiviral particle contains approximately 2000 molecules of p24, or 1.25×10⁴ pseudovirus particles per picogram of p24 protein. EV-based HBV-Antiviruses were quantified via tunable resistive pulse sensing (TRPS, qNano, IZON). Purified EV collections were diluted in 0.2 μm filtered PBS with 0.03% Tween-20 (Thermo Fisher Scientific) prior to qNano analysis. Concentration and size distributions of EV particles were then determined using an NP200 nanopore at a 45.5 mm stretch, and applied voltages between 0.5 and 0.7 V were used to achieve a stable current of 130 nA through the nanopore. Measurements for each EV sample were taken at pressures of 3, 5 and 8 mbar, and considered valid if at least 500 events were recorded, particle rate was linear and root mean squared signal noise was maintained below 10 pA. EV concentrations were then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles/mL. Measurements were analyzed using IZON Control Suite 3.4 software to determine original sample concentrations.

Western blot analysis of HBV-Antiviruses: Expression of Ab and IE fusion proteins on VLPs and EVs was confirmed via western blot analysis of purified particles. Samples of purified HBV-Antiviruses were lysed at 4° C. for 10 minutes with cell lysis buffer (Cell Signaling) before being mixed with NuPage LDS sample buffer (Thermo Fisher Scientific) and boiled at 95° C. for 5 minutes. Differences in oligomerization were determined by running samples in reducing and non-reducing conditions. Under reducing conditions, 5% 2-Mercaptoethanol (Thermo Fisher Scientific) was added to samples to dissociate oligomerized Ab and IE fusion proteins on HBV-Antiviruses. Protein samples were then separated on NuPAGE 4-12% Bis-Tris gels (Thermo Fisher Scientific) and transferred onto a polyvinylidene fluoride (PVDF) membrane (Life Technologies). PVDF membranes were blocked with TRIS-buffered saline with Tween-20 (TBST) and 5% skim milk (Research Products International) for 1 hour, prior to overnight incubation with primary antibody diluted in 5% milk. For fusion display constructs expressing VSV-G-tag, an anti-VSV-G epitope tag rabbit polyclonal antibody (BioLegend, Poly29039) was used at a 1:2000 dilution. The following day, the PVDF membrane was washed 3 times with 1×TBST and stained with a goat-anti-rabbit secondary antibody (IRDye 680) at a 1:5000 dilution for 60 minutes in 5% milk. Post-secondary antibody staining, the PVDF membrane was again washed 3 times with TBST before imaging on a Licor Odyssey scanner. Alternatively, western blot analyses were performed using an automated Simple Western size-based protein assay (Protein Simple) following the manufacturer's protocols. Unless otherwise mentioned, all reagents used here are from Protein Simple. Concentrated samples were lysed as described above, before being diluted 1:10 in 0.1× sample buffer for loading on capillaries. Ab and IE fusion protein expression levels were identified using the same primary rabbit polyclonal antibody at a 1:400 dilution and an HRP conjugated anti-rabbit secondary antibody (Protein Simple). Chemiluminescence signal analysis and absolute quantitation were performed using Compass software (Protein Simple).

Quantitative western blot analysis of HBV-Antiviruses: Quantitative western blot analyses were performed to determine the copies of displayed Ab and IE fusion proteins displayed per particle. P24 ELISA or TRPS (qNano) assays were used to determine HBV-Antivirus sample concentrations. Purified HBV-Antivirus samples were processed and analyzed via western blot under reducing conditions as described above. A synthetic protein standard with known concentration was used to generate a standard curve, from which copy numbers of displayed immune checkpoint on respective particles was determined.

HBV virus stock preparation: HepAD38 cells were cultured on in DMEM supplemented with 3% tetracycline-free FBS and 1× non-essential amino acids (NEAA). After 7 days of culture in tetracycline-free conditions to induce HBV expression, supernatant was collected every other day for 4 weeks and replaced with fresh medium. Collected supernatant was then centrifuged at 1500×g, 4° C. to remove cell debris and filtered with a 0.22 μM filter. Collections were then mixed with 1× polyethylene glycol 8000 solution (PEG, Hampton Research), before being stored at 4° C. for 24 hours to allow fractionation. Supernatant collections were concentrated via PEG precipitation and purified by size exclusion chromatography using Sephacryl S-300 High Resolution Beads (Sigma Aldrich). Concentrated virus stock was stored at −80° C., and a small volume used to determine the concentration of virus via TaqMan-based qPCR.

HBV virus stock quantification: A small aliquot of purified HBV stock virus was quantified to determine the virus concentration in GEQ/mL. HBV DNA was extracted from purified virus using a DNeasy Blood & Tissue Kit (Qiagen) according to the manufacturer's protocol. qPCR for HBV DNA was then performed using SensiFAST Probe Lo-ROX kit (Bioline) and the following primers and probe: 5′-CCGTCTGTGCCTTCTCATCTG-3′ (sense) (SEQ ID NO: 40), 5′-AGTCCAAGAGTCCTCTTATGTAAGACCTT-3′ (antisense) (SEQ ID NO: 41), 5′-/56 FAM/CCG TGT GCA/ZEN/CTT CGCTTC ACCTCT GC/3IABkFQ/-3′ (probe) (SEQ ID NO: 42). PCR was performed using a ViiA7 Real-Time PCR System (ThermoFisher) at the following conditions: (i) denaturation at 50° C. for 2 min followed by 95° C. for 5 min (one cycle); (ii) qPCR at 95° C. for 10 s, 60° C. for 30 s (40 cycles). Quantification was done using a standard curve composed of HBV standard DNA over a range of 10⁶-10⁹ copies.

In vitro HBV neutralization assay in HepG2-NTCP cells: HepG2-NTCP cells were seeded in 96-well plates at a density of 5×10⁴ cells per well in 293T Growth Medium supplemented with 1× non-essential amino acids (NEAA), referred to as “HepG2-NTCP Growth Medium”. Cells reached confluence 24 hours post seeding and medium was changed to DMEM supplemented with 3% FBS, 1×NEAA and 2% DMSO, referred to as “DMSO Treatment Medium”. After 24 hours of DMSO treatment, HBV inoculations were prepared with concentrated HBV stock at 100 GEQ/cell and mixed with HBV-Antivirus samples, at a total volume of 50 μL per well, before being immediately added to cells. Inoculations were prepared in “Inoculum Medium,” DMEM supplemented with 3% FBS, 1×NEAA, 2% DMSO and 4% PEG. Cells were spinoculated for 1 hour via centrifugation at 1000×g, 37° C. 24 hours post-spinoculation, inoculum was removed via vacuum aspiration and cells were washed 5 times with 100 μL PBS, before being cultured in DMSO Treatment Medium. Supernatant and cells were harvested 7 days post-infection for analysis.

In vitro HBV neutralization assay in primary human hepatocytes (PHH): Primary Human Hepatocytes (PHHs) were seeded in 48 well collagen-coated plates at 0.14×10⁶ cells/well in a total volume of 200 μL. Cells were incubated overnight at 37° C. in 5% CO₂. Following incubation, 6 concentrations of each sample were added to cells in triplicate. Virus was diluted to a pre-determined amount (MOI 1000) and incubated with cells and samples for 18 hours at 37° C./5% CO₂. MOI is defined as the number of HBV genome equivalents, as determined by the qPCR quantitation, added per cell in the respective growth format. Following the 18-hour infection, the samples were removed from cells and cells were gently washed five times with DMEM with no additives. 200 μL of assay media with no PEG 8000 was then added to cells. Experimental samples were not added back to the cells, but TDF was added back to the cells. Assay media was replaced on day 1, day 4 and day 7 post infection, as described above. On day 10 post infection the supernatant from each sample was collected and stored at −20° C. until assayed for extracellular DNA by qPCR and HBs antigen by chemiluminescent immunoassay (CLIA). Toxicity was evaluated in parallel by XTT staining.

HBsAg Chemiluminescence Immunoassay: To determine levels of secreted HBsAg, 50 μL of collected supernatants were loaded into 96-well plates provided in HBsAg chemiluminescence immunoassay (CLIA) kits (Autobio Diagnostics Co., Zhengzhou, China). Samples were analyzed per the manufacturer's instructions. Absolute concentrations of HBsAg were determined and relative values were calculated by normalizing to virus-only control wells. Relative HBsAg levels were calculated as 100% in virus-only control wells and as corresponding percentages for samples containing HBV-Antiviruses.

Quantitative PCR Detection of HBV DNA in cell culture supernatant: 10 μL of cell culture supernatant was diluted in 90 μL of buffer containing 40 μg/mL sheared salmon sperm DNA and boiled for 15 minutes. Quantitative real time PCR was performed in 384-well plates using a Bio-Rad CFX384 Touch Realtime-PCR Detection System and CFX Manager software. 4 μL boiled DNA for each sample and serial 10-fold dilutions of a quantitative DNA standard were subjected to real time qPCR using Sso Advanced Universal Probes Supermix (Bio-Rad) and specific DNA oligonucleotide primers (IDT, Coralville, ID) HBV-AD38-qF1 (5′-CCG TCT GTG CCT TCT CAT CTG-3′) (SEQ ID No: 50), HBV-AD38-qR1 (5′-AGT CCA AGA GTC CTC TTA TAC AAG ACC-3′) (SEQ ID No: 51), and HBV-AD38-qP1 (5′-FAM/CCG TGT GCA/ZEN/CTT CGC TTC AC-3′BHQ1) (SEQ ID No: 52). The quantity of HBV DNA in each sample was interpolated from the standard curve by instrument software and data was imported into an Excel spreadsheet for EC50 calculation by linear regression analysis.

HBsAg-specific, in vitro MHC stimulation: HepAD38 cells and HepG2-NTCP cells were cultured together in tet-free medium to induce expression of HBsAg in HepAD38 cells. Soluble IEs or IE HBV-Antiviruses co-displaying HBsAg-specific antibodies and IEs such as TNF-α were then mixed with cells and cultured at 37° C. in 5% CO2 for 48 hours. Cells were then trypsinized and stained using an MHC-I specific primary antibody (APC mouse anti-human HLA-ABC), and differential MIC-I expression patterns were analyzed via flow cytometry.

In vivo HBV-Antivirus treatment of HBV in AAV/HBV murine model: Male C57BL/6 mice are injected with rAAV8-1.3HBV (genotype D HBV, 2.0×10¹⁰ genome copies) via tail veins and monitored at day −14 and −7 for serum HBV DNA and HBsAg levels via qPCR and ELISA, respectively. Beginning on day 0, mice are randomized into groups of 6 based on serum HBV levels and treated with a vehicle control, a characterized neutralizing antibody or HBV-Antiviruses via tail vein IV, twice weekly for 4 weeks, for a total of 8 doses. During treatment, blood samples are taken from mice twice weekly and serum HBV DNA and HBsAg levels determined via qPCR and ELISA, respectively. At day 35 and day 42, serum samples are also analyzed via ELISA for anti-HBs antibody titer. Finally, at day 42 post-treatment, mice are sacrificed and liver HBcAg and HBsAg expression was determined via immunohistochemistry (IHC).

HDV in vitro neutralization in primary human hepatocytes: PHH cells were seeded in 48-well plates, 1.32×10⁵ cells per well in InVitroGRO medium (BioreclamationIVT-S03318) supplemented with 10% FBS, 100 U/mL Penicillin Streptomycin solution. Cells were incubated overnight at 37° C., 5% CO2. The following day, Type D HBV inoculum and Type 1 HDV inoculum are added to cells along with neutralizing compounds. Specifically, neutralizing compounds are diluted 3-fold for a total of 7 test concentrations. 125 μL of each concentration is mixed with virus inoculum and incubated at 37° C. for 1 hour, before adding PEG8000/DMSO solution and transferring the entire mixture, 250 μL to the cells. Final concentration of PEG8000 and DMSO in culture medium is 4% and 2%, respectively. Medium is then aspirated and replaced without neutralizing compound on day 2, 4 and 6 of assay progression. Test article neutralizing IC₅₀ₛ are determined on day 8 via extracellular and intracellular HDV RNA expression levels, as determined by RT-PCR. Cytotoxicity was determined via CCK-8 assay.

Isolation of extracellular HDV RNA and RT-PCR: Extracellular HDV RNA was isolated via PureLink™ Pro 96 viral RNA/DNA Kit (Invitrogen-12280096A) according to the manual. Reverse transcription of HDV total RNA was performed via FastKing RT Kit (With gDNase) (TIANGEN-KR116) according to the manual. In brief, cDNA samples are quantified by HDV primer/probe. A plasmid containing the HDV sequence (genotype I) is used as a standard sample for HDV RNA quantification. A standard curve can be built with the Ct value and the original amount of the plasmid standard, and the quantities of the samples are calculated according to the standard curve with the Ct value.

Isolation of intracellular HDV RNA and RT-PCR: The intracellular HDV RNA was isolated with RNeasy kit (QIAGEN-74182) according to the manual. Reverse transcription of HDV total RNA was performed via FastKing RT Kit (With gDNase) (TIANGEN-KR116) according to the manual. cDNA samples are quantified by HDV primer/probe. A plasmid containing the HDV sequence (genotype I) is used as a standard sample for HDV RNA quantification.

Evaluation of HBV-Antivirus cytotoxicity by CCK-8: On day 8, after culture supernatants are collected, CCK-8 working solution (CCK-8 diluted with fresh culture medium at ratio of 1:9) was added to PHH cells in wells. The plates were incubated at 37° C., 5% CO₂ incubator for 1-2 hours. OD was measured by microplate reader (SepctraMax 340PC384model) (450 nm/630 nm).

### Example 2: Design and Production of HBV-Antiviruses

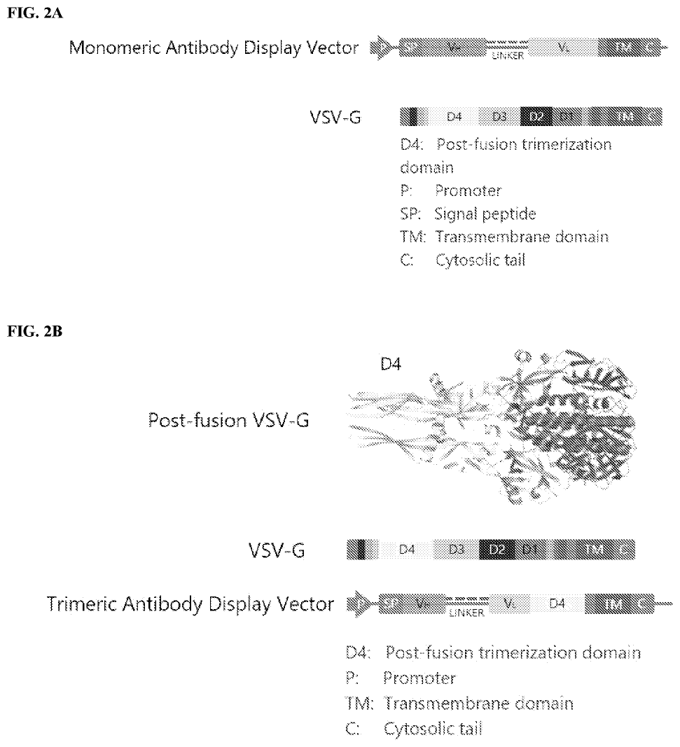

Two different forms of antibody display vectors are designed to display antibodies in monomeric and trimeric forms on the surface of HBV-Antivirus vesicles. Monomeric antibody display vectors express a fusion protein including the desired antibody fused to the transmembrane and cytoplasmic tail regions of VSV-G protein (FIG. 2A). Trimeric antibody display constructs express a fusion protein including the desired antibody fused to the D4 post-fusion trimerization domain, and the transmembrane and cytoplasmic tail regions of VSV-G protein (FIG. 2B). Each display construct type is used to generate monomeric and trimeric HBV-Antiviruses, respectively.

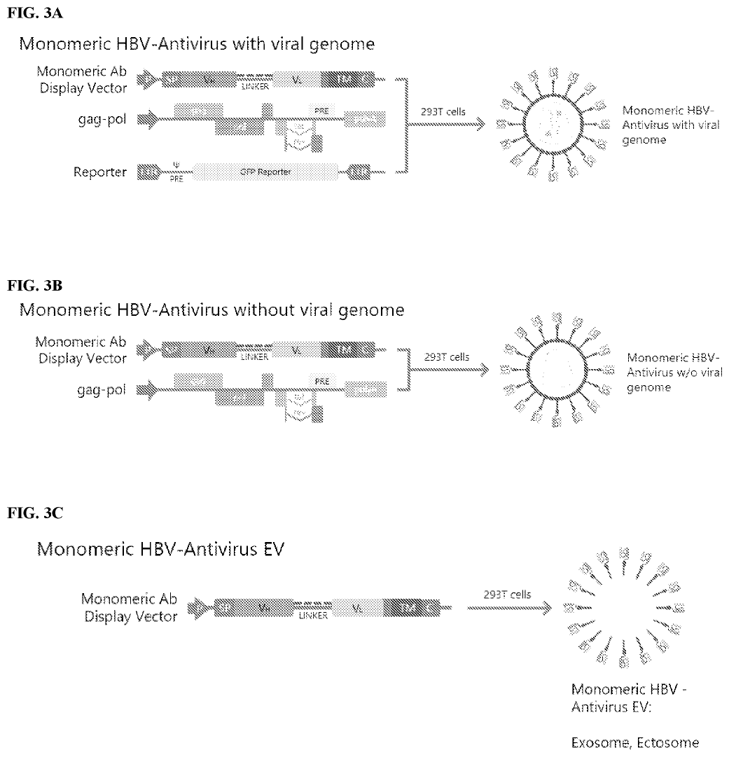

Production of monomeric HBV-Antiviruses: Monomeric HBV-Antiviruses in the form of VLPs containing RNA genomes are produced via co-transfection of HEK 293T cells with monomeric antibody display constructs along with a lentiviral packaging construct expressing essential packaging components, such as Gag-Pol and Rev proteins, and a viral genome transfer vector encoding a GFP/luciferase reporter (FIG. 3A). Alternatively, monomeric HBV-Antiviruses in the form of VLPs without genomes are produced by co-transfecting HEK 293T cells with antibody-display vectors along with a lentiviral packaging construct, without a viral genome transfer vector (FIG. 3B). Finally, monomeric HBV-Antiviruses in the form of ectosomes and exosomes displaying antibodies are produced via transfection of HEK 293T cells with the monomeric antibody-display constructs alone (FIG. 3C).

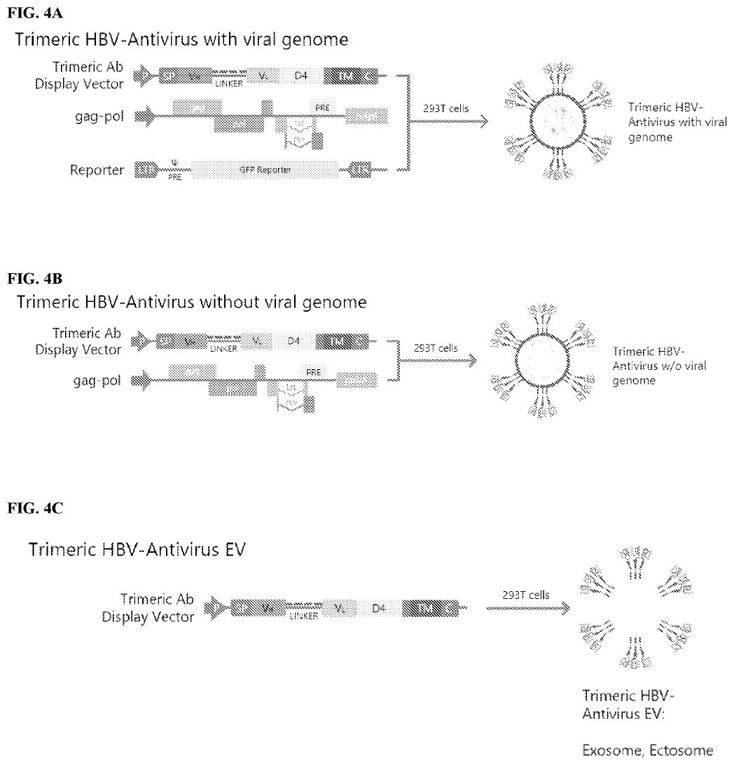

Production of trimeric HBV-Antiviruses: Trimeric HBV-Antiviruses in the form of VLPs containing RNA genomes are produced via co-transfection of HEK 293T cells with trimeric antibody display constructs along with a lentiviral packaging construct expressing essential packaging components, such as Gag-Pol and Rev proteins, and a viral genome transfer vector encoding a GFP/luciferase reporter (FIG. 4A). Alternatively, trimeric HBV-Antiviruses in the form of VLPs without genomes are produced by co-transfecting HEK 293T cells with trimeric antibody-display vectors along with a lentiviral packaging construct, without a viral genome transfer vector (FIG. 4B). Finally, trimeric HBV-Antiviruses in the form of ectosomes and exosomes displaying antibodies are produced via transfection of HEK 293T cells with the trimeric antibody-display constructs alone (FIG. 4C).

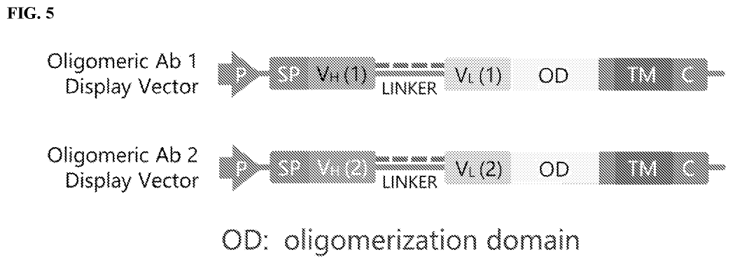

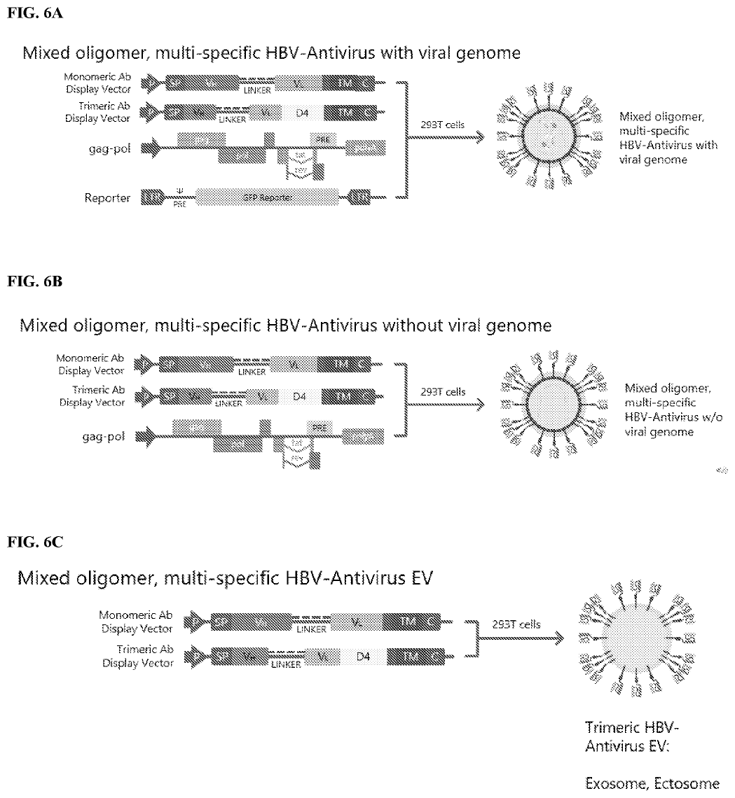

Production of mixed oligomer, multi-specific HBV-Antiviruses: A variety of mixed HBV-Antiviruses co-displaying distinct antibodies in both monomeric and trimeric form are produced. Such HBV-Antiviruses have increased display density and can simultaneously target multiple HBsAg viral epitopes on HBV virions (Table 5). Various oligomerization domains, including the post-fusion trimerization domains of VSV-G and the Dengue E protein, the foldon trimerization domain, leucine zipper dimerization domain, and influenza neuraminidase stem tetramerization domain, are used to display antibodies in various oligomeric patterns on mixed HBV-Antiviruses (Table 6). FIG. 5 illustrates configurations of display vectors for mixed oligomer, multi-specific HBV-Antiviruses. The mixed HBV-Antiviruses can be designed atop VLPs and EVs. To produce mixed, multi-specific HBV-Antiviruses using VLPs with RNA genomes, HEK 293T cells are co-transfected with combinations of distinct monomeric/trimeric antibody display constructs, as well as a lentiviral packaging construct expressing essential packaging components, such as Gag-Pol and Rev proteins, and a viral genome transfer vector encoding a GFP/luciferase reporter (FIG. 6A). Alternatively, mixed HBV-Antiviruses in the form of VLPs without genomes are produced by co-transfecting HEK 293T cells with combinations of distinct monomeric/trimeric antibody-display vectors along with a lentiviral packaging construct, without a viral genome transfer vector (FIG. 6B). Mixed HBV-Antiviruses in the form of ectosomes and exosomes displaying antibodies are produced via transfection of HEK 293T cells with combinations of distinct monomeric/trimeric antibody-display constructs without lentiviral structural constructs or a viral genome transfer vector (FIG. 6C).

TABLE 5
Configurations of Mixed Oligomer, Multi-Specific HBV-Antiviruses

| | HBV Surface Protein Target | Display Oligomerization |
|---|---|---|
| Ab1 | S | Monomeric |
| | Pre-S1 (L) | Multimeric |
| | Pre-S2 (M) | |
| Ab2 | S | Monomeric |
| | Pre-S1 (L) | Multimeric |
| | Pre-S2 (M) | |

TABLE 6
Exemplary Oligomerization Domains

| Oligomerization Domains | Valence |
|---|---|
| VSV-G Protein D4 | Trimer |
| Dengue E Protein fusion domain | Trimer |
| Foldon | Trimer |
| Leucine Zipper | Dimer |
| Influenza Neuraminidase Stem | Tetramer |

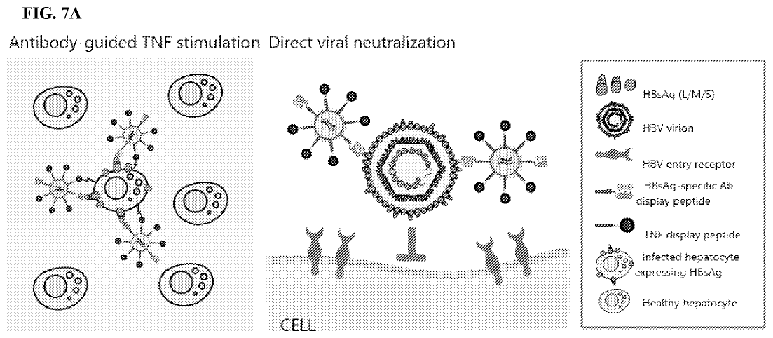

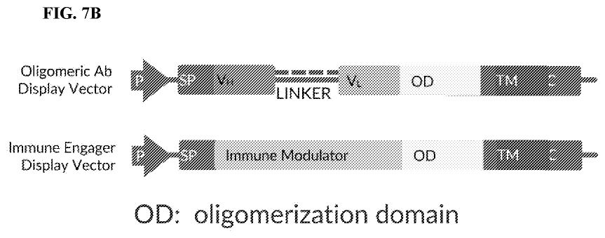

Production of bi-functional, immune-modulating HBV-Antiviruses (IM-HBV-Antiviruses): A variety of mixed HBV-Antiviruses co-displaying both HBV-binding antibodies and immune-modulating molecules (e.g. TNFs), termed IM-HBV-Antiviruses, are produced. These HBV-Antiviruses capture HBV virions and SVPs while potentiating HBsAg-specific antiviral immune responses (FIG. 7A). FIG. 7A illustrates dual action mechanisms of IM-HBV-Antiviruses co-displaying antibodies and TNF which simultaneously mediate inflammatory signaling and viral neutralization. FIG. 7B illustrates configurations of display vectors for HBV-Antiviruses displaying antibodies and immune-modulating molecules. HBV-binding antibodies and immune-modulating molecules are displayed in either monomeric or oligomeric forms (e.g., trimeric) on HBV-Antiviruses (FIG. 7B, Table 7), and the IM-HBV-Antiviruses are designed atop VLPs and EVs. Various oligomerization domains, including the post-fusion trimerization domain of VSV-G and the Dengue E protein, the foldon trimerization domain, leucine zipper dimerization domain, and tetramerization domain from influenza neuraminidase stem, are used to display antibodies and immune modulators in specific oligomeric patterns on mixed IM-HBV-Antiviruses (Table 6).

TABLE 7
Configurations of Mixed, Immune-Modulating
HBV-Antiviruses (IM-HBV-Antiviruses)

| | Display Oligomerization |
|---|---|
| Ab | Monomeric |
| | Multimeric |
| IM | Monomeric |
| | Multimeric |

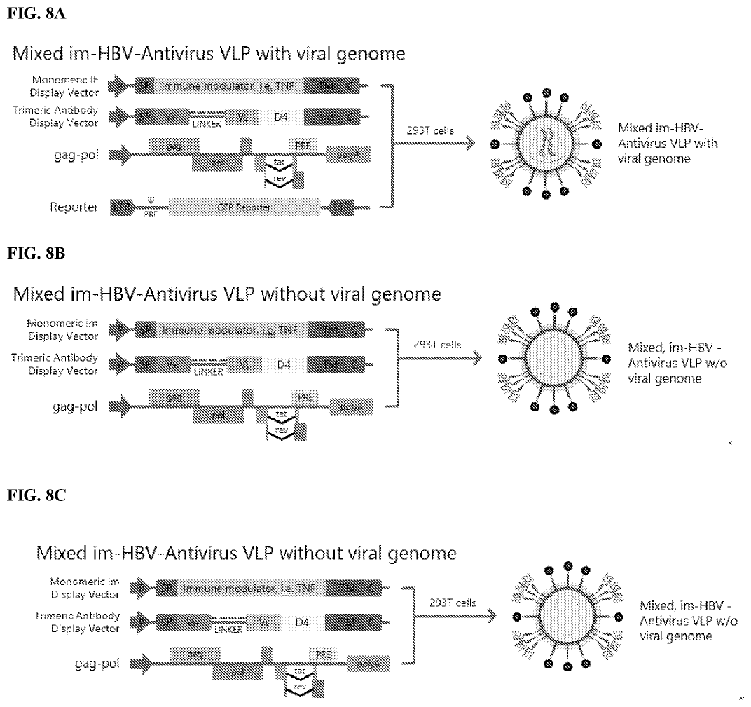

To produce IM-HBV-Antiviruses in the form VLPs with RNA genomes, HEK 293T cells are co-transfected with combinations of antibody and immune modulating display constructs, as well as a lentiviral packaging construct expressing essential packaging components, such as Gag-Pol and Rev proteins, and a viral genome transfer vector encoding a GFP/luciferase reporter (FIG. 8A). Alternatively, IM-HBV-Antiviruses in the form of VLPs without genomes are produced by co-transfecting HEK 293T cells with combinations of antibody and immune modulating display vectors along with a lentiviral packaging construct, without a viral genome transfer vector (FIG. 8B). Finally, IM-HBV-Antiviruses in the form of ectosomes and exosomes are produced via transfection of HEK 293T cells with combinations of distinct antibody and immune modulating display constructs without lentiviral structural constructs or a viral genome transfer vector (FIG. 8C).

### Example 3: Characterization of HBV-Antiviruses

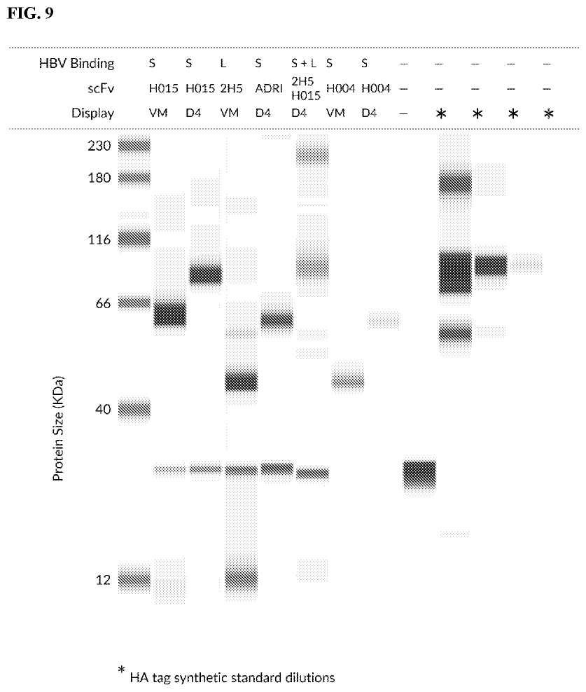

Antibody display on HBV-Antiviruses: Concentrations of VLP- or EV-based HBV-Antiviruses are determined via P24 ELISA or tunable resistive pulse sensing (TRPS, qNano), respectively. Based on these concentrations, the copy numbers of displayed antibodies and immune modulating molecules on particles are determined by quantitative western-blot analysis (FIG. 9, Table 8). Oligomerization patterns of displayed antibodies and immune modulating molecules are determined via non-reducing PAGE analysis.

TABLE 8
Copy Number of Displayed Antibodies

| HBV Antivirus | Copy number per particle (103) |
|---|---|
| αS:H015/VM | 5.0 ± 1.4 |
| αS:H015/D4 | 3.0 ± 1.7 |
| αL:2H5/VM | 3.0 ± 0.3 |
| αS:ADRI/D4 | 1.2 ± 0.7 |
| (αS-αL)/D4 | 2.4 ± 0.4 |
| αS:H004/VM | 4.5 |
| αS:H004/D4 | 5.01 |

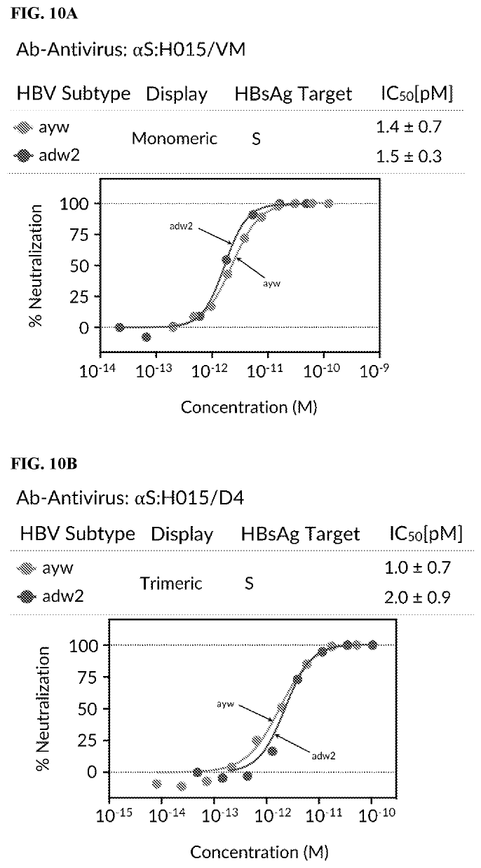

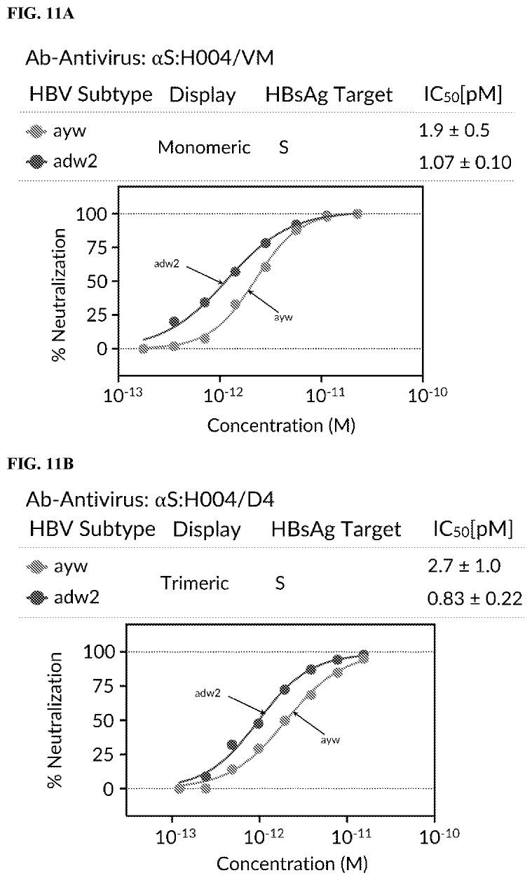

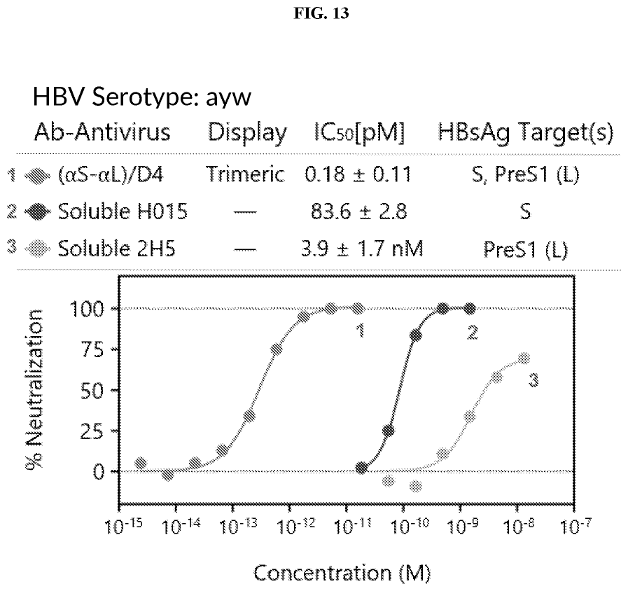

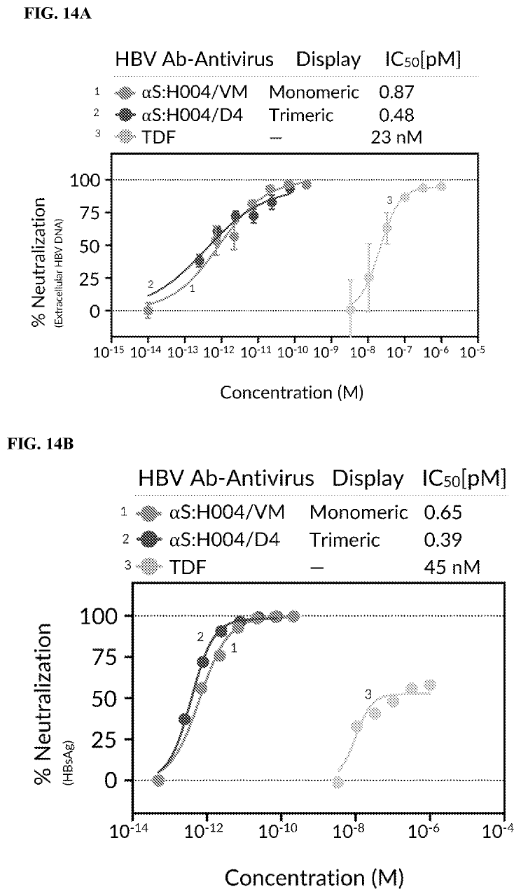

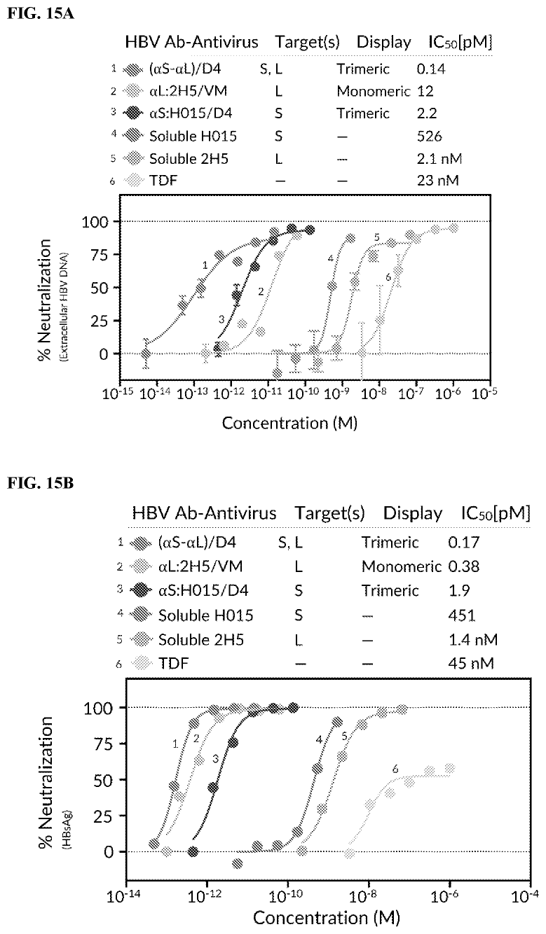

In vitro HBV neutralization by mono-specific HBV-Antiviruses: The neutralization efficacy of HBV-Antiviruses targeting S-HBsAg or Pre-S1 domain of L-HBsAg was analyzed. Monomeric and trimeric HBV-Antiviruses without viral genetic material targeting the antigenic loop of S-HBsAg were generated by displaying a Fab fragment derived from a neutralizing antibody H015, and their neutralization efficacy against HBV were analyzed in an in vitro neutralization assay using HepG2-NTCP cells for infection. Two different subtypes of HBV, ayw and adw2, were used in neutralization assays. The results indicated that monomeric αS:H015/VM and trimeric αS:H015/D4 each displayed 5000±1400 and 3000±1700 copies of H015 Fab per HBV-Antivirus particle, respectively (Table 8). αS:H015/VM neutralized both the ayw and adw2 subtypes of HBV at IC₅₀ₛ of 1.4±0.7 pM and 1.5±0.3 pM, respectively (FIG. 10A), while αS:H015/D4 neutralized both subtypes of HBV at IC₅₀ₛ of 1.0±0.7 pM and 2.0±0.9 pM, respectively (FIG. 10B). In comparison, bivalent, soluble H015 neutralized the ayw subtype of HBV at an IC₅₀ of 83.6±2.8 pM, demonstrating that multivalent display of H015 on HBV-Antiviruses resulted in over 60-fold enhancement of neutralizing potency (FIG. 13). Also, the neutralization ability of αS:H015/D4 against HBV was tested in an in vitro setting infecting primary human hepatocytes (PHH), using soluble H015 and Tenofovir Disoproxil Fumarate (TDF) as positive controls. αS:H015/D4 neutralized HBV infecting PHH at sub picomolar IC₅₀ₛ (FIGS. 15A and 15B) and was at least 50-fold more potent than soluble H015, and at least 1000-fold more potent than TDF. Importantly, αS:H015/D4 was able to fully suppress S antigen expression, unlike TDF.

Other HBV-Antiviruses targeting the S antigen, including monomeric and trimeric HBV-Antiviruses without viral genetic material displaying an scFv derived from a neutralizing antibody H004 were produced. Monomeric αS:H004/VM and trimeric αS:H004/D4 displayed 4500 and 5010 copies of H004 scFv per HBV-Antivirus particle, respectively (Table 8). It was demonstrated that in an in vitro neutralization assay using HepG2-NTCP cells for infection, monomeric αS:H004/VM neutralized both ayw and adw2 subtypes of HBV at IC₅₀ₛ of 1.9±0.5 pM and 1.07±0.10 pM, respectively (FIG. 11A), while trimeric αS:H004/D4 neutralized both subtypes of HBV at IC₅₀ₛ of 2.7±1.0 pM and 0.83±0.22 pM, respectively (FIG. 11B). Analysis was carried out to test αS:H004/VM and αS:H004/D4 against HBV in an in vitro setting infecting PHH, using TDF as a positive control. Both monomeric αS:H004/VM and trimeric αS:H004/D4 were able to neutralize HBV infecting PHH at sub picomolar IC₅₀ₛ. (FIGS. 14A and 14B).

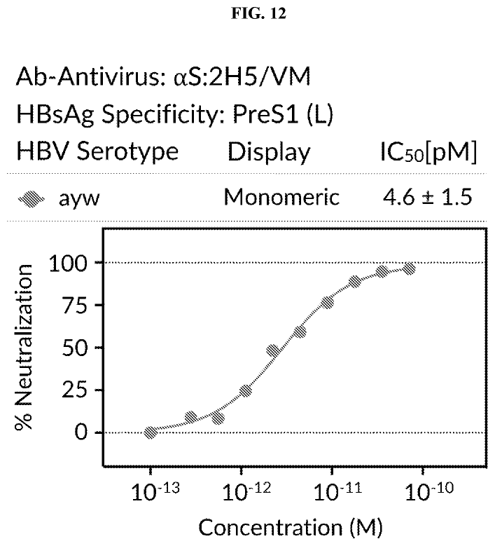

Monomeric HBV-Antiviruses were generated targeting the Pre-S1 domain of the L-HBsAg without viral genetic material by displaying an scFv fragment derived from a non-neutralizing antibody 2H5, and its neutralizing efficacy against HBV was analyzed in an in vitro neutralization assay using HepG2-NTCP cells for infection. The results showed that monomeric αS:2H5/VM displaying 3000±300 copies of 2H5 scFv per HBV-Antivirus particle (Table 8) neutralized HBV at an IC₅₀ of 4.6±1.5 pM (FIG. 12). Meanwhile, bivalent, soluble 2H5 was unable to neutralize the same subtype of HBV in the same in vitro neutralization assay, illustrating that multivalent presentation of antibodies on HBV-Antiviruses can convert non-neutralizing antibodies into potently neutralizing antiviruses. Analysis was carried out to test αS:2H5/VM, soluble 2H5 and TDF, against HBV in an in vitro infection assay using PHH. Again, monomeric αS:2H5/VM HBV-antiviruses were able to neutralize HBV at a low or sub-picomolar IC₅₀, while both soluble 2H5 and TDF neutralized HBV with IC₅₀ₛ in the nanomolar range (FIGS. 15A and 15B).

In vitro HBV neutralization by bi-specific HBV-Antiviruses: Given the distribution of S and Pre-S1 antigens on the HBV particles, the neutralizing potency of HBV-Antiviruses can be further enhanced by displaying bi-specific antibodies recognizing both S and Pre-S1 antigens. To this end, a bi-specific HBV-Antivirus, (αS-αL)/D4, were generated without viral genetic material displaying a tandem non-neutralizing Fab-scFv targeting both the antigenic loop of S-HBsAg and the Pre-S1 domain of L-HBsAg, and then its neutralizing potency against HBV was determined in an in vitro neutralization assay using HepG2-NTCP cells for infection. (αS-αL)/D4 displayed approximately 2400±400 copies of tandem Fab-scFv per HBV-Antivirus particle (Table 8) and was able to neutralize ayw subtype HBV at an IC₅₀ of 0.18±0.11 pM (FIG. 13). This represented a nearly 500-fold enhancement of neutralizing potency from either soluble, bivalent antibody. (αS-αL)/D4 was tested against HBV in a PHH in vitro neutralization assay and the results showed that (αS-αL)/D4 was able to neutralize HBV at an IC₅₀ of around 0.1 pM, consistent with its potency in HepG2-NTCP infection settings (FIGS. 15A and 15B). Notably, (αS-αL)/D4 fully suppressed S antigen expression and was more than 100,000-fold more potent than TDF.

In vitro HBV neutralization by IM-HBV-Antiviruses co-displaying TNF-α: A mixed, IM-HBV-Antivirus, TNF-α/VM:(αS-αL)/D4, is produced co-displaying monomeric TNF-α and a trimeric, tandem Fab-scFv targeting the antigenic loop of S and the Pre-S1 domain of L-HBsAg. The TNF-α/VM:(αS-αL)/D4 HBV-Antiviruses displays more than 10 copies of TNF-α and more than 10 copies of tandem Fab-scFv per particle. The ability of the IM-HBV-Antiviruses in neutralizing HBV is determined using an in vitro neutralization assay using HepG2-NTCP cells for infection and PHH for infection. The results indicate that TNF-α/VM:(αS-αL)/D4 neutralizes HBV at IC₅₀ₛ in the range of low picomolar concentrations.

HBsAg-specific MHC-stimulation by IM-HBV-Antiviruses co-displaying TNF-α: The functionality of IM-HBV-Antiviruses co-displaying TNF-α is determined. TNF-α/VM:(αS-αL)/D4 IM-HBV-Antiviruses are tested using an in vitro MHC I stimulation assay with induced HepAD38 cells expressing HBsAg and HepG2-NTCP cells lacking HBsAg. Adding soluble TNF-α to co-cultured cells result in an upregulation of MHC I in both HBV positive HepAD38 cells and HBV negative HepG2-NTCP cells. Neither naked VLPs nor (αS-αL)/D4 HBV-Antivirus increase MHC I expression in either cell type. TNF-α/VM:(αS-αL)/D4 IM-HBV-Antivirus results in a significant increase in MHC I expression in HBV positive HepAD38 cells but not in HBV negative HepG2-NTCP cells. The data demonstrate the ability of IM-HBV-Antiviruses to mediate antigen-specific immune responses to HBV-infected cells.

### Example 4: Oligomerization Domains and Display Configurations on HBV-Antiviruses

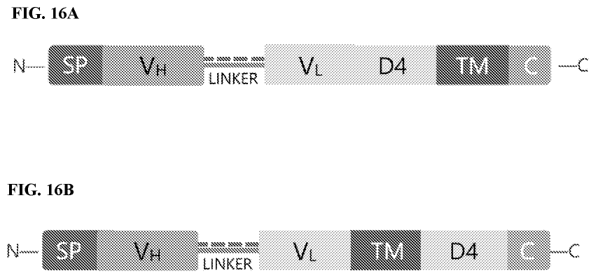

FIGS. 16A-16B illustrate possible D4 configurations for trimeric displays of antibodies on HBV-Antiviruses. FIG. 16A illustrates design of HBV-Antivirus antibody display construct wherein the D4 post-fusion trimerization domain is positioned extracellularly, in between the antibody sequence and the transmembrane anchoring sequence. FIG. 16B illustrates design of HBV-Antivirus antibody display construct wherein the D4 post-fusion trimerization domain is positioned intracellularly, following both the antibody sequence and the transmembrane anchoring sequence.

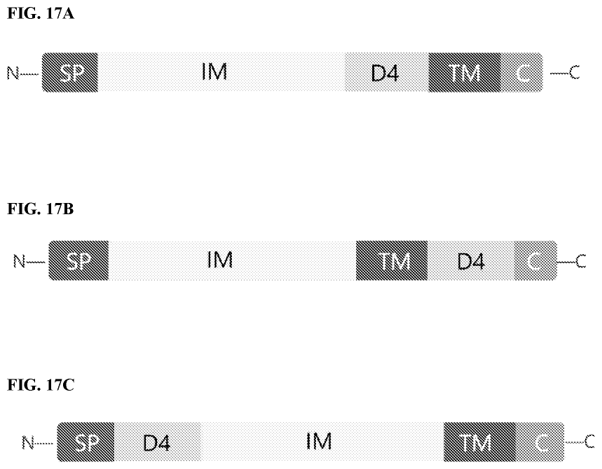

FIGS. 17A-17C illustrate possible D4 configurations for trimeric displays of immune modulators on HBV-Antiviruses. FIG. 17A illustrates design of immune-modulating molecule display construct wherein the D4 post-fusion trimerization domain is positioned extracellularly, in between the immune modulator and the transmembrane anchoring sequence. FIG. 17B illustrates design of immune-modulating molecule display construct wherein the D4 post-fusion trimerization domain is positioned intracellularly, following both the immune modulator and the transmembrane anchoring sequence. FIG. 17C illustrates design of immune-modulating molecule display construct wherein the D4 post-fusion trimerization domain is positioned extracellularly, in front of both the immune modulator and the transmembrane anchoring sequence.

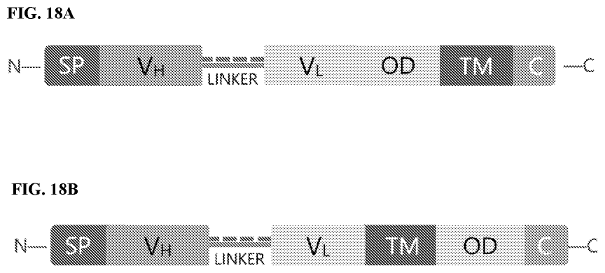

FIGS. 18A-18B illustrate possible oligomerization domains and configurations for antibody display on HBV-Antiviruses. Table 6 shows valences of various oligomerization domains (OD) for the HBV-Antiviruses. FIG. 18A illustrates design of HBV-Antivirus antibody display construct wherein the desired oligomerization domain is positioned extracellularly, in between the antibody sequence and the transmembrane anchoring sequence. FIG. 18B illustrates design of HBV-Antivirus antibody display construct wherein the desired oligomerization domain is positioned intracellularly, following both the antibody sequence and the transmembrane anchoring sequence.

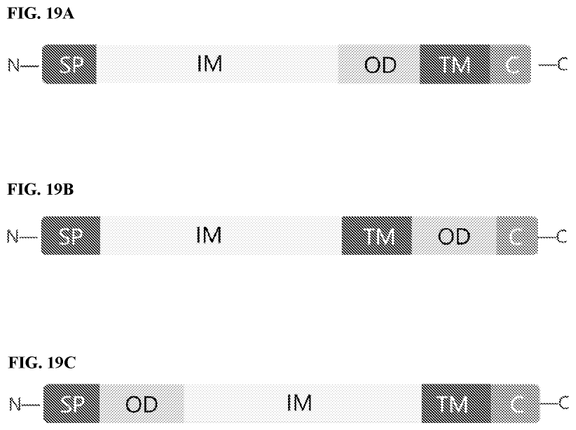

FIGS. 19A-19C illustrate possible oligomerizations and configurations for immune modulator display on HBV-Antiviruses. Table 6 shows valences of various oligomerization domains (OD) for immune modulating HBV-Antiviruses. FIG. 19A illustrates design of immune-modulating molecule display construct wherein the desired oligomerization domain is positioned extracellularly, in between the immune modulator and the transmembrane anchoring sequence. FIG. 19B illustrates design of immune-modulating molecule display construct wherein the desired oligomerization domain is positioned intracellularly, following both the immune modulator and the transmembrane anchoring sequence. FIG. 19C illustrates design of immune-modulating molecule display construct wherein the desired oligomerization domain is positioned extracellularly, in front of both the immune modulator and the transmembrane anchoring sequence.

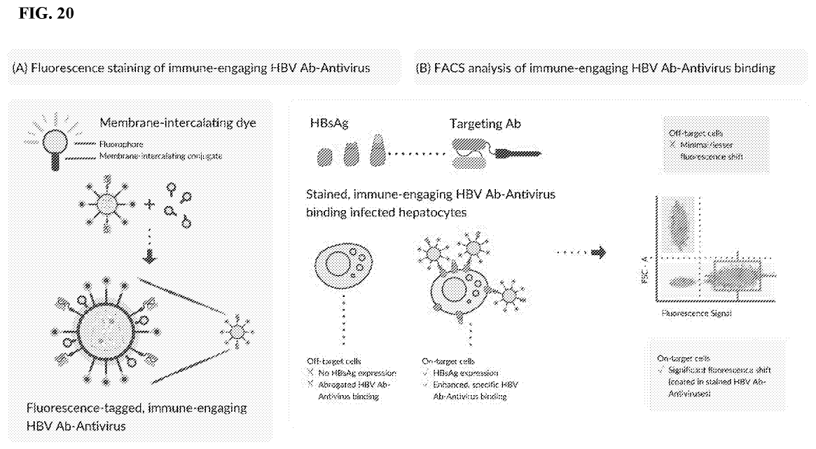

FIG. 20 illustrates a method for determining HBV antigen-specific binding of IM-HBV-Antiviruses. PANEL (A) illustrates a method of labeling IM-HBV-Antiviruses using membrane-intercalating fluorescent dyes. PANEL (B) illustrates analysis of IM-HBV-Antivirus antigen specific binding via FACS analysis.

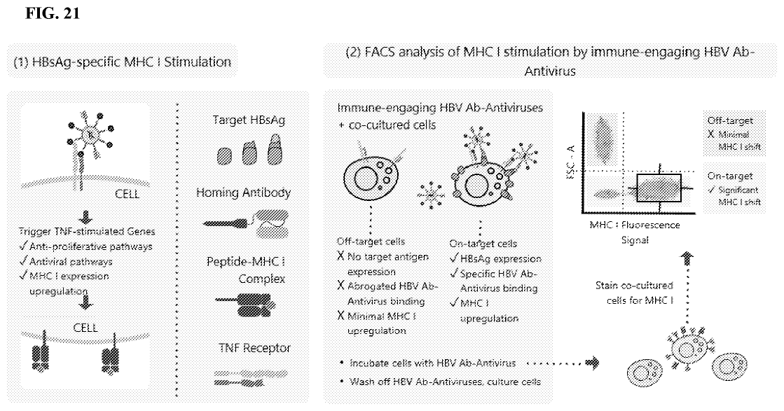

FIG. 21 illustrates functional analysis of antigen specific immune-modulation by IM-HBV-Antiviruses co-displaying TNF-α. PANEL (1) illustrates HBsAg-specific cell targeting by IM-HBV-Antiviruses co-displaying antibodies and TNF-α. TNF-α stimulates inflammatory pathways, including the expression of class I major histocompatibility complex (MHC I). PANEL (2) illustrates functional analysis of IM-HBV-Antivirus antigen-specific inflammatory activity of MHC I expression via FACS analysis.

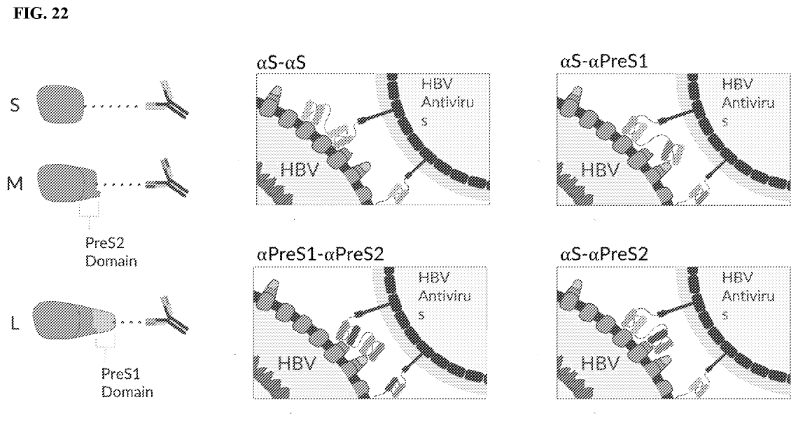

FIG. 22 illustrates HBV antigen specificity combinations in tandem bi-specific HBV-Antiviruses displaying tandem bi-specific antibodies with multiple specificities.

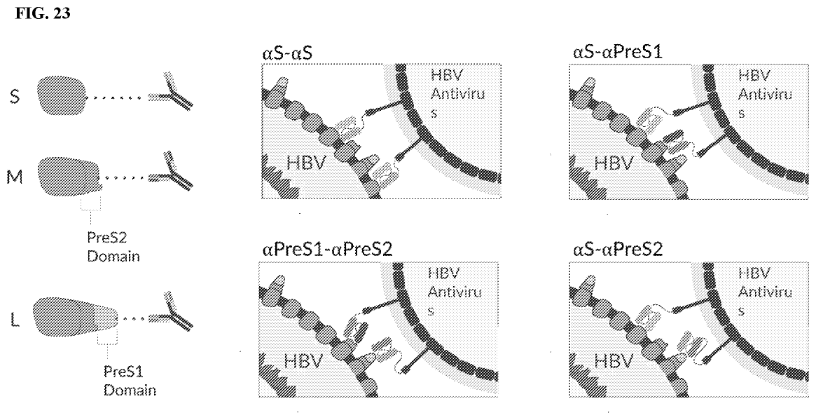

FIG. 23 illustrates HBV antigen specificity combinations in mixed bi-specific HBV-Antiviruses distinct HBV-specific antibodies

### Example 5: In Vivo HBV-Antivirus Treatment of HBV in AAV/HBV Murine Model

Chronic HBV infection in vivo can be resolved by reversing host HBV immune tolerance. HBV-Antiviruses can not only neutralize HBV, but also improve immune clearance and detection of HBV virions and SVPs, which are less than 40 nm in diameter. This is significantly smaller than the threshold for efficient phagocytosis by macrophages and other immune phagocytes, which detect and phagocytose particles around 1 μm in diameter. Thus, larger networks of HBV virions in complex with HBV-Antiviruses can be more efficiently detected by immune cells, allowing HBV-Antiviruses to mediate immune responses against HBV, acting as a therapeutic and a vaccine. To this end, studies are carried out to test the potential of HBV-Antiviruses to serve as neutralizing therapeutics and therapeutic vaccines in a murine AAV/HBV model. In this model, HBV viremia is generated via infection of mice with adeno-associated virus (AAV) carrying a replicable HBV genome, leading to stable viremia and immune tolerance similar to chronic HBV infection in humans. Unlike humanized liver models, mice in this model are immunocompetent. Thus, they can be used to study the immune modulating effects of HBV-Antiviruses on HBV infection. Male C57BL/6 mice with stable AAV/HBV induced viremia are treated with HBV-Antiviruses twice weekly over the course of 4 weeks for a total of 8 doses. During and after the course of treatment, HBV infection in mice is monitored via serum HBV DNA and HBsAg detection, to determine the direct neutralizing effects of HBV-Antiviruses in vivo, as compared to a vehicle control and a neutralizing antibody positive control. Serum anti-HBs antibody titers are determined via ELISA at day 35 and 42 post-treatment, to determine whether HBV-Antivirus treatment could mediate the reversal of HBV immune tolerance and subsequent HBsAg seroconversion. Finally, at day 42 post-treatment, mice are sacrificed and liver dissection performed. Liver HBsAg and HBcAg expression are analyzed via immunohistochemistry (IHC) to determine whether HBV-Antivirus treatment can mediate immune detection and clearance of latently infected cells. The results indicate that HBV-Antiviruses can act a vaccine to facilitate the immune response-mediated detection and clearance of HBV.

### Example 6: HBV-Antiviruses Neutralization of Hepatitis Delta Virus (HDV) In Vitro

Hepatitis Delta virus (HDV) is a satellite virus that can only replicate in the presence of HBV, as it relies on borrowed HBV surface antigens to package virions and infect hepatocytes. HDV co-infection with chronic HBV results in severe complications in HBV disease progression, from acute liver failure to rapid liver cirrhosis and elevated hepatocellular carcinoma risk. Hence, there is a need to develop treatments for both HDV and HBV. Because HDV utilizes the same surface antigens—S, M and L—expressed by HBV, HBV-Antiviruses will similarly be able to target and neutralize HDV, thus making them a potential dual-neutralizing treatment for co-infected individuals. Several HBV-Antiviruses were tested against HDV in an in vitro neutralization assay using primary human hepatocytes. HBV-Antiviruses were incubated with HDV prior to infection, and neutralizing IC₅₀s were determined by measuring intracellular and extracellular HDV RNA levels. Myrcludex B, an approved HBV therapeutic that mimics HBsAg and competes for NTCP binding, was provided as positive control.

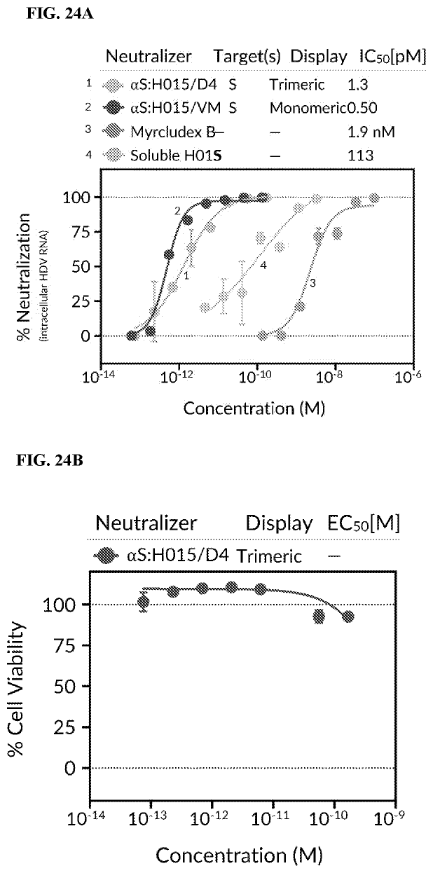

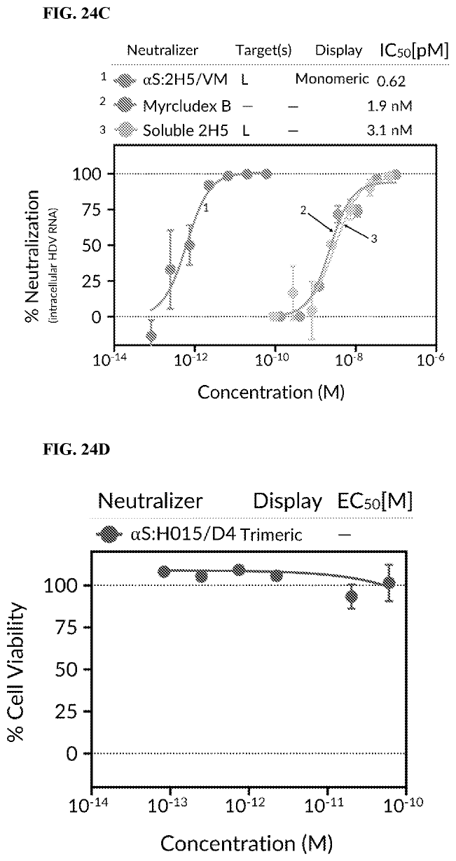

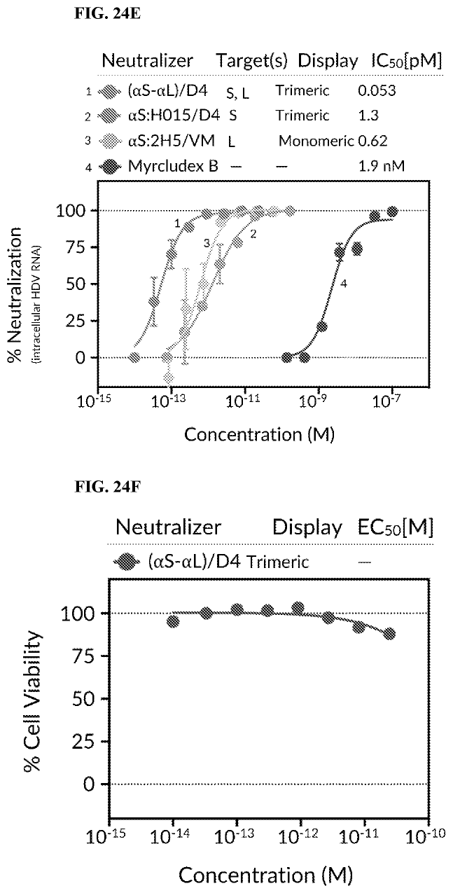

It was found that monomeric αS:H015/VM and trimeric αS:H015/D4 HBV-Antiviruses neutralized HDV in vitro at IC₅₀s of 0.5 and 1.3 pM, respectively (FIG. 24A). This was over 100-fold more potent than soluble, bivalent H015, which neutralized HDV at an IC₅₀ of 113 pM, and over 1000-fold more potent than Myrcludex B, illustrating the potency of multivalent HBV-Antiviruses (FIG. 24A). Furthermore, neither HBV-Antivirus demonstrated any in vitro cytotoxicity measured via CCK-8 (FIG. 24B). Similarly, monomeric αL:2H5/VM neutralized HDV in vitro at an IC₅₀ of 0.62 pM, over 1000-fold more potent than both soluble 2H5 and Myrcludex B, without demonstrating any cytotoxicity at the highest concentrations measured via CCK-8 (FIG. 24C, 24D). Lastly, bi-specific (αS-αL)/D4 was able to neutralize HDV in vitro at IC₅₀ of 0.053 pM, an additional 10-fold increase in neutralizing potency as compared to either of its constituent mono-specific HBV-Antiviruses, and 10,000-fold more potent than Myrcludex B, without demonstrating any cytotoxicity measured via CCK-8 (FIG. 24E, 24F). These results illustrate that HBV-Antiviruses potently neutralize both HBV and HDV, suggesting their potential as a single therapeutic agent capable of treating dangerous HBV and HDV co-infection.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Embodiments

Embodiment 1. An HBV-Antivirus comprising a first fusion protein that comprises a transmembrane polypeptide and an antibody that binds to a surface protein of a hepatitis B virus (HBV) wherein the fusion protein is expressed at a valency of at least about 10 copies on a surface of the antivirus.

Embodiment 2. The HBV-Antivirus of embodiment 1, wherein the antivirus neutralizes the HBV when the first fusion protein is bound to the surface protein of the HBV.

Embodiment 3. The HBV-Antivirus of embodiment 1 or embodiment 2, wherein the antivirus further comprises a second fusion protein.

Embodiment 4. The HBV-Antivirus of embodiment 3, wherein the second fusion protein comprises a transmembrane polypeptide and an immune modulating polypeptide.

Embodiment 5. The HBV-Antivirus of embodiment 4, wherein the immune modulating polypeptide comprises an NK cell activating ligand.

Embodiment 6. The HBV-Antivirus of embodiment 5, wherein the NK cell activating ligand comprises m157, NKG2D ligand, CD70, or CD40.

Embodiment 7. The HBV-Antivirus of embodiment 4, wherein the immune modulating polypeptide comprises an immune checkpoint molecule.

Embodiment 8. The HBV-Antivirus of embodiment 7, wherein the immune checkpoint molecule comprises CTLA4, PD1, OX40, or CD28.

Embodiment 9. The HBV-Antivirus of embodiment 4, wherein the immune modulating polypeptide comprises an inflammatory cytokine.

Embodiment 10. The HBV-Antivirus of embodiment 9, wherein the inflammatory cytokine comprises IL-12, IL-18, TNF-α, or TNF-β.

Embodiment 11. The HBV-Antivirus of embodiment 4, wherein the immune modulating polypeptide comprises a proliferation cytokine.

Embodiment 12. The HBV-Antivirus of embodiment 11, wherein the proliferation cytokine comprises TL-7, IL-15, or IL-21.

Embodiment 13. The HBV-Antivirus of embodiment 3, wherein the second fusion protein comprises a transmembrane polypeptide and an antibody that binds to a surface protein of HBV and the antibody has less than 100% sequence identity to the antibody that binds to a surface protein of HBV of the first fusion protein and wherein the second fusion protein is expressed at least about 10 copies on a surface of the antivirus.

Embodiment 14. The HBV-Antivirus of any one of embodiments 1-13, wherein the antibody of the first fusion protein binds to Large-Hepatitis B surface Antigen (L-HBsAg), Medium-Hepatitis B surface Antigen (M-HBsAg), or Small-Hepatitis B surface Antigen (S-HBsAg).

Embodiment 15. The HBV-Antivirus of embodiment 14, wherein the antibody of the first fusion protein binds to L-HBsAg.

Embodiment 16. The HBV-Antivirus of embodiment 14, wherein the antibody of the first fusion protein binds to M-HBsAg.

Embodiment 17. The HBV-Antivirus of embodiment 14, wherein the antibody of the first fusion protein binds to S-HBsAg.

Embodiment 18. The HBV-Antivirus of any one of embodiments 13-17, wherein the antibody of the second fusion protein binds to Large-Hepatitis B surface Antigen (L-HBsAg), Medium-Hepatitis B surface Antigen (M-HBsAg), or Small-Hepatitis B surface Antigen (S-HBsAg).

Embodiment 19. The HBV-Antivirus of embodiment 18, wherein the antibody of the second fusion protein binds to L-HBsAg.

Embodiment 20. The HBV-Antivirus of embodiment 18, wherein the antibody of the second fusion protein binds to M-HBsAg.

Embodiment 21. The HBV-Antivirus of embodiment 18, wherein the antibody of the second fusion protein binds to S-HBsAg.

Embodiment 22. The HBV-Antivirus of any one of embodiments 1-21, wherein the antibody of the first fusion protein is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

Embodiment 23. The HBV-Antivirus of any one of embodiments 1-22, wherein the antibody of the first fusion protein comprises a neutralizing antibody.

Embodiment 24. The HBV-Antivirus of any one of embodiments 1-23, wherein the antibody of the first fusion protein is a multi-specific antibody.

Embodiment 25. The HBV-Antivirus of embodiment 24, wherein the multi-specific antibody is a bispecific antibody.

Embodiment 26. The HBV-Antivirus of embodiment 25, wherein the bispecific antibody binds to two different epitopes of S-HBsAg.

Embodiment 27. The HBV-Antivirus of embodiment 25, wherein the bispecific antibody binds to S-HBsAg and the Pre-S1 domain of L.

Embodiment 28. The HBV-Antivirus of embodiment 25, wherein the bispecific antibody binds to S-HBsAg and the Pre-S2 domain of M.

Embodiment 29. The HBV-Antivirus of embodiment 24, wherein the multi-specific antibody is a trispecific antibody.

Embodiment 30. The HBV-Antivirus of embodiment 29, wherein the trispecific antibody binds to HBsAg, Pre-S1 domain of L, and Pre-S2 domain of M.

Embodiment 31. The HBV-Antivirus of any one of embodiments 1-30, wherein the antibody of the first fusion protein comprises an amino acid sequence from at least one complementarity determining region of 2H5, ADRI-2F3, H004, H009, H007, H015, H019, or H020.

Embodiment 32. The HBV-Antivirus of any one of embodiments 13-31, wherein the antibody of the second fusion protein is a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

Embodiment 33. The HBV-Antivirus of any one of embodiments 13-32, wherein the antibody of the second fusion protein comprises a neutralizing antibody.

Embodiment 34. The HBV-Antivirus of any one of embodiments 13-33, wherein the antibody of the second fusion protein comprises an amino acid sequence from at least one complementarity determining region of 2H5, ADRI-2F3, H004, H009, H00, H015, H019, or H020.

Embodiment 35. The HBV-Antivirus of any one of embodiments 13-34, wherein the antibody of the second fusion protein is a multi-specific antibody.

Embodiment 36. The HBV-Antivirus of embodiment 35, wherein the multi-specific antibody is a bispecific antibody.

Embodiment 37. The HBV-Antivirus of embodiment 36, wherein the bi-specific antibody binds to 2 distinct epitopes within S-HBsAg.

Embodiment 38. The HBV-Antivirus of embodiment 37, wherein the bispecific antibody binds to S-HBsAg and Pre-S1(L).

Embodiment 39. The HBV-Antivirus of embodiment 37, wherein the bispecific antibody binds to S-HBsAg and Pre-S2(M).

Embodiment 40. The HBV-Antivirus of embodiment 35, wherein the multi-specific antibody is a trispecific antibody.

Embodiment 41. The HBV-Antivirus of embodiment 40, wherein the trispecific antibody binds to S-HBsAg, Pre-S1 domain of L, and Pre-S2 domain of M.

Embodiment 42. The HBV-Antivirus of embodiment 35, wherein the antibody of the first fusion protein is a multi-specific antibody that binds to S-HBsAg and Pre-S1 domain of L and the antibody of the second fusion protein is a multi-specific antibody that binds to HBsAg and Pre-S2 domain of M.

Embodiment 43. The HBV-Antivirus of embodiment 35, wherein the antibody of the first fusion protein is a multi-specific antibody that binds to S-HBsAg and Pre-S2 domain of M and the antibody of the second fusion protein is a multi-specific antibody that binds to S-HBsAg and Pre-S1 domain of L.

Embodiment 44. The HBV-Antivirus of embodiment 35, wherein the antibody of the first fusion protein is a multi-specific antibody that binds to S-HBsAg, Pre-S1 domain of L, and Pre-S2 domain of M and the antibody of the second fusion protein is a multi-specific antibody that binds to S-HBsAg and Pre-S2 domain of M.

Embodiment 45. The HBV-Antivirus of embodiment 35, wherein the antibody of the first fusion protein is a multi-specific antibody that binds to S-HBsAg, Pre-S1 domain of L, and Pre-S2 domain of M and the antibody of the second fusion protein is a multi-specific antibody that binds to S-HBsAg and Pre-S1 domain of L.

Embodiment 46. The HBV-Antivirus of any one of embodiments 1-45, wherein the first fusion protein is monomeric.

Embodiment 47. The HBV-Antivirus of any one of embodiments 1-45, wherein the first fusion protein comprises an oligomerization domain.

Embodiment 48. The HBV-Antivirus of any one of embodiments 3-47, wherein the second fusion protein is monomeric.

Embodiment 49. The HBV-Antivirus of any one of embodiments 3-47, wherein the second fusion protein comprises an oligomerization domain.

Embodiment 50. The HBV-Antivirus of embodiment 49, wherein the oligomerization domain comprises a dimerization domain.

Embodiment 51. The HBV-Antivirus of embodiment 50, wherein the dimerization domain comprises a leucine zipper dimerization domain.

Embodiment 52. The HBV-Antivirus of embodiment 49, wherein the oligomerization domain comprises a trimerization domain.

Embodiment 53. The HBV-Antivirus of embodiment 52, wherein the trimerization domain comprises a post-fusion oligomerization domain of viral surface protein.

Embodiment 54. The HBV-Antivirus of embodiment 52, wherein the trimerization domain comprises a D4 post-fusion trimerization domain of VSV-G protein.

Embodiment 55. The HBV-Antivirus of embodiment 52, wherein the trimerization domain comprises a Dengue E protein post-fusion trimerization domain.

Embodiment 56. The HBV-Antivirus of embodiment 52, wherein the trimerization domain comprises a foldon trimerization domain.

Embodiment 57. The HBV-Antivirus of embodiment 49, wherein the oligomerization domain comprises a tetramerization domain.

Embodiment 58. The HBV-Antivirus of embodiment 57, wherein the tetramerization domain comprises an influenza neuraminidase stem domain.

Embodiment 59. The HBV-Antivirus of embodiment 47, wherein the oligomerization domain comprises an amino acid sequence that has at least 95% sequence identity to an amino acid sequence according to SEQ ID NOs: 17-30.

Embodiment 60. The HBV-Antivirus of any one of embodiments 1-59, wherein the first fusion protein comprises a signal peptide.

Embodiment 61. The HBV-Antivirus of embodiment 60, wherein domains of the first fusion protein are arranged from the N-terminus to the C-terminus in the following orders:
(a) signal peptide, antibody, oligomerization domain, and transmembrane polypeptide;
(b) signal peptide, antibody, transmembrane polypeptide, and oligomerization domain; or
(c) signal peptide, oligomerization domain, antibody, and transmembrane polypeptide.

Embodiment 62. The HBV-Antivirus of embodiment 60 or embodiment 61, wherein the first fusion protein further comprises a cytosolic domain.

Embodiment 63. The HBV-Antivirus of embodiment 62, wherein domains of the first fusion protein are arranged from the N-terminus to the C-terminus in the following orders:
(a) signal peptide, antibody, oligomerization domain, transmembrane polypeptide, and cytosolic domain;
(b) signal peptide, antibody, transmembrane polypeptide, oligomerization domain, and cytosolic domain; or
(c) signal peptide, oligomerization domain, antibody, transmembrane polypeptide, and cytosolic domain.

Embodiment 64. The HBV-Antivirus of any one of embodiments 3-63, wherein the second fusion protein comprises a signal peptide.

Embodiment 65. The HBV-Antivirus of embodiment 64, wherein domains of the second fusion protein are arranged from the N-terminus to the C-terminus in the following orders:
(a) signal peptide, antibody, oligomerization domain, and transmembrane polypeptide;
(b) signal peptide, antibody, transmembrane polypeptide, and oligomerization domain; or
(c) signal peptide, oligomerization domain, antibody, and transmembrane polypeptide.

Embodiment 66. The HBV-Antivirus of embodiment 64 or 65, wherein the second fusion protein further comprises a cytosolic domain.

Embodiment 67. The HBV-Antivirus of embodiment 66, wherein domains of the second fusion protein are arranged from the N-terminus to the C-terminus in the following orders:
(a) signal peptide, antibody, oligomerization domain, transmembrane polypeptide, and cytosolic domain;
(b) signal peptide, antibody, transmembrane polypeptide, oligomerization domain, and cytosolic domain; or
(c) signal peptide, oligomerization domain, antibody, transmembrane polypeptide, and cytosolic domain.

Embodiment 68. The HBV-Antivirus of any one of embodiments 1-67, wherein the transmembrane polypeptide anchors the first fusion protein to a bilayer of the antivirus.

Embodiment 69. The HBV-Antivirus of any one of embodiments 3-68, wherein the transmembrane polypeptide anchors the second fusion protein to a bilayer of the antivirus.

Embodiment 70. The HBV-Antivirus of any one of embodiments 1-69, wherein the transmembrane polypeptide comprises the transmembrane domain of a Vesicular Stomatitis virus glycoprotein (VSV-G).

Embodiment 71. The HBV-Antivirus of any one of embodiments 1-69, wherein the transmembrane polypeptide comprises the transmembrane domain of influenza Hemagglutinin (HA).

Embodiment 72. The HBV-Antivirus of any one of embodiments 1-69, wherein the transmembrane polypeptide comprises the transmembrane domain of HIV surface glycoprotein GP120 or GP41.

Embodiment 73. The HBV-Antivirus of any one of embodiments 1-69, wherein the transmembrane polypeptide comprises the transmembrane domain of measles virus surface glycoprotein hemagglutinin (H) protein.

Embodiment 74. The HBV-Antivirus of any one of embodiments 1-69, wherein the transmembrane polypeptide comprises an amino acid sequence at least about 90% identical to that set forth in SEQ ID NO: 31-39.

Embodiment 75. The HBV-Antivirus of any one of embodiments 1-74, wherein the antivirus is not a lentiviral particle.

Embodiment 76. The HBV-Antivirus of any one of embodiments 1-74, wherein the antivirus does not comprise viral genetic material.

Embodiment 77. The HBV-Antivirus of any one of embodiments 1-74, wherein the antivirus comprises a lipid bilayer.

Embodiment 78. The HBV-Antivirus of any one of embodiments 1-74, wherein the antivirus comprises an enveloped particle.

Embodiment 79. The HBV-Antivirus of any one of embodiments 1-74, wherein the antivirus comprises a virus.

Embodiment 80. The HBV-Antivirus of any one of embodiments 1-74, wherein the antivirus comprises a replication incompetent virus.

Embodiment 81. The HBV-Antivirus of any one of embodiments 1-74, wherein the antivirus comprises a replication competent virus.

Embodiment 82. The HBV-Antivirus of any one of embodiments 1-74, wherein the antivirus comprises a viral-like particle.

Embodiment 83. The HBV-Antivirus of any one of embodiments 1-74, wherein the antivirus comprises an extracellular vesicle.

Embodiment 84. The HBV-Antivirus of any one of embodiments 1-74, wherein the extracellular vesicle comprises an ectosome.

Embodiment 85. The HBV-Antivirus of any one of embodiments 1-74, wherein the extracellular vesicle comprises an exosome.

Embodiment 86. The HBV-Antivirus of any one of embodiments 1-85, wherein the first fusion protein is expressed at a valency of about 10 copies on a surface of the antivirus.

Embodiment 87. The HBV-Antivirus of embodiment 86, wherein the first fusion protein is expressed at a valency of about 10 to 15 copies on a surface of the antivirus.

Embodiment 88. The HBV-Antivirus of any one of embodiments 1-85, wherein the first fusion protein is expressed at a valency of at least about 25 copies on a surface of the antivirus.

Embodiment 89. The HBV-Antivirus of any one of embodiments 1-85, wherein the first fusion protein is expressed at a valency of at least about 50 copies on a surface of the antivirus.

Embodiment 90. The HBV-Antivirus of any one of embodiments 1-85, wherein the first fusion protein is expressed at a valency of at least about 100 copies on a surface of the antivirus.

Embodiment 91. The HBV-Antivirus of any one of embodiments 1-85, wherein the first fusion protein is expressed at a valency of at least about 200 copies on a surface of the antivirus.

Embodiment 92. The HBV-Antivirus of any one of embodiments 1-85, wherein the first fusion protein is expressed at a valency of at least about 400 copies on a surface of the antivirus.

Embodiment 93. The HBV-Antivirus of any one of embodiments 1-85, wherein the first fusion protein is expressed at a valency of at least about 600 copies on a surface of the antivirus.

Embodiment 94. The HBV-Antivirus of any one of embodiments 1-85, wherein the first fusion protein is expressed at a valency of At least about 1000 copies on a surface of the antivirus.

Embodiment 95. The HBV-Antivirus of any one of embodiments 3-94, wherein the second fusion protein is expressed at a valency of about 10 copies on a surface of the antivirus.

Embodiment 96. The HBV-Antivirus of embodiment 95, wherein the second fusion protein is expressed at a valency of about 10 to 15 copies on a surface of the antivirus.

Embodiment 97. The HBV-Antivirus of any one of embodiments 3-94, wherein the second fusion protein is expressed at a valency of at least about 25 copies on a surface of the antivirus.

Embodiment 98. The HBV-Antivirus of any one of embodiments 3-94, wherein the second fusion protein is expressed at a valency of at least about 50 copies on a surface of the antivirus.

Embodiment 99. The HBV-Antivirus of any one of embodiments 3-94, wherein the second fusion protein is expressed at a valency of at least about 100 copies on a surface of the antivirus.

Embodiment 100. The HBV-Antivirus of any one of embodiments 3-94, wherein the second fusion protein is expressed at a valency of at least about 200 copies on a surface of the antivirus.

Embodiment 101. The HBV-Antivirus of any one of embodiments 3-94, wherein the second fusion protein is expressed at a valency of at least about 400 copies on a surface of the antivirus.

Embodiment 102. The HBV-Antivirus of any one of embodiments 3-94, wherein the second fusion protein is expressed at a valency of at least about 600 copies on a surface of the antivirus.

Embodiment 103. The HBV-Antivirus of any one of embodiments 3-94, wherein the second fusion protein is expressed at a valency of at least about 1000 copies on a surface of the antivirus.

Embodiment 104. The HBV-Antivirus of any one of embodiments 1-103, wherein the antivirus comprises a fluorophore expressed on a surface of the antivirus.

Embodiment 105. The HBV-Antivirus of embodiment 104, wherein the fluorophore is conjugated to a membrane-intercalating polypeptide.

Embodiment 106. A composition comprising a nucleic acid sequence that encodes the first fusion protein of any of embodiments 1-105.

Embodiment 107. A composition comprising a nucleic acid sequence that encodes the first fusion protein of any one of embodiments 1-105 and the second fusion protein of any one of embodiments 3-105.

Embodiment 108. The composition of embodiment 107, wherein the composition further comprises a second nucleic acid sequence that encodes one or more packaging viral proteins.

Embodiment 109. The composition of embodiment 108, wherein the one or more packaging viral proteins is a lentiviral protein, a retroviral protein, an adenoviral protein, or combinations thereof.

Embodiment 110. The composition of embodiment 108, wherein the one or more packaging viral proteins comprises gag, pol, pre, tat, rev, or combinations thereof.

Embodiment 111. The composition of any one of embodiments 106-110, further comprising a third nucleic acid sequence that encodes a reporter, a therapeutic molecule, or combinations thereof.

Embodiment 112. The composition of embodiment 111, wherein the reporter is a fluorescent protein or luciferase.

Embodiment 113. The composition of embodiment 112, wherein the fluorescent protein is green fluorescent protein.

Embodiment 114. The composition of embodiment 111, wherein the therapeutic molecule is an immune modulating protein, a cellular signal modulating molecule, a proliferation modulating molecule, or combinations thereof.

Embodiment 115. The composition of any one of embodiments 106-114, wherein the nucleic acid sequence that encodes the first fusion protein and the second nucleic acid sequence and the third nucleic acid sequence are within a same vector.

Embodiment 116. The composition of any one of embodiments 111-115, wherein the nucleic acid sequence that encodes the first fusion protein and the second fusion protein and the second nucleic acid sequence and the third nucleic acid sequence are within a same vector.

Embodiment 117. The composition of any one of embodiments 111-115, wherein the nucleic acid sequence that encodes the first fusion protein and the second nucleic acid sequence and the third nucleic acid sequence are within different vectors.

Embodiment 118. The composition of any one of embodiments 111-115, wherein the nucleic acid sequence that encodes the first fusion protein and the second fusion protein and the second nucleic acid sequence and the third nucleic acid sequence are within different vectors.

Embodiment 119. The composition of any one of embodiments 115-118, wherein the vector is a lentivirus vector, an adenovirus vector, or an adeno-associated virus vector.

Embodiment 120. The composition of any one of embodiments 111-119, wherein the nucleic acid sequence that encodes the first fusion protein and the second fusion protein and the second nucleic acid sequence and the third nucleic acid sequence are DNAs.

Embodiment 121. The composition of any one of embodiments 111-119, wherein the nucleic acid sequence that encodes the first fusion protein and the second fusion protein and the second nucleic acid sequence and the third nucleic acid sequence are mRNAs.

Embodiment 122. A method of treating hepatitis B virus in a subject in need thereof comprising administering to the subject the HBV-antivirus according to any one of embodiments 1-105.

Embodiment 123. The method of embodiment 122, wherein the HBV-antivirus is administered to the subject intravenously.

Embodiment 124. The method of embodiment 122, wherein the HBV-antivirus is administered to the subject through inhalation.

Embodiment 125. The method of embodiment 122, wherein the HBV-antivirus is administered to the subject through intranasal delivery.

Embodiment 126. The method of embodiment 122, wherein the HBV-antivirus is administered to the subject by an intraperitoneal injection.

Embodiment 127. The method of embodiment 122, wherein the HBV-antivirus is administered to the subject by a subcutaneous injection.

Embodiment 128. The method of any one of embodiments 122-127, wherein the HBV-antivirus induces T cell mediated cytotoxicity against viral infected cells.

Embodiment 129. The method of any one of embodiments 122-128, wherein the HBV-antivirus is taken up by an antigen-presenting cell when the HBV-antivirus is bound to the surface protein of the HBV.

Embodiment 130. The method of any one of embodiments 122-128, wherein the HBV-antivirus increases adaptive immune responses against HBV.

Embodiment 131. The method of any one of embodiments 122-130, wherein the administering to the subject of the HBV-antivirus is sufficient to reduce or eliminate the HBV as compared to a baseline measurement of the HBV taken from the subject prior to the administering of the HBV-antivirus.

Embodiment 132. The method of embodiment 131, wherein the reduction is at least about 1-fold, 5-fold, 10-fold, 20-fold, 40-fold, 60-fold, 80-fold, or up to about 100-fold.

Embodiment 133. A method of treating hepatitis B virus in a subject in need thereof comprising administering to the subject a composition of any one of embodiments 106-121.

Embodiment 134. The method of embodiment 133, wherein the composition is administered to the subject through inhalation.

Embodiment 135. The method of embodiment 133, wherein the composition is administered to the subject through intranasal delivery.

Embodiment 136. The method of embodiment 133, wherein the composition is administered to the subject through intratracheal delivery.

Embodiment 137. The method of embodiment 133, wherein the composition is administered to the subject by an intraperitoneal injection.

Embodiment 138. The method of embodiment 133, wherein the composition is administered to the subject by a subcutaneous injection.

Embodiment 139. The method of any one of embodiments 133-138, wherein the administering to the subject of the composition is sufficient to reduce or eliminate the cancer as compared to a baseline measurement of the cancer taken from the subject prior to the administering of the composition.

Embodiment 140. The method of any one of embodiments 133-139, wherein the composition is administered with a liposome.

Embodiment 141. The method of any one of embodiments 133-139, wherein the composition is administered with an adeno-associated virus (AAV).

Embodiment 142. The method of any one of embodiments 133-139, wherein the composition is administered with a lipid nanoparticle.

Embodiment 143. The method of any one of embodiments 133-139, wherein the composition is administered with a polymer.

Embodiment 144. The method of any one of embodiments 133-139, wherein the composition is administered as a naked nucleic acid sequence.

Embodiment 145. The method of any one of embodiments 133-139, wherein the composition is administered as a naked DNA sequence.

Embodiment 146. The method of any one of embodiments 133-139, wherein the composition is administered as a naked mRNA sequence.

Embodiment 147. A pharmaceutical composition comprising (a) the HBV-Antivirus of any one of embodiments 1-105, and (b) a pharmaceutically acceptable excipient.

Embodiment 148. An HBV-Antivirus comprising a multivalent particle displaying at least 10 copies of an antibody that binds to a surface protein of an HBV.

Embodiment 149. A multi-specific HBV-Antivirus comprising a multivalent particle displaying at least 10 copies of a multi-specific antibody that binds to one or more surface proteins of an HBV.

Embodiment 150. A multi-specific HBV-Antivirus comprising a multivalent particle displaying at least 10 copies of a first antibody that binds to a surface protein of an HBV and at least 10 copies of a second antibody that binds to a surface protein of the HBV wherein the second antibody has less than 100% sequence identity to the first antibody.

Embodiment 151. A dual-action, immune-modulating HBV-Antivirus comprising a multivalent particle displaying at least 10 copies of an antibody that binds to a surface protein of an HBV and at least 10 copies of an immune modulating polypeptide.

What is claimed is:

1. A multivalent HBV-Antivirus comprising a first fusion protein that comprises a transmembrane polypeptide and an antibody that binds to a surface protein of a hepatitis B virus (HBV) wherein the first fusion protein is expressed on a surface of the antivirus.

2. The HBV-Antivirus of claim 1, wherein the antivirus neutralizes the HBV when the first fusion protein is bound to a surface protein of the HBV.

3. The HBV-Antivirus of claim 1, wherein the antivirus further comprises a second fusion protein that is expressed on the surface of the antivirus.

4. The HBV-Antivirus of claim 3, wherein the second fusion protein comprises a transmembrane polypeptide and an immune modulating polypeptide.

5. The HBV-Antivirus of claim 4, wherein the immune modulating polypeptide comprises an NK cell activating ligand.

6. The HBV-Antivirus of claim 5, wherein the NK cell activating ligand comprises m157, NKG2D ligand, CD70, or CD40.

7. The HBV-Antivirus of claim 4, wherein the immune modulating polypeptide comprises an immune checkpoint molecule.

8. The HBV-Antivirus of claim 7, wherein the immune checkpoint molecule comprises CTLA4, PD1, OX40, or CD28.

9. The HBV-Antivirus of claim 4, wherein the immune modulating polypeptide comprises an inflammatory cytokine.

10. The HBV-Antivirus of claim 9, wherein the inflammatory cytokine comprises IL-12, IL-18, TNF-α, or TNF-β.

11. The HBV-Antivirus of claim 4, wherein the immune modulating polypeptide comprises a proliferation cytokine.

12. The HBV-Antivirus of claim 11, wherein the proliferation cytokine comprises IL-7, IL-15, or IL-21.

13. The HBV-Antivirus of claim 3, wherein the second fusion protein comprises a transmembrane polypeptide and an antibody that binds to a surface protein of HBV and the antibody has less than 100% sequence identity to the antibody of the first fusion protein that binds to the surface protein of HBV.

14. The HBV-Antivirus of claim 1, wherein the antibody of the first fusion protein binds to Large Hepatitis B surface Antigen (L-HBsAg), Medium Hepatitis B surface Antigen (M-HBsAg), or Small Hepatitis B surface Antigen (S-HBsAg).

15. The HBV-Antivirus of claim 14, wherein the antibody of the first fusion protein binds to L-HBsAg.

16. The HBV-Antivirus of claim 14, wherein the antibody of the first fusion protein binds to M-HBsAg.

17. The HBV-Antivirus of claim 14, wherein the antibody of the first fusion protein binds to S-HBsAg.

18. The HBV-Antivirus of claim 13, wherein the antibody of the second fusion protein binds to Large Hepatitis B surface Antigen (L-HBsAg), Medium Hepatitis B surface Antigen (M-HBsAg), or Small Hepatitis B surface Antigen (S-HBsAg).

19. The HBV-Antivirus of claim 18, wherein the antibody of the second fusion protein binds to L-HBsAg.

20. The HBV-Antivirus of claim 18, wherein the antibody of the second fusion protein binds to M-HBsAg.

21. The HBV-Antivirus of claim 18, wherein the antibody of the second fusion protein binds to S-HBsAg.

22. The HBV-Antivirus of claim 1, wherein the antibody of the first fusion protein comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

23. The HBV-Antivirus of claim 1, wherein the antibody of the first fusion protein comprises a neutralizing antibody.

24. The HBV-Antivirus of claim 1, wherein the antibody of the first fusion protein comprises a multi-specific antibody.

25. The HBV-Antivirus of claim 24, wherein the multi-specific antibody comprises a bispecific antibody.

26. The HBV-Antivirus of claim 25, wherein the bispecific antibody binds to two different epitopes of S-HBsAg.

27. The HBV-Antivirus of claim 25, wherein the bispecific antibody binds to S-HBsAg and the Pre-S1 domain of L-HBsAg.

28. The HBV-Antivirus of claim 25, wherein the bispecific antibody binds to S-HBsAg and the Pre-S2 domain of M-HBsAg.

29. The HBV-Antivirus of claim 24, wherein the multi-specific antibody comprises a trispecific antibody.

30. The HBV-Antivirus of claim 29, wherein the trispecific antibody binds to HBsAg, Pre-S1 domain of L-HBsAg, and Pre-S2 domain of M-HBsAg.

31. The HBV-Antivirus of claim 1, wherein the antibody of the first fusion protein comprises an amino acid sequence from at least one complementarity determining region of 2H5, ADRI-2F3, H004, H009, H007, H015, H019, or H020.

32. The HBV-Antivirus of claim 13, wherein the antibody of the second fusion protein comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

33. The HBV-Antivirus of claim 13, wherein the antibody of the second fusion protein comprises a neutralizing antibody.

34. The HBV-Antivirus of claim 13, wherein the antibody of the second fusion protein comprises an amino acid sequence from at least one complementarity determining region of 2H5, ADRI-2F3, H004, H009, H00, H015, H019, or H020.

35. The HBV-Antivirus of claim 13, wherein the antibody of the second fusion protein comprises a multi-specific antibody.

36. The HBV-Antivirus of claim 35, wherein the multi-specific antibody comprises a bispecific antibody.

37. The HBV-Antivirus of claim 36, wherein the bi-specific antibody binds to two different epitopes of S-HBsAg.

38. The HBV-Antivirus of claim 36, wherein the bispecific antibody binds to S-HBsAg and Pre-S1 domain of L-HBsAg.

39. The HBV-Antivirus of claim 36, wherein the bispecific antibody binds to S-HBsAg and Pre-S2 domain of M-HBsAg.

40. The HBV-Antivirus of claim 35, wherein the multi-specific antibody comprises a trispecific antibody.

41. The HBV-Antivirus of claim 40, wherein the trispecific antibody binds to S-HBsAg, Pre-S1 domain of L-HBsAg, and Pre-S2 domain of M-HBsAg.

42. The HBV-Antivirus of claim 35, wherein the antibody of the first fusion protein comprises a multi-specific antibody that binds to S-HBsAg and Pre-S1 domain of L and the antibody of the second fusion protein comprises a multi-specific antibody that binds to HBsAg and Pre-S2 domain of M.

43. The HBV-Antivirus of claim 35, wherein the antibody of the first fusion protein comprises a multi-specific antibody that binds to S-HBsAg and Pre-S2 domain of M-HBsAg and the antibody of the second fusion protein comprises a multi-specific antibody that binds to S-HBsAg and Pre-S1 domain of L-HBsAg.

44. The HBV-Antivirus of claim 35, wherein the antibody of the first fusion protein comprises a multi-specific antibody that binds to S-HBsAg, Pre-S1 domain of L-HBsAg, and Pre-S2 domain of M-HBsAg and the antibody of the second fusion protein comprises a multi-specific antibody that binds to S-HBsAg and Pre-S2 domain of M-HBsAg.

45. The HBV-Antivirus of claim 35, wherein the antibody of the first fusion protein comprises a multi-specific antibody that binds to S-HBsAg, Pre-S1 domain of L-HBsAg, and Pre-S2 domain of M-HBsAg and the antibody of the second fusion protein comprises a multi-specific antibody that binds to S-HBsAg and Pre-S1 domain of L-HBsAg.

46. The HBV-Antivirus of claim 1, wherein the first fusion protein is monomeric.

47. The HBV-Antivirus of claim 1, wherein the first fusion protein comprises an oligomerization domain.

48. The HBV-Antivirus of claim 3, wherein the second fusion protein is monomeric.

49. The HBV-Antivirus of claim 3, wherein the second fusion protein comprises an oligomerization domain.

50. The HBV-Antivirus of claim 47, wherein the oligomerization domain comprises a dimerization domain.

51. The HBV-Antivirus of claim 50, wherein the dimerization domain comprises a leucine zipper dimerization domain.

52. The HBV-Antivirus of claim 47, wherein the oligomerization domain comprises a trimerization domain.

53. The HBV-Antivirus of claim 52, wherein the trimerization domain comprises a post-fusion oligomerization domain of viral surface protein.

54. The HBV-Antivirus of claim 52, wherein the trimerization domain comprises a D4 post-fusion trimerization domain of VSV-G protein.

55. The HBV-Antivirus of claim 52, wherein the trimerization domain comprises a Dengue E protein post-fusion trimerization domain.

56. The HBV-Antivirus of claim 52, wherein the trimerization domain comprises a foldon trimerization domain.

57. The HBV-Antivirus of claim 47, wherein the oligomerization domain comprises a tetramerization domain.

58. The HBV-Antivirus of claim 57, wherein the tetramerization domain comprises an influenza neuraminidase stem domain.

59. The HBV-Antivirus of claim 49, wherein the oligomerization domain comprises a dimerization domain.

60. The HBV-Antivirus of claim 59, wherein the dimerization domain comprises a leucine zipper dimerization domain.

61. The HBV-Antivirus of claim 49, wherein the oligomerization domain comprises a trimerization domain.

62. The HBV-Antivirus of claim 61, wherein the trimerization domain comprises a post-fusion oligomerization domain of viral surface protein.

63. The HBV-Antivirus of claim 61, wherein the trimerization domain comprises a D4 post-fusion trimerization domain of VSV-G protein.

64. The HBV-Antivirus of claim 61, wherein the trimerization domain comprises a Dengue E protein post-fusion trimerization domain.

65. The HBV-Antivirus of claim 61, wherein the trimerization domain comprises a foldon trimerization domain.

66. The HBV-Antivirus of claim 49, wherein the oligomerization domain comprises a tetramerization domain.

67. The HBV-Antivirus of claim 66, wherein the tetramerization domain comprises an influenza neuraminidase stem domain.

68. The HBV-Antivirus of claim 47, wherein the oligomerization domain comprises an amino acid sequence that has at least 90% sequence identity to an amino acid sequence according to any one of SEQ ID NOs: 17-30.

69. The HBV-Antivirus of claim 49, wherein the oligomerization domain comprises an amino acid sequence that has at least 90% sequence identity to an amino acid sequence according to any one of SEQ ID NOs: 17-30.

70. The HBV-Antivirus of claim 47, wherein the first fusion protein comprises a signal peptide.

71. The HBV-Antivirus of claim 70, wherein domains of the first fusion protein are arranged from the N-terminus to the C-terminus in the following orders:
(a) signal peptide, antibody, oligomerization domain, and transmembrane polypeptide;
(b) signal peptide, antibody, transmembrane polypeptide, and oligomerization domain; or
(c) signal peptide, oligomerization domain, antibody, and transmembrane polypeptide.

72. The HBV-Antivirus of claim 70, wherein the first fusion protein further comprises a cytosolic domain.

73. The HBV-Antivirus of claim 72, wherein domains of the first fusion protein are arranged from the N-terminus to the C-terminus in the following orders:
(a) signal peptide, antibody, oligomerization domain, transmembrane polypeptide, and cytosolic domain;
(b) signal peptide, antibody, transmembrane polypeptide, oligomerization domain, and cytosolic domain; or
(c) signal peptide, oligomerization domain, antibody, transmembrane polypeptide, and cytosolic domain.

74. The HBV-Antivirus of claim 49, wherein the second fusion protein comprises a signal peptide.

75. The HBV-Antivirus of claim 74, wherein domains of the second fusion protein are arranged from the N-terminus to the C-terminus in the following orders:
(a) signal peptide, antibody, oligomerization domain, and transmembrane polypeptide;
(b) signal peptide, antibody, transmembrane polypeptide, and oligomerization domain; or
(c) signal peptide, oligomerization domain, antibody, and transmembrane polypeptide.

76. The HBV-Antivirus of claim 74, wherein the second fusion protein further comprises a cytosolic domain.

77. The HBV-Antivirus of claim 76, wherein domains of the second fusion protein are arranged from the N-terminus to the C-terminus in the following orders:
(a) signal peptide, antibody, oligomerization domain, transmembrane polypeptide, and cytosolic domain;
(b) signal peptide, antibody, transmembrane polypeptide, oligomerization domain, and cytosolic domain; or
(c) signal peptide, oligomerization domain, antibody, transmembrane polypeptide, and cytosolic domain.

78. The HBV-Antivirus of claim 1, wherein the transmembrane polypeptide anchors the first fusion protein to a lipid bilayer of the antivirus.

79. The HBV-Antivirus of claim 4, wherein the transmembrane polypeptide anchors the second fusion protein to a lipid bilayer of the antivirus.

80. The HBV-Antivirus of claim 1, wherein the transmembrane polypeptide of the first fusion protein comprises the transmembrane domain of a Vesicular Stomatitis virus glycoprotein (VSV-G).

81. The HBV-Antivirus of claim 1, wherein the transmembrane polypeptide of the first fusion protein comprises the cytosolic tail of VSV-G.

82. The HBV-Antivirus of claim 1, wherein the transmembrane polypeptide of the first fusion protein comprises the transmembrane domain of influenza Neuraminidase (NA).

83. The HBV-Antivirus of claim 1, wherein the transmembrane polypeptide of the first fusion protein comprises the transmembrane domain of influenza Hemagglutinin (HA).

84. The HBV-Antivirus of claim 1, wherein the transmembrane polypeptide of the first fusion protein comprises the transmembrane domain of Dengue E Protein.

85. The HBV-Antivirus of claim 1, wherein the transmembrane polypeptide of the first fusion protein comprises the transmembrane domain of HIV surface glycoprotein GP120 or GP41.

86. The HBV-Antivirus of claim 1, wherein the transmembrane polypeptide of the first fusion protein comprises the transmembrane domain of measles virus surface glycoprotein hemagglutinin (H) protein.

87. The HBV-Antivirus of claim 4, wherein the transmembrane polypeptide of the second fusion protein comprises the transmembrane domain of a Vesicular Stomatitis virus glycoprotein (VSV-G).

88. The HBV-Antivirus of claim 4, wherein the transmembrane polypeptide of the second fusion protein comprises the cytosolic tail of VSV-G.

89. The HBV-Antivirus of claim 4, wherein the transmembrane polypeptide of the second fusion protein comprises the transmembrane domain of influenza Neuraminidase (NA).

90. The HBV-Antivirus of claim 4, wherein the transmembrane polypeptide of the second fusion protein comprises the transmembrane domain of influenza Hemagglutinin (HA).

91. The HBV-Antivirus of claim 4, wherein the transmembrane polypeptide of the second fusion protein comprises the transmembrane domain of Dengue E Protein.

92. The HBV-Antivirus of claim 4, wherein the transmembrane polypeptide of the second fusion protein comprises the transmembrane domain of HIV surface glycoprotein GP120 or GP41.

93. The HBV-Antivirus of claim 4, wherein the transmembrane polypeptide of the second fusion protein comprises the transmembrane domain of measles virus surface glycoprotein hemagglutinin (H) protein.

94. The HBV-Antivirus of claim 1, wherein the transmembrane polypeptide of the first fusion protein comprises an amino acid sequence of at least about 90% sequence identity to any one of SEQ ID NO: 31-39.

95. The HBV-Antivirus of claim 4, wherein the transmembrane polypeptide of the second fusion protein comprises an amino acid sequence of at least about 90% sequence identity to any one of SEQ ID NO: 31-39.

96. The HBV-Antivirus of claim 1, wherein the antivirus is not a lentiviral particle.

97. The HBV-Antivirus of claim 1, wherein the antivirus does not comprise viral genetic material.

98. The HBV-Antivirus of claim 1, wherein the antivirus comprises a lipid bilayer.

99. The HBV-Antivirus of claim 1, wherein the antivirus comprises an enveloped particle.

100. The HBV-Antivirus of claim 1, wherein the antivirus comprises a virus.

101. The HBV-Antivirus of claim 1, wherein the antivirus comprises a replication incompetent virus.

102. The HBV-Antivirus of claim 1, wherein the antivirus comprises a replication competent virus.

103. The HBV-Antivirus of claim 1, wherein the antivirus comprises a viral-like particle.

104. The HBV-Antivirus of claim 1, wherein the antivirus comprises an extracellular vesicle.

105. The HBV-Antivirus of claim 104, wherein the extracellular vesicle comprises an ectosome.

106. The HBV-Antivirus of claim 104, wherein the extracellular vesicle comprises an exosome.

107. The HBV-Antivirus of claim 1, wherein the first fusion protein is expressed at a valency of about 10 copies on the surface of the antivirus.

108. The HBV-Antivirus of claim 1, wherein the first fusion protein is expressed at a valency of about 10 to 15 copies on the surface of the antivirus.

109. The HBV-Antivirus of claim 1, wherein the first fusion protein is expressed at a valency of at least about 25 copies on the surface of the antivirus.

110. The HBV-Antivirus of claim 1, wherein the first fusion protein is expressed at a valency of at least about 50 copies on the surface of the antivirus.

111. The HBV-Antivirus of claim 1, wherein the first fusion protein is expressed at a valency of at least about 100 copies on the surface of the antivirus.

112. The HBV-Antivirus of claim 1, wherein the first fusion protein is expressed at a valency of at least about 200 copies on the surface of the antivirus.

113. The HBV-Antivirus of claim 1, wherein the first fusion protein is expressed at a valency of at least about 400 copies on the surface of the antivirus.

114. The HBV-Antivirus of claim 1, wherein the first fusion protein is expressed at a valency of at least about 600 copies on the surface of the antivirus.

115. The HBV-Antivirus of claim 1, wherein the first fusion protein is expressed at a valency of at least about 1000 copies on a surface of the antivirus.

116. The HBV-Antivirus of claim 1, wherein the first fusion protein is expressed at a valency of at least about 2000 copies on the surface of the antivirus.

117. The HBV-Antivirus claim 3, wherein the second fusion protein is expressed at a valency of about 10 copies on the surface of the antivirus.

118. The HBV-Antivirus of claim 3, wherein the second fusion protein is expressed at a valency of about 10 to 15 copies on the surface of the antivirus.

119. The HBV-Antivirus of claim 3, wherein the second fusion protein is expressed at a valency of at least about 25 copies on the surface of the antivirus.

120. The HBV-Antivirus of claim 3, wherein the second fusion protein is expressed at a valency of at least about 50 copies on the surface of the antivirus.

121. The HBV-Antivirus of claim 3, wherein the second fusion protein is expressed at a valency of at least about 100 copies on the surface of the antivirus.

122. The HBV-Antivirus of claim 3, wherein the second fusion protein is expressed at a valency of at least about 200 copies on the surface of the antivirus.

123. The HBV-Antivirus of claim 3, wherein the second fusion protein is expressed at a valency of at least about 400 copies on the surface of the antivirus.

124. The HBV-Antivirus of claim 3, wherein the second fusion protein is expressed at a valency of at least about 600 copies on the surface of the antivirus.

125. The HBV-Antivirus of claim 3, wherein the second fusion protein is expressed at a valency of at least about 1000 copies on the surface of the antivirus.

126. The HBV-Antivirus of claim 3, wherein the second fusion protein is expressed at a valency of at least about 2000 copies on the surface of the antivirus.

127. The HBV-Antivirus of claim 1, wherein the antivirus comprises a fluorophore expressed on the surface of the antivirus.

128. The HBV-Antivirus of claim 127, wherein the fluorophore is conjugated to a membrane-intercalating polypeptide.

129. The HBV-Antivirus of claim 1, wherein the antivirus neutralizes a hepatitis delta virus (HDV) when the first fusion protein is bound to an HBV surface protein located on the surface of HDV.

130. The HBV-Antivirus of claim 1, wherein the antibody of the first fusion protein comprises:
(a) a heavy chain variable region (VH) comprising an amino acid sequence having at least 90% sequence identity to an amino acid sequence of any one of SEQ ID Nos: 1, 3, 5, 7, 9, 11, 13, and 15; and
(b) a light chain variable region (VL) comprising an amino acid sequence having at least 90% sequence identity to an amino acid sequence of any one of SEQ ID Nos: 2, 4, 6, 8, 10, 12, 14, and 16.

131. The HBV-Antivirus of claim 1, wherein:
(a) the antibody of the first fusion protein comprises an amino acid sequence from at least one complementarity determining region of 2H5, ADRI-2F3, H004, H009, H007, H015, H019, or H020; and
(b) the transmembrane polypeptide of the first fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein.

132. The HBV-Antivirus of claim 1, wherein:
(a) the antibody of the first fusion protein comprises:
(i) a heavy chain variable region (VH) comprising an amino acid sequence having at least 90% sequence identity to an amino acid sequence of any one of SEQ ID Nos: 1, 3, 5, 7, 9, 11, 13, and 15; and
(ii) a light chain variable region (VL) comprising an amino acid sequence having at least 90% sequence identity to an amino acid sequence of any one of SEQ ID Nos: 2, 4, 6, 8, 10, 12, 14, and 16; and
(b) the transmembrane polypeptide of the first fusion protein comprises an amino acid sequence having at least 90% sequence identity to an amino acid sequence of any one of SEQ ID Nos: 31-39.

133. The HBV-Antivirus of claim 47, wherein:
(a) the antibody of the first fusion protein comprises an amino acid sequence from at least one complementarity determining region of 2H5, ADRI-2F3, H004, H009, H007, H015, H019, or H020;
(b) the transmembrane polypeptide of the first fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and
(c) the oligomerization domain of the first fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain.

134. The HBV-Antivirus of claim 47, wherein:
(a) the antibody of the first fusion protein comprises:
(i) a heavy chain variable region (VH) comprising an amino acid sequence having at least 90% sequence identity to an amino acid sequence of any one of SEQ ID Nos: 1, 3, 5, 7, 9, 11, 13, and 15; and
(ii) a light chain variable region (VL) comprising an amino acid sequence having at least 90% sequence identity to an amino acid sequence of any one of SEQ ID Nos: 2, 4, 6, 8, 10, 12, 14, and 16;
(b) the transmembrane polypeptide of the first fusion protein comprises an amino acid sequence having at least 90% sequence identity to an amino acid sequence of any one of SEQ ID Nos: 31-39; and
(c) the oligomerization domain of the first fusion protein comprises an amino acid sequence having at least 90% sequence identity to an amino acid sequence of any one of SEQ ID Nos: 17-30.

135. The HBV-Antivirus of claim 4, wherein:
(a) the immune modulating polypeptide comprises an NK cell activating ligand, an immune checkpoint molecule, an inflammatory cytokine, or a proliferation cytokine; and
(b) the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein.

136. The HBV-Antivirus of claim 135, wherein the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21.

137. The HBV-Antivirus of claim 136, wherein the immune modulating polypeptide comprises TNF-α.

138. The HBV-Antivirus of claim 49, wherein:
(a) the second fusion protein comprises an immune modulating polypeptide comprising an NK cell activating ligand, an immune checkpoint molecule, an inflammatory cytokine, or a proliferation cytokine;
(b) the transmembrane polypeptide of the second fusion protein comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein; and (c) the oligomerization domain of the second fusion protein comprises a leucine zipper dimerization domain, a D4 post-fusion trimerization domain of VSV-G protein, a Dengue E protein post-fusion trimerization domain, a foldon trimerization domain, or an influenza neuraminidase stem domain.

139. The HBV-Antivirus of claim 138, wherein the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21.

140. The HBV-Antivirus of claim 139, wherein the immune modulating polypeptide comprises TNF-α.

141. The HBV-Antivirus of claim 4, wherein:
(a) the immune modulating polypeptide comprises an NK cell activating ligand, an immune checkpoint molecule, an inflammatory cytokine, or a proliferation cytokine; and
(b) the transmembrane polypeptide of the second fusion protein comprises an amino acid sequence having at least 90% sequence identity to an amino acid sequence of any one of SEQ ID Nos: 31-39.

142. The HBV-Antivirus of claim 141, wherein the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21.

143. The HBV-Antivirus of claim 142, wherein the immune modulating polypeptide comprises TNF-α.

144. The HBV-Antivirus of claim 49, wherein:
(a) the immune modulating polypeptide comprises an NK cell activating ligand, an immune checkpoint molecule, an inflammatory cytokine, or a proliferation cytokine;
(b) the transmembrane polypeptide of the second fusion protein comprises an amino acid sequence having at least 90% sequence identity to an amino acid sequence of any one of SEQ ID Nos: 31-39; and
(c) the oligomerization domain of the second fusion protein comprises an amino acid sequence having at least 90% sequence identity to an amino acid sequence of any one of SEQ ID Nos: 17-30.

145. The HBV-Antivirus of claim 144, wherein the immune modulating polypeptide comprises m157, NKG2D ligand, CD70, CD40, CTLA4, PD1, OX40, CD28, IL-12, IL-18, TNF-alpha, TNF-beta, IL-7, IL-15, or IL-21.

146. The HBV-Antivirus of claim 145, wherein the immune modulating polypeptide comprises TNF-α.

147. The HBV-Antivirus of claim 1, wherein the antibody of the first fusion protein comprises a CDR-H1 according to SEQ ID NO: 53, a CDR-H2 according to SEQ ID NO: 54, a CDR-H3 according to SEQ ID NO: 55, a CDR-L1 according to SEQ ID NO: 77, a CDR-L2 according to SEQ ID NO: 78, and a CDR-L3 according to SEQ ID NO: 79.

148. The HBV-Antivirus of claim 1, wherein the antibody of the first fusion protein comprises a CDR-H1 according to SEQ ID NO: 56, a CDR-H2 according to SEQ ID NO: 57, a CDR-H3 according to SEQ ID NO: 58, a CDR-L1 according to SEQ ID NO: 80, a CDR-L2 according to SEQ ID NO: 81, and a CDR-L3 according to SEQ ID NO: 82.

149. The HBV-Antivirus of claim 1, wherein the antibody of the first fusion protein comprises a CDR-H1 according to SEQ ID NO: 59, a CDR-H2 according to SEQ ID NO: 60, a CDR-H3 according to SEQ ID NO: 61, a CDR-L1 according to SEQ ID NO: 83, a CDR-L2 according to SEQ ID NO: 84, and a CDR-L3 according to SEQ ID NO: 85.

150. The HBV-Antivirus of claim 1, wherein the antibody of the first fusion protein comprises a CDR-H1 according to SEQ ID NO: 62, a CDR-H2 according to SEQ ID NO: 63, a CDR-H3 according to SEQ ID NO: 64, a CDR-L1 according to SEQ ID NO: 86, a CDR-L2 according to SEQ ID NO: 87, and a CDR-L3 according to SEQ ID NO: 88.

151. The HBV-Antivirus of claim 1, wherein the antibody of the first fusion protein comprises a CDR-H1 according to SEQ ID NO: 65, a CDR-H2 according to SEQ ID NO: 66, a CDR-H3 according to SEQ ID NO: 67, a CDR-L1 according to SEQ ID NO: 89, a CDR-L2 according to SEQ ID NO: 90, and a CDR-L3 according to SEQ ID NO: 91.

152. The HBV-Antivirus of claim 1, wherein the antibody of the first fusion protein comprises a CDR-H1 according to SEQ ID NO: 68, a CDR-H2 according to SEQ ID NO: 69, a CDR-H3 according to SEQ ID NO: 70, a CDR-L1 according to SEQ ID NO: 92, a CDR-L2 according to SEQ ID NO: 93, and a CDR-L3 according to SEQ ID NO: 94.

153. The HBV-Antivirus of claim 1, wherein the antibody of the first fusion protein comprises a CDR-H1 according to SEQ ID NO: 71, a CDR-H2 according to SEQ ID NO: 72, a CDR-H3 according to SEQ ID NO: 73, a CDR-L1 according to SEQ ID NO: 95, a CDR-L2 according to SEQ ID NO: 96, and a CDR-L3 according to SEQ ID NO: 97.

154. The HBV-Antivirus of claim 1, wherein the antibody of the first fusion protein comprises a CDR-H1 according to SEQ ID NO: 74, a CDR-H2 according to SEQ ID NO: 75, a CDR-H3 according to SEQ ID NO: 76, a CDR-L1 according to SEQ ID NO: 98, a CDR-L2 according to SEQ ID NO: 99, and a CDR-L3 according to SEQ ID NO: 100.

155. The HBV-Antivirus of claim 13, wherein the antibody of the second fusion protein comprises a CDR-H1 according to SEQ ID NO: 53, a CDR-H2 according to SEQ ID NO: 54, a CDR-H3 according to SEQ ID NO: 55, a CDR-L1 according to SEQ ID NO: 77, a CDR-L2 according to SEQ ID NO: 78, and a CDR-L3 according to SEQ ID NO: 79.

156. The HBV-Antivirus of claim 13, wherein the antibody of the second fusion protein comprises a CDR-H1 according to SEQ ID NO: 56, a CDR-H2 according to SEQ ID NO: 57, a CDR-H3 according to SEQ ID NO: 58, a CDR-L1 according to SEQ ID NO: 80, a CDR-L2 according to SEQ ID NO: 81, and a CDR-L3 according to SEQ ID NO: 82.

157. The HBV-Antivirus of claim 13, wherein the antibody of the second fusion protein comprises a CDR-H1 according to SEQ ID NO: 59, a CDR-H2 according to SEQ ID NO: 60, a CDR-H3 according to SEQ ID NO: 61, a CDR-L1 according to SEQ ID NO: 83, a CDR-L2 according to SEQ ID NO: 84, and a CDR-L3 according to SEQ ID NO: 85.

158. The HBV-Antivirus of claim 13, wherein the antibody of the second fusion protein comprises a CDR-H1 according to SEQ ID NO: 62, a CDR-H2 according to SEQ ID NO: 63, a CDR-H3 according to SEQ ID NO: 64, a CDR-L1 according to SEQ ID NO: 86, a CDR-L2 according to SEQ ID NO: 87, and a CDR-L3 according to SEQ ID NO: 88.

159. The HBV-Antivirus of claim 13, wherein the antibody of the second fusion protein comprises a CDR-H1 according to SEQ ID NO: 65, a CDR-H2 according to SEQ ID NO: 66, a CDR-H3 according to SEQ ID NO: 67, a CDR-L1 according to SEQ ID NO: 89, a CDR-L2 according to SEQ ID NO: 90, and a CDR-L3 according to SEQ ID NO: 91.

160. The HBV-Antivirus of claim 13, wherein the antibody of the second fusion protein comprises a CDR-H1 according to SEQ ID NO: 68, a CDR-H2 according to SEQ ID NO: 69, a CDR-H3 according to SEQ ID NO: 70, a CDR-L1 according to SEQ ID NO: 92, a CDR-L2 according to SEQ ID NO: 93, and a CDR-L3 according to SEQ ID NO: 94.

161. The HBV-Antivirus of claim 13, wherein the antibody of the second fusion protein comprises a CDR-H1 according to SEQ ID NO: 71, a CDR-H2 according to SEQ ID NO: 72, a CDR-H3 according to SEQ ID NO: 73, a CDR-L1 according to SEQ ID NO: 95, a CDR-L2 according to SEQ ID NO: 96, and a CDR-L3 according to SEQ ID NO: 97.

162. The HBV-Antivirus of claim 13, wherein the antibody of the second fusion protein comprises a CDR-H1 according to SEQ ID NO: 74, a CDR-H2 according to SEQ ID NO: 75, a CDR-H3 according to SEQ ID NO: 76, a CDR-L1 according to SEQ ID NO: 98, a CDR-L2 according to SEQ ID NO: 99, and a CDR-L3 according to SEQ ID NO: 100.

163. A method of treating HBV in a subject in need thereof comprising:
(a) selecting an antibody that binds to a surface protein of an HBV; and
(b) expressing the antibody on a surface of a multivalent particle, wherein the multivalent particle has a binding affinity to the surface protein of the HBV that is higher than the binding affinity of a soluble version of the antibody to the surface protein of the HBV.

164. The method of claim 163, wherein the surface protein of the HBV comprises Large Hepatitis B surface Antigen (L-HBsAg), Medium Hepatitis B surface Antigen (M-HBsAg), or Small Hepatitis B surface Antigen (S-HBsAg).

165. The method of claim 163, comprising administering the multivalent particle to the subject.

166. The method of claim 165, wherein the multivalent particle neutralizes the HBV when the multivalent particle binds to the surface protein of the HBV.

167. The method of claim 163, wherein the antibody is expressed in a fusion protein on the surface of the multivalent particle, wherein the fusion protein comprises a transmembrane polypeptide.

168. The method of claim 167, wherein the transmembrane polypeptide comprises the cytosolic tail of VSV-G, the transmembrane domain of VSV-G, the transmembrane domain of influenza Neuraminidase (NA), the transmembrane domain of influenza Hemagglutinin (HA), the transmembrane domain of Dengue E Protein, the transmembrane domain of GP120 or GP41, or the transmembrane domain of measles virus surface glycoprotein hemagglutinin (H) protein.

169. The method of claim 167, wherein the fusion protein comprises an oligomerization domain.

170. The method of claim 169, wherein the oligomerization domain comprises a leucine zipper dimerization domain, a D4 post-fusion trimerization domain of VSV-G protein, a Dengue E protein post-fusion trimerization domain, a foldon trimerization domain, or an influenza neuraminidase stem domain.

171. The method of claim 163, wherein the antibody comprises an amino acid sequence from at least one complementarity determining region of 2H5, ADRI-2F3, H004, H009, H007, H015, H019, or H020.

172. The method of claim 163, wherein the antibody comprises a single chain variable fragment (scFv), a tandem scFv, a single domain antibody, an Fv, a VH domain, a VL domain, a Fab fragment, a monoclonal antibody, F(ab′), F(ab′)2, single chain antibodies, diabodies, or a scFv-Fc.

173. The method of claim 167, wherein the fusion protein is expressed at a valency of about 10 copies on the surface of the multivalent particle.

174. The method of claim 163, wherein the multivalent particle comprises a viral-like particle.

175. The method of claim 163, wherein the multivalent particle comprises an extracellular vesicle.

176. The method of claim 175, wherein the extracellular vesicle comprises an ectosome.

177. The method of claim 175, wherein the extracellular vesicle comprises an exosome.

178. A method of using a multivalent HBV-Antivirus as a treatment vaccine to induce antiviral immunity against HBV or HDV in a subject infected with HBV or HDV comprising administering to the subject the HBV-Antivirus, wherein the HBV-Antivirus comprises a first fusion protein that comprises a transmembrane polypeptide and an antibody that binds to an HBV surface protein, wherein the first fusion protein is expressed on a surface of the HBV-Antivirus.

179. The method of claim 178, wherein the HBV-Antivirus is administered to the subject through inhalation or intranasal delivery to induce protective immunity against future HBV or HDV infections.

180. A method of using a multivalent HBV-Antivirus inactivated vaccine to induce protective immunity against HBV or HDV in a subject in need thereof comprising administering to the subject the HBV-Antivirus inactivated vaccine, wherein the HBV-Antivirus inactivated vaccine comprises an HBV and the HBV-Antivirus, wherein the HBV-Antivirus comprises a first fusion protein that comprises a transmembrane polypeptide and an antibody that binds to a HBV surface protein, wherein the first fusion protein is expressed on a surface of the HBV-Antivirus.

181. The method of claim 180, wherein the HBV-Antivirus is administered to the subject through intranasal or intramuscular delivery.

182. A composition comprising a multivalent HBV-Antivirus wherein the HBV-Antivirus comprises an enveloped particle that displays at least 10 copies of an antibody on a surface of the HBV-Antivirus, wherein the antibody binds to a surface protein of an HBV.

183. A composition comprising a multi-specific, multivalent HBV-Antivirus wherein the HBV-Antivirus comprises an enveloped particle that displays at least 10 copies of a multi-specific antibody on a surface of the HBV-Antivirus, wherein the multi-specific antibody binds to one or more surface proteins of an HBV.

184. A composition comprising a multi-specific, multivalent HBV-Antivirus wherein the HBV-Antivirus comprises an enveloped particle that displays at least 10 copies of a first antibody on a surface of the HBV-Antivirus and at least 10 copies of a second antibody on the surface of the HBV-Antivirus, wherein the first antibody binds to a surface protein of an HBV and the second antibody binds to a surface protein of the HBV, wherein the second antibody has less than 100% sequence identity to the first antibody.

185. A composition comprising a dual-action, immune-modulating HBV-Antivirus wherein the HBV-Antivirus comprises an enveloped particle that displays at least 10 copies of an antibody on a surface of the HBV-Antivirus and at least 10 copies of an immune modulating polypeptide on the surface of the HBV-Antivirus, wherein the antibody binds to a surface protein of an HBV.


SEQUENCE LISTING (101 sequences)

SEQ ID NO: 1 - protein, 121 aa, synthetic construct
SQVQLQQSGPGLVKPSQTLSLTCGISGDSVSSKSAAWNWIRQSPSRGLEWLGRTYYRSKWHNDYAVSVKSRITINPDTSKNQFSLQLNSVTPEDTAVYYCARGQMGALDVWGQGTTVTVSS

SEQ ID NO: 2 - protein, 110 aa, synthetic construct
QSVLTQPPSASGTPGQRVTISCSGSSSNIGSYYVYWYQQFPGTAPKLLIYGNNQRPSGVPDRFSGSKSGTSASLAITGLQAEDEADYYCQSYDSSLSGVIFGGGTKLTVL

SEQ ID NO: 3 - protein, 123 aa, synthetic construct
EVQVLESGGGLVQPGGSLRLSCAASGFRFSSYAMSWVRQAPGKGLEWVSGISGTGENTYYADSVKGRFTISRDNSKNTLYVQMNSLRAEDTAVYYCAKDAILGSGHPWYFHVWGRGTLVTVSS

SEQ ID NO: 4 - protein, 108 aa, synthetic construct
SYVLTQPPSVSVAPGQTARMTCGGNNIGSESVHWFQQKPGQAPVLVVYDDSDRPSGIPERFSGSNSGNTATLTISRVEAGDEADYYCQVWDSSSDHAVFGGGTQLTVL

SEQ ID NO: 5 - protein, 123 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGYFGSGSLYGIDVWGQGTTVTVSS

SEQ ID NO: 6 - protein, 110 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSNHDHPGVVFGGGTKLTVL

SEQ ID NO: 7 - protein, 123 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKTDIKWGATNYGMDVWGQGTTVTVSS

SEQ ID NO: 8 - protein, 109 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDGTRDHLVVFGGGTKLTVL

SEQ ID NO: 9 - protein, 131 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKEIGGFDFRSGSQRSYYYYGVDVWGQGTTVTVSS

SEQ ID NO: 10 - protein, 110 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPPGYTFGQGTKLEIK

SEQ ID NO: 11 - protein, 121 aa, synthetic construct
QVQLVESGGGVAQPGRSLRLSCAASGFSFSRHGMHWVRQAPGKGLEWVAGIWFDGTNDYYTDSVKGRFTISRDNSRSTLYLDINSLRAEDTAVYYCAREDPHLLIATLDLWGLGTLVTVSS

SEQ ID NO: 12 - protein, 108 aa, synthetic construct
DIQVTQSPSSLSAFVGDRVTITCRASQGLTSFINWYQQKPGRAPKLLISSASSLQRGVPSRFTASGSGTHFTLTISSLQPEDFATYYCQQSYGTPALAFGGGTKVDIK

SEQ ID NO: 13 - protein, 120 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAIYMDEAWAFEIWGQGTMVTVSS

SEQ ID NO: 14 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYTISLFTFGQGTKLEIK

SEQ ID NO: 15 - protein, 121 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARHLYRYGYRNYFDYWGQGTLVTVSS

SEQ ID NO: 16 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 17 - protein, 150 aa, synthetic construct
IGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDI

SEQ ID NO: 18 - protein, 130 aa, synthetic construct
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 19 - protein, 115 aa, synthetic construct
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTT

SEQ ID NO: 20 - protein, 100 aa, synthetic construct
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFING

SEQ ID NO: 21 - protein, 84 aa, synthetic construct
IQADGWMCHASKWVTTCDFRWYGPKYITHSIRSFTPSVEQCKESIEQTKQGTWLNPGFPPQSCGYATVTDAEAVIVQVTPHHVL

SEQ ID NO: 22 - protein, 27 aa, synthetic construct
GYIPEAPRDGQAYVRKDGEWVLLSTFL

SEQ ID NO: 23 - protein, 33 aa, synthetic construct
RMKQLEDKVEELLSKQYHLENEVARLKKLVGER

SEQ ID NO: 24 - protein, 33 aa, synthetic construct
RMKQLEDKVEELLSKNYHLENEVARLKKLVGER

SEQ ID NO: 25 - protein, 40 aa, synthetic construct
MNPNQKIITIGSICLVVGLISLILQIGNIISIWISHSIQT

SEQ ID NO: 26 - protein, 75 aa, synthetic construct
MNPNQKIITIGSICMVTGIVSLMLQIGNMISIWVSHSIHTGNQHQSEPISNTNFLTEKAVASVKLAGNSSLCPIN

SEQ ID NO: 27 - protein, 68 aa, synthetic construct
KLCIEAKISNTTTDSRCPTQGEATLVEEQDTNFVCRRTFVDRGHGNGCGLFGKGSLITCAKFKCVTKL

SEQ ID NO: 28 - protein, 83 aa, synthetic construct
IELLKTEVTNPAVLRKLCIEAKISNTTTDSRCPTQGEATLVEEQDTNFVCRRTFVDRGHGNGCGLFGKGSLITCAKFKCVTKL

SEQ ID NO: 29 - protein, 83 aa, synthetic construct
KLCIEAKISNTTTDSRCPTQGEATLVEEQDTNFVCRRTFVDRGHGNGCGLFGKGSLITCAKFKCVTKLEGKIVQYENLKYSVI

SEQ ID NO: 30 - protein, 59 aa, synthetic construct
EAKISNTTTDSRCPTQGEATLVEEQDTNFVCRRTFVDRGHGNGCGLFGKGSLITCAKFK

SEQ ID NO: 31 - protein, 22 aa, synthetic construct
IASFFFIIGLIIGLFLVLRVGI

SEQ ID NO: 32 - protein, 37 aa, synthetic construct
PIELVEGWFSSWKSSIASFFFIIGLIIGLFLVLRVGI

SEQ ID NO: 33 - protein, 53 aa, synthetic construct
DDESLFFGDTGLSKNPIELVEGWFSSWKSSIASFFFIIGLIIGLFLVLRVGIH

SEQ ID NO: 34 - protein, 81 aa, synthetic construct
GMLDSDLHLSSKAQVFEHPHIQDAASQLPDDESLFFGDTGLSKNPIELVEGWFSSWKSSIASFFFIIGLIIGLFLVLRVGI

SEQ ID NO: 35 - protein, 25 aa, synthetic construct
HLCIKLKHTKKRQIYTDIEMNRLGK

SEQ ID NO: 36 - protein, 23 aa, synthetic construct
IITIGSVCMTIGMANLILQIGNI

SEQ ID NO: 37 - protein, 23 aa, synthetic construct
LAIYSTVASSLVLVVSLGAISFW

SEQ ID NO: 38 - protein, 52 aa, synthetic construct
AYGVLFSGVSWTMKIGIGILLTWLGLNSRSTSLSMTCIAVGMVTLYLGVMVQ

SEQ ID NO: 39 - protein, 21 aa, synthetic construct
FIMIVGGLVGLRIVFAVLSIV

SEQ ID NO: 40 - DNA, 21 bp, synthetic construct
  source 1..21: mol_type = other DNA
ccgtctgtgccttctcatctg

SEQ ID NO: 41 - DNA, 29 bp, synthetic construct
  source 1..29: mol_type = other DNA
agtccaagagtcctcttatgtaagacctt

SEQ ID NO: 42 - DNA, 17 bp, synthetic construct
  source 1..17: mol_type = other DNA
  modified_base 1: mod_base = OTHER; note = 56-carboxyfluorescein-ccgtgtgca-ZEN quencher attached to cytidine
  modified_base 17: mod_base = OTHER; note = cytidine-3' Iowa Black FQ
cttcgcttcacctctgc

SEQ ID NO: 43 - protein, 584 aa, synthetic construct
MEFGLSWVFLVALFRGVQSQVQLVESGGGVAQPGRSLRLSCAASGFSFSRHGMHWVRQAPGKGLEWVAGIWFDGTNDYYTDSVKGRFTISRDNSRSTLYLDINSLRAEDTAVYYCAREDPHLLIATLDLWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSGGGGSGGGGSDIQVTQSPSSLSAFVGDRVTITCRASQGLTSFINWYQQKPGRAPKLLISSASSLQRGVPSRFTASGSGTHFTLTISSLQPEDFATYYCQQSYGTPALAFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 44 - protein, 714 aa, synthetic construct
MEFGLSWVFLVALFRGVQSQVQLVESGGGVAQPGRSLRLSCAASGFSFSRHGMHWVRQAPGKGLEWVAGIWFDGTNDYYTDSVKGRFTISRDNSRSTLYLDINSLRAEDTAVYYCAREDPHLLIATLDLWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSGGGGSGGGGSDIQVTQSPSSLSAFVGDRVTITCRASQGLTSFINWYQQKPGRAPKLLISSASSLQRGVPSRFTASGSGTHFTLTISSLQPEDFATYYCQQSYGTPALAFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECVIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 45 - protein, 370 aa, synthetic construct
MEFGLSWVFLVALFRGVQSSQVQLQQSGPGLVKPSQTLSLTCGISGDSVSSKSAAWNWIRQSPSRGLEWLGRTYYRSKWHNDYAVSVKSRITINPDTSKNQFSLQLNSVTPEDTAVYYCARGQMGALDVWGQGTTVTVSSGGGGSGGGGSGGGGSQSVLTQPPSASGTPGQRVTISCSGSSSNIGSYYVYWYQQFPGTAPKLLIYGNNQRPSGVPDRFSGSKSGTSASLAITGLQAEDEADYYCQSYDSSLSGVIFGGGTKLTVLRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 46 - protein, 505 aa, synthetic construct
MEFGLSWVFLVALFRGVQSEVQVLESGGGLVQPGGSLRLSCAASGFRFSSYAMSWVRQAPGKGLEWVSGISGTGENTYYADSVKGRFTISRDNSKNTLYVQMNSLRAEDTAVYYCAKDAILGSGHPWYFHVWGRGTLVTVSSGGGGSGGGGSGGGGSGGGGSSYVLTQPPSVSVAPGQTARMTCGGNNIGSESVHWFQQKPGQAPVLVVYDDSDRPSGIPERFSGSNSGNTATLTISRVEAGDEADYYCQVWDSSSDHAVFGGGTQLTVLVIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 47 - protein, 980 aa, synthetic construct
MEFGLSWVFLVALFRGVQSSQVQLQQSGPGLVKPSQTLSLTCGISGDSVSSKSAAWNWIRQSPSRGLEWLGRTYYRSKWHNDYAVSVKSRITINPDTSKNQFSLQLNSVTPEDTAVYYCARGQMGALDVWGQGTTVTVSSGGGGSGGGGSGGGGSQSVLTQPPSASGTPGQRVTISCSGSSSNIGSYYVYWYQQFPGTAPKLLIYGNNQRPSGVPDRFSGSKSGTSASLAITGLQAEDEADYYCQSYDSSLSGVIFGGGTKLTVLGGGGSGGGGSGGGGSGGGGSQVQLVESGGGVAQPGRSLRLSCAASGFSFSRHGMHWVRQAPGKGLEWVAGIWFDGTNDYYTDSVKGRFTISRDNSRSTLYLDINSLRAEDTAVYYCAREDPHLLIATLDLWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSGGGGSGGGGSDIQVTQSPSSLSAFVGDRVTITCRASQGLTSFINWYQQKPGRAPKLLISSASSLQRGVPSRFTASGSGTHFTLTISSLQPEDFATYYCQQSYGTPALAFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECVIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 48 - protein, 377 aa, synthetic construct
MEFGLSWVFLVALFRGVQSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGYFGSGSLYGIDVWGQGTTVTVSSGGGGSGGGGSGGGGSGGGGSSYVLTQPPSVSVAPGQTARITCGGNNIGSKSVHWYQQKPGQAPVLVVYDDSDRPSGIPERFSGSNSGNTATLTISRVEAGDEADYYCQVWDSNHDHPGVVFGGGTKLTVLRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 49 - protein, 507 aa, synthetic construct
MEFGLSWVFLVALFRGVQSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGYFGSGSLYGIDVWGQGTTVTVSSGGGGSGGGGSGGGGSGGGGSSYVLTQPPSVSVAPGQTARITCGGNNIGSKSVHWYQQKPGQAPVLVVYDDSDRPSGIPERFSGSNSGNTATLTISRVEAGDEADYYCQVWDSNHDHPGVVFGGGTKLTVLVIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 50 - DNA, 21 bp, synthetic construct
  source 1..21: mol_type = other DNA
ccgtctgtgccttctcatctg

SEQ ID NO: 51 - DNA, 27 bp, synthetic construct
  source 1..27: mol_type = other DNA
agtccaagagtcctcttatacaagacc

SEQ ID NO: 52 - DNA, 11 bp, synthetic construct
  source 1..11: mol_type = other DNA
  modified_base 1: mod_base = OTHER; note = 6-carboxyfluorescein-ccgtgtgca-ZEN quencher attached to cytidine
  modified_base 11: mod_base = OTHER; note = cytidine-Black Hole Quencher 1
cttcgcttcac

SEQ ID NO: 53 - protein, 7 aa, synthetic construct
SKSAAWN

SEQ ID NO: 54 - protein, 18 aa, synthetic construct
RTYYRSKWHNDYAVSVKS

SEQ ID NO: 55 - protein, 8 aa, synthetic construct
GQMGALDV

SEQ ID NO: 56 - protein, 5 aa, synthetic construct
SYAMS

SEQ ID NO: 57 - protein, 17 aa, synthetic construct
GISGTGENTYYADSVKG

SEQ ID NO: 58 - protein, 14 aa, synthetic construct
DAILGSGHPWYFHV

SEQ ID NO: 59 - protein, 5 aa, synthetic construct
SYAMS

SEQ ID NO: 60 - protein, 17 aa, synthetic construct
AISGSGGSTYYADSVKG

SEQ ID NO: 61 - protein, 14 aa, synthetic construct
DGYFGSGSLYGIDV

SEQ ID NO: 62 - protein, 5 aa, synthetic construct
SYGMH

SEQ ID NO: 63 - protein, 17 aa, synthetic construct
VISYDGSNKYYADSVKG

SEQ ID NO: 64 - protein, 14 aa, synthetic construct
TDIKWGATNYGMDV

SEQ ID NO: 65 - protein, 5 aa, synthetic construct
SYGMH

SEQ ID NO: 66 - protein, 17 aa, synthetic construct
VISYDGSNKYYADSVKG

SEQ ID NO: 67 - protein, 22 aa, synthetic construct
EIGGFDFRSGSQRSYYYYGVDV

SEQ ID NO: 68 - protein, 5 aa, synthetic construct
RHGMH

SEQ ID NO: 69 - protein, 17 aa, synthetic construct
GIWFDGTNDYYTDSVKG

SEQ ID NO: 70 - protein, 12 aa, synthetic construct
EDPHLLIATLDL

SEQ ID NO: 71 - protein, 7 aa, synthetic construct
SGDYYWS

SEQ ID NO: 72 - protein, 16 aa, synthetic construct
YIYYSGSTYYNPSLKS

SEQ ID NO: 73 - protein, 10 aa, synthetic construct
YMDEAWAFEI

SEQ ID NO: 74 - protein, 5 aa, synthetic construct
SYYWS

SEQ ID NO: 75 - protein, 16 aa, synthetic construct
YIYYSGSTNYNPSLKS

SEQ ID NO: 76 - protein, 13 aa, synthetic construct
HLYRYGYRNYFDY

SEQ ID NO: 77 - protein, 13 aa, synthetic construct
SGSSSNIGSYYVY

SEQ ID NO: 78 - protein, 7 aa, synthetic construct
GNNQRPS

SEQ ID NO: 79 - protein, 11 aa, synthetic construct
QSYDSSLSGVI

SEQ ID NO: 80 - protein, 11 aa, synthetic construct
GGNNIGSESVH

SEQ ID NO: 81 - protein, 7 aa, synthetic construct
DDSDRPS

SEQ ID NO: 82 - protein, 11 aa, synthetic construct
QVWDSSSDHAV

SEQ ID NO: 83 - protein, 11 aa, synthetic construct
GGNNIGSKSVH

SEQ ID NO: 84 - protein, 7 aa, synthetic construct
DDSDRPS

SEQ ID NO: 85 - protein, 13 aa, synthetic construct
QVWDSNHDHPGVV

SEQ ID NO: 86 - protein, 11 aa, synthetic construct
GGNNIGSKSVH

SEQ ID NO: 87 - protein, 7 aa, synthetic construct
DDSDRPS

SEQ ID NO: 88 - protein, 12 aa, synthetic construct
QVWDGTRDHLVV

SEQ ID NO: 89 - protein, 12 aa, synthetic construct
RASQSVSSSYLA

SEQ ID NO: 90 - protein, 7 aa, synthetic construct
GASSRAT

SEQ ID NO: 91 - protein, 11 aa, synthetic construct
QQYSSSPPGYT

SEQ ID NO: 92 - protein, 11 aa, synthetic construct
RASQGLTSFIN

SEQ ID NO: 93 - protein, 7 aa, synthetic construct
SASSLQR

SEQ ID NO: 94 - protein, 10 aa, synthetic construct
QQSYGTPALA

SEQ ID NO: 95 - protein, 11 aa, synthetic construct
RASQSISSYLN

SEQ ID NO: 96 - protein, 7 aa, synthetic construct
AASSLQS

SEQ ID NO: 97 - protein, 10 aa, synthetic construct
QQSYTISLFT

SEQ ID NO: 98 - protein, 11 aa, synthetic construct
RASQGISSWLA

SEQ ID NO: 99 - protein, 7 aa, synthetic construct
AASSLQS

SEQ ID NO: 100 - protein, 9 aa, synthetic construct
QQANSFPLT

SEQ ID NO: 101 - protein, 6 aa, synthetic construct
HHHHHH